SEQUENCE LISTING (23 sequences)

SEQ ID NO: 1 - DNA, 1531 bp, Bifidobacterium longum
  source 1..1531: mol_type = other DNA; sub_species = subspecies infantis
ttttttgtggagggttcgattctggctcaggatgaacgctggcggcgtgcttaacacatgcaagtcgaacgggatccatcaagcttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattctcggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggtgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggtgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctcctttcta

SEQ ID NO: 2 - DNA, 1332 bp, Bifidobacterium longum
  source 1..1332: mol_type = other DNA; sub_species = subspecies infantis
atgggagcgaagatcttccgcaccatttcccaaaggcatctcatggaacccgtcgaacaagcctcgcacgaggggacgttcgacgtggagatcatgatgaaacccggggaaacgatgcagcgcgtcgtcggcttcggcgcgtccctcacggactcctccgcgtatctgctcaacgagacgctggacgatgagagcagaaaacaagccatgacggacctcttcgacccggaccagggcatcggcctgtcgatgctccgcaatccgatgggcgcaagcgattacgccagggatgtctacagctacgacgacatgccggatgggaaacgggacgactcgatggagcatttctccatcgcgcgagacgaacgcaacgtgatacccctgatccacgaggccaaacggctgaaccccgacctgaaggtcgtgatgagcccatggagcgcccccgcatggatgaagaccaacggaagcatgaaggcgggaagcctcagggaagactgccgggaaacctacgccaggtatttcgtccagtgcttgcgcgcctacgggtcccacggcatcgaagtacatgccgtcacgcctcagaatgagcccctgtacgagcccacccattaccccagcatggcgatgagcgcacaggaggagaccgtcttcgtcagggaccatctcaggacggccatgcgcgaagcgggattcaagacgctgatactcggatacgaccacaattgggacagatgcgactatccgctggcgttgcttgacggcgcggccgaatcgttcgatggaatcgcatggcattggtacgcgggcgacccccaatcgcagagcgtcgtgtccgaacgccaccccgggaaattctcctatgtgaccgaagcgtcgggcggagaatggatccccgggttcgaaccggcgtttagccacctggttggcatgatcatccaggcactgaaccatggggcgaatgcgttcgtcctatggaacatcgctttggacgagcatcgcggaccgacggtgcctggattcggggaatccacatgcagggggctgctgcgcgtcgactccgaacgtcgcaaggcgagcaaggagatcgactactacgggctggcgcatttcagccgtcacattcgtccgggggcccatgtcgtccccaccatcgcaacgggaaacacggacggcgcgcgatgcgtggcggcgctcaacgaagatggaagccgcgccatggttcttctgaacgatggggaatctccgataaccgtcagggcggggtgggaggataacgcggccaacgttacgctgacgcctaaagccgttgctacgatcgtatggaggtga

SEQ ID NO: 3 - DNA, 2394 bp, Bifidobacterium longum
  source 1..2394: mol_type = other DNA; sub_species = subspecies infantis
atgagcgagaacatcagcggccagaagctcccctaccgcaacccggatctgccggttgaagaacgcatcgacgacctgctctcccgcatgaccctggaagaaaaggtcgggcagatgatgcagctcgacgcacgctccggcgacttggacgacttgattgtgaacaggcacgtcggctcgatcctgcacaccagtccggacgacctgcctcgagcggccgagaccgtcaacacgaagacacgtctcggcattcccctgatcatcggcgacgactgcatccacggatactccttctggcccggcgcgacgatcttccccgaacaattgggcatggcgacaagctgggatccggacaagattgaatcgatgggccgggcaactgccgaagaggtctctgccacgggtgtgcattggaccttctccccggtgctgtgcatcgcccgcgatacccgctggggacgcgtgggggagaccttcggcgaagatccgacgctgatcggcgaactctccagcgccatcgtcaaaggctaccaaggcggagccaacgccacggaaccattgggccgagacgctatcctcgcctgcgccaaacacttcgcaggctattccgaaactcagggcggccgcgacgcatccgaggctgaactcagccatcgcaagcttgagtcatggttccttccacccttcgaacgcgtagccaaagaaggctgcggcacattcatgctcggatacgaatccattgacggcgtcccggtcaccttcaaccagtggctcctgaaagaaaagctacgtggctcctggaactaccagggcacactcatcaccgactgggacaacgtgggccgctccgtctgggaacagcacgtcaagccagactatacgcatgcggcggcggacgccgtcaagtcaggcaatgacctgatcatgaccacgccacagttctacgaaggcgcgatcgaggccgtggacacaggcctgctcgacgaatcactgatcgatgccgcagtgtcacgcatcctggcgttgaagttccgtctgggactcttcgaagacccgcgtctaccggatcccgaacgtatcaaggtcatcatgggttcggcggaacatcgacaggcgaacctcgagctgacccgagaatccgtggtcctgctgcgcaacgacggctcactgcccctgaacatcgaaaacaacagctccgctgccaacacaatcaaacacatcgccgtcatcggaccgctggccgacgacgcgcaaacccaactcggcgactgggccggcaactccggacaagtcaactggatgcccgacggccatccacgcgaagcgatcaccaccgtactcgacggcatcaaagccatcgcaaaaaagaccggcatcgaagtcgcctacatgcgcggcgcgaacatcgtgaacatcgtgcccgaccccgagggcgaactctatcccgacggacaaccccgacccaaggtagccgtccccgcacccgccgaccagacaatgatcgacgaagccgtagccacagcccgccaatccgacgcaatcgtgaccgtcgtcggcgacgtagtgcaactgatcggcgaaggatgttccaccgccacactcgaactactcggcggacaaaacgcactactcgatgcgctcgccgatgtgtcgagggaaaccgggaagccattggtcgtggtgttggtcagctccaagccgcaggtgcttccaccgtcggtggttggtcccgttcctgtcgtcgccagtctgcgtcccgatggcacgcctatccactgccgtgagagcgagggcggggcaagcgccctgctgtgggctccttcaccgggcatggagggcggtcgcgccatagccgagatcattttcggtctgacggatccatccgggcgtctgccgatcactttcccgcgccatgcgggtcagctgccggtctattacaaccagatcagaggccagcatggcgaccggtacgccgatctgacgcaggatccggcgttcgcgttcggcgagggactcggatacaccacgttcgagtacgatgatccgacgattagcaatgtcccgccttccggcgtcttcaccgcagccgacaccatacatgccgaagtcaccgtgaccaacaccgggaagcgttacggcgtcgaggtcgtgcaggcatatgtcggggacctcgtgacatccaccagctgggccgatcgggaactcaaggcgtttcgacgcgtcgaactgcagccgggcgaaagcaggatcgtctctttcgacattcccgtcgcagactgcaccatcgtcgatccggacgccaaccgcatcgtcgaacccggcgaattcgaactgctcatcggccactccagccgacgagaggacttgaaacgcgccatcttcgccgtagcctga

SEQ ID NO: 4 - DNA, 1761 bp, Bifidobacterium longum
  source 1..1761: mol_type = other DNA; sub_species = subspecies infantis
atggggacgacaggacacagcgacgtgccaaggcccgaacatcccgaaccgcagtggcaaagagacaattggcgctgtctcaacggcacatgggactttcaactcgatctcaactgcagcggacaggacaggggatggtacaggaagcatgacttcgaccagagcatagtcgtgcctttttgtccggaaagcaaactgtcgggtatcgaatacaaggacttcattcactgctgttggtacaggcgatcaatcgaactcagcgaaaaagaactcgccggtatagtcagacttcacttcggcgccgttgattatgaaacgaccgtctacctgaacggggaacgacttggagtacatcgcggaggatactccccattcagtttcgacatccagagaatagcgcacgtgggtgtgaacgaattggtcgtatgcgtgaaggacgaactgcgatctggacgacagccaaggggcaagcaatccgaacgctacgactcatacggctgctgctacacccgcactacaggcatatggcagaccgtatggctggaattcatgccgcagacccacgtcgtttcggcccgctttcatccgaatctatcgcgagacagtgtcggcatcgacctgacggtcaatggccgaacccggcaaagatcacagctggcgaacataaccatcagcttcgaggggaaccaggtgggctctgcgtccatcgccatgggcagtgacagatgctccctggaggtgccacttgagaaaactgtcccatggaccgtcggacaaggcaacctatacgatgtgcgaatcagctacggcgaagacgtcgtctgcagttacttcggcatgcgatccgtggacatcgacggtaaccgagtgctcatcaacggcgaacccgtattccaacgcctcgtcctggaccagggctactacccggaaggcatctatacggccaaggacctggaagaactgtcccgcgacatcgacctgtcgatggcggcgggattcaacggtgcgaggctccaccaaaaggcgttcgaaccaaggttcctgtacgaatgcgaccgcaaaggatacatcgtatggggggaaatggccaactggggtctcgacatcaccacggcatcaggattgaccgccttccttcccgaatggctggaactcctggaaagggacatcaatcatccatcgatcgtcggctggtgcccgttcaacgagacctgggacgtcgacggaacgaaacaagacgatgatgtgctccggcagacatatgcggtcaccaaagccatcgacccatcgcgtccgtgcatcgacaccagcggtatgttccacaccgtcaccgacatctacgacgtgcatgattacgagcaggatcccatacgcttcgcaggcaactacgccggactggatccctcggacggctcgggctacgttgacccattccccgaacgccagcgttaccaaggatgcaaacccttctttgtcagcgaatacggtggcatcaagatagcaagccgcgcacgcgtggacggaggagagtcgtgggggtacggggaggccgctgcgggcgagtcggagttcgtggaacgctaccacggtctaacgaaggctctgttggacaatccggccattttcggcttctgctacacgcagctatatgacgtcgagcaggaatgcaacggactatacacatacgacagaaagcccaaggtcgacatcgcagcggtgcgtgacatcaacatggaacgagccgcaatcgaaaagagttga

SEQ ID NO: 5 - DNA, 984 bp, Bifidobacterium longum
  source 1..984: mol_type = other DNA; sub_species = subspecies infantis
atgggcgctgcagaaaaaagcaggagaatctccacccggagggaaagggcacgatccgaaggcaatgtccaccgcgtcagatcatggttcgtccagttcgggagacaatggcagctgcaggccatgatcttgcctggcatcatcttcatgatcgtcttcaacttcatccccatctacggtttggtcctggcattcaagcaatacacggtggtggacaccatcgaggacgcgccttgggtcggctggcagaatttcgaaactatcctgaaggaccagtatttctggcaatccgtggtcaacaccctgggcatcagcttcatcaaactgacgctgggattcctcctgcccatcgtgttggcggtcatgatctacgaaatcccatggacaccattcaagaagatagtacagacgctgacctatctccctcattttttttcatggatcatcctcggaggcatgctgatcaactggctgtcaaccaacggccttcttaatgaaatccttcaattcctaggcttcaacgcagactcgaactatctgctcgacgccgacaaatactggtggatcgcttccctttcggacgtgtggaaggaagccggatggggaacgatcctgtatctggcgaccatggcgggaatcgacaacgcgttgtacgaggccgcggagatcgacggagcatccaagatccaacgcatatggaacatcaccatcccgggcatccgccagatcatcatcctgaacctcatcctgacggtgtcgggactactgggatccaacctggaccagacgctcgtcctgatgaattcacagaaccagcccaaggctgaggtcatcaactcctacgtctaccgggtgggcctgtccgaaggagacttctcctatgcgacggccgtgggcctcggcgtctcgatcgtgtccacgatcctgctggtagccgtcaacctggtgacgaagaaaatcaacgacaatcagtccgtgctctga

SEQ ID NO: 6 - DNA, 933 bp, Bifidobacterium longum
  source 1..933: mol_type = other DNA; sub_species = subspecies infantis
atgaatgcaaagaccgtgaaaggccttcccgaagggaaacctagtttcgttgcccgatcccagcgcgtgaggcataattcgatcttcttcgacgtagcgaacgccgccctgctgctgttgttcaccattatcatcgtggttccggtgtggaacatcgtcatctcgtcgttcgccacttcggaatccctgtcagaaaacgacttcatcttctggccgaaagcgttgtccctcgacaactacgcggcagtgttctccgacgatggattgtggcagtcgctgttcatctctgtctccagaaccgcggtgggcgtgatcgcgcacacgatgttctgcgccgtgttcgcctatccgttgagcaaggcatatctgaagggaaggtcagtgtactccgcgatgggcatcatcacgatgttcttctccggcggtatgatcccgacctatctgctcatcaagtcgttgggccttctggactccttctgggtctatatcatccccggactgttcagctactacgacgtgatcatcctgatgaacttcttccgacaggtgccgaattcgttgatcgactccgcaaggatcgatggagcgggggagctgcgggtcttctcctcgatcatcctcccgctctccaagccggcgttggcgaccatcgctctcttccacggagttgcccaatggaacgacttcatgaccaccaagctgtacatcaacaacgaggcgttgtatccgctgcagatgaggctctatgagatcatcgtccgttcgcaggcctcggcgcaaatgggcaccgtgggatcgctgatggtcgacaccacgacaagaggaatgcgtctggcgacgatcatcatcaccatcgcgccgatcctcatactctacccgttcgtccaaaggtacttcgtcagcggcacgatgcttggcgcggtgaaaggatga

SEQ ID NO: 7 - DNA, 1626 bp, Bifidobacterium longum
  source 1..1626: mol_type = other DNA; sub_species = subspecies infantis
atgaagtacaaaggagtcttcactaaaatggtcgcggtggctggagcggcctccatgctggtgttggccggatgcggcggcagttcgtcggccaagacggacgacgctacggcgaacttgccagatcccagctacgaagtgtccgcggacactccggcatggaagctggacaagactaaggacaccaagctcacctggtacgtcaacgccgattggtggaacaaatcctggggcgaggatctcgtgaccaagcagatcaaggaagacctgaacctcgacgtcgagttcatcacaggtgataacaccaagctcaacacattcttcgccagcggcgacatgccggacctcatcacgatcttcgactccagctcccaggtggcgcagacggccaacaaatgggctttgccactgcaggatctcgccaagaagtacgatccgtacttcaccaaggtggctcagaagcagacgttggactggtacaagctcaaggacggcaagacctacggctacccctcgtactcgaacacgtcccgggactatgaggacggaaccattcctcccacggacgcgttcatcatccgcaaggacgtgctggacgcgatcggagatgtcgacttcaagaccccggagggattcgtcagcgcgatgaagaccatcaaggagaagttccccgatctcgtcccgttcggcttcaactcgttcgatgcatccaacagctccctcgacgccacattgcagaacatgctcggcgttccgatcatcaaagacggcaagttctacgagaggaacctagacgaggactacgtgacctggctcaaggcgttgaggcaggtacaccaggatagaaacatctccgacgacagcttcgccgacgacgaggaaacgcttaaggaaaaggtgagctcgggcaagtacgccaccatgctgctccaggcgttcccccagaaaggcaccgaacttcaaacctggatgtcctcgaaccccggcaaggaatacgtcgccgtcgacgccatggagtccacggaaggcaacgagccgaccctgtcccaggccggactgagcggatggaccatcacctacatctccaagaagaccaagaacccggcgaaggccatgcagctgtacacgtatctgctcgacgaccccggccagtacctggtgaacttcggcatcgagggaaagacctacaccaagaactccgacggcaccgtctcatggacggacgaggccaacgaagtgcgcaccaacgaaccggagaagttccagaaggaatggcgaatcggcgagttcgtcctgttcagccacgaccggtacaaggccctgaacaaggattcatacgtccaagcgatccgtcagatgcaggactggggacgagactacctcaagccccagttcgagatcgagaacatcagccccgacgcgaacactcctcagtctcggtccctgtccgcgatcaacacgaactggtcgacgaccctggtgtcgatgatccgcgccgaatccgaaaagaaattcgacgaactgctgaccacgtacgagaagttcgagaaggacaacggcatcgaccagatcaacaagatccgcgacgagaagatcgccgcgaacatgaagagactgggcgagtga

SEQ ID NO: 8 - DNA, 675 bp, Bifidobacterium longum
  source 1..675: mol_type = other DNA; sub_species = subspecies infantis
atgaacaacgagaagaaaaccacgccacggcaccgacccgagaccgaggagcgacgccaaagcatcatcgaggccgccgccgacgtgttcggcagcaaaggcacggcgaacggaacgctgcaggaagtggccgacagggtaggaatgaccagggccggggtcctgcattacttcggatcaaaacgaggactgctggaagcggtgctcaggaaccgggactccagcgaagtcgccgaattcgaccaggggcatctgcccaggggggcgcaggcgttccgccacttggtggagaccgcagagttgaacgcgagccgtcccaacgtggtgcgaaccttcgtcaccctgtcggccgaatcagtgacggagaacaacccgggaaacacatacttccacgaacggtacgacggactacgcaacgaactcaaagagcgaatgcaagacatggccgacgaaatgggacgggacctcgacgaggaaaaggccgaagcggcctgcgcgtccatcatcgcggtcatggacggcctgcaattgcaatggctcattgataaagaccacatcgacatgcccagcaccgtggacttcgcgataaaagcgctgatcgcggtggtgttcgatcacaggcaaaccgaaatgggccacgagccgcaaaaagacgaaaggaacgaacaagtatga

SEQ ID NO: 9 - DNA, 1659 bp, Bifidobacterium longum
  source 1..1659: mol_type = other DNA; sub_species = subspecies infantis
atgcattgctcacaaggtagtgaagggcgctcgtccatcggacgcaagctcgccgccgtcgcggcgggtatggtcaccaccgccatgatggtcggatgttcgctgaccgtgggaacggttgcgaatgcacaggaggaggattcccccgttgtggagtctcaggagggtaacggcagcaagcatttcatggtgtactaccgtgcatggcgtgatgtgactatgaaaggcgtgaacaccgatttgccggacgacaattggatctccatgtacgatattccatacggtatcgatgtcgtcaatgtgttcagttatgttccgagtggtcaggaagcggcggcccagccgttctatgacaagctcaagagcgattacgcgccgtatctgcatgcccgtggcatcaaactcgtacgaggtctggattacggcggtgttatggttaacggattcaaaacgtggattgcccagcagggcaagagcgtcgatgatgcgaccgaatcggattatggcgcgtatgccgaccatgtcatcgaaacatacatgacctccgttggccttgacggccttgacattgacatggagacccacccggatgccgcgcaggtggcgatttccgatcaggtcatcacagccctcgccaagcgtatcgggccgaagtcggataatccggaaggtacgatgttcctgtacgacaccaatggcagctacacggctccgttcgcgaacgtcgccgattgcttcgattatgttgcctaccagcagtacggttccgattcgaaccggacggccaaggccgctgccacctacgaacagtttatcgactcgaccaagttcgttcccggcctgaccttccccgaagagggtgacatgaataaccggtggaacgacgcgaccgaaccatacctggacagtcatttctatgatgtggcctcatactcgtacgaccacaatctcggaggcatgttcgtctacgcgctcgaccgcgacggacgcacctattccgacgatgatttggcacacatcaaaccgtcgaatctgatttggacgaagacagccatcgcacaaagccaaggcatgagtctggaaaacgccaagcaggctgccaaccacttccttgaccgtatgtcctataccaaggatgttccagccgaaacgcgccaaaccgtggcagcggccactaacctgtatgaggtgaacaaggcggtgctcggcgcggattggaacgacggatacagcaacacatatgatccgacgcttgaactgtccctgacgtccatcgacaccacggcgcttaccaatgccatagacaaggcggatgcactgctggccgacggcgtaaccgataccgatatgttgaccgaggccagaagcgcggtggtcacgggtctggccggcaaagcgtacaccgaagccgacgttgcctcttggacggcatcgctcaacactgcgatcacagccgcgatggccggcaattccaagccgcagaagcccggagcagacgaaacgaacaagcccggtccgggtcaagaatcggtcgacaggccaaaggtcacaaccggacgtctggcgagcaccggttccgacgtcgctgttgtgctgtcggtcgccacgatgatggtgcttgccggtatcagcgtattcgccgtccgccgccgcggataa

SEQ ID NO: 10 - DNA, 2505 bp, Bifidobacterium longum
  source 1..2505: mol_type = other DNA; sub_species = subspecies infantis
ttgggtattctgaactggaacccggccgcggataaggactcggaacacctgtgctcccgtgtaccgttgcagaccagcatcgccaccaacgccgcgacgcagaaggacccgaacctgccggccgatacggagatgttcaaccttgctggcgactacggcaatgcgttcttcgagtcgttccatgataacaatgtattctcgcaatacctgttcaactactggcagtacacggattattacggcacctggcacggccggccgaccgcgaatgtgccgaagaatttgtacgatgagaaggcccagtccgactggacccagaaatggtttgagttcggcacgttgaacctgctgaacgccgcgcacaaaaacggtgcgaagtcgattgcgacgattttctactcgggcaacgatcgaggtgaacagacctataaggatttgctgcaaggcaaacgtgccgatggcacctatccggtagccgacaagctggtgaaaatcgccaagtactatggctttgacggttacctcgtcaatcaggaattcaacgtaagctccgatgatgtttcagcctatcgggatttcatgaagcagatcgtcgatcaggcatttgcatccagtggtatgactcgacagaatatcctgatggtggtgtcagctaccagaacatgtttaacggtgccaattctccatgggggcaggatgagatcaagggcaaaatctctgattccatcttcctgaatgctgaaggattcggccgattacgccaagagccttggcattgatccaaagtatgcggtgttcgcaggcattgaagcgggtcagaaaaagtttggcaacatcagcaataatgcggattatatgaacgtcaatctcgacgctgacggcaagccctatgtgagtcttgccacgctcggtactgacttcgtctcccatgagcggggcgatgacaagaaggtctatccgaagtatcagaacgaagtattcgaccgtgaacgccgcctgtggaccggttcgtccaccggcgagaagggtacgacggatatctccgattcgtatattgatgatggtacgagcggcgacggctggaagggtttcgcttcgcagatcgtcgagcgctccgtcatcggtggtccggtcttctccacgtcattcaacaccggtcatggtctcgaatggcgtgacggcggcgaacagaccagcggtcagcagtggagtaacatcaacctgcaagacattctgccgacctggcagtggtggattgatgccgactccgacccgctgcaggtcgacttcgactacggcaaggactatgaggcggcacctcgcttcagctacaccaaggttggcggatacgaaggcggcgattcactggtactgagcggcaaactctccagcgacaacactgttcgtctttacaagaccgacttgagcgtcgctgccggttccaaggtcgaactgacctacaacaagctcaacggtgacgactccaagctgcaacttggcctcatcttcgccgacgacaccaaggccgtagtgccggtggacgtggcggatggcacggcaagcaacggctggaagaccgcaagcgtggatttgtcccagtacgcgggcaagaagatcgctaccctgggcctgattatcaaggctggagcgaacccgatcgacaactaccaagtgaacatcggcaagatcaccgtcactgacggtgctgcctacacgcccgctgctccgaccggcttcaaggtcgagcgcgcctacgccgactccggcgaactgaccgtcaagtgggatctcgccgattaccggcatcatccgcggtcgcaccacgccggagtcgtacaccaagtccatccccgctggccatgagtgcgcgttcgccgcccgactcaaccggatgccaagaagtccacgctgaccgccgttgacggctcggccgtggccgacggcaaggctacacgtacggttcaggcaacagtcaagcattcatttggcaatccgctgaccaacgccgaagtggcgttcacgctgcctgagggcgtcacggccgctgatggcaacaccaccgtcaaaaccgatgccgctggtgtggcttcgctgaacgtcgtgtccacgaaggtcggcaagtacacggtgaaggccgcactgggcagcaaggccattggcgagggcgtcaccatcgaattcaccaaggtcgtggccgataagtccggcctgcagaagtcgattgatgcagccaaggccctcaaggaatccggctacacaccgcagtcttggaaggccgttgccgatgcgttgaagaacgccgaagcaattgcctcaaaggacgacgccacgccggatgagatcgccaaggccaaggacatgctggatgcagcggtcaaagctctggtcgcggcgaccccgaacaagcccagccagccggacaaaccgaataagcccggcgacaacaagcctggccagaacaacaacaagaacaatgatagaatggcgcatcggactcttcgttga

SEQ ID NO: 11 - DNA, 1281 bp, Bifidobacterium longum
  source 1..1281: mol_type = other DNA; sub_species = subspecies infantis
atgaggaatatcatgaccagaacactggcatttgtcggtgccgtcgcaatgcttggcagtgtcgtcgcctgcggctccacgaacagctctgacagtggtgatgagaacaccggcgaaatcaccttctggaccaagaacctgaagaccggctacaacgactacttcgtgaacctggtcaaagagttcgagaagcagaaccccggcgccaaggtgaagtgggtcgatcagcccggtgaaagctatgcgaatacagtttcgacagccgcggcatcaggaacgttgcccgatgtgctcgacggtatgccgagcgatgcattcgccttggctgaagctggtgccatcgataacgtctctaaggaatacccgaatatcgaaaaggagtatgctcccggtccgtggtctggcatggttttcaagtctccaactatcgaagaaggtgcttatggatatccgtggtataccactagtagtgccatcttctataacaaaggcattctggcagaatgcggtgtcgataccacgaatctgcctgactcatatgacgagtatttcgctgcagctgatacctttgcacaaaattgcaaggggaagtacagcttcgatgccaacttgccgaatatcggtggtttctatgcctatgccggtaagcaacttaatgatgaccacaccaagtggctcttcaataacaagaagagtgtcgagctactccagaagtacgccgatatgtataaaaatggtgcgctgagtgaagaagcggtttctgccaacggaactcaatcatctgatttgttcaaacagggtgccgtggccactcgtagtggttatgtgtatgatctccaggacatcaaagagaacgcccctgaggtctataagaatcttgctttcgttcctggtttcggtcatgcaagtatcactcctgagatgttgatggtatcctctacttctaagaagaaggctttggcacataagttcgctcagttcgtaactaacaataagaaccaggtggagtttgggaaaacagccaacgtcttcccggccacagctgagggcgttgatgatccctactttacgaatcctggtgctgatgccgatgatcagcgtcaattgattgcgacattgagtaagaacatcaaggatggcttctgtggctatcccgaattcacccctgctgatagcacgacatttaccgagcaagtcagcttggcggcttctggcaaggtcagtcctaaggaagcgctcgatactgcggtgaatgatgccaatacgaggctgcaagactga

SEQ ID NO: 12 - DNA, 978 bp, Bifidobacterium longum
  source 1..978: mol_type = other DNA; sub_species = subspecies infantis
atgactcaggcagcttgggatagtgtccaggttgccggtcggaaggggaagcgtcgtaaggcttccccttccgatgatcaatcggaccgcatgtacaagaacacgccattgctcacccgtctgagcccatggctgttcatctggcctccagtcgtgttggtgattctgttttcaatcattccgttcatcaacactattcgactgtctttcacggattcgacattgctgcgtcccggtaagttcgtcgggctggagcaatatatcagggtctttcatgatccgcgagtcggcaccgcattgcttaattccacggtatacgccctggtcgtggcaccactgatgatgattcttccattaatcatcgcagtgctggtttccggcaaaggacgattggtggggctgttccgcacggccttctatatccctgtggtcgctggttccatcatcacgggtctgatttggaccaacctgttgtctgcccgtggcctggtcaattcagtgttcaatacgctgcattggattcagcagccgattcctttcctcaccgatcgctggctgttgctgttcagctgcatggccgtgacgctgtggaccggcatcggttccaacatggtcatctacctggcgtcgctggccaatatcagtccttcgctctacgaggcggctgatatcgacggcgctggcgcggtacgaaagttcaccagtatcaccatgcctggcgtgcgttcgacgatggtcatgatcctgctgctgtcgacgatttcggccttccgcgtattcaacgaggtttacctgttcacgaatggcaccggcggcattggcggccaggaccgtacgatgagcatgctcatcatgcaggagggcaccggtctgacggcgcgcaccggttatgccagcgccatcagcataatcatgttcttcttgctgggatccgccattctgttgcaaatcgtcgtacaaaagcgaatggagaagtga

SEQ ID NO: 13 - DNA, 846 bp, Bifidobacterium longum
  source 1..846: mol_type = other DNA; sub_species = subspecies infantis
atggcaaaaaacgtgacgagaaaacccatccgcgtgggaaccgtcgtcaagtatgtcgcattgttcattctgctgattatcttcatcgggccgatcatctggcagatttccctggcgttcaaaggacgcggcgacaacatttatgccgttccgccgtatttctggccaagggacttcacctgggacaacttctacagggtattcaccgacattccgattctgacctacctgaagaactccctgatcgtgaccacggtcaatgtgtgtgccaacctgattaccgcgacgatggctggattcgcgttgacccgtgtacgttttcctggtaagagactggtgacgttggtcatcttcggatgcatgctgattcctggtgaaaccgttttggtctcgcagttcttggtcatccgcgcattaggattgcagaacaatctcttcggtgtgattctgcctggcctctgccaggccatgaacatcgtactcatgatcaatgctttctctggcatcccgagggaacttgaagaggccgcagaagtggacggcgccaatatttggcgacggtattggaacgtttgtctgccgcaggtgaagggaacttgcgcggtcattgcgatcttcaccttcgttgcggcgtggaacgacttcatgtggccgattatcgtgctcaccgatgagtcgaaatacactctcaccgtcggtttgaacaggttgcgcggcaccttcttcagcgatcctcgactgattgcagcaggcgccgtagtagccctcatcccgattgtgattttctatctcatcttccagcgctacttcttccagggcgtggaagaaggcggaaccaagggctga

SEQ ID NO: 14 - DNA, 3114 bp, Bifidobacterium longum
  source 1..3114: mol_type = other DNA; sub_species = subspecies infantis
atgtatctcaagattgatcgtgagcttgggcgatgcgagcgcgtgatgcgccagcgcatcacgccacatatccaccggccggtcattcattgcgaagtgagagccttcgacaatccgggagagcctgagccaagcgatgccttcctggcaaaggtgcgggagaatgaagtcgagtttcatgacttcccgctgccgcaggaatggggaaccacatgggggacgacatggtttgagctgactggtgtcattcctcaggatgctttgcatggaaagttcgacttggaacttatggtggaccttggttggcatccggacctcgtcggcatgcacatcgaggggctggtctaccgtcccgacggtagtgccatcaaagccgtgcaccccaggaataactgggttcctctggtcaggggtggccagatagatgatgcgattgcgtccgatggttcgttcacgctgtatcttgaggcggcatcgaatccaatcattctaggtgttccctcgttcgccaagactgagctgggcgccgccgccaccggcaaacgggatcagtcctacatcttccgttcggtggatgtgtgtgtctatgatcggcagcttcaggcgctgtgggctgatctggacgttgtcagccaattggtcaccgagcttccggattcaagtcagtggcgttggaagttggcgaaggcgttgcagcgttcgatgaatatctatgacgagcgagacttgtcgactgttcctgcggcccgcaaagccctggctgacgtgctcgccacacccggtgagaccgttcgcctgcggcatacggcagtcggtcatgcgcacattgactcagcctggctgtggcctgtgcgtgagaccaagcgcaaggttgcccgcaccgttgccaacacattggcgctgatggatcaggatcccgaatatgtttatgcgatgagttctgcccagcagtatgtctggctggaacaggaacatcctgatctctttgagcgggtcaaggctcgtgtcgcagaaggacggttcattcctgtaggcggcatgtgggttgagtcggatggcatgctgccgtccggagaatccttgattcgtcagatttcgtacggtcagcggtatttccgtgatcgcttcggtgtggctccgaagggcatctggctgccggatagctttggctacaacggtcagttcccgcaaatcgcccgtcgtagtggctacgaatggttcctcactcagaagatttcatggagcgacactacgcgtttcccgcatcattccttcatgtgggaaggcatcgatggatcgcgtattttcacgcatttcccgccctccgacaactacggttcccaggtcactgctaaggaactgaagtactccgagtcgaacttcaaagacaaggacttgtcggacaaggccataatgttgtacggctatggagacggtggaggtggtgccacccgcgagatgatggagcgtgtccaccgcttcgagaaccttgagggcattgcctccacgcatatgggttctccggagacctttttcgctgagacgcgtcgggagatggaatgtgaggcaggtgacgagttcccggtgtggaagggcgaactttacctggaactacatcgagctactctgaccagtcaacaggacatgaagcgcggttgccgtggggaggaatccctactgcgcgtcgctgaataccttggcacgatggccatgcttatagttcccggatatgtttacccgaagcaagagctcgatcgtatctggacaacactgttgcttaaccagttccatgatatccttcccggttccgccatcgcctgggtgcaccgtgtggcccgtgaggactatgcccgcgattccgcccgtttgcgtgaaattatcaacgaggcggccggagcactgcgtggtgttgctccgaatgccccggtcatcgctgatgctgtcattccttccttctcgagttccggccctgattggcgtccagtgcccgccgtcgcccggggcgaggcggtagccgttgagaatgagtcggatggttcggtgacgctgaagaatgaggatttgacggttcggattgcccaagacggcaccgttgtgtctgcgtttgaccgtcgctctggccgtgagctcatacctgttggtactgccatgggacgctacgaggtgctgcgtgacgaaccggtgatgtgggacgcctgggatatcgagcgcgatgcattgatgtgtgctgagccagtcaccgattcacatatcatcgatacgagcaccgatgccgacggtgtggcgcgtgtcactgttgaagcacattatggagacaccgtttttgaaactgtgattgcgctggggccggggagcaatcagcttgatttcgctgcgcatgtcgactggcaaaccaaggagcgtttcctcaaagtggacatgccgctcgccattgtggcggacagggcccaatacgaatgccagtatggcatgattagccgtccggtggtcaagaacagtcctggtgacgaagcgcagttcgagaattgttcgcatcgcttcgtgcgcatcgctgacggtgtgtatggtgcctcggtggtcaacgcgacaacctatggttccgacgcctatccactcaacggcgataagctgggggccacgggcaccatgttgcgactgtccttgcttgaaggaccggagtatcccgatcctgatgaggatatgggagagcatgacttccattgggcgcttgctctcggtagcacgcctgcggacaccgttgccgcggcagcacgtattgtcgctccgtcgattgagaacatgcccgttgtggctgcgccggtcaatctggagacgctcgatggttctctcgccattgattggattaagccggcggatgacggatctggtgatgtgattatccgcatctatgaattcgcgggatcgattgccttcgcagtactgcatgcaggcaagtcgctcgaaggcgccacggtccgtgagaccgatgcgctcgaaggtgacgaattgcctgaaggcgagccaaaggcattgcgagacgcaggccccttggaaggcgcgcaacttcgtttcgggccgttccaattcgcgaccctacgagtgcaccgttcctag

SEQ ID NO: 15 - DNA, 1245 bp, Bifidobacterium longum
  source 1..1245: mol_type = other DNA; sub_species = subspecies infantis
atgaaattcggagcgaactacaccccgacccacgattggttccacacgtggctcaatcctgattgggattccgtccatcgggatttggaacagattgcatccatcggcatggatcatgtgcgtctgtttccgctatggccgattctgcagcccaaccgcactctaatcaaccacaagggtcttgaggacgttcgccggatggttgaaatcgccggggaatatggtcttgaggcctatgtcgatgttatccagggacatctgtcgagcttcgacttcatgccttcatgggtggtcacatggcatggcaagaatatgttcactgaccctgatactgttcaaggccaggctgatcttgttgcggccctctatgatgagttggaggacgtgcccactttccgtggcctgaacctgggcaatgaatgcaatcaattcctcggtacccgtcatgcgttgcatatgaccgccacgcctggtgaagtcacgcattggctcgaatcgctgattgctcccgtcaaggaccgcgctgcaaaacaggggcgcgtgctcacgcactgcgaggacgatgccgtatggtatcttgaccgacattctttcaatcctactcacgccgcccgtatcggggacatcaccacgattcattcctgggtgttcaatggggtggctgatcggttcggtgcgtccagcgaacagtgctatcgccacgctcagtatctgaccgagcttgccaaggcctttgcgattgaccctgaacgtaaggtctgggttcaggagattggtgcgcctggcaacgtgttggagcaaggcaatgaggtcgaattcgccgagcagtcggtcaggcgaattctggaatgctccgacgtctatggcattacctggtggtgttctcatgatgtcagctcttcactggctgattttccggtctttgagcatcagcttggtctttttaacgagcatggcgagctcaaggatattggtcgtgtcgtccgcgatgcaattgcatcctatgacgggcatgagcctgaccgcccctcggcggccatagttctgcccgacgatgtggtgagcttggactcgaccgttcgttcctcatgctcgcccaatggcgcagtgttcgactattggatgcagcaggctattgccggcaatcatctagggttcgtatgccagtccatcgccagggactcggacgcgcttgccctgcgtggcatccatgaatgcgtgccgtttgaacaggccttgtcctga

SEQ ID NO: 16 - DNA, 2004 bp, Bifidobacterium longum
  source 1..2004: mol_type = other DNA; sub_species = subspecies infantis
atggctgcagcgaaccgttggattgcattggatggtggccgtgaggcaactctcattccctggccttcgcttgttgagcaaggccggggagaggttcccctcgccgcttcgaaagtggtccttgatgagagtgtcgcccaggagtcgtggggcatcggacgtcatctggccaaagccatcagtgacgcctgcggattgtccgtgacatgtgcccgtggcaatgactggcatggagcgatccgtctgtccattgacaggtcgttacatgagcaggaatactgtctggatatttcccattcagaaggttttgacgccagttcatatctcggagcaggagcgcaggctgcggtgcgcatcacaggtggagacgtcgctggtttgcgccatggcgccgccacgttgaggcaattggtcgttcagtgtgccccgtcactgccggtgctgcatatcgaggatcacccggcctatgcatcgagaggctattatcttgatgtcacgcgtggccgcgtgcctactttggattggctcaagcaatgggctgatttgctggaggagtgcaaatacaatcagctgcagctgtatgtcgagcacagcttcgccttcgatggcatgagcgagacgtggcgcgccaaaggggccatgtcgcccgagaccatcatggaattcgacgagtactgccatgagcgcggcatcgagttagtgccatctctctccagcttcggacacctctatgaagccctgcgcaccaaaggtttgcatgaccttggcgaattgccggagcaagctgaccggccatacagcttcatcgaacgtcagatgcatcacacgctcaacgtcaccgatccgagatccttcgcgttgtcctgtgcgatgattgatgcctacgcgccgttgttctcatctcgtaagttcaatatctgcggcgacgagacctttgacttagggcggggacgctcacacgaggaagccgaacgagtcggtgcaggcacgatgtacgccgattacatcactcggctatgcgagcacgtgcgtgagctaggccgtgaacctatgatgtggggcgatatcgcggtgtccgacccggaagtgctatccaagctacctgtggacgtgaaactgctcaactggcaatattcgccggatgtcactgatcgcgaagtggcgctggtggctgccactggggccaaccagaccgtatgcccggcgacccagacctggaaccgcatgttaccttcgctggataatgcctggcgcaatataaaccgtctgtccaaagccggccttgaccacggtgtgcgcggtttcctgctcaccgactggggcgattacggtcatgtcaacgatcctcgcatggccattcccgccctgatgtatggcgctgaatgttcctggacagccgaacggacagatttcgaatccatcaacaaccgtatcggtgcgttgcgctatggcgaccacggtgagttgctcactgtcatcgcccaggaggcggaggattctaggatcggctttagctggggagacctggtgcgttacgaggaactccgtgctcaagatgatggtgtcaacactgatgtgctctcgtctatttgggaaccaggtcccgactggaagaagcaagcgataggcaagccccttgaagaggtgcgggccatgttcctgcgctggcttgcacctcggcttgagaagtcggccccgggcctgttccagcatatctgtggggccattgcccagggttcgctgccctccggggaacgcggtgcctttctgctggcggctgccggacaggaactgttggacgcggcgggtcgtcgctttgctgcggattctggcgtgatgtcggtgcgtgccgatgacccgaaacgtgatcaattggccgcgcagttggaggaatggtttgaacagtacgtccgaatttggcgttcagtcagcgaggaatcggaattatccaaaatcggccaggtgatctggcggtgcgcggacttgttacgcgccaagcattga

SEQ ID NO: 17 - DNA, 1125 bp, Bifidobacterium longum
  source 1..1125: mol_type = other DNA; sub_species = subspecies infantis
atgggaacctccgccctttccaaggccgtgccaaccgaggtgcggctgcataacaagactatgattatgcgactgctctatcccgatcatgagatgaccagggccgatgtgtcccgtgccaccggactgacgagaatggttgtttccgacattgtttcggaactaatcgataacaaattgattacggaagtgggctttagcaagcctaatggccccggaaagcgtggcatcctccttaaattcgacgcggacagccgttgcgtcgtggcgctagatctctctcagccgtacgtttttcgtggcgcggtgatgaatctactggggcacgtcctgcccagggaagaaagcgttatctcagtcacggataccacgccgttggaagtcgtcggcaagttgtgccggcagttgattgaacggacggacaccgaggtgctaggcatcgcggtgtccgtgcccggcattgttgacgcgcatggtgtcgtgagagaatcagttaacctcggttggaaggacaccgatttgaggagctacctcactgatctgaccggactgcccgtgcacgtggataacgacagtaacgcgcagacgatggccgagctgcgattcggcgccgcggaacccaatatgatgctggtacagatagccaggggggtgggtggcggcatcattctgaatggagacctctatgtggggacttccagggccgccggcgagatcggccatgtagtggttgatcccgagggggttgagtgtgtttgtggcaagcgcgggtgcttggaaacggtgattgcgattccccggcttgtcgcacagatagatgctgatcccatccatcgtgatgagattctgcgacaggccggcgatacgttggggcgtgctttgtcgatgccgttgggtgcgctggacattccactggtcatggtgcaaggggattcatctgtggtcaaccgcagttttctggatagtgccgaacgtgcattgaacaaggctatcgccacggatctgcgcacccatgtgcgagtaaggcgttccgcattgggacaggatgcggcgttgctgggggcatgtgccatggtgctcaccgaacaattggattacatggtgcaactcggcatggcgaaatga

SEQ ID NO: 18 - DNA, 3072 bp, Bifidobacterium longum
  source 1..3072: mol_type = other DNA; sub_species = subspecies infantis
atgacagacgtcacacatgtcgatcgcgcatcgcaggcttggctgaccgacccgacggtgttcgaggtgaaccggactccggcccattccagccacaagtggtacgcccgtgacccccagagcggacaatggtccgatctcaagcagagccttgacggcgagtggcgggtcgaggtcgttcaggccgccgacatcaacctcgaagaggaacccgcgacggccgagtcgttcgacgactcctcgttcgagcgtatccaggttccgggccacctgcagacggccggtctgatgaaccacaagtacgtgaacgtccagtatccgtgggacggccacgagaacccgttggaaccgaacatcccggaaaacaatcacgtcgcgctctaccgcaggaagttcaccgtctccgcccccgtggcaaacgccaagcaggccggcggatcggtgtcgatcgtgttccacggcatggccacggcgatctacgtgtgggtcaacggcgcgttcgtcggctatggcgaggacggcttcacgcccaacgagttcgacatcaccgaactgctgcacgacggggagaacgtcgtggccgtcgcctgctacgaatactccagcgcctcctggcttgaggatcaggacttctggcgtctgcacggcctgttccgctccgtcgaactcgccgcccgcccgcatgtgcacatcgagaacacgcagatcgaagccgattgggatcccgaggccggcaccgcctccctcgacgccgcgctgaccgtgctcaacgcggccgacgcggccacggtccgcgcgaccctgaaggacgccgacggcaacacggtgtggcagacgacgggcgacgcgaaggcgcagaccgcgctctccagcggaccgctgcagggcatcgaaccatggagcgccgaaagcccgacgctgtacgagcttgacgtcgacgtcatcgaccaggcgggcgacgtcatcgaatgcacgtcccagaaggtcggtttccgccgcttccgcatcgaggacggcatcctgaccatcaacggcaagcgcatcgtgttcaagggcgccgaccgccacgagttcgacgccgaacgcggccgcgccatcaccgagcaggacatgatcgatgacgtggtcttctgcaagcgccacaacatcaactccatccgcacctcgcactacccgaaccaggaacgctggtacgaactgtgcgacgagtacggcatctacctgatcgacgagaccaacctcgaagcccacggcagctggtccctgcccggagacgtcctcaccgaggacaccatcgtgccgggcagcaagcgcgaatgggaaggcgcctgcgtcgaccgcgtcaacagcatgatgcgtcgtgactacaaccacccgagcgtgctgatctggtcgctgggcaacgaatcctacgtgggcgacgtgttccgcgccatgtacaagcacgtgcacgacatcgacccgaaccgtccggtgcactacgagggcgtgacccacaaccgtgactacgatgacgtcaccgacatcgagacccgcatgtactcgcatgccgacgagatcgagaagtacctgaaggacgacccgaagaagccgtacctctcctgcgaatacatgcacgccatgggcaactccgtgggcaacatggacgaatacacggcgctcgaacgctacccgaagtatcagggcggcttcatctgggacttcatcgaccaggccatctacgccacccagcccgacggcaccaggagcctgcgctacggcggagacttcggcgaccgtccgtccgactacgagttctccggcgacggcctgctgttcgccgaccgcaagccgtcccccaaggcccaggaagtcaagcaactgtactcgaacgtccacatcgacgtgacgaaggactcggtgtccgtcaagaacgacaacctgttcaccgccaccggcgactacgtgttcgtcctgagcgttctcgccgacggcaagccggtctggcagtccacccgacgtttcgacgtgcccgccggtgagacccgcacgttcgatgtcgcatggccggtggcggcgtaccgcgccgacgcccgcgaactggtgctgcaggtttcgcagcgtctcgccaaggcgaccgattgggccgagagcggctacgagctcgccttcggacagaccgtggtgcccgcggacgccaccgcgacgcccgacacgaagccggccgatgggaccatcaccgtgggccgttggaacgccggcgtgcgaggcgccggacgcgaggtcctgctgtcgcgcacccagggcggcatggtctcctacaccttcgccggcaacgagttcgtgctgcgccgtcccgcaatcaccaccttccgtccgctgaccgacaacgatcgcggtgccggtcacggtttcgagcgcgtccagtggctgggcgccggacgctacgcccgttgcgtggacaacgtgctcgagcagatcgacgacagcacgctcaagggcacgtacacgtacgagctcgccaccgcgcagcgcaccaaggtgaccgtctcctacacggcccacaccgatggccgcgtgaacctgcacgtcgaataccctggagagcagggtgacctgcccaccatcccggcgttcggcatcgaatggacgctgcctgtgcagtacacgaacctgaggttcttcggcaccggcccggaggagacgtacctggaccgcaaacacgccaagctcggcgtgtggagcaccaacgccttcgccgatcatgcgccgtacctcatgccgcaggagacgggcaaccatgaggatgtgcgttgggccgagattaccgacgatcacggccacggcatgcgcgtcagccgcgccgatggcgccgcgccgttcgcggtaagcctgttgccgtactccagcttcatgcttgaggaggctcagcatcaggacgagctgccgaagccgaagcacatgttcctgcgcgtccttgccgcacagatgggcgtcggcggtgatgattcctggatgtcgccggtgcacccccagtaccatatcccggcggacaagccgatcagcctcgacgtcgatctcgagctgatctga

SEQ ID NO: 19 - DNA, 1956 bp, Bifidobacterium longum
  source 1..1956: mol_type = other DNA; sub_species = subspecies infantis
gtgcccacttccgaacataaggccgacgacgcaacgcggcgtcttactctgattcccgccccggtgatattggagtacacccatggcacggctttgatcggtcctctggtgacgatcgaggatgccgatcagtcttggaagacgttgccgatcgagcagctgtccgatgaacttcggcaccgcctcggcgtcaccgtcctgaggcgccgcgcgcacggcaccgtcatatccctgggcttggattcacggctggcccatgacgaatacacgctggatgtgtccgaatcggagggcatccgtgtgcgtggcggcagcgaaagcggactgcgatacggtctgcagacgctgcggcagatcatcgggcagacctcgcgtgccatcccctgcctgcatatccaggacaagcccgcattcccggtgcgcgcctacagtctcgacgtgacacgcggacgagtgccgacgatggagttcctcacctggttcgtcgaccagctggccttatataaatacaatcagttccagctgtatgtcgagcatgccttcgcgttcgtcgagctcagcgaggcgtggcgcggcaccgatccgctcacggccgacgacatcacgtacctggacgagtactgcgcgcgtcgcggcatcgaattggtgccgtcgttggccacgttcgggcacatgtacatgaatctgcgcacccgtgagcatcgcgggctgggcgagttccccgaagacgccgaccgcccgttcagcttcatcgaacgcatggaacatcacacgctgaacgccgccgatcccaaggcccatgatttcgcctcgcgcctgattgaggaatacgcgccgctgttccgttcgaagtccttcaatatcggcggcgatgagacgttcgacttggggcgtggcaggtccgcgcaggacgcgccggaagccggccgcgatgaactgtatgccggtttcgtcagggatttgtgcgaaacgcttgcccgtcatggtcggcagccgatgttgtgggcggacatcgcgctcgaaagcccgcgcacgatggatctgctgcccggcgacatcacgatgctcaactggatgtacgagccgcagatcgacgagagcaagatccagaccatcgccgcgcagggccgtcgacagttcgtgtgccccgcggtgcgggcttggagccggttcttccccgattatgcgggtgcctggctgaacacgtatcacatggcgttggcggggaccaagtacgatgcggaaggcatggtggtcaccgattggggtgattatggccatgtcaacgatccgcgtctgagcgtgccgggcctgtgctatggcgctcagaacgcgtggaatccgatcgagatcgatgcgcatgagatgaaccgtcgaatctccgctctggtgtatggcgacgagtccggtcgcagcatggattgcctcgcccgcatcgactccgatggggtgtcgttcccctgggaccttgccgtgcaggtgctggagctggaatacggttccggcaccggcgcgctgaacatggatgtggcggcgtgcatggaacgttcgagcggcggaaggctcacgctggaccgcacgttgggatgcgatgacgcgcgccggcggatgctccagtggaaccgcgagcgtatcgaacggcgtcgggattgcgatcaggtgctgcttgactgcggcgacgcgttcgccggtctggacaagggaggcctgaccgcggagttcctgtcggtgatgctggacgggcagcgactgttcaacgaacttggcgaggaactgctggcgttggccgacggcaaggacgtcggcagcggggcgaatcgtctcgccgccgatctggagttgtggttcgagcgctatcgcgcacagtggctgtcggtcggacggcatgccgagctcgcacgtatcgcccacgtggtgtggtctctcgcggacattctgcgtaggggcgctctgtaa

SEQ ID NO: 20 - DNA, 1986 bp, Bifidobacterium longum
  source 1..1986: mol_type = other DNA; sub_species = subspecies infantis
atgcagaggtcgaaaggatatgtggctgctatggtgcaggaaccaacattggaatggcatgtcataccggagccgacgaatgtcgagccgctggtgggaacatgctcgttgccgttgtcgggaacggttgtggagcaacgaggcgcggatgatgcggaggccgtgtttgcgcgtcaacttgccgacgacattaagcgcgtatgcggaggccgctggcaggtggcctccggagaggttcagcgggaggtgactttgcggaccagtccttcgctcggtgactggtcgtatgtgcttgaggtctcgccggacggcgttgtgatcaccggttcggggttcgaaggcgttcgcgatggcgtgcagacattgcgtcagattattcgccagattggtttgacgataccgtgcatggtcatcagggatcggccggcgttctcgacccgcggttactatctggacgtgactcgtggccgtgtgccctctatggcttggctgaaatcgtgggtggatcgcctgtgcttctacaagtacaaccaattccagttgtacatcgagcatacgttccagttcaatggtcttagcgaagtgtggcgtggcgccgatccgctgacttcgtccgacattctcgagcttgattcctattgcgcggcgcgtggcatagagctcgtgccctccgtgtcgacgttcgggcaccattacaccgcgctgcggacgcggcaactacgtgatctgggagagtttccggaggatgcggatcgtcctttcagcctgatcgagcgaatgacgcaccatacgctcaacatcaccgatgagcggtcctatgagttttcgacatcattgatcgatgagctgatgccgttgttccgttcgcggaagttcaatatctgcgccgatgagacgtttgatctcggcaaggggaggtcgaagcaggaatcggcgaaacgtggcgttggcgcgatgtatgccgatttcgtcgagcgactgtgccgccatgttgatgattgtgggcatgatgtcatggtgtgggccgatgtcgcgctcgagcaccctgagatcatcgatacgcttcccaagaacatcacctggctgaattggcagtatgagccagacgtggatgatggcacaacggcagctctcgccgatgccggcgcgacgcagatggtgtgcccggcggtgtggtgctggaatgcgctgattccgcggattgatgatgcgtggaataacatcaccaggatggcgcgccatggccgcgcccatgatgtttcgggaatgctggtcactgattggggggatttcgggcacgtcaacgatccccgcatgtcggttccgggcatgatcttcggtgcgcagcaatcctggaatccggatgccgagctcagcgaagccgatatgctgtcacgcatatccactatcgaatacggcgaccatactggtagcgtggtcggtgcgctcaggggcgcttctgccaaaggcggattctcgtggagcgatctcgtcacctacctggaactggatgacggccgtggcggatgcaatacggagatcgtgcgggtcatgggctgtctggaagcgtatcggaatgacttgtcgcagtccggtcaggcaaggttggcggatgctcgcgtttcgatgctgcggacgttgcgtgactccattctcgcgggccgggaattgaacggcaagcttgacgatgcagccaaggatatcacccagctgttccgcgtggccggtgatccctcctccgctgcggtctggtcgttggccatcgacggtcagcgtctgctgaaccgtgtcgggttggcgttgttggccgcgcatggcgtggtgcggcaggatgaggccggaatcgatgcggcgaagttggccgatgaactggaatgctggaccgaacagtattcgaggctctggcatgaggtcagtcggcagtcggaactggcccgcatccaacacgtggtatggcgcgcggcggacgtgctgcgttccatttag

SEQ ID NO: 21 - DNA, 1185 bp, Bifidobacterium longum
  source 1..1185: mol_type = other DNA; sub_species = subspecies infantis
atgacggagaacgggatgatgaatacaaacaatactgtgtgcggcgcgaaccatgacggagcgatgagtctggcggccccaggcgattacggtgtggcctgctaccggattccggcccttgccgaggcgcccaacggctggatcctcgcggcgttcgacgcgcggccccataactgccaggatgccccacaggccaattcgatcgtgcagcgtatctccaaagacggcggccggtcattcgagccgcagcatgttgtggccgccgggcatgatggcgtcgacaaatacgggtattccgatccgtcctatgtggtggaccggcagacgggagaggtgttcctgttcttcgtcaaatcctatgacgccggtttcggaacctcccaggcgggcgccgatccctctgcgcgtacggtgcttcaggccgccgtcaccagttccatcgacaatggcgtgacgtggagcgagccgcgcatcatcaccgccgatatcacgaacagcgaatcatggatttcacggttcgcttcttccggtgccggcatccagctcacgtatggcgaacatgcgggacgcctgatccagcagtacaccatcaaggagctcgacggccgttaccgtgcggtatcggtcttctccgacgatcacggcgcaacttggcatgcgggcacccccgtcggcgatcacatggacgagaacaaagtcgtggaactttccgatggccgtgtgatgctgaactcgcgttcctccgatggaaacggttgccgctatgtcgccatctcccgggacggtggtgccacgtatggtccggtgatccgtgaaacgcagttgcccgatcccgagaacaacgcgcagattgcccgtgcgtttcccgatgcccccgaggggtcggcgcaggccaaggtcctgctgtattcctcctcgtcgccttcggacaggatcgatggtctggtgcgcgtctcgatcgatgacggcaagacctggagtgccggccgacggttcacgacggggccgatggcgtattcggtgatcgccgcattgagccacaaggccggcggcggctatggcctgctgtatgaaggtgataataataacattatgtacacccgtatctcgctcgactggctcaacggccagctgaacgtcgacggaatcggcggttttccgctgtctggtgagggagggtgctga

SEQ ID NO: 22 - DNA, 2349 bp, Bifidobacterium longum
  source 1..2349: mol_type = other DNA; sub_species = subspecies infantis
atgaaactcactttcgatgggatttcttcgcattgggaggaaggaatcccgttcggcaacggtcgcatgggggcggtcctgtgctccgaaccggacgcggatgtgctgtacctcaacgacgacaccctctggtcgggctatccgcatgcggagacctcgccgttgacgcccgagatcgtggccaaagcccgtcaggcgtcgtctcgcggcgattacgtggcggccacgcggatcatccaggacgccacgcagcgggagaaggacgagcagatatacgagccgttcggcacggcttgcatccggtactcgtcggaggccggcgaacggaagcatgtgaagcgcagcctggacctcgccagggccctcgccggcgaatcgttccggctgggcgccgccgacgtccatgtcgacgcatggtgcagcgctcccgatgatctgctggtgtatgagatgtcgtcgagcgcgccggtcgatgcgagcgtgagcgtctccggcacttttctcaagcggacccgagtgtcgtcggggtctgattccgatgcgcgccaggtcaccctcgtcgtcatggggcagatgcccggactcaatgtcgggtcgctggcccatgtcaccgataatccatgggaggacgagcagaacggcatcgggatggcgtatgccggcgccttctccctcaccgtcacgggcggcgagatcacggtgatcgatgatgttctgcagtgctcgggcgtcaccggcttgtcgcttcggttccgcagcctgagcgggttcaagggcagcgccgaacagccggagcgggacatgacggtgctcgccgatcgactgggcgagaccatcgccgcatggccgtccgattcgcgggcgatgcttgaccgtcatatcgccgactaccgccggttcttcgaccgggtcggcgtgcgtctcggcccggcccatgacgacgacgaggaggtgccgttcgccgagatcctgcgctccaaggaggatacgccgcaccgtctggagacgctctccgaggcgatgttcgatttcggccgatacctgctgatctcctcgtccaggccgcatacccagccgtcgaatctgcagggaatctggaaccataaggacttcccgaactggtatagcgcctacacgacgaacatcaacatcgagatgaactattggatgaccggcccatgcgccctcaaggagctcattgaaccgctcgtcgccatgaacagggagctgctggaaccggggcatgaggcggccggcgccattctcggatgcggtggttccgcggtcttccacaatgtcgatatctggcgccgggcgttgccggcgaacggagagccgacgtgggcgttctggccgttcggccaggcgtggatgtgccggaacctgttcgatgagtacctgttcaaccaggatgagtcgtatctggcttccatctggccgatcatgcgggacagcgcgcgattctgcatggattttctgtcggatacggaacacggtctggccccggctccggcgacatcgccggaaaactacttcgtcgtggacggcgaaacgatcgccgtcgcccacaccagcgaaaacaccacggccatcgtacgcaatctgctcgatgatctgatccacgcggcgcaaaccatgcccgacctggacgacggcgacaaggccttggtgcgggaggcggaatccacccgcgcgaagctggcggctgtccgcgtgggatccgatggcaggatcctcgaatggaacgatgaactcgtcgaggcggatccgcatcaccgtcacctgtcccacctgtacgaactgcatccgggcgcaggcatcaccgcgaacaccccgcgtctggaggaggtcgccagaaagtccctcgaagtgcgtggcgacgacggttccggctggagcatcgtgtggcgcatgatcatgtgggcgcgtctgcgggacgctgagcacgccgaacgcatcatcggcatgttcctccgcccggtcgaagccgacgccgaaaccgatctgcttggcggcggcgtgtacgccagcggcatgtgcgcgcatcccccgttccagatcgacggcaatctgggcttccccgcggctctggcggagatgctcgtccagagccatgacggcatggtgcgcatcctgcccgcgttgcccgaggattggcatgaaggcagcttccatggcctgcgcgccaggggaggcctttcggtggacgcctcctggacggacgacgccatcgaatacacgctgcgctgcacgaagcccgcgacgatcaccctgatcgtcgatggcgccgacgcgacgcaggtccgactgtctcccgacgagccgttcaaagggcttgtccgcaggtag

SEQ ID NO: 23 - DNA, 1437 bp, Bifidobacterium longum
  source 1..1437: mol_type = other DNA; sub_species = subspecies infantis
atgagcaatcctgcagatgcgggcatcaacctgaactatctcgcgaacgtgcgcccatcgtcgcgccagcttgcctggcagcgtatggaaatgtacgccttcctgcacttcggcatgaacaccatgacggacagggagtggggccttgggcatgaggatccggcactgttcaacccgcggaacgtggacgtggaccagtggatggacgcgctggtggccggcgggatggccggcgtcatcctgacgtgcaagcaccacgacggattctgcctgtggccgtcccgattgacgcggcacaccgtcgcctcgtcgccgtggcgtgaaggcaagggcgacctcgttcgtgaggtcagcgagtccgccaggcgtcacggactgaagttcggcgtgtacctgtccccgtgggaccgcaccgaggaatcctacggcaagggcaaggcgtacgacgacttctacgtcggccagctgaccgagctgctcacccagtacgggccgatcttctccgtctggcttgatggcgccaacggcgagggcaagaacggcaagacccagtactacgactgggatcgttactacaacgtcatccattcgctccagcccgatgcggtgatttccgtgtgcggccccgacgtgcgctgggccggaaacgaagccgggcatgtgcgcgacaacgaatggagcgtcgtgccccgccgcctgcgttcggcggaactgaccatggagaagtcgcagcaggaggatgacgcatccttcgccaccacggtcagctcccaggacgacgacctcggcagccgtgaggcggtcgccggatacggggacaacgtctgctggtacccggccgaggtcgacacctccatccgccccgggtggttctaccaccagtccgaggacgacaaggtcatgagcgccgatcagctgttcgacctctggctttccgcggtaggcggcaattcatcgttgctgctcaacattcctccctcgccggaaggcctgctcgccgaaccggatgtgcagtcgctcaagggattgggccgccgcgtcagcgagttccgtgaagcgctggcctcggtccgctgcgaagccaggaccagcagcgcctccgaagccgcggcgcatctcgtcgacggggatcgggacacgttctggcgcccggatgccgacgatgcggcccccgccatcacgctcaccctcccgcagcccacgacgatcaacgccatcgtgatcgaggaggccatagagcacggtcagcgcatcgagcatctgcgcgtcacgggtgcgctgcctgacggcaccgaacgcgtgctcggccaggccggcacggtgggttaccggaggatactccgcttcgacgatgtcgaggtgtcctcggtcacccttcacgtggacggttcgaggcttgcgccgatgatcagccgcgcggctgccgtgcgcatctga

(19) United States
(12) Patent Application Publication
(10) Pub. No.: US 2025/0312388 A1
(43) Pub. Date: Oct. 9, 2025

(54) BIFIDOBACTERIUM INFANTIS FORMULATIONS

(71) Applicants: Tahmeed AHMED, Dhaka (BD); Washington University, St. Louis, MO (US); International Centre for Diarrhoeal Disease Research, Bangladesh, Dhaka (BD)

(72) Inventors: Tahmeed AHMED, Dhaka (BD); Jeffrey I. GORDON, St. Louis, MO (US); Michael BARRATT, St. Louis, MO (US); Swetha NAKSHATRI, St. Louis, MO (US); Kazi AHSAN, St. Louis, MO (US)

(21) Appl. No.: 18/728,807

(22) PCT Filed: Jan. 12, 2023
(86) PCT No.: PCT/US2023/060562

§ 371 Date: Jul. 12, 2024

(51) Int. Cl. A61K 35/741; A61P 1/14

(52) U.S. Cl. CPC A61K 35/741; A61P 1/14

(57) ABSTRACT

The current disclosure provides compositions comprising Bifidobacterium longum subspecies infantis (B. infantis) strains with enhanced ability to uptake or utilize N-glycan and plant-based polysaccharides, and methods of using these compositions.

### CROSS-REFERENCE TO RELATED APPLICATIONS

This application is a 35 U.S.C. § 371 national phase application of PCT Application No. PCT/US2023/060562, filed Jan. 12, 2023, which claims priority to U.S. Provisional Patent Application No. 63/298,864, filed Jan. 12, 2022, each of which is incorporated herein by reference in its entirety.

### GOVERNMENTAL RIGHTS

This invention was made with government support under DK030292 awarded by the National Institutes of Health. The government has certain rights in the invention.

### SEQUENCE LISTING

This application contains a Sequence Listing that has been submitted in xml format via EFS-Web and is hereby incorporated by reference in its entirety. The xml copy is named 150601821SEQ, created on Jan. 9, 2025, and is 63,342 bytes in size.

### FIELD OF THE INVENTION

The current invention relates to the field of compositions comprising Bifidobacterium longum subspecies infantis (B. infantis) strains with enhanced ability to utilize N-glycan and plant-based polysaccharides, and methods of using these compositions.

### BACKGROUND OF THE INVENTION

The gut microbiome is a complex ecosystem with diverse microorganisms including bacteria, archaea, viruses, and fungi. More than a 100 trillion microorganisms live within a human body at any given point in time. The gut metagenome carries approximately 150 times more genes than are found in the human genome. The microbiome has a huge impact on the health and well-being of the host. Mechanisms by which these gut microorganisms impact health are manifold and include enhanced nutrient uptake, appetite signaling, competitive protection against harmful microorganisms, production of antimicrobials, and a role in development of the intestinal mucosa and immune system of the host, to a list a few. Imbalances in the microbiome are linked to developmental problems and progression of major human diseases including gastrointestinal diseases, infectious diseases, liver diseases, gastrointestinal cancers, metabolic diseases, respiratory diseases, mental or psychological diseases, and autoimmune diseases.

Addressing microbiome imbalances using probiotics is becoming an important part of treatment plans for relevant disease conditions. The microbiome is not static, however, but evolves with an individual's age, dietary intake, and environmental factors. The microbiota also varies greatly between individuals from different geographical and socioeconomical backgrounds. Therefore, probiotic therapies are not a one-size-fits all approach. The effectiveness of any intervention to address microbiome imbalances is contingent on the various factors that impact the microbiome.

There is therefore a need to understand and tailor probiotic formulations to specific populations and diet contexts.

### SUMMARY

In some aspects, the current disclosure encompasses an isolated strain of Bifidobacterium longum subspecies infantis comprising at least one DNA sequence from Bifidobacterium longum subspecies infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence. In some aspects, the at least one DNA sequence is selected from one or more polynucleotide sequences with more than 60% sequence identity to at least one of SEQ ID NOs. 2-23. In some aspects, the strain comprises at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to SEQ ID NOs. 2-23. In some aspects, the isolated strain comprises one or more polynucleotide sequences with more than 60% sequence identity to each of SEQ ID NOS. 2-23. In some aspects the isolated strain comprises at least one DNA sequence comprising a polynucleotide sequence with more than 60% sequence identity to a DNA sequence from Bifidobacterium longum subspecies infantis of NRRL deposit no. xxxxx, or a DNA sequence that is completely absent from the genomes of related Bifidobacterium isolates, wherein the DNA sequence enhances uptake of N-glycans, plant derived polysaccharides, or both; or enhances utilization of N-glycans, plant derived polysaccharides, or both, compared to a strain of the same background without the at least one DNA sequence.

In some aspects, the current disclosure also encompasses an engineered strain of Bifidobacterium longum subspecies infantis comprising one or more polynucleotide sequences comprising any of SEQ ID NOs. 2-23. In some aspects the engineered strain comprises at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 of SEQ ID NOs. 2-23. In some aspects, the engineered strain comprises each of SEQ ID NOS. 2-23. In some aspects, the engineered strain of Bifidobacterium longum subsp. infantis is an engineered strain of Bifidobacterium longum subsp. infantis ATCC 15697. In some aspects, the engineered strain of Bifidobacterium longum subsp. infantis is an engineered strain of Bifidobacterium longum subsp. infantis EVC001.

In some aspects, the current disclosure also encompasses an isolated strain of Bifidobacterium longum subsp. infantis with NRRL deposit #XXXXX.

In some aspects, the current disclosure also encompasses an isolated strain of Bifidobacterium longum subsp. infantis comprising a genome sequence at least 95%, or at least 96%, or at least 97%, or at least 98%, or at least 99%, identical to the genome sequence as provided in European Nucleotide Archive under study accession number PRJEB45396.

In some aspects, the current disclosure also encompasses a formulation comprising a therapeutically effective quantity of a strain of Bifidobacterium longum subsp. infantis comprising at least one DNA sequence from Bifidobacterium longum subsp. infantis of NRRL deposit no. xxxxx for enhanced uptake, or utilization, or both, of N-glycans, or plant derived polysaccharides, or both. In some aspects, the at least one DNA sequence is selected from one or more polynucleotide sequences with more than 60% sequence identity to at least one of SEQ ID NOs. 2-23. In some aspects, the strain comprises at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to SEQ ID NOs. 2-23. In some aspects, the strain comprises one or more polynucleotide sequences with more than 60% sequence identity to each of SEQ ID NOS. 2-23. In some aspects, the strain of Bifidobacterium longum subsp. infantis is present in an amount of more than 10² cfu per gram of the formulation. In some aspects, the Bifidobacterium longum subsp. infantis strain is in the form of viable cells. In some aspects, the Bifidobacterium longum subsp. infantis strain is in the form of a mixture of viable and non-viable cells. In some aspects, the formulation is formulated for oral administration. In some aspects, the formulation is formulated for orogastric or nasogastric administration. In some aspects, the formulation is in the form of a powder, a capsule, a tablet, a sachet, a liquid, an emulsion, or a suspension. In some aspects, the formulation comprises an ingestible carrier. In some aspects, the ingestible carrier comprises a milk component. In some aspects, the ingestible carrier comprises baby formula or baby food. In some aspects, the ingestible carrier comprises F-75 or F-100 formulas. In some aspects, the ingestible carrier comprises a beverage. In some aspects, the formulation further comprises one or more probiotic, prebiotic, adjuvant, stabilizer, biological compound, dietary supplement, drug or combination thereof. In some aspects, administering the formulation modifies the gut microbiota of a subject in need thereof. In some aspects, the formulation comprises the strain of Bifidobacterium longum subsp. infantis with NRRL deposit #xxxxx. In some aspects, the formulation comprises an engineered strain of Bifidobacterium longum subsp. infantis ATCC 15697 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence. In some aspects, the formulation comprises an engineered strain of Bifidobacterium longum subsp. infantis EVC001 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence.

In some aspects, the current disclosure also encompasses a combination, the combination comprising a therapeutically effective quantity of a strain of Bifidobacterium longum subsp. infantis comprising at least one DNA sequence from Bifidobacterium longum subsp. infantis of NRRL deposit no. xxxxx (genome assembly of the strain is available at European Nucleotide Archive under study accession number PRJEB45396) for enhanced uptake, or utilization, or both, of N-glycans, or plant derived polysaccharides, or both, and a food formulation comprising at least one carbohydrate that can be metabolized by members of the gut microbiota. In some aspects of the combination, the food formulation comprises chickpea flour, peanut flour, soy flour, green banana, and a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months; wherein the composition contains no milk, powdered milk or milk product; wherein the composition has about 300 to about 560 kcal per 100 g of the composition, a protein energy ratio (PER) of about 8% to about 20%, and a fat energy ratio (FER) of about 30% to about 60%, and wherein the amount of protein is at least 11 g per 100 g of the composition and the amount of fat is not more than 36 g per 100 g of the composition; and wherein the chickpea flour, the peanut flour, the soy flour, and the green banana, in total, provide at least 9 g of protein per 100 g of the composition. In some aspects of the combination, the food formulation comprises chickpea flour, peanut flour, soy flour, green banana, and a micronutrient premix, where in the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months; wherein the composition contains no milk, powdered milk or milk product; wherein the composition has about 400 to about 560 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%; and wherein the chickpea flour, the peanut flour, the soy flour, and the green banana, in total, provide at least 9 g of protein per 100 g of the composition. In some aspects of the combination, the food formulation comprises chickpea flour, peanut flour, soy flour, green banana, and a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months; wherein the composition contains no milk, powdered milk or milk product; wherein the composition has about 400 to about 560 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%; wherein some or all the chickpea flour is replaced with a glycan equivalent of chickpea flour, some or all the peanut flour is replaced with a glycan equivalent of peanut flour, some or all the soy flour is replaced with a glycan equivalent of soy flour, or some or all the green banana is replaced with a glycan equivalent of green banana; and wherein the chickpea flour or equivalent, the peanut flour or equivalent, the soy flour or equivalent, and the green banana or equivalent, in total, provide at least 9 g of protein per 100 g of the composition. In some aspects of the combination, the food formulation contains no (a) seeds, nuts or nut butters, (b) cocoa nibs, cocoa powder or chocolate, (c) rice flour or lentil flour, (d) dried fruit, or any combination of (a) to (d). In some aspects, the food formulation further comprises additional ingredients that may be required to achieve compliance with the Codex Alimentarius guidelines established by FAO-WHO for ready-to-use therapeutic foods.

In some aspects, the current disclosure also encompasses a method of treatment, the method comprising administering to a subject in need thereof, a therapeutically effective quantity of a formulation provided herein. In some aspects of the method of treatment, the subject is exhibiting symptoms of or diagnosed with Severe Acute Malnutrition (SAM). In some aspects of the method of treatment, the subject is an infant with a limited breastmilk diet. In some aspects of the method of treatment, the subject is exhibiting symptoms of or diagnosed with necrotizing enterocolitis, nosocomial infections, or enteric inflammation. In some aspects of the method of treatment, the formulation comprises the strain of Bifidobacterium longum subsp. infantis with NRRL deposit #xxxxx. In some aspects of the method of treatment, the strain of Bifidobacterium longum subsp. infantis is an engineered strain of Bifidobacterium longum subsp. infantis EVC001 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence. In some aspects of the method of treatment, the formulation comprises an engineered strain of Bifidobacterium longum subsp. infantis ATCC 15697 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence. In some aspects of the method of treatment, the formulation comprises an engineered strain of Bifidobacterium longum subsp. infantis comprising one or more polynucleotide sequences with more than 60% sequence identity to at least one of SEQ ID NOs. 2-23. In some aspects of the method of treatment, the engineered strain comprises at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to SEQ ID NOs. 2-23. In some aspects of the method of treatment, the engineered strain comprises one or more polynucleotide sequences with more than 60% sequence identity to each of SEQ ID NOS. 2-23. In some aspects of the method of treatment, the engineered strain of Bifidobacterium longum subsp. infantis comprises one or more polynucleotide sequences comprising SEQ ID NOS. 2-23. In some aspects of the method of treatment, the strain of Bifidobacterium longum subsp. infantis is in the form of viable cells. In some aspects of the method of treatment, the strain of Bifidobacterium longum subsp. infantis is in the form of a mixture of viable cells and non-viable cells. In some aspects of the method of treatment, the formulation is formulated for oral administration. In some aspects of the method of treatment, the formulation is formulated for orogastric or nasogastric administration. In some aspects of the method of treatment, the formulation is in the form of a powder, a capsule, a tablet, a sachet, a liquid, an emulsion, or a suspension. In some aspects of the method of treatment, the formulation comprises an ingestible carrier. In some aspects of the method of treatment, the ingestible carrier comprises a milk component. In some aspects of the method of treatment, the ingestible carrier comprises baby formula or baby food. In some aspects of the method of treatment, the ingestible carrier comprises F-75 or F-100 formulas. In some aspects of the method of treatment, the ingestible carrier comprises a beverage. In some aspects of the method of treatment, the ingestible carrier further comprises one or more probiotic, prebiotic, adjuvant, stabilizer, biological compound, dietary supplement, drug or combination thereof. In some aspects of the method of treatment, administering the formulation modifies the gut microbiota of the subject. In some aspects of the method of treatment, the subject is an undernourished child 0-5 years of age. In some aspects, the child is on a limited breast milk diet. In some aspects of the method of treatment, the child is on a no breast milk diet. In some aspects of the method of treatment, the subject is a prospective mother. In some aspects of the method of treatment, the formulation is administered before, during or after pregnancy and combinations thereof including the period of lactation or breastfeeding.

In some aspects, the current disclosure also a method for enhancing uptake, or utilization or both of milk N-glycans, or plant-based polysaccharides, or both, the method comprising administering to a subject in need thereof a therapeutically effective quantity of a formulation as provided herein. In some aspects of the method for enhancing uptake, or utilization or both of milk N-glycans, or plant-based polysaccharides, or both, the subject is exhibiting symptoms of or diagnosed with Severe Acute Malnutrition (SAM). In some aspects of the method for enhancing uptake, or utilization or both of milk N-glycans, or plant-based polysaccharides, or both, the subject is an infant with a limited breastmilk diet. In some aspects of the method for enhancing uptake, or utilization or both of milk N-glycans, or plant-based polysaccharides, or both, the subject is exhibiting symptoms of or diagnosed with necrotizing enterocolitis, nosocomial infections, or enteric inflammation. In some aspects of the method for enhancing uptake, or utilization or both of milk N-glycans, or plant-based polysaccharides, or both, the formulation comprises the strain of Bifidobacterium longum subsp. infantis with NRRL deposit #xxxxx. In some aspects of the method for enhancing uptake, or utilization or both of milk N-glycans, or plant-based polysaccharides, or both, the strain of Bifidobacterium longum subsp. infantis is an engineered strain of Bifidobacterium longum subsp. infantis EVC001 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence.

In some aspects of the method for enhancing uptake, or utilization or both of milk N-glycans, or plant-based polysaccharides, or both, the formulation comprises an engineered strain of Bifidobacterium longum subsp. infantis ATCC 15697 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence. In some aspects of the method for enhancing uptake, or utilization or both of milk N-glycans, or plant-based polysaccharides, or both, the formulation comprises an engineered strain of Bifidobacterium longum subsp. infantis comprising one or more polynucleotide sequences with more than 60% sequence identity to at least one of SEQ ID NOs. 2-23. In some aspects of the method for enhancing uptake, or utilization or both of milk N-glycans, or plant-based polysaccharides, or both, the engineered strain comprises at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to SEQ ID NOs. 2-23. In some aspects of the method for enhancing uptake, or utilization or both of milk N-glycans, or plant-based polysaccharides, or both, the engineered strain comprises one or more polynucleotide sequences with more than 60% sequence identity to each of SEQ ID NOS. 2-23. In some aspects of the method for enhancing uptake, or utilization or both of milk N-glycans, or plant-based polysaccharides, or both, the engineered strain of Bifidobacterium longum subsp. infantis comprises one or more polynucleotide sequences comprising SEQ ID NOS. 2-23. In some aspects of the method for enhancing uptake, or utilization or both of milk N-glycans, or plant-based polysaccharides, or both, the strain of Bifidobacterium longum subsp. infantis is in the form of viable cells. In some aspects of the method for enhancing uptake, or utilization or both of milk N-glycans, or plant-based polysaccharides, or both, the strain of Bifidobacterium longum subsp. infantis is in the form of a mixture of viable cells and non-viable cells. In some aspects of the method for enhancing uptake, or utilization or both of milk N-glycans, or plant-based polysaccharides, or both, the formulation is formulated for oral administration. In some aspects of the method for enhancing uptake, or utilization or both of milk N-glycans, or plant-based polysaccharides, or both, the formulation is formulated for orogastric or nasogastric administration. In some aspects of the method for enhancing uptake, or utilization or both of milk N-glycans, or plant-based polysaccharides, or both, the formulation is in the form of a powder, a capsule, a tablet, a sachet, a liquid, an emulsion, or a suspension.

In some aspects of the method for enhancing uptake, or utilization or both of milk N-glycans, or plant-based polysaccharides, or both, the formulation comprises an ingestible carrier. In some aspects of the method for enhancing uptake, or utilization or both of milk N-glycans, or plant-based polysaccharides, or both, the ingestible carrier comprises a milk component. In some aspects of the method for enhancing uptake, or utilization or both of milk N-glycans, or plant-based polysaccharides, or both, the ingestible carrier comprises baby formula or baby food. In some aspects of the method for enhancing uptake, or utilization or both of milk N-glycans, or plant-based polysaccharides, or both, the ingestible carrier comprises F-75 or F-100 formulas. In some aspects of the method for enhancing uptake, or utilization or both of milk N-glycans, or plant-based polysaccharides, or both, the ingestible carrier comprises a beverage. In some aspects of the method for enhancing uptake, or utilization or both of milk N-glycans, or plant-based polysaccharides, or both, the formulation further comprising one or more probiotic, prebiotic, adjuvant, stabilizer, biological compound, dietary supplement, drug or combination thereof. In some aspects of the method for enhancing uptake, or utilization or both of milk N-glycans, or plant-based polysaccharides, or both, the administering the formulation modifies the gut microbiota of the subject. In some aspects of the method for enhancing uptake, or utilization or both of milk N-glycans, or plant-based polysaccharides, or both, the subject is an undernourished child 0-5 years of age. In some aspects of the method for enhancing uptake, or utilization or both of milk N-glycans, or plant-based polysaccharides, or both, the child is on a limited breast milk diet. In some aspects of the method for enhancing uptake, or utilization or both of milk N-glycans, or plant-based polysaccharides, or both, the child is on a no breast milk diet. In some aspects of the method for enhancing uptake, or utilization or both of milk N-glycans, or plant-based polysaccharides, or both, the subject is a prospective mother. In some aspects of the method for enhancing uptake, or utilization or both of milk N-glycans, or plant-based polysaccharides, or both, the formulation is administered before, during or after pregnancy and combinations thereof including the period of lactation or breastfeeding.

In some aspects, the current disclosure also encompasses a method for modifying the gut microbiota of a subject in need thereof, the method comprising administering to a subject a therapeutically effective quantity of a formulation as disclosed herein. In some aspects of the method for modifying the gut microbiota, the subject is exhibiting symptoms of or diagnosed with Severe Acute Malnutrition (SAM). In some aspects of the method for modifying the gut microbiota, the subject is an infant with a limited breastmilk diet. In some aspects of the method for modifying the gut microbiota, the subject is exhibiting symptoms of or diagnosed with necrotizing enterocolitis, nosocomial infections, enteric inflammation, or diarrheal illness. In some aspects of the method for modifying the gut microbiota, the formulation comprises the strain of Bifidobacterium longum subsp. infantis with NRRL deposit #xxxxx. In some aspects of the method for modifying the gut microbiota, the strain of Bifidobacterium longum subsp. infantis is an engineered strain of Bifidobacterium longum subsp. infantis EVC001 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence. In some aspects of the method for modifying the gut microbiota, the formulation comprises an engineered strain of Bifidobacterium longum subsp. infantis ATCC 15697 comprising at least one DNA sequence from Bifidobacterium longum subsp. infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence.

In some aspects of the method for modifying the gut microbiota, the formulation comprises an engineered strain of Bifidobacterium longum subsp. infantis comprising one or more polynucleotide sequences with more than 60% sequence identity to at least one of SEQ ID NOs. 2-23. In some aspects of the method for modifying the gut microbiota, the engineered strain comprises at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to SEQ ID NOs. 2-23. In some aspects of the method for modifying the gut microbiota, the engineered strain comprises one or more polynucleotide sequences with more than 60% sequence identity to each of SEQ ID NOS. 2-23. In some aspects of the method for modifying the gut microbiota, the engineered strain of Bifidobacterium longum subsp. infantis comprises one or more polynucleotide sequences comprising SEQ ID NOS. 2-23. In some aspects of the method for modifying the gut microbiota, the strain of Bifidobacterium longum subsp. infantis is in the form of viable cells. In some aspects of the method for modifying the gut microbiota, the strain of Bifidobacterium longum subsp. infantis is in the form of a mixture of viable cells and non-viable cells. In some aspects of the method for modifying the gut microbiota, the formulation is formulated for oral administration. In some aspects of the method for modifying the gut microbiota, the formulation is formulated for orogastric or nasogastric administration. In some aspects of the method for modifying the gut microbiota, the formulation is in the form of a powder, a capsule, a tablet, a sachet, a liquid, an emulsion, or a suspension. In some aspects of the method for modifying the gut microbiota, the formulation comprises an ingestible carrier. In some aspects of the method for modifying the gut microbiota, the ingestible carrier comprises a milk component. In some aspects of the method for modifying the gut microbiota, the ingestible carrier comprises baby formula or baby food. In some aspects of the method for modifying the gut microbiota, the ingestible carrier comprises F-75 or F-100 formulas. In some aspects of the method for modifying the gut microbiota, the ingestible carrier comprises a beverage. In some aspects of the method for modifying the gut microbiota, the formulation further comprises one or more probiotic, prebiotic, adjuvant, stabilizer, biological compound, dietary supplement, drug or combination thereof. In some aspects of the method for modifying the gut microbiota, the subject is an undernourished child 0-5 years of age. In some aspects of the method for modifying the gut microbiota, the child is on a limited breast milk diet. In some aspects of the method for modifying the gut microbiota, the child is on a no breast milk diet. In some aspects of the method for modifying the gut microbiota, the microbiota of the child has an impaired capacity to ‘digest’ N-glycans or plant derived polysaccharides. In some aspects of the method for modifying the gut microbiota, the subject is a prospective mother. In some aspects of the method for modifying the gut microbiota, the formulation is administered before, during or after pregnancy and combinations thereof including the period of lactation or breastfeeding. In some aspects of the method for modifying the gut microbiota, the subject is a pre-term infant that has an elevated risk of nosocomial infections or necrotizing enterocolitis. In some aspects of the method for modifying the gut microbiota, the subject has been administered or will be administered a vaccine or an antibiotic.

### BRIEF DESCRIPTION OF THE FIGURES

FIG. 1 is a schematic reconstruction of key HMO utilization genes in Bifidobacterium longum subspecies infantis (B. infantis) strains used in this study. Shown is the reconstruction of loci involved in the utilization of HMOs in two B. infantis strains. EVC001 is a U.S. donor-derived probiotic B. infantis strain; the Bg_2D9 strain was isolated from a 12-month-old healthy Bangladeshi child. (Blon, B. longum locus tag; FL1/FL2, fucosyllactose 1 and fucosyllactose 2; Nan, N-acetylneuraminic acid; TF, transcription factor).

FIG. 2A shows a quantitative PCR (qPCR) assay of the absolute abundance of Bifidobacteria in fecal samples from healthy and SAM Bangladeshi infants/children with qPCR assays directed at the Blon_2348 (NanH2 exo-a-sialidase) gene of B. infantis. Scatterplots (left panels) display the absolute abundance of target genes as normalized log 10 transformed genome equivalents per μg of fecal DNA as a function of age at the time of specimen collection. Samples from healthy infants and children are indicted by green points/shading while those from individuals with SAM are denoted by red. A generalized additive model-derived best fit line (+2 SEM) is shown. Plot difference curves (right panels) depict the estimated difference in fit between healthy compared to SAM based on model predictions. Statistically significant differences in the best fit lines between the two models (healthy vs SAM) are indicated by the areas bounded by red dashed lines.

FIG. 2B shows a quantitative PCR (qPCR) assay of the absolute abundance of Bifidobacteria in fecal samples from healthy and SAM Bangladeshi infants/children with qPCR assays directed at the Lacto-N-tetraose (LNT) ABC transporter permease subunit (Blon_2176). Scatterplots (left panels) display the absolute abundance of target genes as normalized log₁₀ transformed genome equivalents per μg of fecal DNA as a function of age at the time of specimen collection. Samples from healthy infants and children are indicted by green points/shading while those from individuals with SAM are denoted by red. A generalized additive model-derived best fit line (±2 SEM) is shown. Plot difference curves (right panels) depict the estimated difference in fit between healthy compared to SAM based on model predictions. Statistically significant differences in the best fit lines between the two models (healthy vs SAM) are indicated by the areas bounded by red dashed lines.

FIG. 2C shows a quantitative PCR (qPCR) assay of the absolute abundance of Bifidobacteria in fecal samples from healthy and SAM Bangladeshi infants/children with qPCR assays directed at the 16S rDNA gene of Bifidobacteria. Scatterplots (left panels) display the absolute abundance of target genes as normalized log₁₀ transformed genome equivalents per μg of fecal DNA as a function of age at the time of specimen collection. Samples from healthy infants and children are indicted by green points/shading while those from individuals with SAM are denoted by red. A generalized additive model-derived best fit line (±2 SEM) is shown. Plot difference curves (right panels) depict the estimated difference in fit between healthy compared to SAM based on model predictions. Statistically significant differences in the best fit lines between the two models (healthy vs SAM) are indicated by the areas bounded by red dashed lines.

FIG. 2D shows a quantitative PCR (qPCR) assay of the absolute abundance of Bifidobacteria in fecal samples from healthy and SAM Bangladeshi infants/children with qPCR assays directed at the nglA subunit of the N-glycan ABC transport system (nglABC). Fecal samples from healthy children (n=130 samples) or children with SAM (n=102 samples) were assayed. Scatterplots (left panels) display the absolute abundance of target genes as normalized log₁₀ transformed genome equivalents per μg of fecal DNA as a function of age at the time of specimen collection. Samples from healthy infants and children are indicted by green points/shading while those from individuals with SAM are denoted by red. A generalized additive model-derived best fit line (±2 SEM) is shown. Plot difference curves (right panels) depict the estimated difference in fit between healthy compared to SAM based on model predictions. Statistically significant differences in the best fit lines between the two models (healthy vs SAM) are indicated by the areas bounded by red dashed lines.

FIG. 3A shows the study design for SYNERGIE clinical study.

FIG. 3B shows the effect of the interventions on weight-for-age z scores (WAZ) at the end of the study compared to the time of hospital discharge. Bar plots represent group means; error bars represent standard deviations. P values were calculated using the Mann-Whitney U test.

FIG. 3C shows the effect of the interventions on Mid-Upper Arm Circumference (MUAC) at the end of the study compared to the time of hospital discharge. Bar plots represent group means; error bars represent standard deviations. P values were calculated using the Mann-Whitney U test.

FIG. 3D shows the Spearman correlation between fecal levels of lipocalin-2 (LCN-2) and the change in WAZ from hospital discharge to study completion.

FIG. 3E shows the Spearman correlation between levels of fecal interferon-β (IFN-β) and the rate of weight gain in infants between discharge and study completion (Spearman's p and FDR adjusted P values for each correlation are shown in panels D and E).

FIG. 4A shows experimental design for in vivo competition of B. infantis strains in gnotobiotic mice consuming the Mirpur-6 diet±LNT or LNnT.

FIG. 4B shows data for in vivo competition of B. infantis strains in gnotobiotic mice involving the 5-member consortium of B. infantis strains unsupplemented. Absolute abundances (log₁₀ genome equivalents per mg feces) of the different strains, as a function of time (experimental days 4, 8, 12, 18 and 26), were determined by short read shotgun sequencing of fecal DNA. Mean values±SD are plotted. Timepoints at which the absolute abundance of Bg_2D9 was statistically significantly higher than other consortium members was determined using a mixed effects linear model followed by Tukey's multiple comparison test.*, P_{adj}<0.05.

FIG. 4C shows data for in vivo competition of B. infantis strains in gnotobiotic mice involving the 5-member consortium of B. infantis strains LNT supplemented. Absolute abundances (log 10 genome equivalents per mg feces) of the different strains, as a function of time (experimental days 4, 8, 12, 18 and 26) and HMO supplementation, were determined by short read shotgun sequencing of fecal DNA. Mean values±SD are plotted. Timepoints at which the absolute abundance of Bg_2D9 was statistically significantly higher than other consortium members was determined using a mixed effects linear model followed by Tukey's multiple comparison test.*, P_{adj}<0.05.

FIG. 4D shows data for in vivo competition of B. infantis strains in gnotobiotic mice involving the 5-member consortium of B. infantis strains LNnT supplemented. Absolute abundances (log₁₀ genome equivalents per mg feces) of the different strains, as a function of time (experimental days 4, 8, 12, 18 and 26) and HMO supplementation, were determined by short read shotgun sequencing of fecal DNA. Mean values±SD are plotted. Timepoints at which the absolute abundance of Bg_2D9 was statistically significantly higher than other consortium members was determined using a mixed effects linear model followed by Tukey's multiple comparison test.*, P_{adj}<0.05.

FIG. 4E shows data for in vivo competition with the 5-member consortium of B. infantis strains introduced together with a B. bifidum strain isolated from a healthy Bangladeshi infant. Absolute abundances (log 10 genome equivalents per mg feces) of the different strains, as a function of time (experimental days 4, 8, 12, 18 and 26) and HMO supplementation, were determined by short read shotgun sequencing of fecal DNA. Mean values±SD are plotted. Timepoints at which the absolute abundance of Bg_2D9 was statistically significantly higher than other consortium members was determined using a mixed effects linear model followed by Tukey's multiple comparison test. *, P_{adj}<0.05.

FIG. 4F shows experimental design of the study examining colonization of the microbiota of pups whose mothers received a fecal microbiota sample from a SAM donor with or without B. infantis Bg_2D9 and EVC001.

FIG. 4G shows the weight of pups on postnatal days 18 and 35 (means±SD; n=11 pups in the SAM-only group and n=12 pups in the SAM plus B. infantis group, n=1 experiment). ″″″P<0.001; ″″P<0.01, two-way repeated-measures ANOVA followed by Šidák's multiple comparison test.

FIG. 4H shows the results of a 16S rRNA-based analysis of the relative abundances of ASVs assigned to Enterobacteriaceae and bifidobacteria present in the fecal microbiota of P28 pups in the two treatment groups.

FIG. 4I shows the absolute abundances of Bg_2D9 and EVC001 in the feces from pups at P21, P28 and P35 defined by qPCR using strain-specific primers targeting the nglA and epsJ genes, respectively. *** P<0.001, ** P<0.01; Two-tailed Wilcoxon matched-pairs signed rank test.

FIG. 5A schematically depicts unique sugar utilization clusters of B. infantis Bg_2D9: B-glucoside utilization (Bgl) gene cluster in Bg_2D9 and the N-glycan utilization (Ngl) cluster in B. infantis strains included in the gnotobiotic mouse experiment. Predicted transcription factor binding sites (TFBS) are denoted by grey circles.

FIG. 5B shows expression of Ngl cluster genes in the B. infantis Bg_2D9 and EVC001 strains. Mice fed either Mirpur-6, Mirpur-6+1.25% LNT (in their drinking water) or Mirpur-6+1.25% LNnT were colonized with the consortium of five B. infantis strains. In the fourth arm, B. bifidum was included in the consortium and mice were given drinking water supplemented with 1.25% LNnT. Black pixels indicate an absence of ortholog in a strain. Grey pixels show depict low expression (≤10 read counts). Black bars in the leftmost column indicate that the transcription factor has not been characterized.

FIG. 5C shows a schematic of the proposed scheme of N-glycan utilization in B. infantis Bg_2D9.

FIG. 6A shows the relative abundances of top 30 most abundant Amplicon Sequence Variants (ASVs) in the fecal microbiota of Bangladeshi infants who exhibited healthy growth.

FIG. 6B shows the relative abundances of top 30 most abundant Amplicon Sequence Variants (ASVs) in the fecal microbiota of Bangladeshi infants who had SAM.

FIG. 7A shows in vitro growth phenotypes of B. infantis strains in defined low-carbohydrate MRS medium (with Lacto-N-Tetraose) in the presence or absence of different HMOs.

FIG. 7B shows in vitro growth phenotypes of B. infantis strains in defined low-carbohydrate MRS medium (with Lacto-N-Neotetraose) in the presence or absence of different HMOs.

FIG. 7C shows in vitro growth phenotypes of B. infantis strains in defined low-carbohydrate MRS medium (with 2′-Fucosyllactose) in the presence or absence of different HMOs.

FIG. 7D shows in vitro growth phenotypes of B. infantis strains in defined low-carbohydrate MRS medium (with 3′-Sialyllactose) in the presence or absence of different HMOs.

FIG. 7E shows in vitro growth phenotypes of B. infantis strains in defined low-carbohydrate MRS medium (with 6′-Sialyllactose) in the presence or absence of different HMOs.

FIG. 7E shows in vitro growth phenotypes of B. infantis strains in defined low-carbohydrate MRS medium (with lactose) in the presence or absence of different HMOs.

FIG. 7G shows in vitro growth phenotypes of B. infantis strains in defined base media in the presence or absence of different HMOs.

FIG. 8A shows expression of Bgl cluster genes in the B. infantis Bg_2D9 and EVC001 strains.

FIG. 8B shows expression of HMO utilization genes in the B. infantis Bg_2D9 and EVC001 strains.

### DETAILED DESCRIPTION

The present disclosure encompasses compositions and methods of treatment for subjects in need thereof, where the methods of treatment comprise administering a disclosed composition. In some embodiments, the methods of treatment address malnutrition, including undernutrition, in part by modifying the gut microbiota of the subject. The global burden of childhood undernutrition is great, causing 3.1 million deaths annually and accounting for 21% of life years lost among children younger than 5 years. More than 18 million children in this age range are affected by severe acute malnutrition (SAM), the most extreme form of undernutrition. SAM is responsible for nearly half of all undernutrition-related mortality. Various aspects of this invention demonstrate that there is a correlation between childhood malnutrition and deficiencies in components of the gut microbiota whose restoration is associated with improved outcomes for acutely malnourished children. In one aspect the present disclosure encompasses extensive screening and in-depth characterization methods for identification of Bifidobacterium longum subspecies infantis (B. infantis) strains for enhanced survival (fitness) in children who consume diets with limited breastmilk content. While exclusive breastfeeding of infants is recommended by the WHO for the first 6 months, in many low-income settings, gruels, animal milk and complementary foods are often introduced into the diet at an early age for economic and/or cultural reasons. Surprisingly, one strain obtained from these extensive screening efforts exhibits superior fitness over multiple other strains, independent of human milk oligosaccharides supplementation in the population studied. In-depth characterization of the strain helped define DNA sequences involved in the uptake, or utilization or both of N-glycans, or plant-based polysaccharides, or both that were absent in comparator strains of the same background Bifidobacterium longum subspecies infantis isolated to date.

The current disclosure describes isolated and engineered strains of B. infantis comprising one or more of these DNA sequences, and therapeutic formulations or combinations comprising these strains, that when administered into a subject in need thereof, enhance the capacity for uptake or utilization of N-glycans or plant-based polysaccharides. Such treatments improve outcomes for malnourished children, especially those with limited or no breastmilk consumption. In some aspects, the disclosed formulations can be administered in combination with food formulations. Some aspects of this invention further provide methods for modifying gut microbiota, thus providing advantageous outcomes including but not limited to reducing symptoms of, or treating, acute malnutrition, enteric inflammation, necrotizing enterocolitis, and allergies, promoting recolonization of the gut after diarrhea or antibiotic consumption, and improving vaccine performance by administering therapeutically effective quantities of these formulations.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “comprising” means “including, but not necessarily limited to”; it specifically indicates open-ended inclusion or membership in a so-described combination, group, series and the like. The terms “comprising” and “including” as used herein are do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

As used herein, the term “polynucleotide”, which may be used interchangeably with the term “nucleic acid” generally refers to a biomolecule that comprises two or more nucleotides. In some aspects, a polynucleotide comprises at least two, at least five at least ten, at least twenty, at least 30, at least 40, at least 50, at least 100, at least 200, at least 250, at least 500, or any number of nucleotides. For example, the polynucleotides may include at least 500 nucleotides, at least about 600 nucleotides, at least about 700 nucleotides, at least about 800 nucleotides, at least about 900 nucleotides, at least about 1000 nucleotides, at least about 2000 nucleotides, at least about 3000 nucleotides, at least about 4000 nucleotides, at least about 4500 nucleotides, or at least about 5000 nucleotides. A polynucleotide may be single-stranded or double-stranded. In some aspects, a polynucleotide is a site or region of genomic DNA. In some aspects, a polynucleotide is an endogenous gene that is comprised within the genome of an unmodified cell or universal donor cell. In some aspects, a polynucleotide is an exogenous polynucleotide that is not integrated into genomic DNA. In some aspects, a polynucleotide is an exogenous polynucleotide that is integrated into genomic DNA. In some aspects, a polynucleotide is a plasmid. In some aspects, a polynucleotide is a circular or linear molecule.

The term “DNA sequence” refers to a heritable sequence of DNA, i.e., a genomic sequence, with functional significance. The term “gene” can be used to refer to, e.g., a cDNA and/or an mRNA encoded by a genomic sequence, as well as to that genomic sequence.

Nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

The lab strain “Bifidobacterium longum subspecies infantis Bg40721_2D9_SN_2018” refers to an isolated strain of Bifidobacterium longum subspecies infantis available at Professor Jeffery I. Gordon's laboratory at Washington University, School of Medicine at St. Louis and corresponds to NRRL deposit no. xxxx at the ARS Culture Collection (NRRL). A genome assembly of this strain is available in the European Nucleotide Archive under accession number PRJEB45396.

The term “carbohydrate”, as used herein, refers to an organic compound with the formula Cm(H2O)n, where m and n may be the same or different number, provided the number is greater than 3.

The term “glycan” refers to a linear or branched homo- or heteropolymer of two or more monosaccharides linked glycosidically. As such, the term “glycan” includes disaccharides, oligosaccharides and polysaccharides. The term also encompasses a polymer that has been modified, whether naturally or otherwise; non-limiting examples of such modifications include acetylation, alkylation, esterification, etherification, oxidation, phosphorylation, selenization, sulfonation, or any other manipulation.

The term “N-glycan,” as used herein, refers to a polymer of sugars that has been released from a glycoconjugate but was formerly linked to the glycoconjugate via a nitrogen linkage (see definition of N-linked glycan below). “N-linked glycans” are glycans that are linked to a glycoconjugate via a nitrogen linkage. A diverse assortment of N-linked glycans exist.

The term “plant-based polysaccharides” as used herein refers to polysaccharides derived from plants. Generally, plant-based polysaccharides consist of large insoluble polymers, like cell wall components, small soluble oligosaccharides, like monomers (e.g. glucose) and dimers (e.g. cellobiose), and large soluble polysaccharides. Suitably, the polysaccharide is non-animal, i.e., is not obtained or derived from animals or the microbiome. In some aspects, plant-based polysaccharides comprise plant-derived beta-glycans.

As used herein, the term “malnutrition” refers to one or more forms of undernutrition—for example, wasting (low weight-for-length), stunting (low length-for-age), underweight (low weight-for age), deficiencies in vitamins and minerals, etc. A subject in need of treatment for malnutrition may also be referred to herein as a malnourished subject.

A length-for-age Z Score (LAZ) refers to the number of standard deviations of the actual length of a child from the median length of the children of his/her age as determined from the standard sample. This is prefixed by a positive sign (+) or a negative sign (−) depending on whether the child's actual length is more than the median length or less than the median length. The terms length and height are used interchangeably herein. Therefore, length-for-age Z Score (LAZ) and height-for-age Z Score (HAZ) refer to the same measurement.

A weight-for-age Z score (WAZ) refers to the number of standard deviations of the actual weight of a child from the median weight of the children of his/her age as determined from the standard sample. This is prefixed by a positive sign (+) or a negative sign (−) depending on whether the child's actual weight is more than the median weight or less than the median weight.

A weight-for-length Z score (WLZ) refers to the number of standard deviations of the actual weight of a child from the median weight of the children of his/her length as determined form the standard sample. This is prefixed by a positive sign (+) or a negative sign (−) depending on whether the child's actual weight is more than the median weight or less than the median weight for the same length. The terms length and height are used interchangeably herein. Therefore, weight-for-height Z score (WHZ) and weight-for-length Z score (WLZ) refer to the same measurement.

A mid-upper-arm-circumference score (MUAC) is an independent anthropometric measurement used to identify malnutrition.

Moderate acute malnutrition (MAM) is defined by a WHZ less than or equal to −2 and greater than or equal to −3.

Severe acute malnutrition (SAM) is defined by a WHZ less than-3 and/or bipedal edema, and/or a mid-upper arm circumference (MUAC) less than 11.5 cm.

As used herein, a “healthy child” has a LAZ and WLZ consistently no more than 1.5 standard deviations below the median calculated from a World Health Organization (WHO) reference healthy growth cohort as described in WHO Multicentre Reference Study (MGRS), 2006 (www.who.int/childgrowth/mgrs/en).

As used herein, “statistically significant” is a p-value <0.05, <0.01, <0.001, <0.0001, or <0.00001.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilization (i.e., not worsening) of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

As used herein, the term “effective amount” means an amount of a substance (e.g. a composition including formulations and combinations of the present disclosure) that leads to measurable and beneficial effects for the subject administered the substance, i.e., significant efficacy. As used herein the term “therapeutically effective amount” refers to an amount of the formulation or therapeutic combination that alleviates, in whole or in part, symptoms associated with the disorder or condition, or halts or slows further progression or worsening of those symptoms, or prevents or provides prophylaxis for the disorder or condition. A therapeutically effective amount is also one in which any toxic or detrimental effects of compositions of the invention are outweighed by the therapeutically beneficial effects.

As used herein, the term “raw banana” refers to an unripe, green banana in the genus Musa. “Raw bananas” are also referred to as “green bananas” in the art, and the terms are used interchangeably herein. As is understood in the art, raw bananas are processed (e.g., baked, boiled, steamed, etc.) after which the pulp may or may not be dried prior to use.

The term “modifying” as used in the phrase “modifying the gut microbiota” is to be construed in its broadest interpretation to mean a change in the representation of microbes in the gastrointestinal tract of a subject. The change may be a decrease or an increase in the presence of a particular microbial strain, species, genus, family, order, or class. In some aspects, “modifying the gut microbiota” can “repair the gut microbiota” or “improve gut microbiota health”. To “repair the gut microbiota of a subject,” which is synonymous with “improve gut microbiota health,” means to change the microbiota of a subject, in particular the relative abundances of age- and health-discriminatory taxa, in a statistically significant manner towards chronologically-age matched reference healthy subjects. The term encompasses complete repair and levels of repair that are less than complete. The term also encompasses preventing or lessening a change in the relative abundances of age- and health-discriminatory taxa, wherein the change would have been significantly greater absent intervention.

As used herein the term “enhanced uptake” is intended to mean that the presence of the DNA sequence enhances the active transport of N-glycans, plant-derived polysaccharides, or both into the bacterial cell compared to the same cell, or a cell of a similar background without the DNA sequence. In some aspects, the DNA sequence is known (based on assays known to a person of ordinary skill in the art including but not limited to binding assays, assays using glycan-recognizing probes comprising one or more of antibodies, lectins, carbohydrate molecules coupled with enzyme assays, immunohistochemistry, confocal microscopy, electron microscopy and flow cytometry) or predicted (based on sequence homology studies or curation using mcSEED analysis) to increase binding and intracellular transport of N-glycans, or plant derived oligosaccharides, or both by the microbe.

As used herein the term “enhanced utilization” is intended to mean that the presence of the DNA sequence enhances one or more of transport of N-glycans, transport of plant-derived polysaccharides, or both into the bacterial cell, and their subsequent metabolic processing [or metabolism]. In some aspects the DNA sequence is known (based on assays known to a person of ordinary skill in the art including but not limited to carbohydrate fermentation assays or glycan-recognizing probes comprising one or more of antibodies, lectins, carbohydrate molecules or enzyme assays) or predicted to (based on sequences homology studies or curation using mcSEED analysis) to increase microbial breakdown of N-glycans or plant derived oligosaccharides, or both.

As used herein, the term “subject” refers to a mammal. In some aspects, a subject is non-human primate or rodent. In some aspects, a subject is a human. In some aspects, a subject has, is suspected of having, or is at risk for, a disease or disorder. In some aspects, a subject has one or more symptoms of a disease or disorder. In particular aspects, a subject is malnourished.

### I. Compositions

i. Isolated and Engineered Strains

In one aspect, the present disclosure encompasses isolated strains of Bifidobacterium longum subspecies infantis (B. infantis) comprising at least one DNA sequence from Bifidobacterium longum subspecies infantis of NRRL deposit no. xxxxx that enhances uptake of N-glycans, plant derived polysaccharides, or both; or enhances utilization of N-glycans, plant derived polysaccharides, or both, compared to a strain of the same background without the at least one DNA sequence. In another aspect, the present disclosure encompasses isolated strains of Bifidobacterium longum subspecies infantis (B. infantis) comprising at least one DNA sequence from the genome assembly published in the European Nucleotide Archive under study accession number PRJEB45396, that enhances uptake of N-glycans, plant derived polysaccharides, or both; or enhances utilization of N-glycans, plant derived polysaccharides, or both, compared to a strain of the same background without the at least one DNA sequence.

In some aspects, the DNA sequence can comprise one or more polynucleotide sequences from a predicted B-glucoside utilization cluster (Bgl, SEQ ID NOS. 2) or an N-glycan utilization cluster (Ngl, SEQ ID NOS. 3) of genes or both. In some aspect the DNA sequence may comprise one or more of any of the multiple intracellular exo-acting glycoside hydrolase (GH) including but not limited to Bga2A, Hex1, Hex2, NanH2, BiAfcA, BiAfcB (SEQ ID NOS 18-SEQ ID NOS 23 respectively). In some aspects the strain of B. infantis may comprise all or portions of polynucleotide sequence from the Bgl cluster, the Ngl cluster and one or more GH or any other DNA sequence that is known to or predicted to directly or indirectly enhance the ability of the subject to uptake or utilize N-glycans, plant-based polysaccharides, or both from B. infantis NRRL deposit #XXXX (genome assembly available at European Nucleotide Archive under study accession number PRJEB45396).

The Bgl cluster comprises (i) three glycoside hydrolases (GHs) [a hypothetical glucan endo-β-1,6-glucosidase belonging to glycoside hydrolase family 30 (GH30)-SEQ ID NOS. 4, an exo-β-1,4/6-glucosidase (GH3)-SEQ ID NOS. 5, and a hypothetical β-galactosidase (GH2 family)-SEQ ID NOS. 6]; (ii) an ABC transport system [encoded by bglY, bglZ, bglX-SEQ ID NOS. 7-9] and (iii) a TetR family transcriptional regulator [bglT, SEQ ID NOS. 10]. In some aspects, the DNA sequence may comprise nucleotide sequences from any or all of the elements of the Bgl cluster. In some aspects the DNA sequence may comprise one or more polynucleotide sequences that are at least 60% (e.g., at least 65%, 70%, 75%, 80%, 85%, 90%, 95%, 97%, 98%, 99%, or 100%) identical to any of SEQ ID NOS. 2, 4-10. In some aspects the DNA sequence may comprise one or more polynucleotide sequences that are at least about 60% to about 70%, or about 70% to about 80%, or about 80% to about 90%, or about 90% to about 100% identical to any of SEQ ID NOS. 2, 4-10.

The Ngl cluster in the B. infantis deposit #XXXX genome (genome assembly available at European Nucleotide Archive under study accession number PRJEB45396) contains two endo-β-N-acetylglucosaminidases: EndoBI-2 (SEQ ID. NOS 11) and EndoBB-2 (GH85-SEQ ID NOS. 12). The Ngl cluster also contains genes encoding (i) an ABC transport system (NglABC) or Blon_2378-2380 predicted to transport N-glycans (SEQ ID NOS. 13); (ii) GHs involved in degradation of (complex)N-glycans, namely a-mannosidase, Mna 38 (GH38-SEQ ID NOS. 14), a homolog of the biochemically-characterized β-mannosidase, BIMan5B (GH5_18-SEQ ID NOS. 15), a β-N-acetylglucosaminidase, Hex3 (GH20; SEQ ID NOS. 16), and (iii) a transcriptional regulator (NgIR) from the ROK family, NgIR (SEQ ID NOS. 17). In some aspects, the DNA sequence may comprise polynucleotide sequences from any or all of the elements of the Ngl cluster. In some aspects the DNA sequence may comprise one or more polynucleotide sequences that are at least 60% (e.g., at least 65%, 70%, 75%, 80%, 85%, 90%, 95%, 97%, 98%, 99%, or 100%) identical to any of SEQ ID NOS. 3, 11-17. In some aspects the DNA sequence may comprise one or more polynucleotide sequences that are at least about 60% to about 70%, or about 70% to about 80%, or about 80% to about 90%, or about 90% to about 100% identical to any of SEQ ID NOS. 3, 11-17.

In addition to the Bgl and Ngl clusters, extensive characterization of B. infantis NRRL deposit #xxxx (genome assembly available as European Nucleotide Archive under study accession number PRJEB45396) also provided additional DNA sequences that are predicted to or known to enhance uptake, or utilization, or both of N-glycans, or plant derived polysaccharides, or both. These include multiple intracellular exo-acting glycoside hydrolase (GH) including but not limited to Bga2A, Hex1, Hex2, NanH2, BiAfcA, BiAfcB (SEQ ID NOS 18-SEQ ID NOS 23 respectively). In some aspects, the DNA sequence may comprise polynucleotide sequences from any or all of the elements of the Ngl cluster. In some aspects the DNA sequence may comprise one or more polynucleotide sequences that are at least 60% (e.g., at least 65%, 70%, 75%, 80%, 85%, 90%, 95%, 97%, 98%, 99%, or 100%) identical to any of SEQ ID NOS. 18-23. In some aspects the DNA sequence may comprise one or more polynucleotide sequences that are at least about 60% to about 70%, or about 70% to about 80%, or about 80% to about 90%, or about 90% to about 100% identical to any of SEQ ID NOS. 18-23. In some aspects the DNA sequence may comprise additional polynucleotide sequences that are known to or predicted to enhance uptake, or utilization or both, of N-glycans, or plant derived polysaccharides.

In some aspects, the isolated strain as disclosed herein may comprise at least 2, at least 3, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to any of SEQ ID NOs. 2-23. In some aspects, the strain comprises one of more polynucleotide sequences that are at least 60% (e.g., at least 65%, 70%, 75%, 80%, 85%, 90%, 95%, 97%, 98%, 99%, or 100%) identical to any of SEQ ID NOS. 2-23. In some aspects, the current disclosure encompasses an isolated strain comprising a DNA sequence at least 60% identical to a DNA sequence from the genome of the isolated B. infantis strain (NRRL deposit no. xxxx, genome assembly available at European Nucleotide Archive under study accession number PRJEB45396), but absent from the genomes of related Bifidobacterium isolates.

In some aspect the isolated strain may be Bifidobacterium longum subspecies infantis (B. infantis) ID number Bg40721_2D9_SN_2018. A genome assembly of this strain is available in the European Nucleotide Archive under study accession number PRJEB45396 and a type strain is available at Professor Jeffery I. Gordon's laboratory at Washington University, School of Medicine at St. Louis. Additionally, the strain will be deposited to the ARS Culture Collection (NRRL): deposit #XXXX. In one aspect, the current disclosure encompasses an isolated strain of Bifidobacterium longum subsp. infantis comprising a genome sequence at least 95%, or at least 96%, or at least 97%, or at least 98%, or at least 99%, identical to the genome sequence as provided in European Nucleotide Archive under study accession number PRJEB45396.

In some aspects, the current disclosure also encompasses an engineered strain of Bifidobacterium comprising a DNA sequence as disclosed herein. In some aspects, the strain of B. infantis is an engineered strain of B. infantis ATCC 15697. In some aspects, the strain of B. infantis is an engineered strain of B. infantis EVC001. In some aspects, the engineered strain may comprise one or more polynucleotide sequences comprising any of SEQ ID NOs. 2-23. In some aspects, the engineered strain may comprise at least 2, at least 3, at least 4, at least 5, at least 6, at least 7, at least 8, at least 9, at least 10, at least 11, at least 12, at least 13, at least 14, at least 15, at least 16, at least 17, at least 18 at least 19 or at least 20 of SEQ ID NOs. 2-23. In some aspects, the engineered strain may comprise at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences that are at least 60% (e.g., at least 65%, 70%, 75%, 80%, 85%, 90%, 95%, 97%, 98%, 99%, or 100%) identical to any of SEQ ID NOS. 2-23. In some aspects, the engineered strain may comprise additional polynucleotide sequences that are known to or predicted to enhance uptake, or utilization, or both of N-glycans, or plant derived polysaccharides, or both. In some aspects, the engineered strain comprises a DNA sequence at least 60% identical to a DNA sequence from the genome of the isolated B. infantis strain (NRRL deposit no. xxxx), but absent from the genomes of related Bifidobacterium isolates. A genome assembly of this strain is available in the European Nucleotide Archive under accession number PRJEB45396.

In some aspects, the one or more DNA sequences in the isolated or engineered strain may be operably linked to regulatory sequences comprising but not limited to promoters, terminators, enhancer elements that may increase or decrease the expression of the DNA sequence. In some aspects, the regulatory polynucleotide sequence may be endogenous to the host B. infantis strain. In some aspects, the regulatory sequence may be endogenous to B. infantis (deposit no. xxxx). In some aspects, the regulatory sequence can be heterologous to the strain. In some aspects the regulatory element may be an artificially designed sequence. In some aspects the regulatory element may be from another species, the sequence capable of changing the expression of the DNA sequence.

ii. Formulations

The term formulation as used herein, can refer to any composition comprising at least a strain of Bifidobacterium longum subspecies infantis (B. infantis) comprising at least a DNA sequence from Bifidobacterium longum subspecies infantis NRRL deposit #XXXX characterized to enhance uptake, or utilization, or both of N-glycans, or plant derived polysaccharides, or both. A genome assembly of this strain is available in the European Nucleotide Archive under accession number PRJEB45396. In one aspect, the current disclosure encompasses a formulation comprising an isolated strain of Bifidobacterium longum subsp. infantis comprising a genome sequence at least 95%, or at least 96%, or at least 97%, or at least 98%, or at least 99%, or more identical to the genome sequence as provided in European Nucleotide Archive under study accession number PRJEB45396.

In some aspects, the current disclosure encompasses a formulation comprising a therapeutically effective quantity of a strain of B. infantis comprising at least one DNA sequence as disclosed above for enhanced uptake or utilization of N-glycans or plant derived polysaccharides. In some aspects, the formulation may comprise an isolated or an engineered strain comprising one or more polynucleotide sequences comprising any of SEQ ID NOs. 2-23. In some aspects, the formulation may comprise a strain comprising at least 2, at least 3, at least 4, at least 5, at least 6, at least 7, at least 8, at least 9, at least 10, at least 11, at least 12, at least 13, at least 14, at least 15, at least 16, at least 17, at least 18 at least 19 or at least 20 of SEQ ID NOs. 2-23. In some aspects, the formulation may comprise a strain comprising at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences that are at least 60% (e.g., at least 65%, 70%, 75%, 80%, 85%, 90%, 95%, 97%, 98%, 99%, or 100%) identical to any of SEQ ID NOS. 2-23. In some aspects, the engineered strain may comprise additional polynucleotide sequences that are known to or predicted to enhance uptake, or utilization or both, of N-glycans, or plant derived polysaccharides, or both.

In some aspect the formulations may comprise Bifidobacterium longum subspecies infantis (B. infantis) lab ID number Bg40721_2D9_SN 2018. A genome assembly of this strain is available in the European Nucleotide Archive under study accession number PRJEB45396 and a type strain is available at Professor Jeffery I. Gordon's laboratory at Washington University, School of Medicine at St. Louis. In one aspect, the current disclosure encompasses a formulation comprising an isolated strain of Bifidobacterium longum subsp. infantis comprising a genome sequence at least 95%, or at least 96%, or at least 97%, or at least 98%, or at least 99%, or 100% identical to the genome sequence as provided in European Nucleotide Archive under study accession number PRJEB45396. Additionally, the strain will be deposited to the ARS Culture Collection (NRRL) and can be identified using the NRRL deposit #XXXX.

In some aspects, the formulation may comprise an engineered strain of Bifidobacterium longum subspecies infantis ATCC 15697 comprising at least one DNA sequence from Bifidobacterium longum subspecies infantis of NRRL deposit no. #XXXX that enhances uptake of N-glycans, plant derived polysaccharides, or both; or enhances utilization of N-glycans, plant derived polysaccharides, or both, compared to a strain of the same background without the at least one DNA sequence. In some aspects, the formulation comprises an engineered strain of Bifidobacterium longum subspecies infantis EVC001 comprising at least one DNA sequence from Bifidobacterium longum subspecies infantis of NRRL deposit no. #XXXX that enhances uptake of N-glycans, plant derived polysaccharides, or both; or enhances utilization of N-glycans, plant derived polysaccharides, or both, compared to a strain of the same background without the at least one DNA sequence.

In some aspects, the formulation comprises more than about 10², or more than about 10³, or more than about 10⁵, or more than about 10⁷, or more than about 10⁹, or more than about 10¹¹, or more than about 10¹³ cfu per gram of B. infantis ID number Bg40721_2D9_SN_2018 (NRRL deposit #XXXX). In some aspects, the formulation may comprise more than about 10², or more than about 10³, or more than about 10⁵, or more than about 10⁷, or more than about 10⁹, or more than about 10¹¹, or more than about 10¹³ cfu of per gram of an isolated B. infantis strain as disclosed herein. In some aspects, the formulation may comprise more than about 10², or more than about 10³, or more than about 10⁵, or more than about 10⁷, or more than about 10⁹, or more than about 10¹¹, or more than about 10¹³ cfu of per gram of an engineered B. infantis strain as disclosed herein. In some aspects, the formulation may comprise more than about 10², or more than about 10³, or more than about 10⁵, or more than about 10⁷, or more than about 10⁹, or more than about 10¹¹, or more than about 10¹³ cfu per gram of a combination of strains of B. infantis comprising at least one of the DNA sequences as disclosed herein.

In some aspects, the formulation may comprise viable B. infantis cells. In some aspects, the formulation may comprise a mixture of viable and non-viable cells.

In some aspects the formulation may further comprise additional strains thus forming a mixture of probiotic strains. As used herein, the term “probiotic” refers to any live microorganism which when administered to a subject in adequate amounts confers a health benefit. In some aspect the probiotic microorganism is an isolated or engineered strain of B. infantis. In some aspects the additional probiotic strains may include one of more of naturally occurring or engineered strains particular but non-limiting examples of which include Arthrobacter agilis, Arthrobacter citreus, Arthrobacter globiformis, Arthrobacter leuteus. Arthrobacter simplex, Azotobacter chroococcum, Azotobacter paspali, Azospirillum brasiliencise, Azospriliium lipoferum, Bacillus brevis, Bacillus macerans, Bacillus pumilus, Bacillus polymyxa, Bacillus subtilis, Bacteroides lipolyticum, Bacteroides succinogenes, Brevibacterium lipolyticum, Brevibacterium stationis, Bacillus laterosporus, Bacillus bifidum, Bacillus laterosporus, Bifidophilus infantis, Streptococcus thermophilous, Bifidophilus longum, Bifidobacteria animalis, Bifidobacteria bifidus, Bifidobacteria breve, Bifidobacteria longum, Kurtha zopfil, Lactobacillus paracasein, Lactobacillus acidophilus, Lactobacillus planetarium, Lactobacillus salivarius, Lactobacillus rueteri, Lactobacillus bulgaricus, Lactobacillus helveticus, Lactobacillus casei, Lactobacillus rhamnosus. Lactobacillus sporogenes, Lactococcus lactis, Myrothecium verrucaris, Prevotella spp., Prevotella copri, Pseudomonas calcis, Pseudomonas dentrificans, Pseudomonas flourescens, Pseudomonas glathei, Phanerochaete chrysosporium, Saccharomyces boulardii, Streptmyces fradiae, Streptomyces cellulosae, Stretpomyces griseoflavus and combinations thereof.

In some aspects the formulation may comprise a viable mixture of probiotic cells. In some aspects the formulation may comprise non-viable mixture of probiotic cells. In some aspects the formulation may comprise a mixture of viable and non-viable mixture of pro-biotic cells.

In some aspects the formulation may further comprise an ingestible carrier, prebiotic material, an excipient, an adjuvant, stabilizers, a biological compound, dietary supplements, proteins, a vitamin, a drug, a vaccine or a combination thereof. Non-limiting examples of ingestible carriers include milk components, baby formula, baby food including but not limited to F-75 or F-100 formulas used for the management of malnutrition, human milk oligosaccharides, breast milk, sugar, flavor enhancers. “Prebiotic” means one or more non-digestible food substance that promotes the growth of health beneficial micro-organisms, or probiotics in the intestines. They are not broken down in the stomach, or upper intestine or absorbed in the GI tract of the person ingesting them, but they are fermented by the gastrointestinal microbiota or by probiotics. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof. Non-limiting examples of proteins include dairy based proteins, plant-based proteins, animal-based proteins and artificial proteins. Dairy based proteins include, for example, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, folic acid and biotin) essential in minute amounts for normal growth and activity of the body and obtained naturally from plant and animal foods or synthetically made, pro-vitamins, derivatives, analogs. Non-limiting examples of excipients include binders, emulsifiers, diluents, fillers, disintegrants, effervescent disintegration agents, preservatives, antioxidants, flavor-modifying agents, lubricants and glidants, dispersants, coloring agents, pH modifiers, chelating agents, and release-controlling polymers. Non-limiting list of adjuvants include potassium alum, aluminum hydroxide, aluminum phosphate, calcium phosphate hydroxide, paraffin oil, adjuvant 65, killed bacteria of the species Bordetella pertussis, Mycobacterium bovis, toxoids, plant saponins from quillaja and soybean, cytokines: IL-1, IL-2, IL-1, Freund's complete adjuvant, Freund's incomplete adjuvant and squalene.

In some aspects, the strains of the current disclosure can be formulated for any route of administration, for example oral, gastric, orogastric, nasogastric, implanted, buccal, and rectal.

A strain of the disclosure, or a combination of strains of the disclosure, may be formulated in unit dosage form as a solid, semi-solid, liquid, capsule, powder, emulsions, suspensions, tablets and suitably packaged. In some aspects, the formulations disclosed herein may be encapsulated. These formulations are a further aspect of the invention. In some aspect the formulations may be mixed with liquids for suitable for orogastric or nasogastric delivery. Usually, the amount of a strain of the invention, or a combination of strains of the invention, is between 0.1-95% by weight of the formulation, or between 0.1-1% or 1%-10% or 10%-20%, or 20%-30%, or 30%-40%, or 40%-50%, or 50%-60%, or 60%-70%, or 70%-80% or 80%-90% or 90%-99% by weight of the formulation. Methods of formulating compositions are discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

iii. Combinations

In some aspects, the current disclosure also encompasses combinations of a therapeutically effective quantity of a strain of Bifidobacterium longum subspecies infantis comprising at least one DNA sequence from Bifidobacterium longum subspecies infantis of deposit no. xxxxx (NRRL) (genome assembly available at the European Nucleotide Archive under study accession number PRJEB45396) for enhanced uptake, or utilization or both of N-glycans, or plant derived polysaccharides or both, as disclosed herein and a food formulation comprising at least one carbohydrate that can be metabolized by members of the gut microbiota. Exemplary food formulations or compositions suitable for use may be disclosed in US 2022/0312817, the entire contents of which are hereby incorporated by reference.

In some aspects, the combinations as disclosed herein may be formulated as a single formulation comprising both, a formulation comprising a strain of B. infantis comprising at least one DNA sequence as disclosed herein and a food formulation comprising at least one carbohydrate that can be metabolized by members of the gut microbiota. In some aspects, the combinations as disclosed herein may be formulated separately, with a formulation comprising an isolated or engineered strain as disclosed herein and a second separate formulation comprising a food formulation comprising at least one carbohydrate that can be metabolized by members of the gut microbiota. The separate formulation could then be administered simultaneous, or the administration may be staggered to maximize benefits.

In some aspects, the food formulation as disclosed herein is an edible composition that impacts the subject's gut microbiota in a manner to modulate expression of nucleic acids encoding proteins in particular enzyme families, such that physiological parameters of the subject are improved, e.g., ponderal growth or rate of ponderal growth. Components of the food formulation and some exemplary formulations are provided below.

### (a) Food Formulation Comprising Chickpea Flour, Peanut Flour, Soy Flour, Raw Banana

In one aspect, a food formulation of the present disclosure comprises chickpea flour, peanut flour, soy flour, and raw banana, wherein the chickpea flour, the peanut flour, the soy flour, and the raw banana provide at least 8.5 g of protein per 100 g of the food formulation. In preferred aspects, the food formulation contains no cow's milk or powdered cow's milk, or no milk or powdered milk of any kind, or no milk, powdered milk, or milk product of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (a) no seed, nuts, and nut butter, and/or (b) no cocoa nibs, cocoa powder or chocolate, and/or (c) no rice flour and lentil flour, and/or (d) no dried fruit. In another example, food formulations of the present disclosure comprising chickpea flour, peanut flour, soy flour, and raw banana may contain no milk or powdered milk of any kind and (a) no seed, nuts, and nut butter, and/or (b) no cocoa nibs, cocoa powder or chocolate, and/or (c) no rice flour and lentil flour, and/or (d) no dried fruit.

In some aspects, the chickpea flour, the peanut flour, the soy flour, and the raw banana, in total, provide 8.5 g to about 15 g of protein per 100 g of the food formulation. In some aspects, the chickpea flour, the peanut flour, the soy flour, and the raw banana, in total, provide about 9 g to about 15 g of protein per 100 g of the food formulation. In some aspects, the chickpea flour, the peanut flour, the soy flour, and the raw banana, in total, provide about 10 g to about 15 g of protein per 100 g of the food formulation. In some aspects, the chickpea flour, the peanut flour, the soy flour, and the raw banana, in total, provide about 11 g to about 15 g of protein per 100 g of the food formulation. In some aspects, the chickpea flour, the peanut flour, the soy flour, and the raw banana, in total, provide about 9 g to about 12 g of protein per 100 g of the food formulation. In some aspects, the chickpea flour, the peanut flour, the soy flour, and the raw banana, in total, provide about 10 g to about 12 g of protein per 100 g of the food formulation. In some aspects, the chickpea flour, the peanut flour, the soy flour, and the raw banana, in total, provide about 11 g to about 12 g of protein per 100 g of the food formulation. In some aspects, the chickpea flour, the peanut flour, the soy flour, and the raw banana, in total, provide about 12 g to about 15 g of protein per 100 g of the food formulation. In some aspects, the chickpea flour, the peanut flour, the soy flour, and the raw banana, in total, provide about 12 g to about 14 g of protein per 100 g of the food formulation. In some aspects, the chickpea flour, the peanut flour, the soy flour, and the raw banana, in total, provide about 13 g to about 15 g of protein per 100 g of the food formulation. In other aspects, the chickpea flour, the peanut flour, the soy flour, and the raw banana, in total, provide 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, or about 15 g of protein per 100 g of the food formulation.

In each of the above aspects, the weight ratio of the chickpea flour to the peanut flour to the soy flour to the raw banana may vary. Typically, chickpea flour has about 20% protein by weight, peanut flour has about 50% protein by weight, soy flour has about 50% protein by weight, and raw banana has about 1% protein by weight. The weight percentages of protein in each ingredient may vary however, depending upon the varietal of plant and, in the case of the flours, the method used to manufacture the flour. In some aspects, the weight ratio is about 1:about 1:about 0.8: about 1.9, respectively (chickpea flour: peanut flour: soy flour: raw banana), or a weight ratio adjusted as needed to reflect differences in the ingredients.

In an exemplary aspect, a food formulation of the present disclosure comprises about 9-11 g of chickpea flour, about 9-11 g of peanut flour, about 7-9 g of soy flour, and about 17-21 g of raw banana. In preferred aspects, the food formulation contains no cow's milk or powdered cow's milk, or no milk or powdered milk of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (i) no seed, nuts, and nut butter, and/or (ii) no cocoa nibs, cocoa powder or chocolate, and/or (iii) no rice flour and lentil flour, and/or (iv) no dried fruit. In another example, food formulations of the present disclosure comprising chickpea flour, peanut flour, soy flour, and raw banana may contain no milk or powdered milk of any kind and (i) no seed, nuts, and nut butter, and/or (ii) no cocoa nibs, cocoa powder or chocolate, and/or (iii) no rice flour and lentil flour, and/or (iv) no dried fruit.

In another exemplary aspect, a food formulation of the present disclosure comprises about 10 g of chickpea flour, about 10 g of peanut flour, about 8 g of soy flour, and about 19 g of raw banana. In preferred aspects, the food formulation contains no cow's milk or powdered cow's milk, or no milk or powdered milk of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (i) no seed, nuts, and nut butter, and/or (ii) no cocoa nibs, cocoa powder or chocolate, and/or (iii) no rice flour and lentil flour, and/or (iv) no dried fruit. In another example, food formulations of the present disclosure comprising chickpea flour, peanut flour, soy flour, and raw banana may contain no milk or powdered milk of any kind and (i) no seed, nuts, and nut butter, and/or (ii) no cocoa nibs, cocoa powder or chocolate, and/or (iii) no rice flour and lentil flour, and/or (iv) no dried fruit.

### (b) Food Formulation Comprising Glycan Equivalents of Chickpea Flour, Peanut Flour, Soy Flour, Raw Banana

In another aspect, a food formulation of the present disclosure is a food formulation of (a), wherein some or all the chickpea flour, the peanut flour, the soy flour, and/or the raw banana is replaced with a glycan equivalent thereof. As used herein, a “glycan equivalent” refers to a food formulation with a similar glycan content. The term “similar” generally refers to a range of numerical values, for instance, ±0.5-1%, ±1-5% or ±5-10% of the recited value, that one would consider equivalent to the recited value, for example, having the same function or result. Because a glycan equivalent has a similar glycan content to the ingredient it is replacing, it may be substituted about 1:1. For instance, if 3 g of chickpea flour is to be replaced with a glycan equivalent thereof, one of skill in the art would use about 3 g of the chickpea glycan equivalent. A glycan equivalent may be defined in terms of its monosaccharide content and optionally by an analysis of the glycosidic linkages. Methods for measuring monosaccharide content and analyzing glycosidic linkages are known in the art.

In some aspects, some or all the chickpea flour is replaced with a glycan equivalent of chickpea flour. For instance, a food formulation of (a) may comprise a glycan equivalent of about 0.5 g or more of chickpea flour. In another example, a food formulation of (a) may comprise a glycan equivalent of about 1 g, about 2 g, about 3 g, about 4 g, about 5 g, about 6 g, about 7 g, about 8 g, about 9 g, or about 10 g of chickpea flour. In another example, a food formulation of (a) may comprise a glycan equivalent of about 0.1 g to about 10 g of chickpea flour, or about 0.5 to about 5 g of chickpea flour. In another example, a food formulation of (a) may comprise a glycan equivalent of about 1 g to about 10 g of chickpea flour, or about 1 g to about 5 g of chickpea flour, or about 2.5 g to about 7.5 g of chickpea flour, to about 5 g to about 10 g of chickpea flour. In further aspects, some or all the peanut flour is also replaced with a glycan equivalent of peanut flour, some or all the soy flour is also replaced with a glycan equivalent of soy flour, and/or some or all the raw banana is also replaced with a glycan equivalent of raw banana.

In some aspects, some or all the peanut flour is replaced with a glycan equivalent of peanut flour. For instance, a food formulation of (a) may comprise a glycan equivalent of about 0.5 g or more of peanut flour. In another example, a food formulation of (a) may comprise a glycan equivalent of about 1 g, about 2 g, about 3 g, about 4 g, about 5 g, about 6 g, about 7 g, about 8 g, about 9 g, or about 10 g of peanut flour. In another example, a food formulation of Section I (a) may comprise a glycan equivalent of about 0.1 g to about 10 g of peanut flour, or about 0.5 to about 5 g of peanut flour. In another example, a food formulation of (a) may comprise a glycan equivalent of about 1 g to about 10 g of peanut flour, or about 1 g to about 5 g of peanut flour, or about 2.5 g to about 7.5 g of peanut flour, to about 5 g to about 10 g of peanut flour. In further aspects, some or all the chickpea flour is also replaced with a glycan equivalent of chickpea flour, some or all the soy flour is also replaced with a glycan equivalent of soy flour, and/or some or all the raw banana is also replaced with a glycan equivalent of raw banana.

In some aspects, some or all the soy flour is replaced with a glycan equivalent of soy flour. For instance, a food formulation of (a) may comprise a glycan equivalent of about 0.5 g or more of soy flour. In another example, a food formulation of (a) may comprise a glycan equivalent of about 1 g, about 2 g, about 3 g, about 4 g, about 5 g, about 6 g, about 7 g, or about 8 g of soy flour. In another example, a food formulation of (a) may comprise a glycan equivalent of about 0.1 g to about 8 g of soy flour, or about 0.5 to about 5 g of soy flour. In another example, a food formulation of (a) may comprise a glycan equivalent of about 1 g to about 8 g of soy flour, or about 1 g to about 4 g of soy flour, or about 2 g to about 6 g of soy flour, to about 4 g to about 8 g of soy flour. In further aspects, some or all the chickpea flour is also replaced with a glycan equivalent of chickpea flour, some or all the peanut flour is also replaced with a glycan equivalent of peanut flour, and/or some or all the raw banana is also replaced with a glycan equivalent of raw banana.

In some aspects, some or all the raw banana is replaced with a glycan equivalent of raw banana. For instance, a food formulation of (a) may comprise a glycan equivalent of about 0.5 g or more of raw banana. In another example, a food formulation of (a) may comprise a glycan equivalent of about 1 g, about 2 g, about 3 g, about 4 g, about 5 g, about 6 g, about 7 g, about 8 g of raw banana, about 9 g of raw banana, about 10 g of raw banana, about 11 g of raw banana, about 12 g of raw banana, about 13 g of raw banana, about 14 g of raw banana, about 15 g of raw banana, about 16 g of raw banana, about 17 g of raw banana, about 18 g of raw banana, or about 19 g of raw banana. In another example, a food formulation of (a) may comprise a glycan equivalent of about 0.1 g to about 8 g of raw banana, or about 0.5 to about 5 g of raw banana. In another example, a food formulation of (a) may comprise a glycan equivalent of about 1 g to about 8 g of raw banana, or about 1 g to about 4 g of raw banana, or about 2 g to about 6 g of raw banana, to about 4 g to about 8 g of raw banana. In further aspects, some or all the chickpea flour is also replaced with a glycan equivalent of chickpea flour, some or all the peanut flour is also replaced with a glycan equivalent of peanut flour, and/or some or all the soy flour is also replaced with a glycan equivalent of soy flour.

### (c) Micronutrient Premix

A micronutrient premix in a food formulation of the present disclosure is present in an amount that provides at least 60% of the recommended daily allowance (RDA), for a given age group, of minimally vitamin A, vitamin C, vitamin D, vitamin E, vitamin B, calcium, copper, iron, magnesium, manganese, phosphorus, potassium, and zinc. The RDA of vitamin A, vitamin C, vitamin D, vitamin E, vitamin B, calcium, copper, iron, magnesium, manganese, phosphorus, potassium, and zinc, for various age groups, is known in the art. Given that different age groups may have different RDA's, it will be appreciated by a person of skill in the art that certain food formulations may not be suitable for subjects of all ages. For example, a food formulation with 60% of the Vitamin C RDA for a subject 7-12 months in age (e.g., 40 mg) will not contain at least 60% of the Vitamin C RDA for a subject 21 years of age (e.g., 75-90 mg). The term “vitamin “B,” as used herein, is inclusive of all B vitamins, unless otherwise specified. Although food formulations of the present disclosure are described as comprising a micronutrient premix, the addition of each vitamin and mineral separately, or the use of multiple premixes, is also contemplated and encompassed by the aspects described herein. Similarly, in alternative aspects, the micronutrient premix can be formulated separately and administered as a distinct food formulation in conjunction with a food formulation comprising chickpea flour or a glycan equivalent thereof, peanut flour or a glycan equivalent thereof, soy flour or a glycan equivalent thereof, raw banana or a glycan equivalent thereof.

In various aspects, a micronutrient premix provides at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% of the recommended daily allowance (RDA), for a given age group, of minimally vitamin A, vitamin B, vitamin C, vitamin D, vitamin E, calcium, copper, iron, magnesium, manganese, phosphorous, potassium and zinc. In certain aspects, a micronutrient premix provides more than 100% of the RDA, for a given age group, of minimally vitamin A, vitamin B, vitamin C, vitamin D, vitamin E, calcium, copper, iron, magnesium, manganese, phosphorous, potassium and zinc. In a specific aspect, the micronutrient premix provides at least 75% of the recommended daily allowance (RDA), for a given age group, of minimally vitamins A, C, D and E, all B vitamins, calcium, copper, iron, magnesium, manganese, phosphorous, potassium and zinc. The RDA of vitamins and minerals for different age groups is well known in the art.

In a specific aspect, a micronutrient premix provides at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 77%, at least 78%, at least 79%, or at least 80% of the recommended daily allowance (RDA) for children aged 12-24 months of vitamin A, vitamin B, vitamin C, vitamin D, vitamin E, calcium, copper, iron, magnesium, manganese, phosphorous, potassium and zinc.

In another specific aspect, the micronutrient premix provides at least 70% of the recommended daily allowance (RDA) for children aged 12-24 months of minimally vitamin A, vitamin B, vitamin C, vitamin D, vitamin E, calcium, copper, iron, magnesium, manganese, phosphorous, potassium and zinc.

In another specific aspect, the micronutrient premix provides at least 75% of the recommended daily allowance (RDA) for children aged 12-24 months of minimally vitamin A, vitamin B, vitamin C, vitamin D, vitamin E, calcium, copper, iron, magnesium, manganese, phosphorous, potassium and zinc.

A micronutrient premix may further comprise vitamins and minerals in addition to the vitamin A, vitamin B, vitamin C, vitamin D, vitamin E, calcium, copper, iron, magnesium, manganese, phosphorous, potassium and zinc.

In an exemplary aspect, a food formulation of the present disclosure contains vitamin A, vitamin C, vitamin D, vitamin E, vitamin B, calcium, copper, iron, magnesium, phosphorus, potassium, and zinc in the amounts listed in Table A and Table B. In a preferred aspect, a food formulation of the present disclosure contains the nutrients of Table A in the amounts listed in Table A. In another preferred aspect, a food formulation of the present disclosure contains the nutrients of Table B in the amounts listed in Table B. In yet another preferred aspect, a food formulation of the present disclosure contains the nutrients of both Table A and Table B, in the amounts listed in Table A and Table B respectively.

TABLE A
Vitamin Premix

| | | | Units of |
|---|---|---|---|
| | | | Measurement per |
| | Minimum | Maximum | gram of the |
| Nutrients | Amount | Amount | Vitamin Premix |
| Vitamin A | 12655.013 | 16170.294 | IU |
| Thiamine Mononitrate | 6.765 | 8.644 | mg |
| Vitamin B12 | 11.700 | 17.550 | mcg |
| Vitamin B2 - Riboflavin | 5.485 | 7.008 | mg |
| Pyridoxine Hydrochloride | 6.153 | 7.863 | mg |
| Vitamin C | 236.250 | 301.875 | mg |
| Sodium | 29.213 | 37.327 | mg |
| Calcium D-Pantothenate | 20.798 | 26.574 | mg |
| Vitamin D3 | 7593.960 | 9703.599 | IU |
| Vitamin E (as E Acetate) | 120.690 | 154.215 | IU |
| Folic acid | 2531.007 | 3234.065 | mcg |
| Vitamin K1 | 405.009 | 584.991 | mcg |
| Niacinamide | 60.750 | 77.625 | mg |

For a 100 g food formulation, 160 mg of the Vitamin Premix is used. Accordingly, to calculate the amount of a given mineral in a 100 g food formulation, the amounts listed above are multiplied by 160.

In an exemplary aspect, a food formulation of the present disclosure contains the micronutrients in Table B, in the amounts in Table B.

TABLE B
Mineral Premix

| | Minimum | Maximum | Units of Measurement per |
|---|---|---|---|
| Nutrients | Amount | Amount | gram of the mineral premix |
| Calcium | 170.000 | 216.000 | Mg |
| Phosphorus | 93.000 | 118.000 | Mg |
| Calcium | 0.000 | 0.000 | Q.S. |
| Copper | 0.181 | 0.231 | Mg |
| Iodine | 52.945 | 67.652 | Mcg |
| Iron | 3.169 | 4.049 | Mg |
| Magnesium | 27.163 | 34.708 | mg |
| Manganese | 0.543 | 0.694 | mg |
| Potassium (K) | 89.342 | 114.159 | Mg |
| Selenium | 11.770 | 15.040 | Mcg |
| Zinc | 2.415 | 3.085 | Mg |

For a 100 g food formulation, 2.982 g of the Mineral Premix is used. Accordingly, to calculate the amount of a given mineral in a 100 g food formulation, the amounts listed above are multiplied by 2.982.

### (d) Macronutrient Content

In each of the aforementioned aspects, a food formulation may comprise about 300 kcal to about 560 kcal per 100 g of the food formulation, a protein energy ratio (PER) of about 8% to about 20%, and a fat energy ratio (FER) of about 30% to about 60%. In some aspects, a food formulation may comprise about 350 kcal to about 560 kcal per 100 g of the food formulation, a protein energy ratio (PER) of about 8% to about 20%, and a fat energy ratio (FER) of about 30% to about 60%. In other aspects, a food formulation may comprise about 400 kcal to about 560 kcal per 100 g of the food formulation, a protein energy ratio (PER) of about 8% to about 12%, and a fat energy ratio (FER) of about 45% to about 60%. In an exemplary aspect, a food formulation may comprise about 400 to about 560 kcal per 100 g of the food formulation, about 20 g to about 36 g of fat per 100 g of the food formulation, about 11 g to about 16 g of protein per 100 g of the food formulation, a protein energy ratio (PER) of about 8% to about 12%, and a fat energy ratio (FER) of about 45% to about 60%. Carbohydrates and sugars may provide the remainder of the energy content. For instance, if a food formulation has a PER of 10% and a FER of 50%, then the carbohydrate+sugar-to-energy ratio may be 40%.

In one aspect, a food formulation of the disclosure provides about 300 kcal, about 310 kcal, about 320 kcal, about 330 kcal, about 340 kcal, or about 350 kcal per 100 g of the food formulation. In another aspect, a food formulation of the disclosure provides about 350 kcal, about 360 kcal, about 370 kcal, about 380 kcal, about 390 kcal, or about 400 kcal per 100 g of the food formulation. In another aspect, a food formulation of the disclosure provides about 400 kcal, about 410 kcal, about 420 kcal, about 430 kcal, about 440 kcal, or about 450 kcal per 100 g of the food formulation. In another aspect, a food formulation of the disclosure provides about 460 kcal, about 470 kcal, about 480 kcal, about 490 kcal, or about 500 kcal per 100 g of the food formulation. In another aspect, a food formulation of the disclosure provides about 500 kcal, about 510 kcal, about 520 kcal, about 530 kcal, about 540 kcal, about 550 kcal, or about 560 kcal per 100 g of the food formulation. In another aspect, a food formulation of the disclosure provides about 400 kcal to about 560 kcal, about 420 kcal to about 560 kcal, about 440 kcal to about 560 kcal, about 460 kcal to about 560 kcal, about 480 kcal to about 560 kcal or about 500 kcal to about 560 kcal per 100 g of the food formulation. In another aspect, a food formulation of the disclosure provides about 300 kcal to about 450 kcal per 100 g of the food formulation. In another aspect, a food formulation of the disclosure provides about 300 kcal to about 425 kcal per 100 g of the food formulation. In another aspect, a food formulation of the disclosure provides about 300 kcal to about 400 kcal per 100 g of the food formulation. In another aspect, a food formulation of the disclosure provides about 300 kcal to about 350 kcal per 100 g of the food formulation. In another aspect, a food formulation of the disclosure provides about 350 kcal to about 450 kcal per 100 g of the food formulation. In another aspect, a food formulation of the disclosure provides about 350 kcal to about 400 kcal per 100 g of the food formulation. In another aspect, a food formulation of the disclosure provides about 325 kcal to about 425 kcal per 100 g of the food formulation. In another aspect, a food formulation of the disclosure provides about 400 kcal to about 500 kcal per 100 g of the food formulation, about 420 kcal to about 500 kcal per 100 g of the food formulation, about 440 kcal to about 500 kcal per 100 g of the food formulation, about 460 kcal to about 500 kcal per 100 g of the food formulation, or about 480 kcal to about 500 kcal per serving 100 g of the food formulation. In still another aspect, a food formulation of the disclosure provides about 400 kcal to about 480 kcal per 100 g of the food formulation, about 400 kcal to about 460 kcal per 100 g of the food formulation, or about 400 kcal to about 440 kcal per 100 g of the food formulation. In another aspect, a food formulation of the present disclosure provides about 400 kcal to about 420 kcal, about 400 kcal to about 410 kcal, about 405 kcal to about 415 kcal, or about 410 kcal to about 420 kcal per 100 g of the food formulation. In another aspect, a food formulation of the present disclosure provides about 400 kcal to about 415 kcal, about 400 kcal to about 410 kcal, or about 405 kcal to about 415 kcal per 100 g of the food formulation.

In each of the above aspects, a food formulation may comprise about 11 g, about 12 g, about 13 g, about 14 g, about 15 g, or about 16 g of protein per 100 g of the food formulation. For instance, a food formulation may comprise about 11.1 g, about 11.2 g, about 11.3 g, about 11.4 g, about 11.5 g, about 11.6 g, about 11.7 g, about 11.8 g, about 11.9 g of protein per 100 g of the food formulation. In another example, a food formulation may comprise about 12 g, about 12.1 g, about 12.2 g, about 12.3 g, about 12.4 g, about 12.5 g, about 12.6 g, about 12.7 g, about 12.8 g, about 12.9 g, or about 13 g of protein per 100 g of the food formulation. In another example, a food formulation may comprise about 11 g to about 13 g, about 11 g to about 12.5 g, about 11 g to about 12 g, about 11.5 g to about 13 g, about 11.5 g to about 12.5 g, or about 11.5 g to about 12 g protein per 100 g of the food formulation.

In each of the above aspects, a food formulation may comprise about 20, about 21, about 22, about 23, about 24 or about 25 g of fat per 100 g of the food formulation. In another example, a food formulation may comprise about 26 g, about 27 g, about 28 g, about 29 g, or about 30 g of fat per 100 g of the food formulation. In another example, a food formulation may comprise about 20 g, about 20.1 g, about 20.2 g, about 20.3 g, about 20.4 g, about 20.5 g, about 20.6 g, about 20.7 g, about 20.8 g, about 20.9 g of fat per 100 g of the food formulation. In another example, a food formulation may comprise about 21 g, about 21.1 g, about 21.2 g, about 21.3 g, about 21.4 g, about 21.5 g, about 21.6 g, about 21.7 g, about 21.8 g, about 21.9 g, or about 22 g fat per 100 g of the food formulation. In another example, a food formulation may comprise about 20 g to about 22 g, about 20 g to about 21.5 g, about 20 g to about 21 g, about 20.5 g to about 22 g, about 20.5 g to about 21.5 g, or about 20.5 g to about 21 g fat per 100 g of the food formulation.

As used herein, the term “protein energy ratio” is an expression of the protein content of a food formulation, expressed as the proportion of the total energy provided by the protein content. In each of the above aspects, a food formulation of the disclosure may have a PER of about 8%, about 8.5%, about 9%, about 9.5%, about 10%, about 10.5%, about 11%, about 11.5%, or about 12%. In another example, a food formulation may have a PER of about 11.1%, about 11.2%, about 11.3%, about 11.4%, about 11.5%, about 11.6%, about 11.7%, about 11.8%, or about 11.9%. In another example, a food formulation of the disclosure may have a PER of about 8.5% to about 12%, about 9% to about 12%, about 9.5% to about 12%, about 10% to about 12%, or about 10.5% to about 12%. In another example, a food formulation may have a PER of about 11% to about 12%, about 11.1% to about 12%, about 11.2% to about 12%, about 11.3% to about 12%, about 11.4% to about 12%, about 11.5% to about 12%, about 11.6% to about 12%. In another example, a food formulation may have a PER of about 11% to about 11.6%, about 11.1% to about 11.6%, about 11.2% to about 11.6%, about 11.3% to about 11.6%, or about 11.4% to about 11.6%. In another example, a food formulation may have a PER of about 11% to about 11.8%, about 11.1% to about 11.8%, about 11.2% to about 11.8%, about 11.3% to about 11.8%, or about 11.4% to about 11.8%. In another example, a food formulation may have a PER of about 12%, about 12.5%, about 13%, about 13.5%, about 14%, about 14.5% or about 15%. In another example, a food formulation may have a PER of about 15%, about 15.5%, about 16%, about 16.5%, about 17%, about 17.5%, about 18%, about 18.5%, about 19%, about 19.5%, or about 20%. In another example, a food formulation may have a PER of about 8% to about 20%, about 8% to about 15%, or about 8% to about 12%. In another example, a food formulation may have a PER of about 10% to about 20%, about 10% to about 15%, or about 10% to about 12%. In another example, a food formulation may have a PER of about 12% to about 20%, or about 12% to about 15%

As used herein, the term “fat energy ratio” is an expression of the fat content of a food formulation, expressed as the proportion of the total energy provided by the fat content. In each of the above aspects, a food formulation may have a FER of about 30%, about 31%, about 32%, about 33%, about 34%, or about 35%. In each of the above aspects, a food formulation may have a FER of about 35%, about 36%, about 37%, about 38%, about 39%, or about 40%. In another example, a food formulation may have a FER of about 40%, about 41%, about 42%, about 43%, about 44%, or about 45%. In another example, a food formulation may have a FER of about 45%, about 46%, about 47%, about 48%, about 49%, or about 50%. In another example, a food formulation may have a FER of about 51%, about 52%, about 53%, about 54%, or about 55%. In another example, a food formulation may have a FER of about 56%, about 57%, about 58%, about 59%, or about 60%. In another example, a food formulation may have a FER of about 45.5%, about 45.6%, about 45.7%, about 45.8%, about 45.9%, or about 46%. In another example, a food formulation may have a FER of about 46.1%, about 46.2%, about 46.3%, about 46.4%, about 46.5% about 46.6%, about 46.7%, about 46.8%, about 46.9%. In another example, a food formulation may have a FER of about 47%, about 47.1%, about 47.2% about 47.3%, about 47.4%, about 47.5%, about 47.6%, about 47.7%, about 47.8%, about 47.9%, or about 48%. In another example, a food formulation of the disclosure may have a FER of about 30% to about 50% or about 30% to about 45%. In another example, a food formulation of the disclosure may have a FER of about 30% to about 40% or about 30% to about 35%. In another example, a food formulation of the disclosure may have a FER of about 35% to about 50% or about 35% to about 45%. In another example, a food formulation of the disclosure may have a FER of about 45% to about 55% or about 45% to about 50%. In another example, a food formulation may have a FER of about 46% to about 55% or about 46% to about 50%. In another example, a food formulation may have a FER of about 46% to about 48%, or about 46% to about 47%. In another example, a food formulation of the disclosure may have a FER of about 45.5% to about 48%, about 45.5% to about 47.5%, or about 45.5% to about 47%. In another example, a food formulation of the disclosure may have a FER of about 46% to about 47.5%, or about 46% to about 46.5%.

In each of the above aspects, a food formulation may comprise a varying amount of carbohydrate. In one example, a food formulation may comprise about 15 g, about 15.1 g, about 15.2 g, about 15.3 g, about 15.4 g, or about 15.5 g of carbohydrate per 100 g of the food formulation, excluding added sugar. In another example, a food formulation may comprise about 15.6 g, about 15.7 g, about 15.8 g, about 15.9 g, or about 16 g of carbohydrate per 100 g of the food formulation, excluding added sugar. In one example, a food formulation may comprise about 16 g, about 16.1 g, about 16.2 g, about 16.3 g, about 16.4 g, about 16.5 g, or about 16.6 g of carbohydrate per 100 g of the food formulation, excluding added sugar. In one example, a food formulation may comprise about 16.5 g, about 16.6 g, about 16.7 g, about 16.8 g, about 16.9 g, or about 17 g of carbohydrate per 100 g of the food formulation, excluding added sugar. In one example, a food formulation may comprise about 17.1 g, about 17.2 g, about 17.3 g, about 17.4 g, about 17.5 g, about 17.6 g, about 17.7 g, about 17.8 g, about 17.9 g, about 18 g of carbohydrate per 100 g of the food formulation, excluding added sugar. In one example, a food formulation may comprise about 15 g to about 18 g, about 15 g to about 17.5 g, about 15 g to about 17 g, or about 15 g to about 16.5 g of carbohydrate per 100 g of the food formulation, excluding added sugar. In one example, a food formulation may comprise about 15.5 g to about 18 g, about 15.5 g to about 17.5 g, about 15.5 g to about 17 g, about 15.5 g to about 16.5 g of carbohydrate per 100 g of the food formulation, excluding added sugar. In one example, a food formulation may comprise about 16 g to about 18 g, about 16 g to about 17.5 g, about 16 g to about 17 g carbohydrate, excluding added sugar. When added sugar is included in the amount of carbohydrate, the value increases by about 27-28 grams. So, for instance, a food formulation with about 15 g to about 18 g carbohydrate, excluding added sugar, will have about 42 g to about 46 g of carbohydrate per 100 g of the food formulation when sugar is included. The term “total carbohydrate” is used herein to refer to a carbohydrate amount that includes added sugar.

In each of the above aspects, a food formulation may comprise a varying amount of fiber. In one example, a food formulation may comprise about 3.5 g, about 3.6 g, about 3.7 g, about 3.8 g, about 3.9 g, or about 4 g of fiber per 100 g of food formulation. In another example, a food formulation may comprise about 4.1 g, about 4.2 g, about 4.3 g, about 4.4 g, about 4.5 g, about 4.6 g, about 4.7 g, about 4.8 g, or about 4.9 g of fiber per 100 g of food formulation. In another example, a food formulation may comprise about 5 g, about 5.1 g, about 5.2 g, about 5.3 g, about 5.4 g, or about 5.5 g of fiber per 100 g of food formulation. In another example, a food formulation may comprise about 3.5 g to about 5.5 g, about 3.5 g to about 5 g, about 3.5 g to about 4.5 g of fiber per 100 g of food formulation. In another example, a food formulation may comprise about 4 g to about 5.5 g, about 4 g to about 5 g, about 4 g to about 4.5 g, about 4.5 g to about 5.5 g, or about 4.5 g to about 5 g of fiber per 100 g of food formulation.

### (e) Additional Ingredients

Food formulations of the present disclosure may further comprise one or more additional ingredient listed in Table C.

TABLE C

| Ingredients | What They Do | Names Found on Product Labels |
|---|---|---|
| Preservatives | Prevent food spoilage from | Ascorbic acid, citric acid, sodium |
| | bacteria, molds, fungi, or yeast | benzoate, calcium propionate, sodium |
| | (antimicrobials); slow or prevent | erythorbate, sodium nitrite, calcium |
| | changes in color, flavor, or | sorbate, potassium sorbate, BHA, BHT, |
| | texture and delay rancidity | EDTA, tocopherols (Vitamin E) |
| | (antioxidants); maintain | |
| | freshness | |
| Sweeteners | Add sweetness with or without | Sucrose (sugar), glucose, fructose, sorbitol, |
| | the extra calories | mannitol, corn syrup, high fructose corn |
| | | syrup, saccharin, aspartame, sucralose, |
| | | acesulfame potassium (acesulfame-K), |
| | | neotame |
| Color Additives | Offset color loss due to exposure | FD&C Blue Nos. 1 and 2, FD&C Green |
| | to light, air, temperature | No. 3, FD&C Red Nos. 3 and 40, FD&C |
| | extremes, moisture and storage | Yellow Nos. 5 and 6, Orange B, Citrus Red |
| | conditions; correct natural | No. 2, annatto extract, beta-carotene, grape |
| | variations in color; enhance | skin extract, cochineal extract or carmine, |
| | colors that occur naturally; | paprika oleoresin, caramel color, fruit and |
| | provide color to colorless and | vegetable juices, saffron (Note: Exempt |
| | “fun” foods | color additives are not required to be |
| | | declared by name on labels but may be |
| | | declared simply as colorings or color |
| | | added) |
| Flavors and | Add specific flavors (natural and | Natural flavoring, artificial flavor, and |
| Spices | synthetic) | spices |
| Flavor Enhancers | Enhance flavors already present | Monosodium glutamate (MSG), |
| | in foods (without providing their | hydrolyzed soy protein, autolyzed yeast |
| | own separate flavor) | extract, disodium guanylate or inosinate |
| Fat Replacers | Provide expected texture and a | Olestra, cellulose gel, carrageenan, |
| (and components | creamy “mouth-feel” in reduced- | polydextrose, modified food starch, |
| of formulations | fat foods | microparticulated egg white protein, guar |
| used to replace | | gum, xanthan gum, whey protein |
| fats) | | concentrate |
| Nutrients | Replace vitamins and minerals | Thiamine hydrochloride, riboflavin |
| | lost in processing (enrichment), | (Vitamin B₂), niacin, niacinamide, folate or |
| | add nutrients that may be lacking | folic acid, beta carotene, potassium iodide, |
| | in the diet (fortification) | iron or ferrous sulfate, alpha tocopherols, |
| | | ascorbic acid, Vitamin D, amino acids (L- |
| | | tryptophan, L-lysine, L-leucine, L- |
| | | methionine, L-cysteine, L-threonine) |
| Emulsifiers | Allow smooth mixing of | Soy lecithin, mono- and diglycerides, egg |
| | ingredients, prevent separation | yolks, polysorbates, sorbitan monostearate |
| | Keep emulsified products stable, | |
| | reduce stickiness, control | |
| | crystallization, keep ingredients | |
| | dispersed, and to help products | |
| | dissolve more easily | |
| Stabilizers and | Produce uniform texture, | Gelatin, pectin, guar gum, carrageenan, |
| Thickeners, | improve “mouth-feel” | xanthan gum, whey |
| Binders, | | |
| Texturizers | | |
| pH Control | Control acidity and alkalinity, | Lactic acid, citric acid, ammonium |
| Agents and | prevent spoilage | hydroxide, sodium carbonate |
| acidulants | | |
| Leavening Agents | Promote rising of baked goods | Baking soda, monocalcium phosphate, |
| | | calcium carbonate |
| Anti-caking | Keep powdered foods free- | Calcium silicate, iron ammonium citrate, |
| agents | flowing, prevent moisture | silicon dioxide |
| | absorption | |
| Humectants | Retain moisture | Glycerin, sorbitol |
| Firming Agents | Maintain crispness and firmness | Calcium chloride, calcium lactate |
| Enzyme | Modify proteins, | Enzymes, lactase, papain, rennet, chymosin |
| Preparations | polysaccharides and fats | |
| Gases | Serve as propellant, aerate, or | Carbon dioxide, nitrous oxide |
| | create carbonation | |

In some aspects, a food formulation further comprises at least one sweetener. In one aspect, a food formulation further comprises sugar (i.e. sucrose), and optionally one or more additional sweetener. The amount of sugar may vary. In one example, a food formulation comprises up to about 30 g of sugar per 100 g of the food formulation. In another example, a food formulation comprises about 0.1 g to about 30 g of sugar, or about 1 g to about 30 g of sugar, per 100 g of the food formulation. In another example, a food formulation comprises about 10 g to about 30 g of sugar per 100 g of the food formulation. In another example, a food formulation comprises about 20 g to about 30 g of sugar per 100 g of the food formulation. In another example, a food formulation comprises about 25 g to about 30 g of sugar per 100 g of the food formulation. In another example, a food formulation comprises about 27 g to about 30 g of sugar, or about 28 g to about 30 g of sugar, per 100 g of the food formulation. In another example, a food formulation comprises about 27 g, 27.1 g, 27.2 g, 27.3 g, 27.4 g, 27.5 g, 27.6 g, 27.7 g, 27.8 g, 27.9 g or 28 g of sugar per 100 g of the food formulation. In another example, a food formulation of the disclosure comprises about 28 g, 28.1 g, 28.2 g, 28.3 g, 28.4 g, 28.5 g, 28.6 g, 28.7 g, 28.8 g, 28.9 g or 29 g of sugar per 100 g of the food formulation. In another example, a food formulation of the disclosure comprises about 29 g, 29.1 g, 29.2 g, 29.3 g, 29.4 g, 29.5 g, 29.6 g, 29.7 g, 29.8 g, 29.9 g or 30 g of sugar per 100 g of the food formulation.

In some aspects, a food formulation further comprises at least one fat. A fat may be an animal fat, or more preferably a vegetable oil. In some aspects, a fat is chosen from avocado oil, canola oil, coconut oil, corn oil, cottonseed oil, flaxseed oil, grape seed oil, hemp seed oil, olive oil, palm oil, peanut oil, rice bran oil, safflower oil, soybean oil, or sunflower oil. In further aspects, one fat provides at least 50% by weight (wt %) of the total fat in the food formulation. For instance, one fat may provide about 50%, about 55%, about 60%, about 65%, about 70%, about 75%, about 80%, about 85%, about 90%, or about 95% by weight of the total fat in the food formulation. In one example the fat is soybean oil. In one example the fat is canola oil. In still further aspects, two or more fats provide at least 50% by weight of the fat in the food formulation. For instance, two or more fats may provide about 50%, about 55%, about 60%, about 65%, about 70%, about 75%, about 80%, about 85%, about 90%, or about 95% by weight of the total fat in the food formulation. In one example, at least one fat is soybean oil or canola oil. In one example, the fat is soybean oil and canola oil.

In other aspects, a food formulation further comprises soybean oil, and the soybean oil provides at least 50% by weight of the total fat in the food formulation. In further aspects, the soybean oil provides at least 75% by weight of the total fat in the food formulation. In still further aspects, the soybean oil provides at least 90% by weight of the total weight of fat in the food formulation. In still further aspects, the soybean oil provides at least 95% by weight of the total fat in the food formulation. In each of the above aspects, the food formulation may further comprise a fat chosen from animal fat or vegetable oil.

In still other aspects, a food formulation further comprises about 20 g of soy bean oil. In one aspect, a food formulation comprises about 15 g, about 16 g, about 17 g, about 18 g, about 19 g, about 20 g, or about 21 g of soybean oil per 100 g of the food formulation. In another aspect, a food formulation further comprises about 15 g to about 21 g, about 16 g to about 21 g, about 17 g to about 21 g, about 18 g to about 21 g, about 19 g to about 21 g, about 20 g to about 21 g, about 15 g to about 20 g, about 16 g to about 20 g, about 17 g to about 20 g, about 18 g to about 20 g, or about 19 g to about 20 g of soybean oil per 100 g of the food formulation. In still another aspect, a food formulation of the disclosure comprises about 17 g, 17.1 g, 17.2 g, 17.3 g, 17.4 g, 17.5 g, 17.6 g, 17.7 g, 17.8 g, 17.9 g or 18 g of soybean oil per 100 g of the food formulation. In still yet another aspect, a food formulation of the disclosure comprises about 18 g, 18.1 g, 18.2 g, 18.3 g, 18.4 g, 18.5 g, 18.6 g, 18.7 g, 18.8 g, 18.9 g or 19 g of soybean oil per 100 g of the food formulation. In still yet another different aspect, a food formulation further comprises about 19 g, 19.1 g, 19.2 g, 19.3 g, 19.4 g, 19.5 g, 19.6 g, 19.7 g, 19.8 g, 19.9 g or 20 g of soybean oil. In a different aspect, a food formulation of the disclosure comprises about 20 g, 20.1 g, 20.2 g, 20.3 g, 20.4 g, 20.5 g, 20.6, 20.7 g, 20.8 g, 20.9 g or 21 g of soybean oil per 100 g of the food formulation.

### (f) Exemplary Food Formulations

In one aspect, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix. In another aspect, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix. In preferred aspects, the micronutrient premix referenced in this paragraph contains the nutrients listed in Table A and Table B in the amount specified in Table A and Table B, respectively.

In some aspects, a food formulation of the present disclosure as described in this section (f), has total protein of about 11.6 g, total fat of about 20.8 g, total carbohydrate of about 46.2 g, and total fiber of about 4.5 g. For example, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g. In another example, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g. In preferred aspects, the micronutrient premix referenced in this paragraph contains the nutrients listed in Table A and Table B in the amount specified in Table A and Table B, respectively.

In exemplary aspects, a food formulation of the present disclosure as described in this section (f), has a protein energy ratio (PER) of about 11.4, a fat energy ratio (FER) of about 46.0, and total calories of about 400 to about 560 kcal per 100 g of the food formulation. For example, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, wherein the food formulation has a protein energy ratio (PER) of about 11.4, a fat energy ratio (FER) of about 46.0, and total calories of about 400 to about 560 kcal per 100 g of the food formulation. In another example, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, wherein the food formulation has a protein energy ratio (PER) of about 11.4, a fat energy ratio (FER) of about 46.0, and total calories of about 400 to about 560 kcal per 100 g of the food formulation. In yet another example, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g, wherein the food formulation has a protein energy ratio (PER) of about 11.4, a fat energy ratio (FER) of about 46.0, and total calories of about 400 to about 560 kcal per 100 g of the food formulation. In still another example, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g, wherein the food formulation has a protein energy ratio (PER) of about 11.4, a fat energy ratio (FER) of about 46.0, and total calories of about 400 to about 560 kcal per 100 g of the food formulation. In preferred aspects, the micronutrient premix referenced in this paragraph contains the nutrients listed in Table A and Table B in the amount specified in Table A and Table B, respectively.

Food formulations of the present disclosure may be formulated into a beverage, a food or a supplement. Non-limiting examples include a bar, a paste, a gel, a cookie, a cracker, a powder, a pellet, a powdered drink to be reconstituted, a blended beverage, a carbonated beverage, and the like. When food formulations of the present disclosure are intended to be administered and consumed by humans, the ingredients in the food formulations are typically Food Chemicals Codex (FCC) purity or U.S. Pharmacopeia (USP)—National Formulary quality, as appropriate, and free from foreign materials. In some aspects, a food formulation may be a therapeutic food. In some aspects, a food formulation may be a ready-to-use food. The term “ready-to-use food” refers to a food that comes ready to use as provided. Specifically, a ready-to-use food doesn't require reconstitution or refrigeration, and stays fresh for at least 6 months, preferably one year, or more preferably two years. In some aspects, a food formulation may be a ready-to-use therapeutic food, as defined in U.S. Department of Agriculture, “Commercial Item Description: Ready-to-Use Therapeutic Food (RUTF)” A-A-20363B (2012), which is designed to meet the guidelines established at the FAO-WHO 45th session of the Codex Alimentarius Commission (Nov. 21, 2022).

### II. Methods

In some aspects, the current disclosure encompasses a method of treatment, the method comprising administering to a subject in need thereof, a therapeutically effective quantity of a composition as disclosed in Section I. In some aspects, the methods disclosed herein may be used in the prevention or treatment of malnutrition, Severe Acute Malnutrition (SAM), necrotizing enterocolitis, nosocomial infections, enteric inflammation, inflammatory disorders, immunodeficiency, inflammatory bowel disease, irritable bowel syndrome, cancer (particularly of the gastrointestinal and immune systems), diarrheal disease, antibiotic associated diarrhea, pediatric diarrhea, appendicitis, allergies, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anemia, disorders of the blood coagulation system, renal disease, disorders of the central nervous system, hepatic disease, ischemia, nutritional disorders, osteoporosis, endocrine disorders, epidermal disorders, psoriasis, acne vulgaris, panic disorder, behavioral disorder and/or post-traumatic stress disorders. In some aspects, the current disclosure also encompasses a method for modifying, repairing, or improving the gut microbiota of a subject in need thereof by administration of a therapeutically effective quantity of a composition as provided in Section I, to a subject in need thereof. In some aspects, the current disclosure also encompasses administration of a therapeutically effective quantity of the disclosed compositions to a subject in need thereof, to enhance the uptake, or utilization, or both of milk N-glycans, or plant-derived polysaccharides, or both.

As used herein the term “therapeutically effective quantity” refers to an amount of the formulation that alleviates, in whole or in part, symptoms associated with the disorder or condition, or halts or slows further progression or worsening of those symptoms, or prevents or provides prophylaxis for the disorder or condition. An “effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects. In some aspects the therapeutically effective quantity may be a quantity that results in reduction in biomarkers of enteric inflammation in the subject. In some aspects the therapeutically effective quantity may be an amount that results in increases in the levels of beneficial plasma protein biomarkers. In some aspects the therapeutically effective quantity may be a quantity that results in significant improvement in ponderal growth as evidenced from weight-for-age z score (WAZ) or mid-upper arm circumference (MUAC) or any other objective measure known in the art. In some aspects the therapeutically effective quantity may be an amount that is sufficient to bring about improvement in musculoskeletal and brain development as demonstrated by objective measures known in the art. In some aspects the therapeutically effective quantity may be amounts that result in enhanced colonization of the beneficial probiotic populations in the gut as demonstrated by various objective means used in the art including but not limited to fecal cultures, genomic analysis of fecal or intestinal swabs. In some aspects, the therapeutically effective quantity may be an amount of the formulation that when administered in conjunction with a vaccine, improves the immunogenicity and efficacy of the vaccine for the subject. In some aspects, the therapeutically effective quantity may be an amount of the formulation that improves the overall health of the subject, as measured by objective measures known in the art.

In some aspects, the amount of a composition administered to a subject and the frequency of administration may vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Additionally, strain formulations as disclosed herein may be combined with food formulations as described in Section I (iii). The two formulations may be administered together, or the administration may be staggered. Amounts of food formulations administered can vary and may be determined by a person of skill in the art. A detailed description of suitable amounts of food formulation for administration is provided in US 2022/0312817, the entire contents of which are hereby incorporated by reference.

As discussed above, administration can be oral, gastric, orogastric, nasogastric, implanted, buccal, and rectal. In some aspects the formulations in section I may be administered orally as any one of but not limited to a solid, semi-solid, liquid, capsule, powder, emulsions, suspensions and tablet or combinations thereof. In some aspects the formulations in section I may be administered, mixed with any one of but not limited to water, juice, gruel, milk, breast milk, baby food, baby formula including F-75 and F-100 or any other commercially available formula, beverage, food products, fruits and vegetables, raw foods and cooked foods. In some aspects the formulations may be administered once daily. In some aspects the formulations may be administered more than once daily. In some aspects the formulations in section I may be administered orogastrically. In some aspect the formulations may be administered nasogastrically.

Compositions described herein can be administered in a variety of methods well known in the arts. Administration can include, for example, methods involving oral ingestion, direct injection, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 um), nanospheres (e.g., less than 1 um), microspheres (e.g., 1-100 um), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

In some aspects, the methods disclosed herein comprise administration of therapeutically effective quantities of the formulations in a subject exhibiting symptoms of or diagnosed with malnutrition. A subject in need of treatment for malnutrition may have a LAZ ≤1, a MUAC≤1, a WAZ≤1, a WLZ≤1, deficiencies in vitamins and minerals, or any combination thereof. In some embodiments, a subject in need of treatment for malnutrition has a LAZ≤1, ≤2, or ≤3. In some embodiments, a subject in need of treatment for malnutrition has a MUAC≤1, ≤2, or ≤3. In some embodiments, a subject in need of treatment for malnutrition has a WAZ≤1, ≤2, or ≤3. In some embodiments, a subject in need of treatment for malnutrition has a WLZ≤1, ≤2, or ≤3. In some embodiments, a subject in need of treatment for malnutrition has a LAZ≤2, a MUAC≤2, a WAZ≤2, a WLZ≤2, or any combination thereof. In some embodiments, a subject in need of treatment for malnutrition has a WAZ≤1.5 and a WLZ≤1.5. In some embodiments, a subject in need of treatment for malnutrition has a WAZ≤2 and a WLZ≤2. In some embodiments, the subject has moderate acute malnutrition. In some embodiments, the subject has severe acute malnutrition (SAM). In some aspects the subject is a child or an infant who consume diets with limited breastmilk content. As used herein the term “limited breastmilk diet” is where breastmilk comprises less than 50% of an infant's total caloric intake. In some aspects breastmilk may comprise 0% of the infant's total caloric intake. In some aspects breastmilk may comprise less than 10% of the infant's total caloric intake. In some aspects breastmilk may comprise less than 20% of the total caloric intake. In some aspects breastmilk may comprise less than 30% of the total caloric intake. In some aspects breastmilk may comprise less than 40% of the total caloric intake. In some aspects breastmilk may comprise less than 50% of the total caloric intake. In some aspects the child is exhibiting one or more of the symptoms including but not limited to a very low weight-for-height (WHZ, less than 3 z-scores below the median WHO growth standards) or a mid-upper arm circumference (MUAC) of less than 11.5 cm, visible severe wasting, or nutritional oedema. In some aspects the child is an infant of age 0-24 months. In some aspect the child is of 0-5 years of age. In some aspects the child is from a underdeveloped or developing country. In some aspects the child is from a developed country. In some aspects the child is from an household below the poverty line for a particular country or earning an income below the objective measure of poverty defined for the country of residence. In some aspect the child is exhibiting symptoms of or has been clinically diagnosed with malnutrition.

In some aspects, the present disclosure encompasses methods of treating malnutrition. In some aspects, the method of treating malnutrition encompasses administering to a subject in need thereof, a therapeutically effective amount of an isolated strain, an engineered strain or a formulation or combination thereof, the strain comprising at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to SEQ ID NOs. 2-23.

In some aspects the present disclosure also encompasses methods for modifying, repairing or improving the health of the gut microbiota of a subject in need thereof. As used herein the term “modifying the gut microbiota” means any intervention that results in change in the gut microbiome as measured by one of many methods available in the art. The change may be a decrease or an increase in the presence of a particular microbial strain, species, genus, family, order, or class. These methods to monitor gut microbiota are well known in the art and may include but are not restricted to fecal cultures, genomic analysis of the feces, or analysis of fecal or intestinal swabs. In some aspects, the present disclosure encompasses methods for repairing or improving the health of the gut microbiota of a subject in need thereof. The “health” of a subject's gut microbiota may be defined by relative abundances of microbial community members, expression of microbial genes, biomarkers, mediators of gut barrier function. To “repair the gut microbiota of a subject,” which is synonymous with “improve gut microbiota health,” means to change the microbiota of a subject, in particular the relative abundances of age- and health-discriminatory taxa, in a statistically significant manner towards chronologically-age matched reference healthy subjects. The term encompasses complete repair (i.e., the measure of gut microbiota health does not deviate by 1.5 standard deviation or more) and levels of repair that are less than complete. The term also encompasses preventing or lessening a change in the relative abundances of age- and health-discriminatory taxa, wherein the change would have been significantly greater absent intervention. A subject with a gut microbiota in need of repair (e.g., a microbiota in “disrepair”, an “immature” gut microbiota, etc.) has a measure of gut microbiota health that deviates by 1.5 standard deviation or more (e.g., 2 std. deviation, 2.5 std. deviation, 3 std. deviation, etc.) from that of chronologically-age matched subjects, wherein the term “chronological age” means the amount of time a subject has lived from birth. Subjects five years or younger are grouped (or binned) by month. Subjects older than 5 years may be grouped by longer intervals of time (e.g., months or years). In some embodiments, a subject with a gut microbiota in need of repair is a subject with malnutrition, SAM, a subject at risk of malnutrition, a subject with a diarrheal disease, a subject recently treated for diarrheal disease (e.g., within 1 week, 2 weeks, 3 weeks, 4 weeks, 5 weeks, 6 weeks, 7 weeks, or 8 weeks), a subject recently treated with antibiotics (e.g., within 1 week, 2 weeks, 3 weeks, 4 weeks, 5 weeks, 6 weeks, 7 weeks, or 8 weeks), a subject undergoing treatment with an antibiotic, a subject who will be undergoing treatment with an antibiotic with about 1-4 weeks or about 1-2 weeks.

In some aspects the subject may be an individual clinically diagnosed with a disease or disorder or syndrome or exhibiting symptoms of disease or disorder or syndrome. In some aspects the subject may be a healthy individual.

The aforementioned methods are not limited to subjects of a particular age. In one aspect, a subject may be less than six months of age. In one aspect, a subject may be at least six months of age. In one example, a subject may be at least six months of age. In another example, a subject may be eighteen years or younger. In still other examples, a subject may be≤15 years, ≤14 years, ≤13 years, ≤12 years, ≤11 years, ≤10 years, ≤9 years, ≤8 years, ≤7 years, ≤6 years, ≤5 years, ≤4 years, ≤3 years, ≤2 years. In still other examples, a subject may be six months to five years of age, six months to 2 years of age, or six months to 18 months of age. In some aspects the subject is a pre-term baby. In some aspects the subject may be an animal. In some aspect the animal may be a mouse model.

An additional aspect of this invention is a method of improving immunogenicity and efficacy of a vaccine in children who consume diets with limited breast milk, the method comprising administration of effective amounts of the compositions detailed in section I of

### DETAILED DESCRIPTION

Microbiome can transfer from mother to infant. In some aspects of the invention, the compositions detailed in section I, may be administered to women during pregnancy to facilitate colonization of the probiotic in the infant gut.

In some aspects, effective amounts of the formulations detailed in section I may be administered prophylactically to reduce the occurrence of malnutrition in children growing up in an household below the poverty line of a particular country or earning an income below the objective measure of poverty defined for the country of residence. In some aspects, the compositions disclosed herein may be administered to “improve a subject's health”. To “improve a subject's health” means to change one or more aspects of a subject's health in a statistically significant manner towards chronologically-age matched reference healthy subjects, as well as to prevent or lessen a change in one or more aspects of the subject's health wherein the change would have been significantly greater absent intervention. The improved aspect of the subject's health may be growth or rate of growth, for example as measured by a score on an anthropometric index; signs or symptoms of disease; relative abundances of health discriminatory plasma proteins, including but not limited to biomarkers, mediators of gut barrier function, bone growth, neurodevelopment, acute and inflammation, and the like. Those in need of treatment to improve their health include those already with a disease, condition, or disorder as well as those prone to have the disease, condition or disorder or those in which the disease, condition or disorder is to be prevented.

### Examples

### Example 1: Determining the Abundance of B. infantis in Bangladeshi Infants with Severe Acute Malnutrition (SAM)

### Methods:

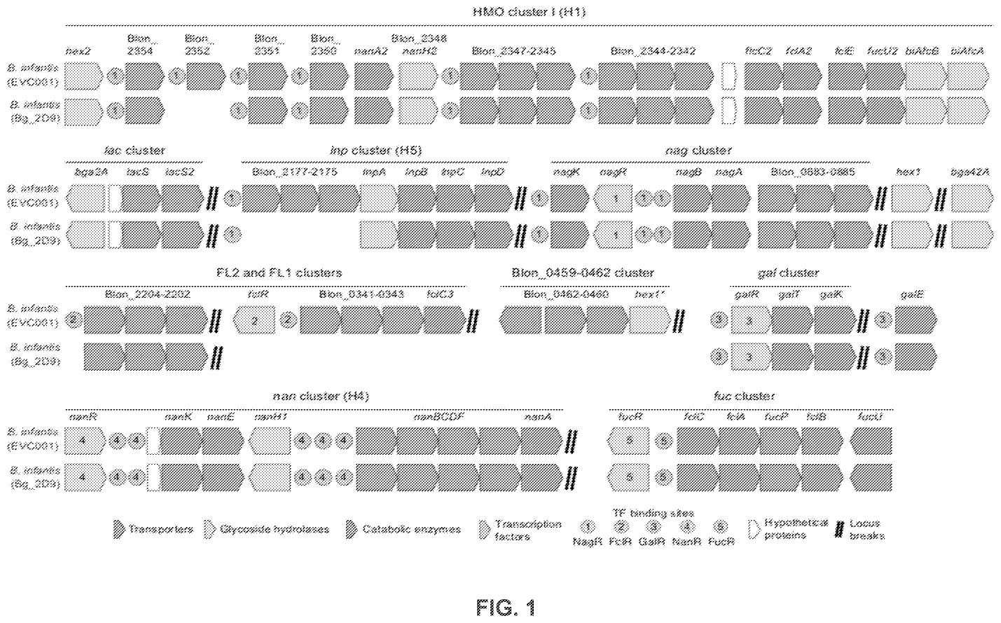

A multiplex qPCR assay was designed to quantify the abundances of Bifidobacterium longum subsp. infantis (B. infantis) using DNA isolated from fecal samples collected from (i) 3-24-month-old Bangladeshi children with SAM (n=102) and (ii) age-matched non-wasted children (WLZ≥−2) (n=49). All children lived in Mirpur, an urban slum of Dhaka, Bangladesh. Primers used to measure total bifidobacterial load were targeted to the 16S rDNA gene. B. infantis abundance was measured using PCR primers directed to the nanH2/exo-α-sialidase gene (Blon_2348) in the H1 locus that is uniquely present in this subspecies (see FIG. 1 and Table 1). The specificity of targeting for both sets of primers was confirmed using a reference collection of cultured gut bacterial strains with sequenced genomes.

TABLE 1
Characteristics of qPCR primers used in this study

| | Primer/ | | Tm |
|---|---|---|---|
| Target organism/gene | probe | Sequence (5′-3′) | (º C.)* |
| Bifidobacterium spp/ | Forward | GCG TGC TTA ACA CAT GCA AGT C | 64.1 |
| 16S rDNA | primer | | |
| | Reverse | CAC CCG TTT CCA GGA GCT ATT | 63.7 |
| | primer | | |
| | Probe | TCA CGC ATT ACT CAC CCG TTC GCC | 69.7 |
| B. infantis/ | Forward | ATA CAG CAG AAC CTT GGC CT | 65.9 |
| Blon_2348 | primer | | |
| | Reverse | GCG ATC ACA TGG ACG AGA AC | 64.8 |
| | primer | | |
| | Probe | TTT CAC GGA TCA CCG GAC CAT ACG | 69.6 |
| B. infantis/ | Forward | GCA CAC CTG CAA TCA GAG CC | 67.7 |
| Blon_2176 | primer | | |
| | Reverse | AGG CAC CAT TAC CCC GTC TG | 68.2 |
| | primer | | |
| | Probe | ATC ACG ATG GCG ATG GCG G | 69.2 |
| B. infantis | Forward | CTG TTC GCG CTT GAT GC | 54.9 |
| EVC001/putative | Primer | | |
| glycosyltransferase | Reverse | CAA TCT TCA CCG AAA GCA AGA C | 54.4 |
| (EpsJ) | primer | | |
| | Probe | AAA GCT TTG CCC AAG CTT GCC C | 61.6 |

DNA was prepared from fecal samples as previously described (J L Gehrig et al Effects of microbiota-directed foods in gnotobiotic animals and undernourished children. Science 365, eaau4732 (2019)), adjusted to 1.5-2 ng/μL and stored in −80° C. before use. PCR reaction mixtures contained (i) 900 nmol forward and reverse primers and 250 nmol Taqman probes for Blon_2348 and Blon_2176 assays, and 150 nM of both 16S rDNA primers for Bifidobacterium spp, (ii) 5 μL Taqman™ Multiplex Master Mix, (iii) 2.5 μL genomic DNA (≤7.5 ng total DNA mass) and (iv) nuclease-free water to make up 10 μL total reaction volume. Assays were performed in duplicate in a 384-well plate format using an Applied Biosystems Quantstudio 6 Flex qPCR instrument. Temperature parameters were as follows: 50° C. for 2 minutes and 95° C. for 10 minutes followed by 40 cycles of 95° C. for 10 seconds and 60° C. for 30 seconds. Standard curves were generated for every PCR plate run using seven serial 10-fold dilutions of purified B. infantis type strain (ATCC15697). Based on the slope and intercept derived from linear regression for cycle thresholds against copy number for the reference, the abundance of each of the three targets was calculated for each sample on the plate. Amplification efficiencies were calculated from the slope [94.3±2.1% (mean±SD) for the Bifidobacterium genus 16S rDNA assay, 89.6±3.1% for Blon 2348 and 87.8-2.3% for the Blon_2176 assay].

Raw data were normalized for input DNA concentration and expressed in genome equivalents per μg of fecal DNA. [Note that since in silico alignment of the 16S rDNA primers against the whole genome of B. infantis ATCC 15697 using the NCBI Primer-BLAST program (J. Ye, G. et al. Primer-BLAST: a tool to design target-specific primers for polymerase chain reaction. BMC bioinformatics 13, (1), 1-11 (2012)) identified four identical target sequences, calculation of total Bifidobacterium abundance in fecal samples was based on the assumption that four copies of this gene are present in each B. infantis genome, whereas abundances of the other PCR targets (Blon_2348, Blon_2176 and EpsJ) were each based on a single copy/genome]. Due to non-normal distribution (Shapiro-Wilk Normality Test P<0.001) qPCR target abundance data were log 10-transformed and the Mann-Whitney U test was used to determine statistical significance of differences between the healthy and SAM children in age bins of 3-12 months, 12-24 months and 18-24 months. Logo-transformed individual qPCR target abundance data were fitted with a generalized additive model using the “gam” function from the “mgcv 1.8-31” package in R as a Gaussian family. A generalized cross validation (GCV) method was used for smoothing, and age at the time of sample collection was used as a predictor to compare the healthy vs. SAM models. The “plot_diff” function from “itsadug 2.4” package in R was utilized to generate difference curves for the two groups.

### Results

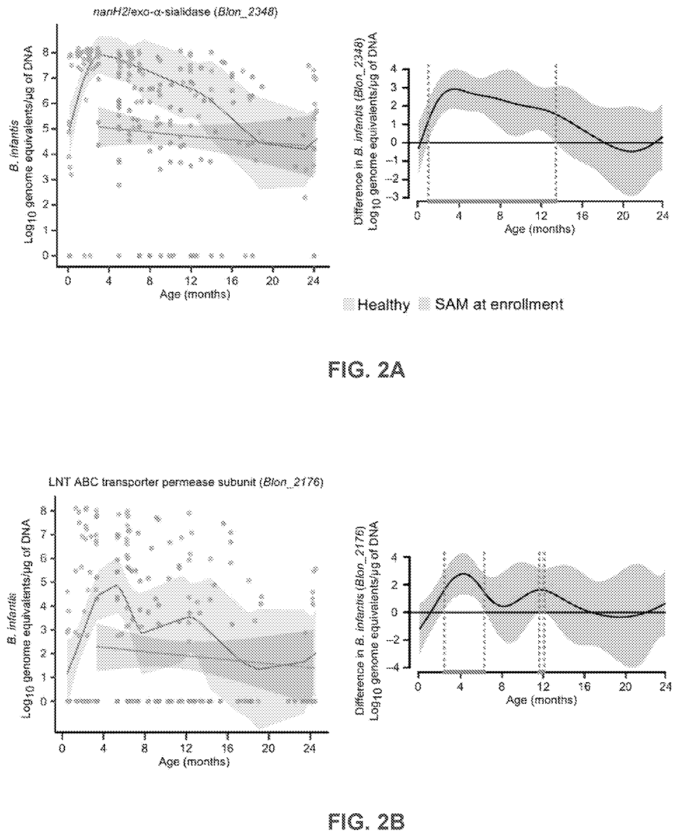

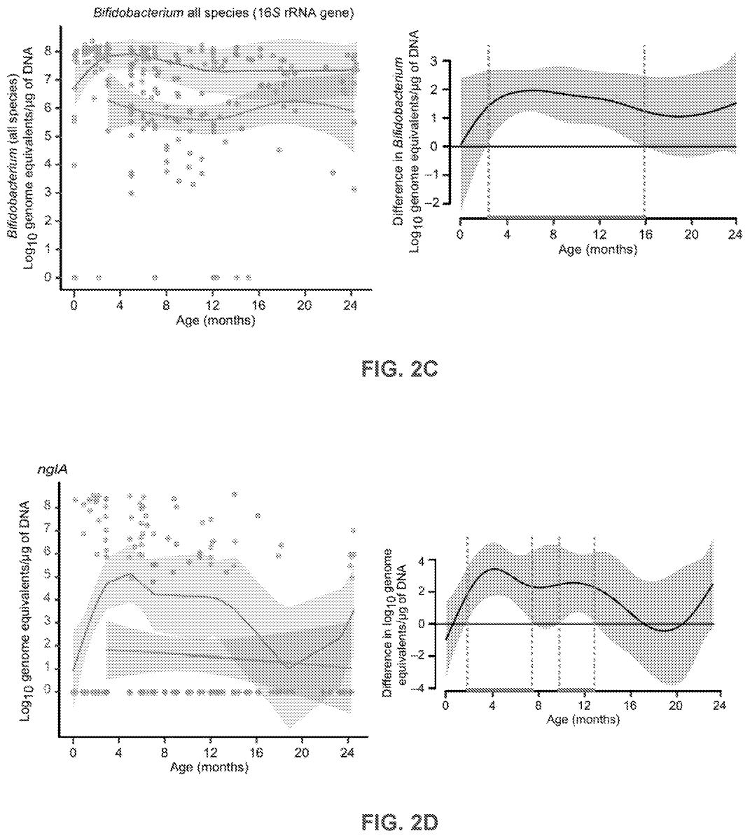

In this cross-sectional study of healthy breastfed Bangladeshi infants and children, maximal levels of B. infantis were documented in feces by the end of the first postnatal month, with no statistically significant diminution in its absolute abundance through 12-months-of-age (green points in FIG. 2A). During this period, ˜75% of all bifidobacterial strains detected in fecal samples from healthy children were B. infantis (Blon_2348-positive). The abundance of B. infantis then declines progressively, by >4 orders of magnitude, between 12 and 24 months as breast milk declines as a proportion of the diet, and the contribution by plant-derived glycans increases (FIG. 2A). The overall abundance of members of the genus Bifidobacterium remained high throughout the 24-month period (FIG. 2C); this can be attributed to the rise in other bifidobacterial species.

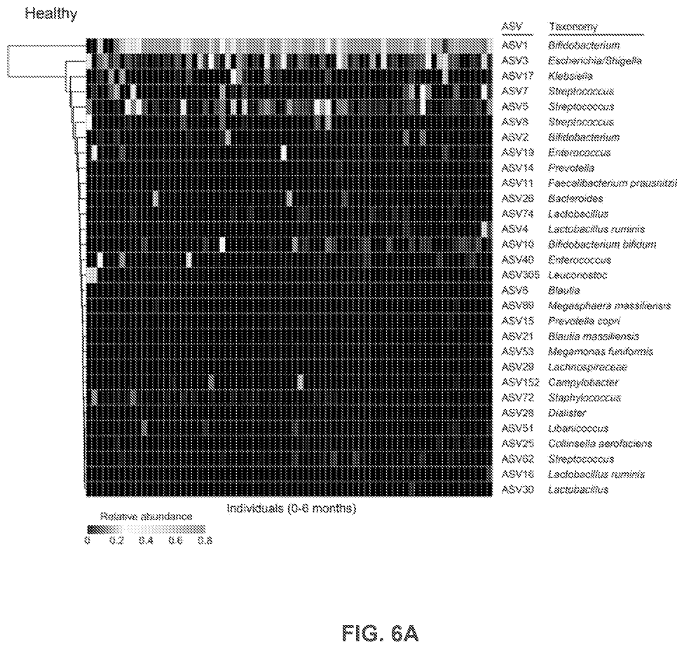

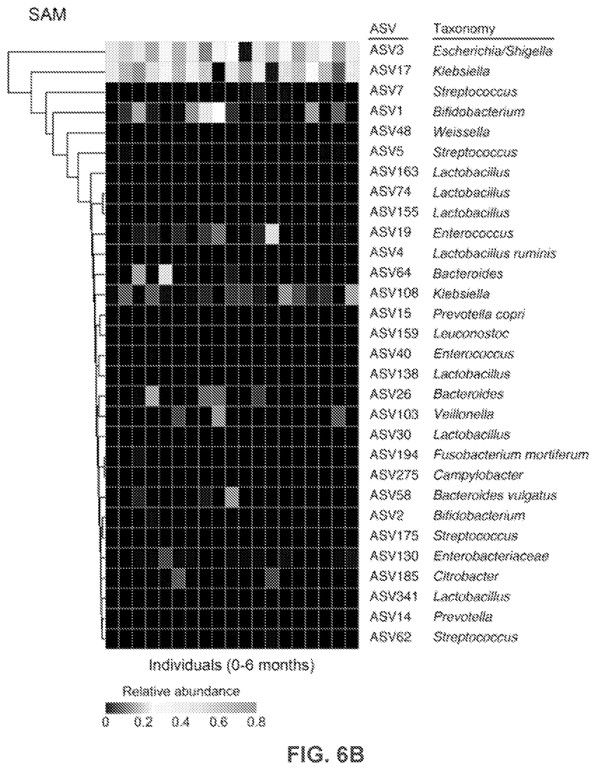

The nanH2 exo-α-sialidase (Blon_2348)-based qPCR assay disclosed that fecal levels of B. infantis were on average 2-3 orders of magnitude lower in 3-12-month-old children with SAM compared to their healthy counterparts (P<0.001; Mann-Whitney U test; FIG. 2A, red points) while no significant differences were evident between 12-24 months (P=0.9; Mann-Whitney U test). These findings are consistent with the observation that Bangladeshi infants with SAM on average receive less than 10% of the recommended daily volume of breastmilk. Total bifidobacterial abundances were also significantly lower in fecal samples from children with SAM when compared to their age-matched healthy counterparts (FIG. 2C); notably, compared to healthy infants, the fecal communities of infants with SAM were dominated by Escherichia, Shigella, Klebsiella and Streptococcus species (FIG. 6).

A qPCR assay using another set of primers directed against the Blon_2176 gene (in the Inp cluster, (FIG. 1) which encodes the permease component of the ABC transporter for the prominent lacto-N-biose type I [Gal (β1-3) GlcNAc]-containing tetrasaccharide in human breast milk, LNT, revealed that its mean absolute abundance was >1000-fold lower in both healthy and SAM infants compared to the abundance of Blon_2348 (FIG. 2B). This finding suggests that the representation of this transporter may vary substantially across Bangladeshi B. infantis strains. Moreover, there was a significant difference in the percentage of fecal samples from SAM compared to healthy Bangladeshi children that lacked detectable levels of this transporter gene (38% versus 66%; P<0.05; two proportion Z test).

### Example 2: Colonization of Bangladeshi Infant Guts with SAM by Milk-Adapted B. Infantis EVC001

Given the deficiency of B. infantis in the microbiota of Mirpur infants with SAM, a pilot single-blind, randomized clinical trial was performed to assess the extent to which EVC001, a commercially available USA infant-derived B. infantis strain with intact H1-H5 gene cluster, could colonize their intestines. Infants between 2- and 6-months-old [4.1+1.1 (mean+SD)] with WLZ<−3 who were free of edema, or those presenting with a kwashiorkor-like manifestation that included bipedal edema were eligible for enrollment in the SYNERGIE trial (SYNbiotic for Emergency Relief of Gut Instability and Enteropathy) after they had completed a standardized protocol for initial management of SAM.

### Synergie Trial Design

The SYNbiotic for Emergency Relief of Gut Instability and Enteropathy (SYNERGIE) study was approved by the Institutional Review Board of the International Centre for Diarrhoeal Disease Research, Bangladesh (ICCDDR,B) and registered at ClinicalTrials.gov (“Pilot of a Prebiotic and Probiotic Trial in Young Infants With Severe Acute Malnutrition” NCT03666572). The study was conducted between as a single-blind randomized trial involving 2-6 month-old infants presenting with a WLZ score<−3 or bilateral pedal edema who had completed an acute phase management protocol for SAM (Ahmed T. et al. Mortality in severely malnourished children with diarrhoea and use of a standardised management protocol. Lancet 5, 1919-1922 (1999)) in the in-patient ward of Dhaka Hospital at ICDDR.B.

Sixty-two enrolled infants were subsequently randomly assigned to one of three treatment groups. At enrollment, there were no statistically significant differences between the three groups with respect to socio-demographic or clinical characteristics (see Table 2). Forty-two of the 62 infants had bilateral pedal edema, but there were no significant differences in the representation of this kwashiorkor-like phenotype among the three arms.

TABLE 2
Socio-demographic, anthropometric and clinical characteristics
of infants assigned to the 3 intervention groups

| | Placebo | | EVC001 | | EVC001 + LNnT | | |
|---|---|---|---|---|---|---|---|
| Characteristic | (n = 21) | | (n = 20) | | (n = 21) | | P value |
| Age in days (median, | 121 | (92, 143.5) | 120 | (85.25, 127) | 130 | (83.5, 155.5) | 0.62 |
| IQR) | | | | | | | |
| Sex: male, n (%) | 11 | (52.4%) | 11 | (55.5%) | 13 | (61.9%) | 0.816 |
| Gestational age | 38 | (36, 40) | 38 | (36, 38.75) | 36 | (35, 38.5) | 0.713 |
| (weeks) (median, | | | | | | | |
| IQR) | | | | | | | |
| Birth weight (kg) | 2.6 | (1.9, 3.00) | 2.7 | (2.4, 3.2) | 2.9 | (2.42, 3.50) | 0.662 |
| (median, IQR) | (n = 15) | | (n = 11) | | (n = 18) | | |
| Mother's age (year) | 23 | (18, 28.5) | 24 | (20, 26.75) | 23 | (19, 26) | 0.896 |
| (median, IQR) | | | | | | | |
| Maternal education <5 | 8 | (38.1%) | 8 | (40.0%) | 9 | (42.9%) | 0.952 |
| years | | | | | | | |
| Housewife mother (%) | 14 | (66.7%) | 15 | (75%) | 18 | (85.7%) | 0.358 |
| Body weight on | 4.10 | (3.46, 5.40) | 4.25 | (3.47, 4.39) | 4.39 | (4.09, 5.22) | 0.42 |
| admission (kg) | (n = 7) | | (n = 8) | | (n = 5) | | |
| (median, IQR) | | | | | | | |
| Length on admission | 59 | (55.75, 61) | 57.80 | (56.75, 59.50) | 60.5 | (57.35, 63.25) | 0.41 |
| (cm) (median, IQR) | | | | | | | |
| Admission WAZ | −3.56 | (−4.5, −3.01) | −3.86 | (−4.27, −3.14) | −3.48 | (−4.07, −2.63) | 0.528 |
| (median, IQR) | (n = 7) | | (n = 8) | | (n = 5) | | |
| Admission WLZ | −4.13 | (−4.34, −3.41) | −3.84 | (−3.94, −3.59) | −3.62 | (−4.32, −3.38) | 0.597 |
| (median, IQR) | (n = 7) | | (n = 8) | | (n = 5) | | |
| Admission LAZ | −2.14 | (−2.73, −1.19) | −1.68 | (−2.86, −0.88) | −1.28 | (−2.56, −0.42) | 0.33 |
| (median, IQR) | | | | | | | |
| Presence of bilateral | 14 | (66.7%) | 12 | (60%) | 16 | (76.2%) | 0.542 |
| pedal edema (%) | | | | | | | |
| Duration of diarrhea, | 3 | (1, 4) | 2.5 | (2, 4) | 3 | (2, 5) | 0.952 |
| days (median, IQR) | | | | | | | |
| Presence of cough on | 2 | (9.5%) | 2 | (10%) | 2 | (9.5%) | 0.998 |
| admission (%) | | | | | | | |
| Percentage of breast | 21.03 | (12.63, 28.33) | 19.69 | (7.94, 28.22) | 16.53 | (12.33, 27.5) | 0.833 |
| milk intake | (n = 10) | | (n = 12) | | (n = 6) | | |
| (median, IQR) | | | | | | | |
| Breast milk intake | 135.0 | (74.02, 204.00) | 141.41 | (41.81, 274.8) | 111.05 | (88.80, 185.06) | 0.973 |
| (g/day) | (n = 10) | | (n = 12) | | (n = 6) | | |
| (median, IQR) | | | | | | | |
| Energy intake from | 66.46 | (33.37, 113.76) | 84.84 | (25.09, 164.88) | 66.63 | (53.28, 111.04) | 0.854 |
| breast milk | (n = 10) | | (n = 12) | | (n = 6) | | |
| (Kcal/day) (median, | | | | | | | |
| IQR) | | | | | | | |
| Percentage of | 8.96 | (5.89, 16.03) | 11.80 | (4.76, 17.24) | 9.51 | (7.21, 16.48) | 0.961 |
| nutrition intake from | (n = 10) | | (n = 12) | | (n = 6) | | |
| breast milk (median, | | | | | | | |
| IQR) | | | | | | | |
| Duration of | 11 | (9, 14) | 12.5 | (9, 23.5) | 11 | (10, 17) | 0.917 |
| hospitalization, days | | | | | | | |
| (median, IQR) | | | | | | | |
| Duration of NRU | 7 | (5.5, 9) | 6.5 | (5, 10) | 7 | (6, 10) | 0.895 |
| stay, days (median, | | | | | | | |
| IQR) | | | | | | | |

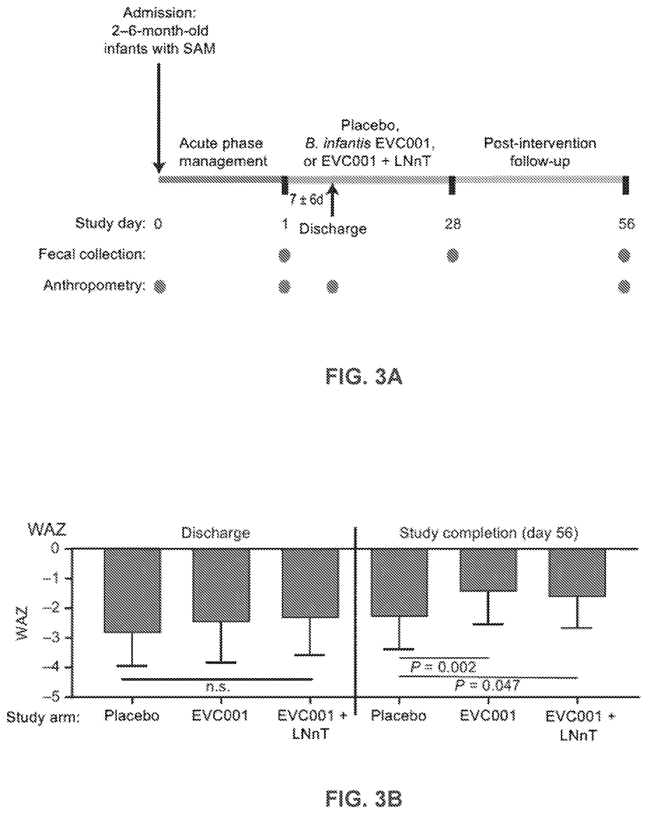

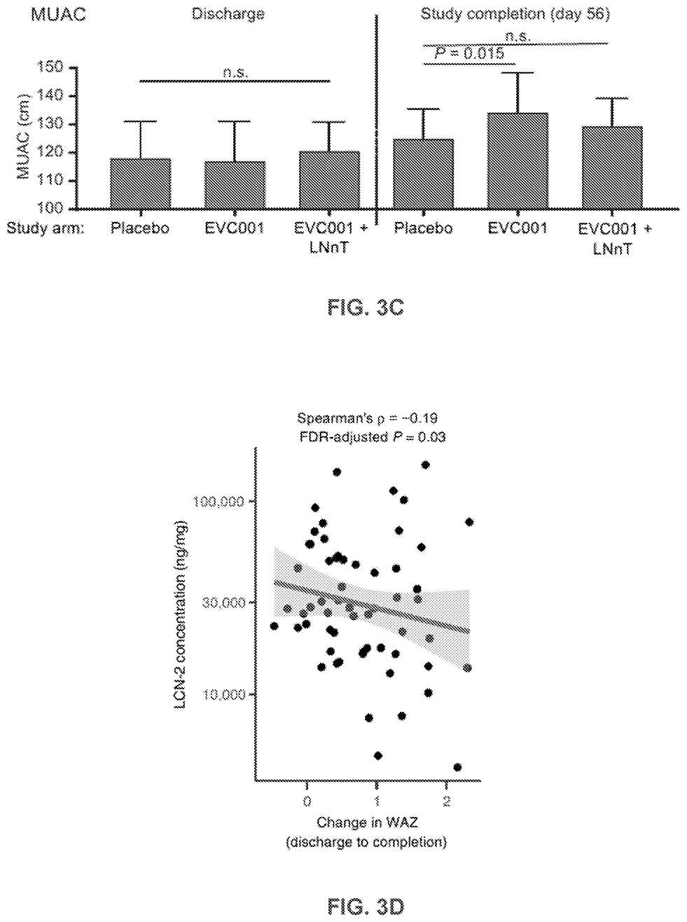

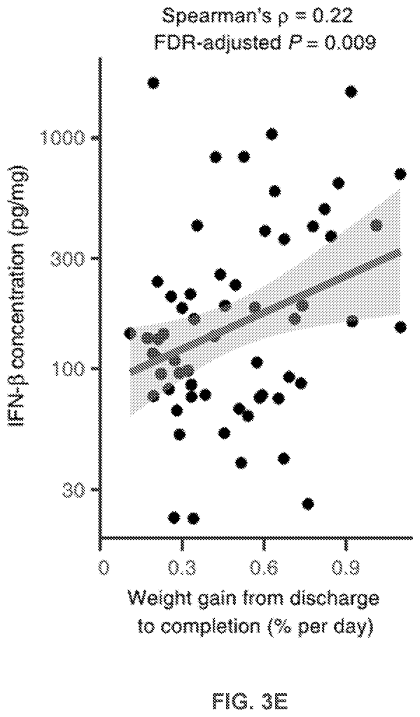

Infants were transferred to Nutrition Rehabilitation Unit (NRU), enrolled and randomized to receive either B. infantis EVC001, B. infantis EVC001 plus Lacto-N-neotetraose [LNnT]), or placebo (lactose) alone for 4 weeks after which time they were followed for 4 more weeks (see FIG. 3A, Table 3). B. infantis EVC001 was administered as a single daily dose mixed with 5 mL of milk (breastmilk, F-100 or formula).

TABLE 3
Supplements used in the study

| Supplement | Schedule | Preservation |
|---|---|---|
| Placebo (lactose, 625 mg) | Once daily | Stored at −20° C. |
| B. infantis (EVC001) 8 | Once daily (one sachet | Stored at −20° C. |
| billion CFU/dose | with 5 ml of breast milk, | |
| | F-100 or infant formula) | |
| Prebiotic LNnT (1.6 | Mixed with each feed | At room |
| g/sachet) | At hospital: 1.6 g/200 | temperature |
| | mL of F-100 | |
| | At home: 1.6 g/120 mL | |
| | feed twice daily | |

Each sachet containing 1.6 gm of LNnT was mixed with 200 mL of F-100 (WHO, 2002) and administered daily after the completion of the antibiotic component of in-patient acute phase management protocol. The protocol for discharge from the NRU was that the participant had achieved a WLZ≥−2. While at home, 1.6 g LNnT was given twice daily by the caregiver, each time mixed with 120 mL of feed (breastmilk or F-100). Refrigerated storage of the probiotic, consumption of LNnT and morbidity were all monitored twice a week by field research assistants. The primary outcome measure was the abundance of B. infantis in the feces of study participants as measured by qPCR during, after 28 days of supplementation (EVC001 versus placebo, and EVC001+LNnT versus placebo). Secondary outcome measures included assessment of the baseline bacterial composition of the gut microbiota of participants, changes in their anthropometric indices and changes in biomarkers of intestinal inflammation. The amount of breast milk consumed was measured by the test weighing method (i.e., weighing before and after feeding) at the time of enrollment. Breastfeeding was encouraged between feeds throughout the study. Infants were provided F-100 infant formula at home in addition to their allotted LNnT supplements in cases where they were not breastfed. (Note that a separate group of non-malnourished infants (WLZ≥−1) who were hospitalized with infections and treated with antibiotics, were also administered EVC001 in the SYNERGIE study; the results of this analysis will be reported separately).

Fecal samples and anthropometric data were obtained prior to the start of supplementation (day 1), the end of supplementation (day 28) and 4-weeks after cessation of treatment (day 56). Swabs of feces were placed in pre-labeled buffered tubes (Zymo Research) that were flash frozen in liquid nitrogen within 20 minutes of defecation. Samples were stored at −80° C. prior to being shipped to Evolve BioSystems, Inc. (Davis, CA) where assays of EVC001 colonization and biomarkers of intestinal inflammation were performed.

### Statistical Analysis of Clinical Data

Clinical data were entered into pre-tested Clinical Record Forms (CRFs) using SPSS (20.0 version, Armonk, NY). Demographic, clinical and socioeconomic data were expressed as median and interquartile range (IQR) for asymmetric quantitative data. For categorical data, frequency with proportional estimates was used. A Kruskal-Wallis H test was used to assess the statistical significance of differences between the three arms. Mann-Whitney U tests were used to determine statistically significant differences in anthropometric measures between pairs of treatment groups at the indicated time points.

Due to the presence of bipedal edema in 42 of the 62 infants at enrollment, it was not possible to compare weights between groups until edema had resolved at the time of hospital discharge. At discharge, there were no significant differences in weight-for-age 7. scores (WAZ) or mid-upper arm circumference (MUAC) between the intervention groups (P=0.361 and P=0.624 respectively, Kruskal-Wallis test; Table 4a). However, at study completion (day 56), WAZ and MUAC in infants treated with EVC001 were significantly greater than for infants in the placebo arm, indicating an improvement in ponderal growth (WAZ P=0.002, MUAC P=0.015, Mann-Whitney U test; FIG. 3B,C, see Table 4b). Notably, LNnT did not improve the benefits of EVC001 on either WAZ or MUAC; indeed only the probiotic intervention produced a significant increase in MUAC at study completion compared placebo (P=0.047; Mann-Whitney U test; FIG. 3B,C, see Table 4b).

TABLE 4a
Effect of the intervention on weight-for-age z-score (WAZ) and
mid-upper arm circumference (MUAC) - Kruskal-Wallis test

| | | | EVC001 + | P |
|---|---|---|---|---|
| | Placebo | EVC001 | LNnT | value* |
| WAZ on discharge | −2.73 | −2.48 | −2.52 | 0.361 |
| (median, IQR) | (−3.45, −2.35) | (−3.38, −1.41) | (−3.44, −1.13) | |
| WAZ on study | −2.26 | −1.30 | −1.81 | 0.01 |
| completion | (−2.90, −1.81) | (−1.85, −1.02) | (−2.59, −0.81) | |
| (median, IQR) | | | | |
| MUAC (mm) on | 120 | 115 | 120 | 0.624 |
| discharge (median, IQR) | (111.5, 125) | (108.5, 128.8) | (120, 129) | |
| MUAC (mm) on study | 126 | 132.9 | 130 | 0.049 |
| completion (median, | (120.3, 130) | (125.5, 140) | (120, 139) | |
| IQR) | | | | |

TABLE 4b
Effect of the intervention on weight-for-age z-score (WAZ) and
mid-upper arm circumference (MUAC) - Mann-Whitney U test

| | P value^{#} |
|---|---|
| Study completion WAZ | |
| Placebo vs EVC001 | 0.002 |
| Placebo vs EVC001 + LNnT | 0.047 |
| Bifido vs EVC001 + LNnT | 0.434 |
| Study completion MUAC | |
| Placebo vs EVC001 | 0.015 |
| Placebo vs EVC001 + LNnT | 0.295 |
| Bifido vs EVC001 + LNnT | 0.16 |

V4-16S rDNA Amplicon Sequencing and Analyses (Clinical Trial)

DNA was extracted from fecal swab samples using the ZymoBIOMICS 96 MagBead DNA kit (Zymo Research). Extracted DNA was quantified using QuantIT dsDNA Assay kit, high sensitivity (ThermoFisher Scientific, Waltham, MA) according to the manufacturer's protocol. Variable region 4 of the 16S rRNA gene was amplified using barcoded 515F and 806R primers. Barcoded amplicons were sequenced (Illumina MiSeq, paired-end 250 nt reads). The three datasets were demultiplexed, denoised, and amplicon sequence variants (ASVs) identified using DADA2 (B. J. Callahan, et al. DADA2: High- resolution sample inference from Illumina amplicon data. Nat. Methods. 13, 581-583. (2016)). After merging, ASVs underwent taxonomic analysis using a pre-trained Naive Bayes classifier supplied by QIIME2 (v2019.7). The classifier was trained on the Greengenes 13_8 99% OTUs, trimmed to contain only the V4 region.

### Quantification of Gut Inflammatory Biomarkers (Clinical Trial)

A previous study showed that EVC001 colonization of healthy USA breastfed infants reduced levels of gut inflammatory biomarkers (B. Henrick et al. Restoring Bifidobacterium longum subspecies infantis EVC001 to the infant gut microbiome significantly reduces intestinal inflammation. Curr. Dev. Nutr. 3, nzz049.OR12-01-19 (2019)). Therefore, the total abundance of B. infantis (as defined by the Blon_2348-targeted qPCR assay) to levels of myeloperoxidase (MPO), calprotectin, and lipocalin-2 (LCN-2), as well as pro-inflammatory cytokines (IFNγ, IL-17A, IL-1ß and IL-6) in fecal samples collected at the beginning and end of the intervention period was compared. Calprotectin and Lipocalin-2 (LCN-2/NGAL) were quantified from 80 mg of stool diluted 1:10 in Meso Scale Discovery (MSD; Rockville, MD) diluent using R-PLEX. Fecal cytokine levels (IFNγ, IL-17A, IL-1ß and IL-6) were quantified using the U-Plex Inflammation Panel 1 Kit (human) according to the manufacturer's instructions. Plates were read on a Sector Imager 2400 using MSD Discovery Workbench analysis software. Standards and samples were measured in duplicate and blank values were subtracted from all readings. Myeloperoxidase (MPO) was measured using commercially available ELISA kits (Alpco, Salem, NH, USA). B. infantis abundance at day 28 was significantly negatively correlated with levels of IL-1ß (Spearman's p=−0.34, FDR-adjusted P value=0.033) and calprotectin (Spearman's p=−0.41, FDR-adjusted P value=0.01).

Over half of the SAM infants in the SYNERGIE study were not receiving any breastmilk at the time of hospital admission [15/21 (71%) of the children who were subsequently randomized to the synbiotic arm, 8/20 (40%) in the probiotic arm, and 11/21 (52%) in the placebo arm]. Even among those infants who were receiving breastmilk at the time of admission, consumption was only 18±13% of the recommended daily volume for aged-matched healthy infants. This raised the question of whether the limited durability of colonization with the USA-derived EVC001 strain reflected the reduced prevalence of breast feeding and amount of breast milk consumed by SAM infants. Therefore, microbial communities of Bangladeshi children were tapped to search for B. infantis strains that may have a competitive advantage over other endogenous B. infantis strains as well as the USA infant-derived EVC001.

### Example 3: Mining Microbiota for B. infantis Strains Adapted to Mirpur Infant Feeding Practices

Microbial Community SEED (mcSEED) ((D. A. Rodionov Micronutrient requirements and sharing capabilities of the human gut microbiome. Front. Microbiol. 10, 1316 (2019)) was used to characterize the genomic features of 10 B. infantis strains; six of these had been cultured from fecal samples collected from three healthy and one undernourished infants/children aged 6-24 months living in Mirpur during this study, two strains from Malawian infants (MC1, MC2), a USA donor-derived type strain (ATCC 15697), plus EVC001 (see Table 5).

TABLE 5
Origin of bifidobacterial strains and genome assembly characteristics

| | | Genome assembly characteristics | | | | | | |
|---|---|---|---|---|---|---|---|---|
| | | | | | | Length of | | |
| | | Coverage | Coverage | | N50 contig | largest | Assembly | |
| Bacterial strain | | (fold) | (fold) | # of | length | contig | length | Assembly |
| ID | Origin | Illumina | Pacbio | contigs | (bp) | (bp) | (bp) | Type |
| B. longum subsp. | Evolve | | | 1 | 2832850 | 2832850 | 2832850 | |
| infantis EVC001 | BioSystems | | | | | | | |
| B. longum subsp. | ATCC | | | | | | | |
| infantis ATCC | type | | | | | | | |
| 15697 | strain | | | | | | | |
| B. longum subsp. | Bangladesh | 105 | 285 | 32 | 213778 | 438104 | 2740504 | Hybrid |
| infantis | (MAL-ED | | | | | | | |
| JG_Bg463.m5.93_JG | cohort)* | | | | | | | |
| B. longum subsp. | Bangladesh | 781 | 1555 | 2 | 2505499 | 2505499 | 2511042 | Hybrid |
| infantis | (MDCF | | | | | | | |
| Bg40721_2D9_SN_2018 | healthy | | | | | | | |
| | cohort)* | | | | | | | |
| B. longum subsp. | Bangladesh | 744 | 2428 | 3 | 2499328 | 2499328 | 2505490 | Hybrid |
| infantis | (MDCF | | | | | | | |
| Bg40721_2C3_SN_2018 | healthy | | | | | | | |
| | cohort)* | | | | | | | |
| B. longum subsp. | Bangladesh | 50 | | 48 | 129199 | 386355 | 2623489 | Illumina |
| infantis | (MDCF | | | | | | | |
| Bg41721_1E9_SN_2018 | healthy | | | | | | | |
| | cohort)* | | | | | | | |
| B. longum subsp. | Bangladesh | 50 | | 47 | 135821 | 316574 | 2624708 | Illumina |
| infantis | (MDCF | | | | | | | |
| Bg41721_1G8_SN_2018 | healthy | | | | | | | |
| | cohort)* | | | | | | | |
| B. longum subsp. | Bangladesh | 57 | 166 | 6 | 1925067 | 1925067 | 2622864 | Hybrid |
| infantis | (SAM | | | | | | | |
| PS064_13.C6_Bang_JG | study)* | | | | | | | |
| B. longum subsp. | Malawi | 173 | 171 | 43 | 185503 | 389004 | 2597522 | Hybrid |
| infantis | Twin | | | | | | | |
| Malawi_264A_MC1 | Study# | | | | | | | |
| B. longum subsp. | Malawi | 55 | 214 | 4 | 2383706 | 2383706 | 2594947 | Hybrid |
| infantis | Twin | | | | | | | |
| Malawi_264A_MC2 | Study# | | | | | | | |
| B. longum subsp. | Bangladesh | 57 | 249 | 11 | 1610067 | 1610067 | 2405437 | Hybrid |
| suis | (SAM | | | | | | | |
| PS131.S11.17_F6Bang_JG | study)* | | | | | | | |
| B. breve | Bangladesh | 57 | 211 | 1 | 2359653 | 2359653 | 2359653 | Hybrid |
| PS155.S09_23A9_JG_2018 | (SAM | | | | | | | |
| | study)* | | | | | | | |
| B. breve | Peru | 253 | 275 | 16 | 228906 | 455968 | 2406753 | Hybrid |
| PE1C332.m20.82_Peru_JG | (MAL-ED | | | | | | | |
| | cohort)* | | | | | | | |
| B. longum | USA | 208 | 318 | 38 | 149237 | 433496 | 2402824 | Hybrid |
| STL_TW14.1_LFYP82 | Twin | | | | | | | |
| | study\hat{ } | | | | | | | |
| B. bifidum | | 100 | | 60 | 197978 | 377496 | 2485423 | Illumina |
| Bg41221_3D10_SN_2018 | | | | | | | | |

### Methods

### Culturing of Fecal Samples

Fecal samples, collected from 6-24-month-old Bangladeshi children that had been enrolled in the MDCF, MAL-ED and SAM clinical studies (see Table 5 for the origin of each isolate), were pulverized in liquid nitrogen and a ˜0.1 g aliquot of each sample was transferred to a Coy chamber (Coy Laboratory Products, Grass Lake, MI) under anaerobic conditions (atmosphere of 75% N₂, 20% CO₂, and 5% H2). Samples were diluted 1:10 (wt/vol) with reduced PBS (PBS/0.05% L-cysteine-HCl) in 50 ml conical plastic tubes containing 5 mL of 2 mm-diameter glass beads (VWR). Tubes were gently vortexed, and the resulting slurry was passed through a 100 um-pore diameter nylon cell strainer (BD Falcon). 500 μL of each clarified fecal sample was added to 4.5 mL of PBS and a dilution series of 1:10, 1:100, 1:1000, and 1:10,000 was prepared in PBS. LYBHI (brain-heart infusion medium supplemented with 0.5% yeast extract) agar plates were streaked with 100 L of each dilution. Plates were incubated for 2-3 days at 37° C. under anaerobic conditions. Colonies were picked into 96 deep-well plates (Thermo Fisher Scientific) containing 600 μL of Wilkins-Chalgren broth and incubated overnight at 37° C. (Isolate stocks were prepared by combining 50 μL of culture with 50 μL of PBS/30% glycerol in shallow 96-well plates. Stocks were frozen at −80° C. for future use). A 500 μL aliquot of each culture was transferred to 2 mL screw cap tubes and pelleted by centrifugation. The resulting supernatant was discarded and DNA was extracted from pellets with phenol: chloroform. V4-16S rDNA amplicons were generated by PCR and sequenced (Illumina MiSeq; paired-end 250 nt reads). Clonal isolates whose V4-16S rDNA sequences shared >97% sequence identity with Bifidobacteria were subjected to full-length 16S rDNA gene sequencing using primers 8F and 1391R (Turner S. et al., Investigating deep phylogenetic relationships among cyanobacteria and plastids by small subunit rRNA sequence analysis. J Eukaryot Microbiol. 1999 46, 327-38 (1999)).

### Identification of Unique Strains by Genome Sequencing

Cryopreserved stocks of bacteria were brought into the COY chamber, struck on MRS agar plates for single colonies, incubated overnight at 37° C. under anaerobic conditions, replated on MRS-agar: a single colony was picked into 6 mL of MRS broth and incubated at 37° C. to late log phase. Genomic DNA was isolated from cell pellets (J. L. Gehrig et al., Effects of microbiota-directed foods in gnotobiotic animals and undernourished children. Science 365, eaau4732 (2019)) libraries were prepared for shotgun sequencing (TruSeq Nano DNA Library Prep Kit, Illumina), pooled and multiplex sequencing was performed an Illumina Nextseq instrument (2×150 bp reads). Raw reads were demultiplexed (bcl2fastq) and pre-processed to remove low-quality bases and reads (trim galore, v0.4.5). Quality-controlled reads were then subsampled to a depth of ˜100-fold coverage using bbtools (v38.26). Paired end reads corresponding to each genome were assembled using Spades with the careful option (v3.13.0). Isolates sharing ≥99% nucleotide sequence identity in their full length 16S rRNA genes and ≥96% nucleotide sequence identity throughout their genomes [NUCmer (Kurtz et al. Versatile and open software for comparing large genomes. Genome Biol. 5, R12 (2004))] were defined as unique strains.

### Pacbio and Illumina Hybrid Assemblies

Cryopreserved stocks of organisms for long-read sequencing/assembly were struck onto Blood Heart Infusion medium and incubated overnight. Single colonies were picked, inoculated into 6 mL of liquid MRS medium, and incubated for 2 days. Turbid cultures were transferred to 15 mL conical tubes and pelleted by centrifugation. DNA was recovered using a high molecular weight genomic DNA extraction kit (MagAttract HMW, Qiagen). Purified DNA was prepared for long-read sequencing using the SMRTbell Template Prep Kit (v1.0, PacBio) and Barcoded Adapter Kit (v8A, PacBio) and whole genome sequencing was performed [PacBio Sequel System; read length, 3681□861 (mean±SD)nt]. Sequencing reads were demultiplexed and converted from raw bam to fastq format (SMRT Tools software, v5.1.0 or 6.0.0). Short reads generated from the Illumina sequencer and long reads for each isolate were co-assembled using Unicycler (v0.4.7). For both short-read and hybrid assemblies, assembly quality statistics were generated using Quast (v4.5). Open reading frames were identified and annotated using Prokka (v1.12). Additional functional annotation was added based on homology to entries in the microbial community SEED (mcSEED) database (Gehrig J. L. et al. Effects of microbiota-directed foods in gnotobiotic animals and undernourished children. Science 365, eaau4732 (2019), Rodionov D. A. et. al. Micronutrient requirements and sharing capabilities of the human gut microbiome. Front. Microbiol. 10, 1316 (2019)).

### In Silico Reconstructions and Phenotype Predictions

Subsystems-based, context-driven functional assignments of genes, curation and reconstruction of bifidobacterial carbohydrate metabolic pathways were performed in the web-based mcSEED environment, a private clone of the publicly available SEED platform (Overbeek R. et. al. The subsystems approach to genome annotation and its use in the project to annotate 1000 genomes. Nucleic Acids Res. 33, 5691-702 (2005)). The mcSEED platform includes: (i) 336 genomes representing 15 species of bifidobacteria isolated from the human gut and (ii) a collection of curated subsystems capturing utilization of mono-, oligo-, polysaccharides and other carbohydrates in bifidobacteria. Data on functional roles (transporters, glycoside hydrolases, catabolic enzymes, transcriptional regulators) involved in bifidobacterial sugar metabolism were collected by extensive literature search using PaperBLAST (Price M. N. and Arkin A. P., PaperBLAST: Text mining papers for information about homologs. mSystems. 2, e00039-17 (2017).), and by exporting information from the Carbohydrate Active Enzyme (CAZy) (V. Lombard, H. Golaconda Ramulu, E. Drula, P. M. Coutinho, B. Henrissat, The carbohydrate-active enzymes database (CAZy) in 2013. Nucleic Acids Res. 42, D490-D495 (2014)), Transporter Classification (TCDB) (Saier M. H. et. al., The Transporter Classification Database (TCDB): recent advances. Nucleic Acids Res. 44, D372-379 (2016)) and RegPrecise (56) databases. Reconstruction of regulons and prediction of transcription factor binding sites was performed as described previously (Khoroshkin M.S. et al., Transcriptional regulation of carbohydrate utilization pathways in the Bifidobacterium Genus. Front Microbiol. 7, 120 (2016)).

mcSEED-based in silico metabolic reconstructions provided predictions for the ability of strains to synthesize amino acids and B-vitamins and utilize various carbohydrates.

### Results

### Carbohydrate Utilization

The chemical diversity of dietary and host-derived polysaccharides in the gut ecosystem is matched by a multitude of species-to-species variations in sugar utilization networks-extracellular degradation of polysaccharides, uptake and biochemical transformations of oligo- and monosaccharides, and regulatory mechanisms involved in feeding of carbohydrates into central carbon metabolism. An integrated subsystems-based approach was applied to systematically map carbohydrate utilization pathways and assign corresponding phenotypes for 15 bifidobacterial isolates. Overall, the analyzed strains were predicted to be able to utilize 38 out of 63 carbohydrates classified as monosaccharides (including aldoses, ketoses, sugar acids, sugar alcohols, and Amadori adducts), di- and oligosaccharides, and selected polysaccharides.

HMO transporters—In silico metabolic reconstructions was used to compare the representation of candidate HMO transporters. All strains had (i) biochemically characterized LNnT transporters (Blon_2345-2347 and Blon_2342-2344) (Garrido et al. Oligosaccharide binding proteins from Bifidobacterium longum subsp. infantis reveal a preference for host glycans. PLOS One 6 (2011)), (ii) the in vivo characterized fucosylated HMO transporter FL2 (Blon_2202-2204) (Sakanaka M. et al. Evolutionary adaptation in fucosyllactose uptake systems supports bifidobacteria-infant symbiosis. Sci Adv 5, eaaw7696. (2019)), and (iii) two paralogs of a substrate-specific component of putative HMO transporters with unknown specificity encoded within the H1 locus (Blon_2350, Blon_2354) (Sela D. A. et al., The genome sequence of Bifidobacterium longum subsp. infantis reveals adaptations for milk utilization within the infant microbiome. Proc. Natl. Acad. Sci. USA 105, 18964-9 (2008)) (see Table 6). Other known or candidate HMO transporters were mosaically distributed among 10 analyzed B. infantis strains (includes the 6 Bangladeshi, 2 Malawian and 2 USA donor derived strains; see Table 6). The fucosylated HMO transporter FL1 (Blon_0341-0343) and associated transcriptional regulator FclR were identified in 6 strains, including the USA donor-derived ATCC 15697 type strain and EVC001. The biochemically characterized LNT transporter GltABC (Blon_2175-77) and a paralog of the substrate-binding component of ABC transporters with unknown specificity (Blon_2352) were present in five strains, including EVC001 but not in Bg_2D9. The Blon_0459-0642 gene cluster, encoding the B. breve LNnT transporter Blon_0460-0462 and an additional paralogue of Hex1, named Hex1*, was present only in the ATCC 15697 and EVC001 strains. Finally, LNB/GNB transporter Blon_0883-0885 was present in all but two B. infantis strains (Table 6).

TABLE 6
Representation of HMO transporters in selected strains

| | Locus tag (name) | | | | | |
|---|---|---|---|---|---|---|
| | Blon_2175-2177 | | | | Blon_0460-0462; | Blon_2202-2204 |
| | (GltABC) | Blon_0883-0885 | Blon_2345-2347 | Blon_2342-2344 | Bbr_1554 | (FL2) |
| | Specificity | | | | | |
| | LNT; LNB; | | | LNnT | | 2′FL; 3FL; |
| | GNB | LNB; GNB | LNnT | (low affinity) | LNnT | LDFT; LNFPI |
| B. longum subsp. | + | + | + | + | + | + |
| infantis EVC001 | | | | | | |
| B. longum subsp. | + | + | + | + | + | + |
| infantis ATCC | | | | | | |
| 15697 | | | | | | |
| B. longum subsp. | + | − | + | + | − | + |
| infantis | | | | | | |
| JG_Bg463.m5.93_JG | | | | | | |
| B. longum subsp. | − | + | + | + | − | + |
| infantis | | | | | | |
| Bg40721_2D9_SN_2018 | | | | | | |
| B. longum subsp. | − | + | + | + | − | + |
| infantis | | | | | | |
| Bg40721_2C3_SN_2018 | | | | | | |
| B. longum subsp. | − | + | + | + | − | + |
| infantis | | | | | | |
| Bg41721_1E9_SN_2018 | | | | | | |
| B. longum subsp. | − | + | + | + | − | + |
| infantis | | | | | | |
| Bg41721_1G8_SN_2018 | | | | | | |
| B. longum subsp. | − | + | + | + | − | + |
| infantis | | | | | | |
| PS064_13.C6_Bang_JG | | | | | | |
| B. longum subsp. | − | + | + | + | − | + |
| infantis | | | | | | |
| Malawi_264A_MC1 | | | | | | |
| B. longum subsp. | + | − | + | + | − | + |
| infantis | | | | | | |
| Malawi_264A_MC2 | | | | | | |
| B. longum subsp. | + | − | + | + | − | ‘−* |
| suis | | | | | | |
| PS131.S11.17_F6 | | | | | | |
| Bang JG | | | | | | |
| B. breve | + | − | − | − | + | − |
| PS155.S09_23A9_JG_2018 | | | | | | |
| B. breve | + | − | − | − | + | − |
| PE1C332.m20.82_Peru_JG | | | | | | |
| B. longum | − | − | − | − | − | − |
| STL_TW14.1_LFYP82 | | | | | | |
| B. bifidum | ‘+\hat{ } | − | − | − | − | − |
| Bg41221_3D10_SN_2018 | | | | | | |

| | Locus tag (name) | | | | |
|---|---|---|---|---|---|
| | Blon_0341-0343 | | | | |
| | (FL1) | Blon_2350 | Blon_2351 | Blon_2352 | Blon_2354 |
| | Specificity | | | | |
| | 2′FL; 3FL | UnK | Unk | Unk | Unk |
| B. longum subsp. | + | + | + | + | + |
| infantis EVC001 | | | | | |
| B. longum subsp. | + | + | + | + | + |
| infantis ATCC | | | | | |
| 15697 | | | | | |
| B. longum subsp. | − | + | + | − | + |
| infantis | | | | | |
| JG_Bg463.m5.93_JG | | | | | |
| B. longum subsp. | − | + | + | − | + |
| infantis | | | | | |
| Bg40721_2D9_SN_2018 | | | | | |
| B. longum subsp. | − | + | + | − | + |
| infantis | | | | | |
| Bg40721_2C3_SN_2018 | | | | | |
| B. longum subsp. | + | + | + | − | + |
| infantis | | | | | |
| Bg41721_1E9_SN_2018 | | | | | |
| B. longum subsp. | + | + | − | + | + |
| infantis | | | | | |
| Bg41721_1G8_SN_2018 | | | | | |
| B. longum subsp. | + | + | + | − | + |
| infantis | | | | | |
| PS064_13.C6_Bang_JG | | | | | |
| B. longum subsp. | − | + | + | + | + |
| infantis | | | | | |
| Malawi_264A_MC1 | | | | | |
| B. longum subsp. | + | + | + | + | + |
| infantis | | | | | |
| Malawi_264A_MC2 | | | | | |
| B. longum subsp. | − | + | + | − | + |
| suis | | | | | |
| PS131.S11.17_F6 | | | | | |
| Bang JG | | | | | |
| B. breve | − | − | − | − | − |
| PS155.S09_23A9_JG_2018 | | | | | |
| B. breve | − | − | − | − | − |
| PE1C332.m20.82_Peru_JG | | | | | |
| B. longum | − | − | − | − | − |
| STL_TW14.1_LFYP82 | | | | | |
| B. bifidum | − | − | − | − | − |
| Bg41221_3D10_SN_2018 | | | | | |

(+ transporter genes present, − transporter gene(s) absent, +\hat{ } ortholog of Blon_2177 present but predicted to transport only LNB and GNB, −* = genes absent (probably) due to a genome assembly error, UnK unknown)

Utilization of HMOs—The B. infantis strains analyzed possess multiple genomic clusters involved in utilization of major HMOs and their constituent disaccharides (LNB, GNB, lactose) and monosaccharides (glucose, galactose, fucose, N-acetylglucosamine and NANa). These loci are shown in schematic form for strains 2D9 and EVC001 in FIG. 1 and described in detail for all analyzed strains in Table 6 and 7. The HMO cluster I (H1) is a characteristic feature of all B. infantis strains (Sela D.A. et al., The genome sequence of B. longum subsp. infantis reveals adaptations for milk utilization within the infant microbiome. Proc. Natl. Acad. Sci. USA 105, 18964-9 (2008)). It encodes a set of glycoside hydrolases required for utilization of LNT, LNnT, and sialylated/fucosylated HMOs (Hex2, NanH2, BiAfcA, BiAfcB), two ABC transporters for type II HMOs such as LNnT (Blon_2342-2344, Blon_2345-2347), four additional copies of substrate-binding components of possible HMO transporters (Blon_2350, Blon_2351, Blon_2352, Blon_2354), and the fucose catabolism enzymes FclA2, FclC2, FclE, and FucU2 ((James K. et al., B. breve UCC2003 metabolises the human milk oligosaccharides lacto-N-tetraose and lacto-N-neo-tetraose through overlapping, yet distinct pathways. Sci. Rep. 6, 38560 (2016), Sela D. A. et al. An infant- associated bacterial commensal utilizes breast milk sialyloligosaccharides. J. Biol. Chem. 286, 11909-11918 (2011), Sela D.A. et al., B. longum subsp. infantis ATCC 15697 a- Fucosidases are active on fucosylated human milk oligosaccharides. Appl. Environ. Microbiol. 78, 795-803 (2012), Garrido D. et al., A novel gene cluster allows preferential utilization of fucosylated milk oligosaccharides in B. longum subsp. longum SC596. Sci Rep 6, 35045 (2016)). The lac cluster encodes a β-1,4-galactosidase (Bga2A, GH2) and two paralogs of lactose permease LacS. The Inp cluster (also known as H5) encodes a LNT transporter Blon_2175-2177 (Garrido D. et al. Oligosaccharide binding proteins from B. longum subsp. infantis reveal a preference for host glycans. PLOS One 6 (2011)) and the LNB/GNB catabolic enzymes (Kitaoka M. et al., Novel putative galactose operon involving Lacto-N-Biose Phosphorylase in B. longum. Appl. Environ. Microbiol. 71, 3158-3162 (2005)). Genes of two other GHs connected to LNT utilization (the β-1,3/4/6-galactosidase, Bga42A from the GH42 family and the β-1,3/4/6-N-acetylglucosaminidase Hex1 from the GH20 family), are scattered across the B. infantis genome (Yoshida E. et al., B. longum subsp. infantis uses two different β-galactosidases for selectively degrading type-1 and type-2 human milk oligosaccharides. Glycobiology 22, 361-368 (2012), Viborg A. H. et al., Distinct substrate specificities of three glycoside hydrolase family 42 β-galactosidases from B. longum subsp. infantis ATCC 15697. Glycobiology 24, 208 216 (2014)). The FL1 and FL2 gene clusters encode two distinct ABC transporters for fucosylated HMOs (Sakanaka M. et al., Evolutionary adaptation in fucosyllactose uptake systems supports bifidobacteria-infant symbiosis. Sci Adv 5, eaaw7696. (2019)). The Blon_0459-0462 gene cluster encodes a homolog of the LNnT transporter that has been characterized in B. breve (James K. et al., B. breve UCC2003 metabolises the human milk oligosaccharides lacto-N-tetraose and lacto-N- neo-tetraose through overlapping, yet distinct pathways. Sci. Rep. 6, 38560 (2016)) and a second paralog of the Hex1 catabolic enzyme. Finally, the nag, gal, nan (also known as H4), and fuc gene clusters encode catabolic enzymes and uptake transporters required for utilization of N-acetyl-glucosamine (GlcNAc), galactose, N-acetylneuraminic acid (NANa), and fucose, respectively.

TABLE 7
Representation of glycoside hydrolases involved in HMO utilization

| | Locus tag (name) | | | | | |
|---|---|---|---|---|---|---|
| | Blon_2334 | Blon_2016 | Blon_0732 | Blon_0459 | Blon_2355 | Blon_2348 |
| | (Bga2A) | (Bga42A) | (Hex1) | (Hex1*) | (Hex2) | (NanH2) |
| | Function | | | | | |
| | β-1,4- | β-1,3/4/6- | β-1,3/4/6-N- | β-1,3/4/6-N- | β-1,3/4-N- | α-2,3/6- |
| | galactosidase | galactosidase | acetylglucosaminidase | acetylglucosaminidase | acetylglucosaminidase | sialidase |
| | (GH2) | (GH42) | (GH20) | (GH20) | (GH20) | (GH33) |
| B. longum | + | + | + | + | + | + |
| subsp. infantis | | | | | | |
| EVC001 | | | | | | |
| B. longum | + | + | + | + | + | + |
| subsp. infantis | | | | | | |
| ATCC 15697 | | | | | | |
| B. longum | + | + | + | − | + | + |
| subsp. infantis | | | | | | |
| JG_Bg463.m5.93_JG | | | | | | |
| B. longum | + | + | + | − | + | + |
| subsp. infantis | | | | | | |
| Bg40721_2D9_SN_2018 | | | | | | |
| B. longum | + | + | + | − | + | + |
| subsp. infantis | | | | | | |
| Bg40721_2C3_SN_2018 | | | | | | |
| B. longum | + | + | + | − | + | + |
| subsp. infantis | | | | | | |
| Bg41721_1E9_SN_2018 | | | | | | |
| B. longum | + | + | + | − | + | + |
| subsp. infantis | | | | | | |
| Bg41721_1G8_SN_2018 | | | | | | |
| B. longum | + | + | + | − | + | + |
| subsp. infantis | | | | | | |
| PS064_13.C6_Bang_JG | | | | | | |
| B. longum | + | + | + | − | + | + |
| subsp. infantis | | | | | | |
| Malawi_264A_MC1 | | | | | | |
| B. longum | + | + | + | − | + | + |
| subsp. infantis | | | | | | |
| Malawi_264A_MC2 | | | | | | |
| B. longum | + | + | + | − | + | + |
| subsp. suis | | | | | | |
| PS131.S11.17 F6 Bang_JG | | | | | | |
| B. breve | + | + | − | + | − | − |
| PS155.S09_23A9_JG_2018 | | | | | | |
| B. breve | + | + | − | + | − | − |
| PE1C332.m20.82_Peru_JG | | | | | | |
| B. longum | + | + | + | − | − | − |
| STL_TW14.1_LFYP82 | | | | | | |
| B. bifidum | + | + | + | − | − | − |
| Bg41221_3D10_SN_2018 | | | | | | |

| | Locus tag (name) | | | | | |
|---|---|---|---|---|---|---|
| | Blon_2335 | Blon_2336 | BbgIII | BbhI | BbhII | AfcA |
| | (BiAfcA) | (BiAfcB) | (BBPR_0482) | (BBPR_1529) | (BBPR_1018) | (Blon_2335) |
| | Function | | | | | |
| | | | Extracellular | Extracellular | Extracellular | Extracellular |
| | α-1,2-L- | α-1,3/4-L- | β-1,4- | β-1,3-N- | β-1,6-N- | α-1,2-L- |
| | fucosidase | fucosidasc | galactosidase | acctylglucosaminidase | acctylglucosaminiclase | fucosidase |
| | (GH95) | (GH29) | (GH2) | (GH20) | (GH20) | (GH95) |
| B. longum | + | + | − | − | − | − |
| subsp. infantis | | | | | | |
| EVC001 | | | | | | |
| B. longum | + | + | − | − | − | − |
| subsp. infantis | | | | | | |
| ATCC 15697 | | | | | | |
| B. longum | + | + | − | − | − | − |
| subsp. infantis | | | | | | |
| JG_Bg463.m5.93_JG | | | | | | |
| B. longum | + | + | − | − | − | − |
| subsp. infantis | | | | | | |
| Bg40721_2D9_SN_2018 | | | | | | |
| B. longum | + | + | − | − | − | − |
| subsp. infantis | | | | | | |
| Bg40721_2C3_SN_2018 | | | | | | |
| B. longum | + | + | − | − | − | − |
| subsp. infantis | | | | | | |
| Bg41721_1E9_SN_2018 | | | | | | |
| B. longum | + | + | − | − | − | − |
| subsp. infantis | | | | | | |
| Bg41721_1G8_SN_2018 | | | | | | |
| B. longum | + | + | − | − | − | − |
| subsp. infantis | | | | | | |
| PS064_13.C6_Bang_JG | | | | | | |
| B. longum | + | + | − | − | − | − |
| subsp. infantis | | | | | | |
| Malawi_264A_MC1 | | | | | | |
| B. longum | + | + | − | − | − | − |
| subsp. infantis | | | | | | |
| Malawi_264A_MC2 | | | | | | |
| B. longum | + | + | − | − | − | − |
| subsp. suis | | | | | | |
| PS131.S11.17 F6 Bang_JG | | | | | | |
| B. breve | + | − | − | − | − | − |
| PS155.S09_23A9_JG_2018 | | | | | | |
| B. breve | + | − | − | − | − | − |
| PE1C332.m20.82_Peru_JG | | | | | | |
| B. longum | − | − | − | − | − | − |
| STL_TW14.1_LFYP82 | | | | | | |
| B. bifidum | − | + | + | + | + | + |
| Bg41221_3D10_SN_2018 | | | | | | |

| | Locus tag (name) | | | | |
|---|---|---|---|---|---|
| | AfcB | SiaBB1 | SiaBB2 | LnbB | LnbX |
| | (Blon_2336) | (BBPR_1793) | (BBPR_1794) | (BBPR_1438) | (BLLJ_1505) |
| | Function | | | | |
| | Extracellular | Extracellular | Extracellular | Extracellular | Extracellular |
| | α-1,3/4-L- | α-2,3/6- | α-2,3/6/8- | lacto-N- | lacto-N- |
| | fucosidase | sialidase | sialidase | biosidase | biosidase |
| | (GH29) | (GH33) | (GH33) | (GH20) | (GH136) |
| B. longum | − | − | − | − | − |
| subsp. infantis | | | | | |
| EVC001 | | | | | |
| B. longum | − | − | − | − | − |
| subsp. infantis | | | | | |
| ATCC 15697 | | | | | |
| B. longum | − | − | − | − | − |
| subsp. infantis | | | | | |
| JG_Bg463.m5.93_JG | | | | | |
| B. longum | − | − | − | − | − |
| subsp. infantis | | | | | |
| Bg40721_2D9_SN_2018 | | | | | |
| B. longum | − | − | − | − | − |
| subsp. infantis | | | | | |
| Bg40721_2C3_SN_2018 | | | | | |
| B. longum | − | − | − | − | − |
| subsp. infantis | | | | | |
| Bg41721_1E9_SN_2018 | | | | | |
| B. longum | − | − | − | − | − |
| subsp. infantis | | | | | |
| Bg41721_1G8_SN_2018 | | | | | |
| B. longum | − | − | − | − | − |
| subsp. infantis | | | | | |
| PS064_13.C6_Bang_JG | | | | | |
| B. longum | − | − | − | − | − |
| subsp. infantis | | | | | |
| Malawi_264A_MC1 | | | | | |
| B. longum | − | − | − | − | − |
| subsp. infantis | | | | | |
| Malawi_264A_MC2 | | | | | |
| B. longum | − | − | − | − | − |
| subsp. suis | | | | | |
| PS131.S11.17 F6 Bang_JG | | | | | |
| B. breve | − | − | − | − | − |
| PS155.S09_23A9_JG_2018 | | | | | |
| B. breve | − | − | − | − | − |
| PE1C332.m20.82_Peru_JG | | | | | |
| B. longum | − | − | − | − | − |
| STL_TW14.1_LFYP82 | | | | | |
| B. bifidum | + | + | + | + | + |
| Bg41221_3D10_SN_2018 | | | | | |

(+ gene present, − gene absent)

HMO utilization gene loci in B. infantis contain five genes encoding predicted transcription factors, including the local regulators FclR, FucR, GalR and NanR of FL1, fuc, gal and nan gene clusters, respectively, and the predicted global regulator NagR located in the nag gene cluster (FIG. 1, Table 8). It was previously predicted that NagR controls the utilization of GlcNAc and LNB in B. longum, B. infantis, B. breve, and B. bifidum by repressing their nag and Inp gene clusters (Khoroshkin M.S. et al., Transcriptional regulation of carbohydrate utilization pathways in the B. Genus. Front Microbiol. 7, 120 (2016)). This prediction was later experimentally confirmed in B. breve; GlcNAc-6P is the effector molecule for NagR (James, K. et al., B. breve UCC2003 employs multiple transcriptional regulators to control metabolism of particular human milk oligosaccharides. Appl. Environ. Microbiol. 84, e02774-17 (2018)). The current analysis was expanded from the NagR regulon reconstruction to the H1 loci in the six Bangladeshi B. infantis strains and found additional candidate NagR-binding sites in promoter regions of Blon_2344, Blon_2347, Blon_2350, Blon_2351, Blon 2352, and Blon_2354 (FIG. 1, Table 8). Based on these results, it is hypothesized that the presence of GlcNAc-6P in the cell induces expression of the nag and Inp genes, as well as most H1 genes in B. infantis.

TABLE 8
Predicted transcription factor binding sites in the promoter regions of genes
involved in glycan utilization in B. infantis strains used in this study. (^^ Position relative
to the first codon, # score > 5.0 = strong site, 5.0 > score > 4.5 = weak site)

| | | | | | | |
|---|---|---|---|---|---|---|
| reg- | gene_ | operon_ | | | pos- | |
| ulator_ | locus_ | locus_ | operon_ | | ition | score |
| locus_tag | tag^ | tags^ | names | site | ^^ | # |

NagR; ROK family

| | | | | | | | |
|---|---|---|---|---|---|---|---|
| | Blon_0880 | Blon_0879 | Blon_0879 | nagK | TTTGTTAAGATagTTgtCAAt | −78 | 5.55 |
| B. longum subsp. | | Blon_0880 | Blon_0880 | nagR | TTTGTTAAtgATacTAACAAt | −189 | 6.02 |
| infantis ATCC 15697 | | | | | TTgGTgAAGtTTCaTAACAAt | −107 | 4.94 |
| | | Blon_0881 | Blon_0881- | nagB- | aTTGTTAtGAAaCTTcACCAA | -251 | 4.94 |
| | | | Blon_0882- | nagA- | | | |
| | | | Blon_0883- | Blon_08 | | | |
| | | | Blon_0884- | 83-0885 | | | |
| | | | Blon_0885 | | | | |
| | | | | | aTTGTTAgtATcaTTAACAAA | −169 | 6.02 |
| | | Blon_2177 | Blon_2177- | gltABC- | aTTGTTAgttgggTTgACAAt | −93 | 5.46 |
| | | | Blon_2176- | InpABC | | | |
| | | | Blon_2175- | D | | | |
| | | | Blon_2174- | | | | |
| | | | Blon_2173- | | | | |
| | | | Blon_2172- | | | | |
| | | | Blon_2171 | | | | |
| | | Blon_2344 | Blon_2344- | Blon_23 | aTTGTTAgGcATgTTgACAAA | −106 | 5.45 |
| | | | Blon_2343- | 44-2342 | | | |
| | | | Blon_2342 | | | | |
| | | Blon_2347 | Blon_2347- | Blon_23 | TaTGTTAAGgAcgTTgACAAA | −103 | 5.32 |
| | | | Blon_2346- | 47-2345 | | | |
| | | | Blon_2345 | | | | |
| | | Blon_2350 | Blon_2350- | Blon_23 | TaTGTTAAGAATgTTgACgAA | −105 | 4.47 |
| | | | Blon_2349- | 50- | | | |
| | | | Blon_2348 | nanA2- | | | |
| | | | | nanH2 | | | |
| | | Blon_2351 | Blon_2351 | Blon_23 | TaTGTTAAGAATgTTgACgAA | −105 | 4.47 |
| | | | | 51 | | | |
| | | Blon_2352 | Blon_2352 | Blon_23 | TaTCTTAAGAATgTTgACgAA | −105 | 4.47 |
| | | | | 52 | | | |
| | | Blon_2354 | Blon_2354 | Blon_23 | TaTGTTAAGgcTgTTgACAgt | −106 | 4.36 |
| | | | | 54 | | | |
| B. longum subsp. | N_01959 | N_00369 | N_00369 | Blon_23 | TaTGTTAAGgcTgTTgACAgt | −108 | 4.36 |
| infantis EVC001 | | | | 54 | | | |
| | | N_00370 | N_00370 | Blon_23 | TaTCTTAAGAATgTTgACgAA | −107 | 4.47 |
| | | | | 52 | | | |
| | | N_00371 | N_00371 | Blon_23 | TaTGTTAAGAATgTTgACgAA | −107 | 4.47 |
| | | | | 51 | | | |
| | | N_00372 | N_00372- | Blon_23 | | −107 | 4.47 |
| | | | N_00373- | 50- | | | |
| | | | N_00374 | nanA2- | | | |
| | | | | nanH2 | | | |
| | | N_00375 | N_00375- | Blon_23 | TaTGTTAAGgAcgTTgACAAA | −105 | 5.32 |
| | | | N_00376- | 47-2345 | | | |
| | | | N_00377 | | | | |
| | | N_00378 | N_00378- | Blon_23 | aTTGTTAgGcATgTTgACAAA | −126 | 5.45 |
| | | | N_00379- | 44-2342 | | | |
| | | | N_00380 | | | | |
| | | N_00557 | N_00557- | gltABC- | aTTGTTAgttgggTTgACAAt | −95 | 5.46 |
| | | | N_00558- | InpABC | | | |
| | | | N_00559- | D | | | |
| | | | N_00560- | | | | |
| | | | N_00561- | | | | |
| | | | N_00562- | | | | |
| | | | N_00563 | | | | |
| | | N_01958 | N_01958- | nagB- | aTTGTTAtGAAaCTTcACcAA | −249 | 4.94 |
| | | | N_01957- | nagA- | | | |
| | | | N_01956- | Blon_08 | | | |
| | | | N_01955- | 83-0885 | | | |
| | | | N_01954 | | | | |
| | | | | | aTTGTTAgtATcaTTAACAAA | −167 | 6.02 |
| | | N_01959 | N_01959 | nagR | TTTGTTAAtgATacTAACAAt | −191 | 6.02 |
| | | | | | TTgGTgAAGtTTCaTAACAAt | −109 | 4.94 |
| | | N_01960 | N_01960 | nagK | TTTGTTAAGATagTTgtCAAt | −76 | 5.55 |
| B. longum | BILO543B | BILO543B3 | BILO543B3 | nagK | TTTGTTAAGATagTTgtCAAt | −78 | 5.55 |
| subsp. infantis | 32D0_ | 2D0_04665 | 2D0_04665 | | | | |
| Bg40721_2D9_SN_2018 | 04670 | BILO543B3 | BILO543B3 | nagR | TTTGTTAAtgATacTAACAAt | −189 | 6.02 |
| | | 2D0_04670 | 2D0_04670 | | | | |
| | | | | | TTaGTgAAGtTTCaTAACAAt | −107 | 5.01 |
| | | BILO543B3 | BILO543B3 | nagB- | aTTGTTAtGAAaCTTcACtAA | −251 | 5.01 |
| | | 2D0_04675 | 2D0_04675- | nugA- | | | |
| | | | BILO543B3 | Blon_08 | | | |
| | | | 2D0_04680- | 83-0885 | | | |
| | | | BILO543B3 | | | | |
| | | | 2D0_04685- | | | | |
| | | | BILO543B3 | | | | |
| | | | 2D0_04690- | | | | |
| | | | BILO543B3 | | | | |
| | | | 2D0_04695 | | | | |
| | | | | | aTTGTTAgtATcaTTAACAAA | −169 | 6.02 |
| | | BILO543B3 | BILO543B3 | InpABC | | −51 | 5.85 |
| | | 2D0_09500 | 2D0_09500- | D | | | |
| | | | BILO543B3 | | | | |
| | | | 2D0_09495- | | | | |
| | | | BILO543B3 | | | | |
| | | | 2D0_09490- | | | | |
| | | | BILO543B3 | | | | |
| | | | 2D0_09485 | | | | |
| | | BILO543B3 | BILO543B3 | Blon_23 | aTTGTTAgGcATgTTgACAAA | −124 | 5.45 |
| | | 2D0_10425 | 2D0_10425- | 44-2342 | | | |
| | | | BILO543B3 | | | | |
| | | | 2D0_10420- | | | | |
| | | | BILO543B3 | | | | |
| | | | 2D0_10415 | | | | |
| | | BILO543B3 | BILO543B3 | Blon_23 | TaTGTTAAGyAcgTTgACAAA | −103 | 5.32 |
| | | 2D0_10440 | 2D0_10440- | 47-2345 | | | |
| | | | BILO543B3 | | | | |
| | | | 2D0_10435- | | | | |
| | | | BILO543B3 | | | | |
| | | | 2D0_10430 | | | | |
| | | BILO543B3 | BILO543B3 | Blon_23 | TaTCTTAAGAATgTTgACgAA | −123 | 4.47 |
| | | 2D0_10455 | 2D0_10455- | 50- | | | |
| | | | BILO543B3 | nanA2- | | | |
| | | | 2D0_10450- | nanH2 | | | |
| | | | BILO543B3 | | | | |
| | | | 2D0_10445 | | | | |
| | | BILO543B3 | BILO543B3 | Blon_23 | TaTGTTAAGAATgTTgACgAA | −105 | 4.47 |
| | | 2D0_10460 | 2D0_10460 | 51 | | | |
| | | BILO543B3 | BILO543B3 | Blon_23 | TaTGTTAAGgcTgTTgACAgt | −106 | 4.36 |
| | | 2D0_10465 | 2D0_10465 | 54 | | | |
| B. longum | BILO9e02a | BILO9e02a2 | BILO9e02a2 | nagB- | aTTGTTAtGAAaCTTcACCAA | −249 | 4.94 |
| subsp. infantis | 2a1_00978 | a1_00977 | a1_00977- | nagA- | | | |
| Bg41721_1G8_SN_2018 | | | BILO9e02a2 | Blon_08 | | | |
| | | | a1_00976- | 83-0885 | | | |
| | | | BILO9e02a2 | | | | |
| | | | a1_00975- | | | | |
| | | | BILO9e02a2 | | | | |
| | | | a1_00974- | | | | |
| | | | BILO9e02a2 | | | | |
| | | | a1_00973 | | | | |
| | | | | | aTTGTTAgtATcaTTAACAAA | −167 | 6.02 |
| | | BILO9e02a2 | BILO9e02a2 | nagR | TTTGTTAAtgATacTAACAAt | −191 | 6.02 |
| | | a1_00978 | a1_00978 | | | | |
| | | | | | TTgGTgAAGtTTCaTAACAAt | −109 | 4.94 |
| | | BILO9e02a2 | BILO9e02a2 | nagK | TTTGTTAAGATagTTgtCAAt | −76 | 5.55 |
| | | a1_00979 | a1_00979 | | | | |
| | | BILO9e02a2 | BILO9e02a2 | Blon_23 | TaTGTTAAGgcTgTTgACAAt | −108 | 4.79 |
| | | a1_01309 | a1_01309 | 54 | | | |
| | | BILO9e02a2 | BILO9e02a2 | Blon_23 | TaTGTTAAGAATgTTgACgAA | −107 | 4.47 |
| | | a1_01310 | a1_01310 | 51 | | | |
| | | BILO9e02a2 | BILO9e02a2 | Blon_23 | TaTGTTAAGAATgTTgACgAA | −107 | 4.47 |
| | | a1_01311 | a1_01311- | 50- | | | |
| | | | BILO9e02a2 | nanA2- | | | |
| | | | a1_01312- | nanH2 | | | |
| | | | BILO9e02a2 | | | | |
| | | | a1_01313 | | | | |
| | | BILO9e02a2 | BILO9e02a2 | InpABC | aTTGTTAgttAagTTgACAAt | −453 | 5.85 |
| | | a1_01636 | a1_01636- | D | | | |
| | | | BILO9e02a2 | | | | |
| | | | a1_01637- | | | | |
| | | | BILO9e02a2 | | | | |
| | | | a1_01638- | | | | |
| | | | BILO9e02a2 | | | | |
| | | | a1_01639 | | | | |
| | | not_anno | not_annotate | Blon_23 | TaTGTTAAGgAcgTTgACAAA | −105 | 5.32 |
| | | tated_ | d Prokka | 47-2345 | | | |
| | | Prokka | | | | | |
| B. longum | BILO16373 | BILO163738 | BILO163738 | gltABC- | aTTGTTAgttgggTTgACAAt | −93 | 5.46 |
| subsp. infantis | 828_01190 | 28_00611 | 28_00611- | InpABC | | | |
| JG_Bg463.m5.93_JG | | | BILO163738 | D | | | |
| | | | 28_006110- | | | | |
| | | | BILO163738 | | | | |
| | | | 28_00609- | | | | |
| | | | BILO163738 | | | | |
| | | | 28_00608- | | | | |
| | | | BILO163738 | | | | |
| | | | 28_00607- | | | | |
| | | | BILO163738 | | | | |
| | | | 28_00606- | | | | |
| | | | BILO163738 | | | | |
| | | | 28_00605 | | | | |
| | | BILO163738 | BILO163738 | Blon_23 | aTTGTTAgGcATgTTgACAAA | −124 | 5.45 |
| | | 28_01003 | 28_01003- | 44-2342 | | | |
| | | | BILO163738 | | | | |
| | | | 28_01002- | | | | |
| | | | BILO163738 | | | | |
| | | | 28_01001 | | | | |
| | | BILO163738 | BILO163738 | Blon_23 | TaTGTTAAGgAcgTTgACAAA | −103 | 5.32 |
| | | 28_01006 | 28_01006- | 47-2345 | | | |
| | | | BILO163738 | | | | |
| | | | 28_01005- | | | | |
| | | | BILO163738 | | | | |
| | | | 28_01004 | | | | |
| | | BILO163738 | BILO163738 | Blon_23 | TaTGTTAAGAATgTTgACgAA | −105 | 4.47 |
| | | 28_01009 | 28_01009- | 50- | | | |
| | | | BILO163738 | nunA2- | | | |
| | | | 28_01008- | nanH2 | | | |
| | | | BILO163738 | | | | |
| | | | 28_01007 | | | | |
| | | BILO163738 | BILO163738 | Blon_23 | TaTGTTAAGAATgTTgACgAA | −105 | 4.47 |
| | | 28_01010 | 28_01010 | 51 | | | |
| | | BILO163738 | BILO163738 | Blon_23 | TaTGTTAAGgcTgTTgACAAt | −106 | 4.79 |
| | | 28_01011 | 28_01011 | 54 | | | |
| | | BILO163738 | BILO163738 | nagK | TTTGTTAAGATagTTgtCAAt | −77 | 5.55 |
| | | 28_01189 | 28_01189 | | | | |
| | | BILO163738 | BILO163738 | nagR | TTTGTTAAt.gATacTAACAAt | −189 | 6.02 |
| | | 28_01190 | 28_01190 | | | | |
| | | | | | TTaGTgAAGtTTCaTAACAAt | −107 | 5.01 |
| | | BILO163738 | BILO163738 | nagB- | aTTGTTAtGAAaCTTcACtAA | −251 | 5.01 |
| | | 28_01191 | 28_01191- | nagA | | | |
| | | | BILO163738 | | | | |
| | | | 28_01192 | | | | |
| | | | | | aTTGTTAgtATcaTTAACAAA | −169 | 6.02 |
| B. longum | BILO14587 | BILO145876 | BILO163738 | nagK | TTTGTTAAGATagTTgtCAAt | −78 | 5.55 |
| subsp. infantis | 6ef_01017 | ef_01016 | 28_01189 | | | | |
| PS064_13.C6_Bang_JG | | BILO145876 | BILO145876 | nagR | TTTGTTAAtgATacTAACAAt | −189 | 6.02 |
| | | ef_01017 | ef_01017 | | | | |
| | | | | | TTgGTgAAGtTTCaTAACAAt | −107 | 4.94 |
| | | BILO145876 | BILO145876 | nagB- | aTTGTTAtGAAaCTTcACCAA | −251 | 4.94 |
| | | ef_01018 | ef_01018- | nagA- | | | |
| | | | BILO145876 | Blon_08 | | | |
| | | | ef_01019- | 83-0885 | | | |
| | | | BILO145876 | | | | |
| | | | cf_01020- | | | | |
| | | | BILO145876 | | | | |
| | | | ef_01021- | | | | |
| | | | BILO145876 | | | | |
| | | | ef_01022 | | | | |
| | | | | | aTTGTTAgtATcaTTAACAAA | −169 | 6.02 |
| | | BILO145876 | BILO145876 | InpABC | aTTGTTAgttAagTTgACAAt | −451 | 5.85 |
| | | ef_01972 | ef_01972- | D | | | |
| | | | BILO145876 | | | | |
| | | | ef_01971- | | | | |
| | | | BILO145876 | | | | |
| | | | ef_01970- | | | | |
| | | | BILO145876 | | | | |
| | | | ef_01969 | | | | |
| | | BILO145876 | BILO145876 | Blon_23 | aTTGTTAgGcATgTTgACAAA | −124 | 5.45 |
| | | ef_02161 | ef_02161- | 44-2342 | | | |
| | | | BILO145876 | | | | |
| | | | ef_02160- | | | | |
| | | | BILO145876 | | | | |
| | | | ef_02159 | | | | |
| | | BILO145876 | BILO145876 | Blon_23 | TaTGTTAAGgAcgTTgACAAA | −103 | 5.32 |
| | | ef_02164 | ef_02164- | 47-2345 | | | |
| | | | BILO145876 | | | | |
| | | | ef_02163- | | | | |
| | | | BILO145876 | | | | |
| | | | ef_02162 | | | | |
| | | BILO145876 | BILO145876 | Blon_23 | TaTGTTAAGAATgTTgACgAA | −123 | 4.47 |
| | | cf_02167 | cf_02167- | 50- | | | |
| | | | BILO145876 | nanA2- | | | |
| | | | ef_02166- | nanH2 | | | |
| | | | BILO145876 | | | | |
| | | | ef_02165 | | | | |
| | | BILO145876 | BILO145876 | Blon_23 | TaTGTTAAGAATaTTgACgAA | −105 | 4.55 |
| | | ef_02168 | ef_02168 | 51 | | | |
| | | BILO145876 | BILO145876 | Blon_23 | TaTGTTAAGgcTgTTgACAAt | −106 | 4.79 |
| | | cf_02169 | cf_02169 | 54 | | | |

MnaR; LacI family

| | | | | | | | |
|---|---|---|---|---|---|---|---|
| B. longum subsp. | Blon_0874 | Blon_0868 | Blon_0868- | mna38*- | tTaCTAAAGCGCTTTAGtcT | −117 | 5.49 |
| infantis ATCC 15697 | | | Blon_0869 | mna38 | | | |
| | | Blon_0876 | Blon_0876- | mna_12 | gAaCTAAAGCGgTTTAGAAT | −36 | 6.2 |
| | | | Blon_0875 | 5-manI | | | |
| | | Blon_2380 | Blon_2380- | Blon_23 | ATaCTAAAGCGgTTTAGtTa | −138 | 5.86 |
| | | | Blon_2379- | 80-2378- | | | |
| | | | Blon_2378- | blMan5 | | | |
| | | | Blon_2377 | B | | | |
| | | Blon_2468 | Blon_2468 | endoBI- | ATaCTAAAGCGaTTTAGtTc | −123 | 5.75 |
| | | | | 1 | | | |
| B. longum subsp. | N_01965 | N_00253 | N_00253 | endoBI- | ATaCTAAAGCGaTTTAGtTc | −125 | 5.75 |
| infantis EVC001 | | | | 1 | | | |
| | | N_00345 | N_00345- | Blon_23 | ATaCTAAAGCGgTTTAGtTa | −140 | 5.86 |
| | | | N_00346- | 80-2378- | | | |
| | | | N_00347- | blMan5 | | | |
| | | | N_00348 | B | | | |
| | | N_01963 | N_01963- | mna_12 | gAaCTAAAGCGgTTTAGAAT | −71 | 6.20 |
| | | | N_01964 | 5-manI | | | |
| | | N_01971 | N_01971- | mna38*_ | tTaCTAAAGCGCTTTAGtcT | −115 | 5.49 |
| | | | N_01970 | mna38 | | | |
| B. longum | BILO543B | BILO543B3 | BILO543B3 | mnaR | AgaCTAAAaCatTTTAGtTg | −126 | 4.56 |
| subsp. infantis | 32D0_ | 2D0_04640 | 2D0_04640 | | | | |
| Bg40721_2D9_SN_2018 | 04640 | BILO543B3 | BILO543B3 | mna_12 | cAaCTAAAGCGgTTTAaAAT | −68 | 5.71 |
| | | 2D0_04650 | 2D0_04650- | 5-manI | | | |
| | | | BILO543B3 | | | | |
| | | | 2D0_04645 | | | | |
| | | BILO543B3 | BILO543B3 | Blon_23 | gTaCTAAAaCGgTTTAGtTa | −138 | 5.23 |
| | | 2D0_10585 | 2D0_10585- | 80-2378- | | | |
| | | | BILO543B3 | b1Man5 | | | |
| | | | 2D0_10580- | B | | | |
| | | | BILO543B3 | | | | |
| | | | 2D0_10575- | | | | |
| | | | BILO543B3 | | | | |
| | | | 2D0_10570 | | | | |
| B. longum | BILO9e02a | BILO9e02a2 | BILO9e02a2 | mna 12 | gAaCTAAAGCGgTTTAGAAT | −71 | 6.20 |
| subsp. infantis | 2a1_01962 | a1_00982 | a1 00982- | 5-manI | | | |
| Bg41721_1G8_SN_2018 | | | BILO9e02a2 | | | | |
| | | | a1_00983 | | | | |
| | | BILO9e02a2 | BILO9e02a2 | Blon_23 | ATaCTAAAtCGgTTTAGtTa | −140 | 5.38 |
| | | a1_01282 | a1_01282- | 80-2378- | | | |
| | | | BILO9e02a2 | b1Man5 | | | |
| | | | a1_01283- | B | | | |
| | | | BILO9e02a2 | | | | |
| | | | a1_01284- | | | | |
| | | | BILO9e02a2 | | | | |
| | | | a1_01285 | | | | |
| | | BILO9c02a2 | BILO9c02a2 | mna38*- | tTaCTAAAGCGCTTTAGtcT | −115 | 5.49 |
| | | a1_01968 | a1_01968- | mna38 | | | |
| | | | BILO9e02a2 | | | | |
| | | | a1_01967 | | | | |
| B. longum | BILO16373 | BILO163738 | BILO163738 | mna38*- | tTaCTAAAGCGCTTTAGtcT | −117 | 5.49 |
| subsp. infantis | 828_01184 | 28_01173 | 28_01173- | mna38 | | | |
| JG_Bg463.m5.93_JG | | | BILO163738 | | | | |
| | | | 28_01174 | | | | |
| | | BILO163738 | BILO163738 | Blon_23 | tTaCTAAAcCCaTTTACtTa | −198 | 4.93 |
| | | 28_01175 | 28_01175- | 80-2378- | | | |
| | | | BILO163738 | b1Man5 | | | |
| | | | 28_01176- | B | | | |
| | | | BILO163738 | | | | |
| | | | 28_01177- | | | | |
| | | | BILO163738 | | | | |
| | | | 28_01178 | | | | |
| | | BILO163738 | BILO163738 | mna 12 | cAaCTAAAGCGgTTTAaAAT | −68 | 5.71 |
| | | 28_01186 | 28_01186- | 5-manI | | | |
| | | | BILO163738 | | | | |
| | | | 28_01185 | | | | |
| B. longum | BILO14587 | BILO145876 | BILO145876 | endoBI- | ATaCTAAAGCGaTTTAGtTc | −123 | 5.75 |
| subsp. infantis | 6ef_01011 | ef_00034 | ef_00034 | 1 | | | |
| PS064_13.C6_Bang_JG | | BILO145876 | BILO145876 | mna38*- | tTaCTAAAGCGCTTTAGtcT | −117 | 5.49 |
| | | ef_01005 | ef_01005- | mna38 | | | |
| | | | BILO145876 | | | | |
| | | | ef_01006 | | | | |
| | | BILO145876 | BILO145876 | mna_12 | gAaCTAAAGCGgTTTAGAAT | −69 | 6.20 |
| | | ef_01013 | ef_01013- | 5-manI | | | |
| | | | BILO145876 | | | | |
| | | | ef_01012 | | | | |
| | | BILO145876 | BILO145876 | Blon_23 | ATaCTAAAGCGgTTTAGtTa | −138 | 5.86 |
| | | cf_02188 | cf_02188- | 80-2378- | | | |
| | | | BILO145876 | b1Man5 | | | |
| | | | ef_02187- | B | | | |
| | | | BILO145876 | | | | |
| | | | ef_02186- | | | | |
| | | | BILO145876 | | | | |
| | | | ef_02185 | | | | |

NglR; ROK family

| | | | | | | | |
|---|---|---|---|---|---|---|---|
| B. longum | BILO543B | BILO543B3 | BILO543B3 | mna38- | TATa TTTACATTGGAAACATA | -77 | 6.92 |
| subsp. infantis | 32D0_ | 2D0_04560 | 2D0_04560- | nglABC- | | | |
| Bg40721_2D9_SN_2018 | 04590 | | BILO543B3 | hex3- | | | |
| | | | 2D0_04565- | b1Man5b | | | |
| | | | BILO543B3 | *-nglR- | | | |
| | | | 2D0_04570- | hypo | | | |
| | | | BILO543B3 | | | | |
| | | | 2D0_04575- | | | | |
| | | | BILO543B3 | | | | |
| | | | 2D0_04580- | | | | |
| | | | BILO543B3 | | | | |
| | | | 2D0_04585- | | | | |
| | | | BILO543B3 | | | | |
| | | | 2D0_04590- | | | | |
| | | | BILO543B3 | | | | |
| | | | 2D0_04595 | | | | |

BglT; TetR family

| | | | | | | | |
|---|---|---|---|---|---|---|---|
| B. longum | BILO543B | BILO543B3 | BILO543B3 | bglXYZ- | TACTTACTTACAACTAACTA | −117 | 7.38 |
| subsp. infantis | 32D0_ | 2D0_06910 | 2D0_06910- | bgn_30- | | | |
| Bg40721_2D9_SN_2018 | 06885 | | BILO543B3 | hypo- | | | |
| | | | 2D0_06905- | bgrT- | | | |
| | | | BILO543B3 | gluD- | | | |
| | | | 2D0_06900- | GH2 | | | |
| | | | BILO543B3 | | | | |
| | | | 2D0_06895- | | | | |
| | | | BILO543B3 | | | | |
| | | | 2D0_06890- | | | | |
| | | | BILO543B3 | | | | |
| | | | 2D0_06885- | | | | |
| | | | BILO543B3 | | | | |
| | | | 2D0_06880- | | | | |
| | | | BILO543B3 | | | | |
| | | | 2D0_06875 | | | | |

### Example 4: Invitro Growth Experiments with Bangladeshi Strains Selected from the mcSEED Analysis

Based on the mcSEED observations, a series of in vitro growth experiments were performed using four Bangladesh B. infantis strains plus EVC001.

### Methods

B. infantis strains were streaked from frozen stocks onto Brain Heart Infusion (BHI) blood agar plates which were incubated for 48 hours at 37° C. under anaerobic conditions. Three colonies of each strain were used to generate three individual overnight monocultures cultures in 1 mL of low-carbohydrate minimal De Man/Rogosa/Sharp (MRS) medium (lcMRS) in a 96-well plate. 10 mL of an aqueous stock solution of filter-sterilized 10% (w/v) glucose was added to 40 mL of lcMRS to make lcMRS+glucose medium. 50 μL of the culture was added to 1 mL of lcMRS+glucose medium in a 96-well plate and these subcultures were incubated under anaerobic conditions for 16 hours at 37° C. the OD₆₀₀ of the subcultures was then recorded and each was adjusted to an OD₆₀₀ of 0.3 in lcMRS+glucose broth in a fresh 96-well plate. Lactose or different HMOs |Lacto-N-tetraose (LNT; Evolve Biosystems), Lacto-N-neotetraose (LNnT; Glycom A/S), 2′fucosyllactose (2′FL; Glycosyn), 3′sialyllactose (3′SL; Genechem) and 6′ sialyllactose (6′SL; Genechem)] were dissolved in distilled water at 100 g/L and filter sterilized. A 30 μL aliquot of each HMO stock solution was added to 120 μL of lcMRS and mixed (final HMO concentration 2% w/v). 5 μL of the OD₆₀₀ standardized subcultures were used to inoculate the lcMRS+carbohydrate medium into 96-well plates, Growth at 37° C. under anaerobic conditions was monitored over 30 hours by measuring OD600 every 15 minutes using a Gen5 Microplate Reader (Biotek). Experiments were conducted with three biological replicates. The significance of observed differences in OD600 values at 30 hours were calculated using a one-way ANOVA with a Dunnett's post hoc test (using strain EVC001 as the reference control group).

### Results

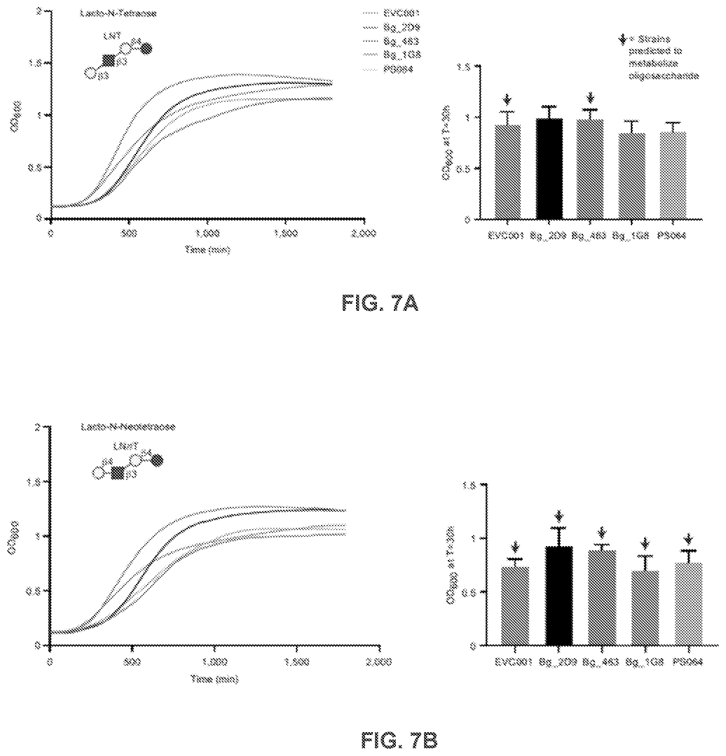

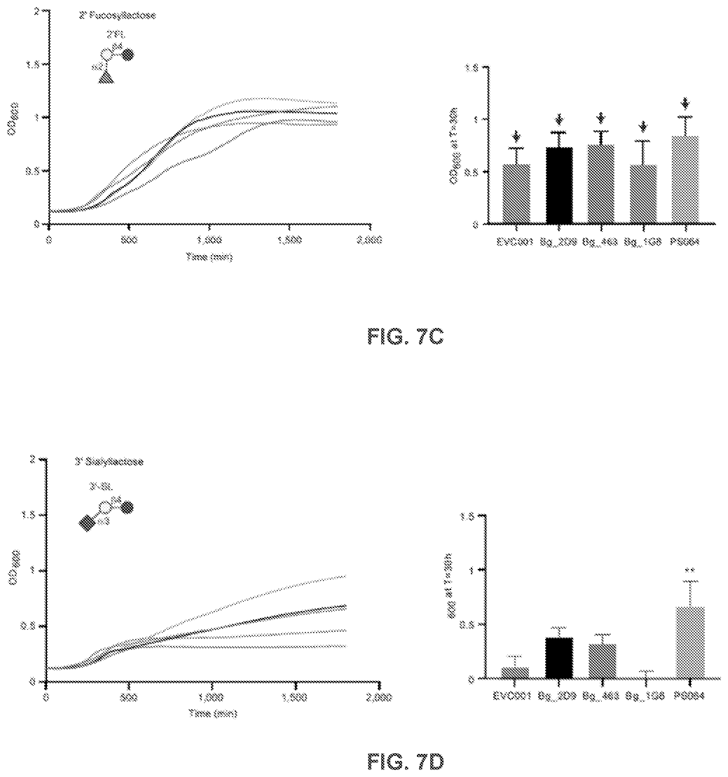

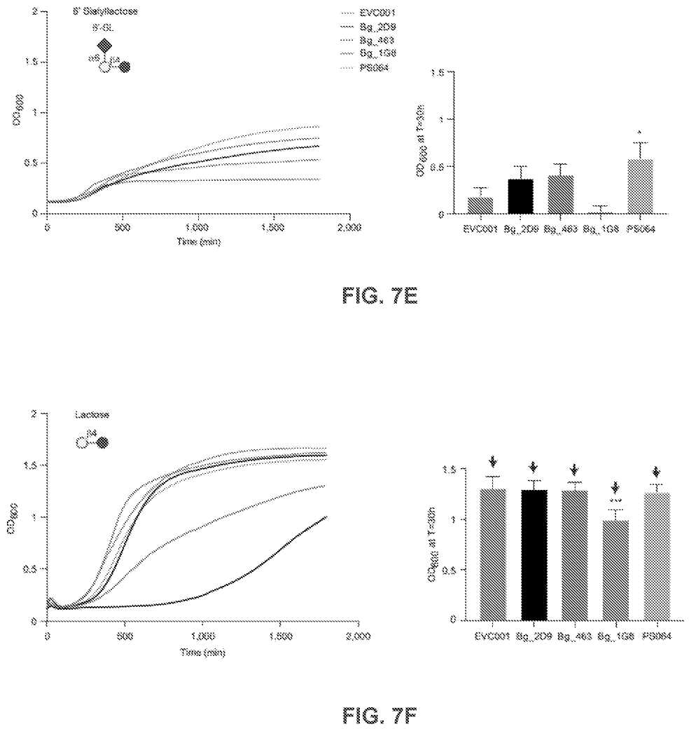

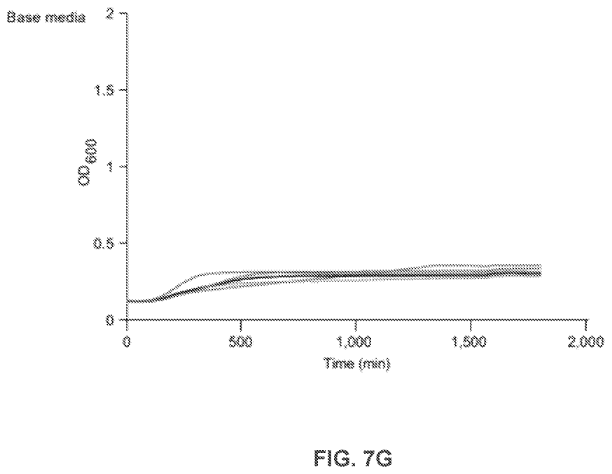

Each of the strains tested exhibited slower growth rates in the presence of sialylated HMOs (3′-SL and 6′-SL; FIG. 7D,E) compared to neutral or fucosylated carbohydrate structures (FIG. 7A-C). [To date, no SL-specific transporter has been characterized in bifidobacteria, though the exo-a-sialidase NanH2 (Blon_2348, GH33) that is ubiquitous among B. infantis strains cleaves the sialic acid residue from both 3′-SL and 6′-SL].

It was observed that a given strain's propensity to grow in the presence of LNnT and 2′-FL was generally closely linked the presence of their transporters, Blon_2345-2347 and Blon_2202-2204 respectively, in addition to the full complement of downstream catabolic enzymes (see Table 8).

The biochemically-characterized LNT transporter GltABC (Blon_2175-77) (Garrido D. et al., Oligosaccharide binding proteins from B. longum subsp. infantis reveal a preference for host glycans. PLOS One 6 (2011)) was absent in Bg_2D9 but present in all the other strains, including EVC001. An unanticipated result was the comparable growth of strains in the presence of LNT (FIG. 7A), suggesting that Bg_2D9 has an alternative mechanism for LNT uptake, or utilization, or both whose efficiency is comparable to the canonical LNT transporter, at least in vitro in the absence of competition. Based on these findings, a study was performed in gnotobiotic mice colonized with a consortium of five B. infantis strains where this canonical LNT transporter was either present (EVC001, Bg_463) or absent (Bg_2D9, Bg_1G8, PS064).

### Example 5: In Vivo Competition Between Divergent B. Infantis Strains in Gnotobiotic Mice Fed HMO-Supplemented Bangladeshi Diets

### Methods:

All mouse experiments were carried out using protocols approved by the Washington University in St. Louis Institutional Animal Care and Use Committee (IACUC). Mice were housed in plastic flexible film gnotobiotic isolators (Class Biologically Clean Ltd., Madison, WI) at 23° C. under a strict 12-hour light cycle (lights on at 0600h).

### Construction of the Mirpur-6 Diet

Based on extensive knowledge of Bangladeshi complementary feeding practices, including quantitative 24-hour dietary recall surveys conducted at the Mirpur site (see (MAL-ED Network Investigators (2014) for a description of methods) a ‘Mirpur-6 diet’ was prepared by Dyets, Inc. (Bethlehem, PA) to be representative of the contribution of milk and complementary foods consumed by 6-month-old infants living in Mirpur (Table 9).

TABLE 9
Composition of the un-supplemented Mirapur-6 diet
Mirpur-6 Diet

| Ingredients | % by weight |
|---|---|
| Cooked Rice (parboiled) | 21.73 |
| Cooked Lentils (masoor) | 14.49 |
| Whole milk powder (as breast-milk substitute) | 32.6 |
| Cooked Potato | 7.24 |
| Cooked Spinach | 7.24 |
| Cooked Onion (yellow) | 4.71 |
| Soybean oil | 5.43 |
| Sweet pumpkin | 5.43 |
| Salt (iodized) | 0.36 |
| Turmeric | 0.36 |
| Garlic | 0.36 |
| Total | 99.95 |

In brief, rice (parboiled, long grain) and red lentils (masoor dal) were each cooked separately with an equal weight of water at 100° C. in a steam-jacketed kettle until partially cooked (still firm) and then set aside. Market fresh potatoes, spinach and yellow onions were washed, chopped in a vertical cutter mixer and cooked in the kettle without added water at 70° C. until soft. Sweet pumpkin (Calabaza variety) was chopped and boiled in the steam-jacketed kettle until soft and then strained. At this point, all of the cooked ingredients were combined, whole bovine milk powder (Franklin Farms East, Bethlehem, PA), soybean oil, salt, turmeric and garlic were added and the resulting diet was mixed extensively and allowed to cool. Diets were dried on trays overnight at 30° C. and pelleted by extrusion (½″ diameter; California Pellet Mill, CL5). Dried pellets were weighed into 250 g portions, placed in a paper bag with an inner wax-lining which in turn was placed in a plastic bag. The plastic bag was vacuumed sealed and its contents were sterilized by gamma irradiation (30-50 kGy; Sterigenics, Rockaway, NJ). Sterility was confirmed using culture-based assays, as described in (11). Nutritional analysis of the diet was performed by Nestlé Purina Analytical Laboratories; St. Louis, MO (Table 10).

TABLE 10
Nutritional analysis of the un-supplemented Mirpur-6 diet

| Moisture, vacuum oven, 100° C. | 8.59% | |
|---|---|---|
| Protein, combustion (N × 6.25) | 17.40% | |
| Fat by GC | 20.1 g/100 g | |
| Saturated Fatty Acids | 8.99 g/100 g | |
| Monounsaturated Fatty Acids | 4.5 g/100 g | |
| Polyunsaturated Fatty Acids | 4.92 g/100 g | |
| Omega 3 Fatty Acids | 0.523 g/100 g | |
| Omega 6 Fatty Acids | 4.4 g/100 g | |
| Trans Fatty Acids | 0.43 g/100 g | |
| Total dietary fiber | 2.77% | |
| Insoluble dietary fiber | 1.88% | |
| Soluble dietary fiber | 0.88% | |
| Carbohydrate (by calculation) | 48.70% | |
| Calories, bomb calorimetry | 4.93 | kcal/g |
| Vitamin A | <715 | IU A/lb |
| Vitamin C | 47.4 | ppm |
| Viatmin D | <0.5 | IU D/g |
| Vitamin E | <0.4 mg/100 g | |
| Thiamin (B1) | 2.67 | ppm |
| Riboflavin (B2) | 6.47 | ppm |
| Niacin (B3) | 19.4 | ppm |
| Pantothenic acid (B5) | 16.1 | ppm |
| Pyridoxine (B6) | 0.96 | ppm |
| Biotin (B7) | 0.134 | ppm |
| Folic Acid (B9) | 0.335 | ppm |
| Zinc | 20.4 | ppm |

### Colonization of Mice

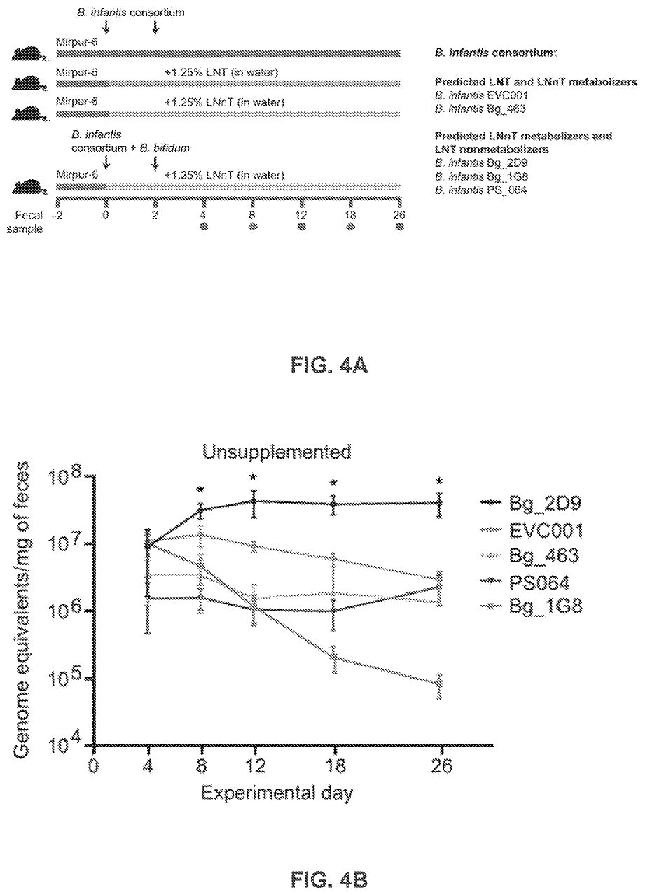

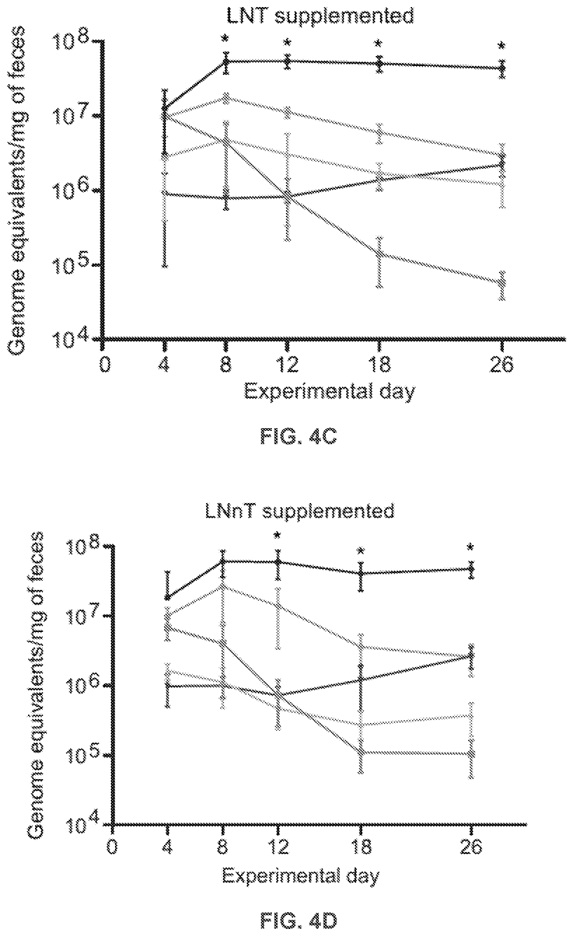

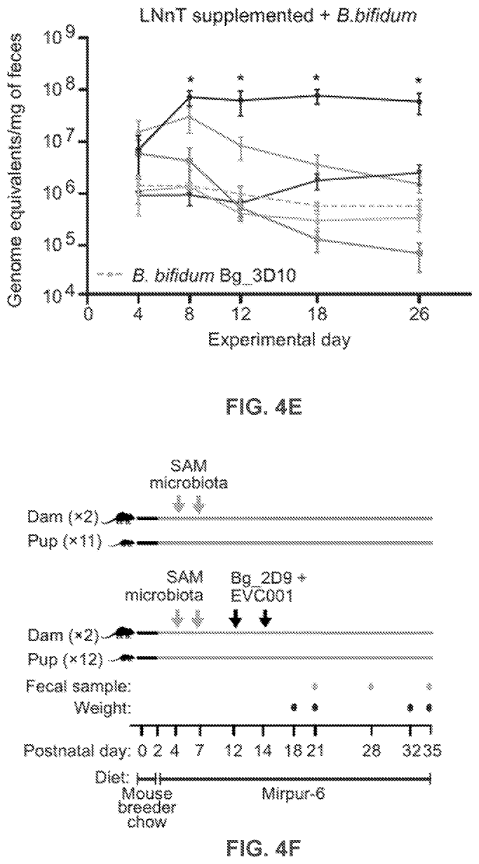

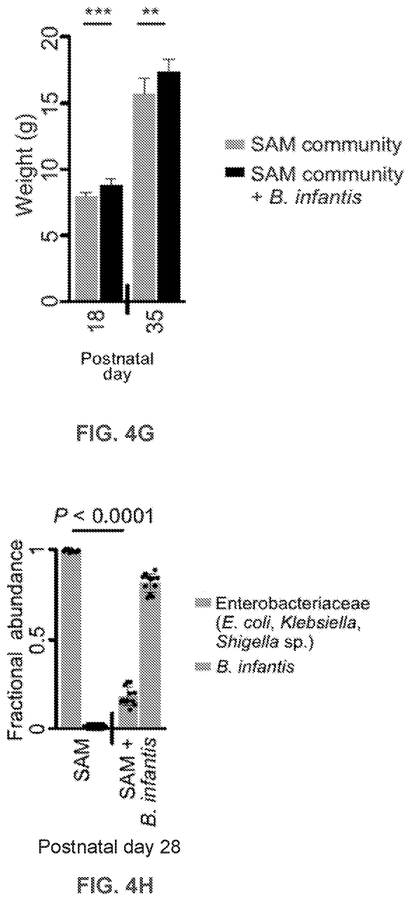

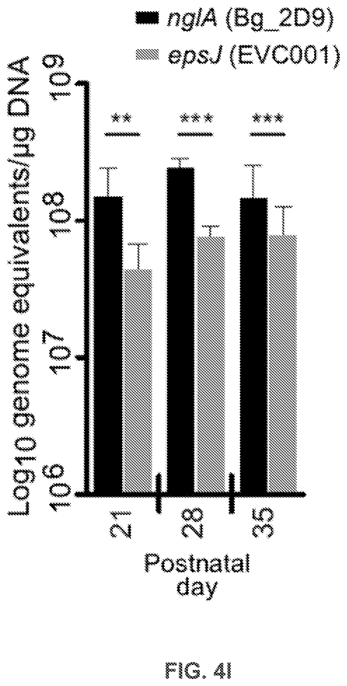

The four B. infantis strains that had been isolated from Bangladeshi children were combined with EVC001 prior to gavage. For one arm of the experiment, these consortium of B. infantis strains were supplemented with a B. bifidum strain derived from a Bangladesh child fecal sample (B. bifidum_41221_3D10). Frozen stocks of the cultured strains were thawed inside the Coy chamber and 100 μL of the stock was spread on agar plates containing MRS agar and 0.05% L-cysteine-HCl. Plates were incubated at 37° C. under anaerobic conditions for 48 h. Single colonies were handpicked and transferred into 5 mL of MRS broth. Liquid cultures were subsequently incubated at 37° C. under anaerobic conditions for 24 h, after which time a 100 μL aliquot was withdrawn to measure OD600. All liquid monocultures were then normalized to the lowest OD600 among the strains (0.6) and equal volumes of each organism was pooled to generate the consortium mixture used for the experiment described in FIG. 4A. Glass crimp vials (Wheaton) were filled with 800 μL of 1:1 mixture of sterile PBS/30% glycerol/0.05% L-cysteine hydrochloride and the pooled strains, sealed and immediately stored in −80° C. until use within a week.

5-week-old germ-free male C57BL/6 mice were fed the Mirpur-6 diet for 2 days prior to gavage with a defined consortium; this was followed by a second gavage of the same consortium two days later. Fecal specimens were collected every 48 hours from all animals in all treatment groups. Throughout the experiment, all animals in all treatment groups were provided the Mirpur-6 diet ad libitum. Mice received autoclaved water with or without LNT or LNnT: the dose of LNT or LNnT administered was equivalent to that consumed if the Mirpur-6 diet had been supplemented with 12.5 g/L (1.25%) HMO. Non-fasted animals were euthanized by cervical dislocation on experimental day 28.

### Experimental Set-Up

The animals were divided into four groups after 2 days of consumption of the ‘Mirpur-6’ diet. The drinking water of one group of animals was supplemented with 12.5 g/L LNT, another group received with 12.5 g/L LNnT, while a third control group received unsupplemented water. In these three arms of the experiment, mice (n=6-7/group) were subsequently colonized with the consortium of five B. infantis strains followed 2 days later by a second gavage with the same consortium (see FIG. 4A for experimental design).

In a fourth arm, mice were colonized with the 5-member B. infantis consortium plus a B. bifidum strain (Bg_3D10) that was cultured from the fecal microbiota of a healthy, 12-month-old Mirpur child; these mice were treated with LNnT supplemented drinking water (FIG. 4A). Analysis of the genome of this B. bifidum strain indicated that it contains genes encoding membrane-bound extracellular lacto-N-biosidase I (LnbB) and extracellular exo-β-(1-3)-N-acetylglucosamidase (BbhI) which endow it with the capacity to degrade LNT and LNnT (35, 36)], resulting in release of Lacto-N-Biose and lactose. In the case of LNnT, B. bifidum first removes the terminal galactose from the non-reducing end via extracellular β-1,4-galactosidase BbgIII (GH2); subsequently, the GlcNAc residue is cleaved by exo-β-(1-3)-N-acetylglucosamidase BbhI, resulting in the liberation of Gal, GlcNAc, and lactose that can subsequently be utilized by B. bifidum itself, or potentially through cross-feeding by other community members. Therefore, studies with expanded ‘dimensionality’ of the staged competition to one involving a consortium member could be performed, that could potentially limit the amount of LNnT available to other consumers through a mechanism that is not dependent upon its ability to directly import this HMO. Mice in all four treatment groups were fed the Mirpur-6 diet ad libitum for 4 weeks; every 2 days fecal samples were collected and water, with or without HMO supplementation, was replenished daily.

Quantifying Absolute Abundances of B. infantis and B. bifidum Strains

The absolute abundances of B. infantis strains in fecal samples collected from colonized mice were defined by short read shotgun sequencing of community DNA (COPRO-Seq; 59). To determine absolute abundance, 6.7×10⁶ cells of Alicyclobacillus acidiphilus DSM 14558 and 29.8×10⁶ cells of Agrobacterium radiobacter DSM 30147 were added to each weighed frozen fecal pellet collected from each animal on study days 4, 8, 12, 18, 26 (60). Fecal pellets were then subjected to bead beading for 4 minutes (Mini-BeadBeater-8, BioSpec) in a mixture containing 500 μL of extraction buffer [200 mM NaCl, 200 mM Tris (pH 8), 20 mM EDTA], 210 μL of 20% SDS, 500 μL of phenol/chloroform/isoamyl alcohol (pH 7.9) (25:24:1; Ambion), and 250 μL of 0.1-mm zirconia beads (BioSpec Products). Samples were centrifuged at 4° C. for 4 minutes at 3,220×g. The aqueous phase was collected, nucleic acids were purified using QIAquick columns (Qiagen) and eluted from the columns into 10 mM Tris-Cl, pH 8.5. DNA concentration was quantified (Quant-iT dsDNA assay kit, broad sensitivity; ThermoFisher), and adjusted to 0.75 ng/μL with UltraPure water (Milli-Q). COPRO-Seq libraries were prepared using the Nextera DNA Library Prep kit protocol (Illumina) and custom barcoded primers (61). Barcoded libraries were sequenced on an Illumina NextSeq instrument [75 nt single-end reads; 2.71±1.38×10⁶ reads/sample (mean±S.D.)].

Reads were de-multiplexed and mapped to the sequenced whole genomes of the five B. strains, plus five “distractor” genomes (Lactobacillus ruminis ATCC 27782, Olsenella uli DSM 7084, Pasteurella multocida USDA-ARS-USMARC 60385, Prevotella dentalis DSM 3688 and Staphylococcus saprophyticus ATCC 15305). The proportion of total reads mapping to the five distractor genomes for each sample was used to set a conservative threshold (mean±2SD) for colonization of an organism in the animals. For each member of the community, absolute abundance was calculated by multiplying the normalized counts of strains with abundance of Alicyclobacillus acidiphilus (cell number per normalized count) divided by the sample weight (62). Mixed-effects linear models followed by Tukey's post-hoc test was applied to test for significant interaction of time and abundance of the strains.

### Microbial RNA-Seq

Cecal contents harvested from gnotobiotic mice at the time of euthanasia were flash frozen in liquid nitrogen and stored at −80° C. For RNA extraction, cecal samples were kept on ice and the following reagents added in the following order: (i) 250 μL of acid-washed glass beads (212-300 um; Millipore Sigma; G1277), (ii) 500 μL of 2X Buffer B (200 mM NaCl, 20 mM EDTA), (iii) 210 μL of 20% SDS, and (iv) 500 μL phenol: chloroform: isoamyl alcohol (125:24:1, pH 4.5; ThermoFisher, AM9720). The mixture was homogenized using a bead beater (Mini-BeadBeater-8, BioSpec) at room temperature for a total of 5 minutes, with a pause for 2 minutes on ice after the first three minutes. The mixture was centrifuged (7000 ×g for 10 minutes at 4° C.) and RNA was isolated from 500 μL of the aqueous phase using a previously described protocol (63). RNA integrity and fragment size were assessed [4200 TapeStation System (Agilent)] followed by elimination of genomic DNA by using two sequential DNAase treatments [Baseline-ZERO DNase (Lucigen) and Turbo DNAse (Invitrogen)]. Absence of genomic DNA was verified by qPCR using primers against the B. spp 16S rDNA (28). Total RNA was purified using the MEGAclear Transcription Clean-Up Kit (ThermoFisher, AM1908), quantified using Qubit RNA BR Assay Kit (Invitrogen) and 1 μg was depleted of ribosomal RNA using the Ribo-Zero (Epidemiology/Bacteria) kit (Illumina) followed by ethanol precipitation. The SMARTer Stranded RNASeq kit (Takara Bio USA) was used to prepare double-stranded complementary DNA and indexed libraries. Libraries were sequenced using an Illumina NextSeq platform [70-nt unidirectional reads; 5.3×10⁷±2.8×10⁶ reads/sample (mean±SD); n=26 samples]. The first five cycles of sequencing were omitted as this library preparation strategy introduces three non-templated deoxyguanines. Reads were demultiplexed, checked for quality using FastQC and were mapped to the genomes of the members of the consortia. Transcript counts were normalized and analyzed using the DESeq2 package in R (version 4.0.2; 64) at the level of individual strains. For each strain, the raw count data was fitted to a negative binomial model using the DESeq2 workflow and statistical tests were performed to identify differentially expressed genes in the following groups of animals: (i) LNT-supplemented animals compared to their unsupplemented counterparts, (ii) LNnT-supplemented animals compared to their unsupplemented counterparts, and, (iii) animals fed the LNnT-supplemented diet and colonized with or without B. bifidum.

### Pangenome Analysis

A pangenome analysis of the five B. infantis strains used in the gnotobiotic mouse experiments was performed using Roary 3.12.0 (Page A. J. et al Roary: rapid large-scale prokaryote pan genome analysis. Bioinformatics 31, 3691-3693 (2015)). Genomes in the gff format with mcSEED-derived annotations were inputted and the pangenome with a 95% minimum percentage identity cut-off for Blastp (Altschul S.F. and Lipman D. J. et al. Protein database searches for multiple alignments. Proc Natl Acad Sci USA. 87, 5509-5513 (1990)) was generated using the following command:

$$roary - p 16 - e - n - i 95 - f 95 \_percent * . gff$$

267 genes unique to B. longum subsp. infantis Bg40721_2D9_SN_2018 were manually screened and genomic clusters corresponding to carbohydrate metabolism were identified.

### Results

There were no significant differences in body weights (measured every 2 days) between the four treatment groups (P>0.05, two-way repeated measures ANOVA). The absolute abundance of each B. infantis strain in each of the treatment groups was determined on experimental days at days 4, 8, 12, 18 and 26 by short read shotgun sequencing of fecal DNA. Strikingly, the Bg_2D9 strain, which lacks the LNT transporter (Blon_2175-2177), became the dominant member over time on this diet, achieving an absolute abundance on day 18 that was >10-fold higher than each of other community members in the three groups that had received 5-member consortium (FDR adjusted P<0.01, mixed effects model followed by Tukey's multiple comparison test) (FIG. 4B-D). During the first 18 days of colonization, EVC001 was the second most abundant strain in the three defined communities comprised exclusively of B. infantis isolates, after which time it no longer exhibited a competitive advantage (FIG. 4B-D). In the 6-member community containing B. bifidum, Bg_2D9 came to dominate by day 8 and maintained its significantly higher absolute abundance compared to each of the other strains for the duration of the experiment (FIG. 4E).

These in vivo studies establish that (i) the presence of the Blon_2175-2177 LNT uptake transporter in the EVC001 and Bg_463 strains does not confer a fitness advantage in this diet/experimental context even when LNT is present at levels ˜10-fold higher that what is normally found in breast milk, (ii) the Bg_2D9 strain exhibits superior fitness in the absence or presence of LNT or LNnT, and (iii) the potential for cross-feeding on products of extracellular HMO metabolism by B. bifidum does not alter the superior fitness of Bg_2D9 over other community members.

### Mechanistic Analysis

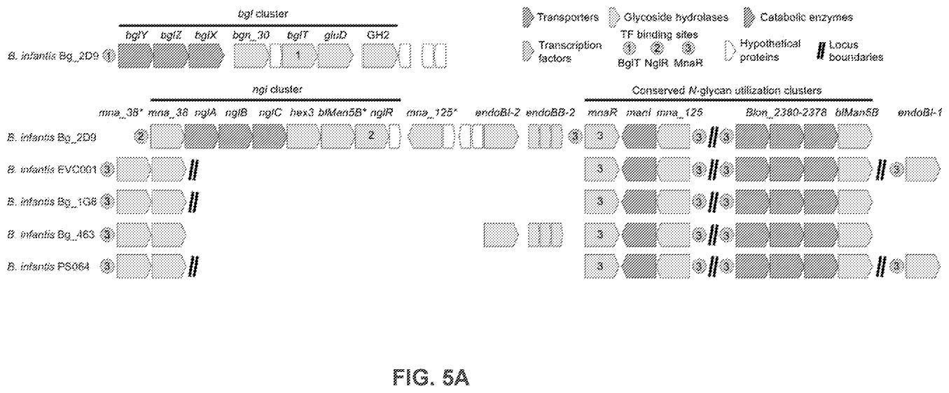

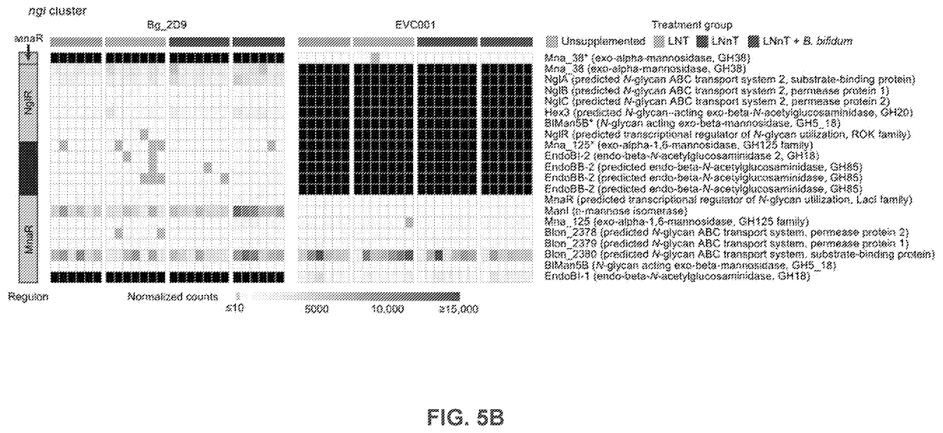

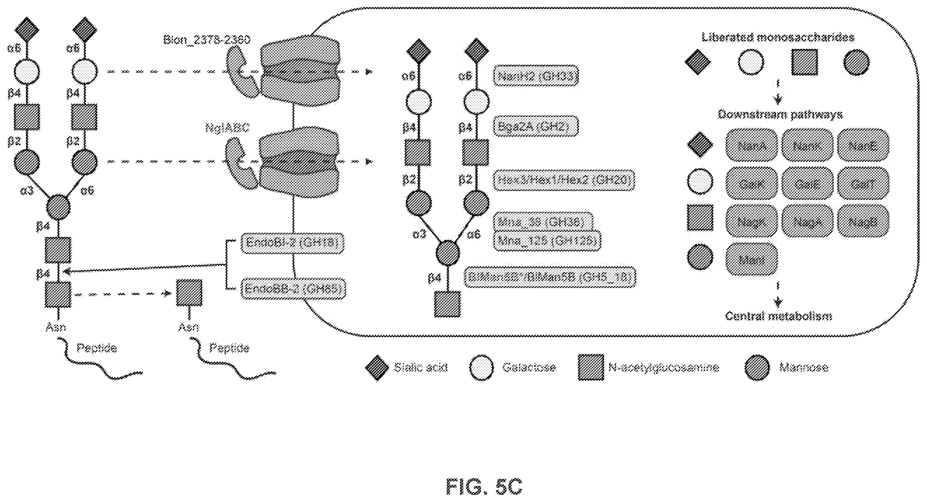

Comparisons of the Bg_2D9 genome and those of other consortium members—To identify features that might explain the competitive advantage of the Bg_2D9 strain, genome to genomes comparisons were made, of each of the other four B. infantis strains in the consortium. Using a minimum 95% threshold of identity for orthologous genes, 267 genes that were unique to this strain were identified, most of which coded for short hypothetical proteins or mobile elements. Among these however, were a predicted β-glucoside utilization cluster (Bgl) and an N-glycan utilization cluster (Ngl) (FIG. 5A, Table 11, 12 and 13).

TABLE 11
Representation of glycoside hydrolases and transporters involved in N-glycan utilization

| | Glycoside hydrolases | | | | | |
|---|---|---|---|---|---|---|
| | Locus tag (name) | | | | | |
| | | | BILO543B32D0_04625- | | | |
| | Blon_2468 | BILO543B32D0_04620 | BILO543B32D0_04635 | Blon_0868 | Blon_0869 | Blon_0876 |
| | (EndoBI-1) | (EndoBI-2) | (EndoBB-2) | (Mna_38*) | (Mna_38) | (Mna_125) |
| | Function or specificity | | | | | |
| | Endo-β-N- | Endo-β-N- | Endo-β-N- | α- | α- | α- |
| | acetylglucosaminidase | acetylglucosaminidase | acetylglucosaminidase | mannosidase | mannosidase | mannosidase |
| | (GH18) | (GH18) | (GH85) | (GH38) | (GH38) | (GH125) |
| B. longum | + | − | − | + | + | + |
| subsp. infantis | | | | | | |
| EVC001 | | | | | | |
| B. longum | + | − | − | + | + | + |
| subsp. infantis | | | | | | |
| ATCC 15697 | | | | | | |
| B. longum | − | + | + | + | + | + |
| subsp. infantis | | | | | | |
| JG_Bg463.m5.93_JG | | | | | | |
| B. longum | − | + | + | − | + | + |
| subsp. infantis | | | | | | |
| Bg40721_2D9_SN_2018 | | | | | | |
| B. longum | − | + | + | + | + | + |
| subsp. infantis | | | | | | |
| Bg40721_2C3_SN_2018 | | | | | | |
| B. longum | − | − | − | + | + | + |
| subsp. infantis | | | | | | |
| Bg41721_1E9_SN_2018 | | | | | | |
| B. longum | − | − | − | + | + | + |
| subsp. infantis | | | | | | |
| Bg41721_1G8_SN_2018 | | | | | | |
| B. longum | + | − | − | + | + | + |
| subsp. infantis | | | | | | |
| PS064_13.C6_Bang_JG | | | | | | |
| B. longum | − | + | + | + | + | + |
| subsp. infantis | | | | | | |
| Malawi_264A_MC1 | | | | | | |
| B. longum | + | + | + | + | + | + |
| subsp. infantis | | | | | | |
| Malawi_264A_MC2 | | | | | | |
| B. longum | − | − | − | + | + | + |
| subsp. suis | | | | | | |
| PS131.S11.17_F6 Bang_JG | | | | | | |
| B. breve | − | − | − | + | + | + |
| PS155.S09_23A9_JG_2018 | | | | | | |
| B. breve | − | + | + | + | + | + |
| PE1C332.m20.82_Peru_JG | | | | | | |
| B. longum | − | − | − | − | − | − |
| STL_TW14.1_LFYP82 | | | | | | |
| B. bifidum | − | − | − | − | − | − |
| Bg41221_3D10_SN_2018 | | | | | | |

| | Glycoside hydrolases | | | | |
|---|---|---|---|---|---|
| | Locus tag (name) | | | | |
| | | | Blon_0459; | | |
| | Blon_2377 | BILO543B32D0_04585 | Blon_0732 | Blon_2355 | BILO543B32D0_04580 |
| | (BlMan5B) | (BlMan5B*) | (Hex1) | (Hex2) | (Hex3) |
| | Function or specificity | | | | |
| | β- | β- | β-1,3/4/6-N- | β-1,3/4-N- | β-N- |
| | mannosidase | mannosidase | acetylglucosaminidase | acetylglucosaminidase | acetylglucosaminidase |
| | (GH5_18) | (GH5_18) | (GH20) | (GH20) | (GH20) |
| B. longum | + | − | + | + | − |
| subsp. infantis | | | | | |
| EVC001 | | | | | |
| B. longum | + | − | + | + | − |
| subsp. infantis | | | | | |
| ATCC 15697 | | | | | |
| B. longum | + | − | + | + | − |
| subsp. infantis | | | | | |
| JG_Bg463.m5.93_JG | | | | | |
| B. longum | + | + | + | + | + |
| subsp. infantis | | | | | |
| Bg40721_2D9_SN_2018 | | | | | |
| B. longum | + | + | + | + | + |
| subsp. infantis | | | | | |
| Bg40721_2C3_SN_2018 | | | | | |
| B. longum | + | − | + | + | − |
| subsp. infantis | | | | | |
| Bg41721_1E9_SN_2018 | | | | | |
| B. longum | + | − | + | + | − |
| subsp. infantis | | | | | |
| Bg41721_1G8_SN_2018 | | | | | |
| B. longum | + | − | + | + | − |
| subsp. infantis | | | | | |
| PS064_13.C6_Bang_JG | | | | | |
| B. longum | + | − | + | + | − |
| subsp. infantis | | | | | |
| Malawi_264A_MC1 | | | | | |
| B. longum | + | + | + | + | + |
| subsp. infantis | | | | | |
| Malawi_264A_MC2 | | | | | |
| B. longum | + | − | + | + | − |
| subsp. suis | | | | | |
| PS131.S11.17_F6 Bang_JG | | | | | |
| B. breve | + | − | + | − | − |
| PS155.S09_23A9_JG_2018 | | | | | |
| B. breve | + | − | + | − | − |
| PE1C332.m20.82_Peru_JG | | | | | |
| B. longum | − | − | + | − | − |
| STL_TW14.1_LFYP82 | | | | | |
| B. bifidum | − | − | + | − | − |
| Bg41221_3D10_SN_2018 | | | | | |

| | Glycoside hydrolases | | | | Transporters | |
|---|---|---|---|---|---|---|
| | Locus tag (name) | | | | | |
| | | | | | | BILO543B32D004565- |
| | Blon_2334 | Blon_2348 | Blon_2335 | Blon_2336 | Blon_2378- | BILO543B32D0_04575 |
| | (Bga2A) | (NanH2) | (BiAfcA) | (BiAfcB) | 2380 | (NglABC) |
| | Function or specificity | | | | | |
| | β-1,4- | α-2,3/6- | α-1,2-L- | α-1,3/4-L- | N-glycans; | |
| | galactosidase | sialidase | fucosidase | fucosidase | α-mannose | N- |
| | (GH2) | (GH33) | (GH95) | (GH29) | oligosaccharides | glycans |
| B. longum | + | + | + | + | + | − |
| subsp. infantis | | | | | | |
| EVC001 | | | | | | |
| B. longum | + | + | + | + | + | − |
| subsp. infantis | | | | | | |
| ATCC 15697 | | | | | | |
| B. longum | + | + | + | + | + | − |
| subsp. infantis | | | | | | |
| JG_Bg463.m5.93_JG | | | | | | |
| B. longum | + | + | + | + | + | + |
| subsp. infantis | | | | | | |
| Bg40721_2D9_SN_2018 | | | | | | |
| B. longum | + | + | + | + | + | + |
| subsp. infantis | | | | | | |
| Bg40721_2C3_SN_2018 | | | | | | |
| B. longum | + | + | + | + | + | − |
| subsp. infantis | | | | | | |
| Bg41721_1E9_SN_2018 | | | | | | |
| B. longum | + | + | + | + | + | − |
| subsp. infantis | | | | | | |
| Bg41721_1G8_SN_2018 | | | | | | |
| B. longum | + | + | + | + | + | − |
| subsp. infantis | | | | | | |
| PS064_13.C6_Bang_JG | | | | | | |
| B. longum | + | + | + | + | + | − |
| subsp. infantis | | | | | | |
| Malawi_264A_MC1 | | | | | | |
| B. longum | + | + | + | + | + | + |
| subsp. infantis | | | | | | |
| Malawi_264A_MC2 | | | | | | |
| B. longum | + | + | + | + | + | − |
| subsp. suis | | | | | | |
| PS131.S11.17_F6 Bang_JG | | | | | | |
| B. breve | + | − | + | − | + | − |
| PS155.S09_23A9_JG_2018 | | | | | | |
| B. breve | + | − | + | − | + | − |
| PE1C332.m20.82_Peru_JG | | | | | | |
| B. longum | + | − | − | − | − | − |
| STL_TW14.1_LFYP82 | | | | | | |
| B. bifidum | + | − | − | + | + | − |
| Bg41221_3D10_SN_2018 | | | | | | |

TABLE 12
Representation of glycoside hydrolases and transporters in the Bgl cluster

| | Glycoside hydrolases | | | Transporters |
|---|---|---|---|---|
| | Locus tag (name) | | | |
| | | | | BILO543B32D0_06910- |
| | BILO543B32D0_06895 | BILO543B32D0_06880 | BILO543B32D0_06875 | BILO543B32D0_06900 |
| | (Bgn_30) | (GluD) | (GH2) | (BglXYZ) |
| | Function or specificity | | | |
| | Endo-β-1,6- | β-1,3/4- | Hypothetical | |
| | glucanase | glucosidase | glycoside hydrolase | |
| | (GH30) | (GH3) | (GH2) | β-glucosides |
| B. longum subsp. infantis EVC001 | − | − | − | − |
| B. longum subsp. infantis ATCC 15697 | − | − | − | − |
| B. longum subsp. infantis | − | − | − | − |
| JG_Bg463.m5.93_JG | | | | |
| B. longum subsp. infantis | + | + | ± | + |
| Bg40721_2D9_SN_2018 | | | | |
| B. longum subsp. infantis | + | + | + | + |
| Bg40721_2C3_SN_2018 | | | | |
| B. longum subsp. infantis | − | − | − | − |
| Bg41721_1E9_SN_2018 | | | | |
| B. longum subsp. infantis | − | − | − | − |
| Bg41721_1G8_SN_2018 | | | | |
| B. longum subsp. infantis | − | − | − | − |
| PS064_13.C6_Bang_JG | | | | |
| B. longum subsp. infantis | + | + | + | + |
| Malawi_264A_MC1 | | | | |
| B. longum subsp. infantis | − | − | − | − |
| Malawi_264A_MC2 | | | | |
| B. longum subsp. suis | − | − | − | − |
| PS 131.S11.17_F6Bang_JG | | | | |
| B. breve PS 155.S09_23A9_JG_2018 | − | − | − | − |
| B. breve PE1C332.m20.82_Peru_JG | + | + | + | + |
| B. longum STL_TW14.1_LFYP82 | − | − | − | − |
| B. bifidum By41221_3D10_SN_2018 | − | − | − | − |

TABLE 13
Representation of Ngl and Bgl loci in 336 bifidobacterial genomes

| Locus | Bgl | Ngl |
|---|---|---|
| B. adolescentis strain Km 4 | − | − |
| B. adolescentis strain AL12-4 | − | − |
| B. adolescentis strain 6 | − | − |
| B. adolescentis strain ca_0067 | − | − |
| B. adolescentis L2-32 | − | − |
| B. adolescentis strain P2P3 | − | − |
| B. adolescentis strain 70B | − | − |
| B. adolescentis strain 487B | − | − |
| B. adolescentis strain AM14-37 | − | − |
| B. adolescentis strain LMG 10733 | − | − |
| B. adolescentis strain MGYG-HGUT-02395 | − | − |
| B. adolescentis strain BIO5485 | − | − |
| B. adolescentis strain AL46-7 | − | − |
| B. adolescentis strain 22L | − | − |
| B. adolescentis strain 1892B | − | − |
| B. adolescentis strain ZJ2 | − | − |
| B. adolescentis strain AF15-3 | − | − |
| B. adolescentis strain BBMN23 | − | − |
| B. adolescentis strain TM06-4 | − | − |
| B. adolescentis strain AM13-11 | − | − |
| B. adolescentis LFYP80 | − | − |
| B. adolescentis strain 1-11 | − | − |
| B. adolescentis ATCC 15703 | − | − |
| B. adolescentis strain 150 | − | − |
| B. stercoris JCM 15918 | − | − |
| B. angulatum DSM 20098 | − | − |
| B. angulatum strain GT102 | − | − |
| B. animalis subsp. lactis CNCM I-2494 | − | − |
| B. animalis subsp. lactis Bl-04 | − | − |
| B. animalis subsp. lactis KLDS2.0603 | − | − |
| B. animalis subsp. lactis strain HN019 | − | − |
| B. animalis subsp. lactis BS 01 | − | − |
| B. animalis subsp. lactis ATCC 27673 | − | − |
| B. animalis strain Probio-M8 | − | − |
| B. animalis subsp. lactis Bi-07 | − | − |
| B. animalis subsp. lactis BB-12 | − | − |
| B. animalis subsp. lactis strain S7 | − | − |
| B. animalis subsp. lactis RH | − | − |
| B. animalis subsp. lactis V9 | − | − |
| B. animalis subsp. lactis DSM 10140 | − | − |
| B. animalis subsp. lactis strain IDCC4301 | − | − |
| B. animalis subsp. lactis B94 | − | − |
| B. animalis subsp. lactis AD011 | − | − |
| B. animalis subsp. lactis B420 | − | − |
| B. animalis subsp. lactis Bl12 | − | − |
| B. animalis subsp. lactis BF052 | − | − |
| B. animalis subsp. lactis BLC1 | − | − |
| B. bifidum strain BIOML-A8 | − | − |
| B. bifidum strain BIOML-A3 | − | − |
| B. bifidum strain MGYG-HGUT-02396 | − | − |
| B. bifidum strain TM04-12 | − | − |
| B. bifidum ATCC 29521 = JCM I255 = DSM 20456 | − | − |
| B. bifidum strain 324B | − | − |
| B. bifidum strain BF3 | − | − |
| B. bifidum S17 | − | − |
| B. bifidum strain ICIS-643 | − | − |
| B. bifidum LMG 13195 strain JCM 7004 | − | − |
| B. bifidum BGN4 | − | − |
| B. bifidum strain BIOML-A9 | − | − |
| B. bifidum NCIMB 4117I | − | − |
| B. bifidum strain 156B | − | − |
| B. bifidum strain ICIS-202 | − | − |
| B. bifidum strain 1 G1971 | − | − |
| B. bifidum strain BIOML-A6 | − | − |
| B. bifidum strain ca_0067 | − | − |
| B. bifidum strain LMG 11582 | − | − |
| B. bifidum strain S6 | − | − |
| B. bifidum strain 85B | − | − |
| B. bifidum strain LMG 11041 | − | − |
| B. bifidum strain BSD2780061688st1_G1 | − | − |
| B. bifidum strain ICIS-310 | − | − |
| B. bifidum strain 791 | − | − |
| B. bifidum strain MJR8628B | − | − |
| B. bifidum strain BIOML-A4 | − | − |
| B. bifidum strain PRI 1 | − | − |
| B. bifidum PRL2010 | − | − |
| B. bifidum strain TMC 3115 | − | − |
| B. bifidum strain AM36-IAC | − | − |
| B. bifidum strain NCTC13001 | − | − |
| B. bifidum Bg41221_3D10_SN_2018 | − | − |
| B. breve DPC 6330 | − | − |
| B. breve MCC 0305 | − | − |
| B. breve JCM 7019 | + | − |
| B. breve MCC 1340 | − | − |
| B. breve MCC 1604 | − | − |
| B. breve 689b | − | − |
| B. breve 2L | − | − |
| B. breve JCM 7017 | − | − |
| B. breve MCC 1094 | − | − |
| B. breve UCC2003 | + | − |
| B. breve MCC 0476 | − | − |
| B. breve MCC 1605 | − | − |
| B. breve 12L | − | − |
| B. breve NCFB 2258 | − | − |
| B. breve MCC 0121 | − | − |
| B. breve MCC 1454 | − | − |
| B. breve MCC 1128 | − | − |
| B. breve CECT 7263 | − | − |
| B. breve ACS-071-V-Sch8b | − | − |
| B. breve HPH0326 | − | − |
| B. breve DSM 20213 = JCM1192 | − | − |
| B. breve BBJG463 | − | − |
| B. breve MCC 1114 | − | − |
| B. breve PS 155.S09_23A9_JG_2018 | − | − |
| B. breve PE1C332.m20.82_Peru_JG | + | − |
| B. catenulatum DSM 16992 | − | − |
| B. catenulatum JG_Bg468_v2 | − | − |
| B. catenulatum BPSS39 | − | − |
| B. catenulatum strain 1899B | − | − |
| B. kashiwanohense PV20-2 | − | − |
| B. kashiwanohense JCM 15439 = DSM 21854 | − | − |
| B. dentium JCM 1195 = DSM 20436 | − | − |
| B. dentium Bd1 | − | − |
| B. dentium ATCC 27679 | − | − |
| B. dentium JCVIHMP022 | − | − |
| B. dentium LFYP24 | − | − |
| B. dentium ATCC 27678 | − | − |
| B. gallicum DSM 20093 | − | − |
| B. longum 239-2 | − | − |
| B. longum I897B | − | − |
| B. longum STL_TW14.1_LFYP82 | − | − |
| B. longum subsp. infantis strain LH_665 | − | − |
| B. longum subsp. infantis strain NCTC11817 | − | − |
| B. longum subsp. infantis BIBI401272845a | − | − |
| B. longum subsp. infantis BIC1401111250 | − | − |
| B. longum subsp. infantis strain Bi-26 | − | − |
| B. longum subsp. infantis R0033 | − | − |
| B. longum subsp. infantis strain LH_23 | − | − |
| B. longum subsp. infantis EK3 | − | − |
| B. longum subsp. infantis strain LH_666 | − | − |
| B. longum subsp. infantis strain IN-F29 | − | + |
| B. longum subsp. infantis strain 1888B | − | − |
| B. longum subsp. infantis strain TPY12-1 | − | − |
| B. longum subsp. infantis strain NCTC13219 | − | − |
| B. longum subsp. infantis BIBI401242951 | − | − |
| B. longum subsp. infantis BIC1206122787 | − | − |
| B. longum subsp. infantis strain LH_664 | − | − |
| B. longum subsp. infantis BIC1401212621b | − | − |
| B. longum subsp. infantis strain IN-07 | − | − |
| B. longum subsp. infantis BIC1307292462 | − | − |
| B. longum subsp. infantis strain BIO5478 | − | − |
| B. longum subsp. infantis BIC1401212621a | − | − |
| B. longum subsp. infantis strain BT1 | − | − |
| B. longum subsp. infantis strain BI-G201 | − | − |
| B. longum subsp. infantis BIBI401272845b | − | − |
| B. longum subsp. infantis EVC001 | − | − |
| B. longum subsp. infantis ATCC 15697 | − | − |
| B. longum subsp. infantis JG_Bg463.m5.93_JG | − | − |
| B. longum subsp. infantis Bg40721_2D9_SN_2018 | + | + |
| B. longum subsp. infantis Bg40721_2C3_SN_2018 | + | + |
| B. longum subsp. infantis Bg41721_1E9_SN_2018 | − | − |
| B. longum subsp. infantis Bg41721_1G8_SN_2018 | − | − |
| B. longum subsp. infantis PS064_13.C6_Bang_JG | − | − |
| B. longum subsp. infantis Malawi_264A_MC1 | + | − |
| B. longum subsp. infantis Malawi_264A_MC2 | − | + |
| B. longum subsp. longum N6D05 | − | − |
| B. longum subsp. longum 72B | − | − |
| B. longum subsp. longum strain LO-21 | − | − |
| B. longum subsp. longum N2E12 | − | − |
| B. longum subsp. longum strain VMKB44 | − | − |
| B. longum subsp. longum AF08-2 | − | − |
| B. longum subsp. longum strain BLOI2 | − | − |
| B. longum subsp. longum DPC6320 | − | − |
| B. longum subsp. longum AF03-20 | − | − |
| B. longum subsp. longum DPC6317 | − | − |
| B. longum subsp. longum 17-1B | − | − |
| B. longum subsp. longum strain 7 | − | − |
| B. longum subsp. longum 7-1B | − | − |
| B. longum subsp. longum AM11-5 | − | − |
| B. longum subsp. longum AF05-16 | − | − |
| B. longum subsp. longum TF01-22 | − | − |
| B. longum subsp. longum APC1462 | − | − |
| B. longum subsp. longum strain LO-06 | − | − |
| B. longum subsp. longum D2957 | − | − |
| B. longum subsp. longum AF26-10 | − | − |
| B. longum subsp. longum MCC10007 | − | − |
| B. longum subsp. longum strain DS32_3 | − | − |
| B. longum subsp. longum DSM 20219 | − | − |
| B. longum subsp. longum APC1472 | − | − |
| B. longum subsp. longum AF13-34 | − | − |
| B. longum subsp. longum W35-1 | − | − |
| B. longum subsp. longum Indica | − | − |
| B. longum subsp. longum strain LO-C29 | − | − |
| B. longum subsp. longum strain AH1206 | − | − |
| B. longum subsp. longum 35B | − | − |
| B. longum subsp. longum 379 | − | − |
| B. longum subsp. longum CMW7750 | − | − |
| B. longum subsp. longum DPC6323 | − | − |
| B. longum subsp. longum AF27-1BH | − | − |
| B. longum subsp. longum APC1480 | − | − |
| B. longum subsp. longum TF06-45A | − | − |
| B. longum subsp. longum AF30-12 | − | − |
| B. longum subsp. longum TF07-39 | − | − |
| B. longum subsp. longum DS7_3 | − | − |
| B. longum subsp. longum strain BORI | − | − |
| B. longum subsp. longum ICIS-505 | − | − |
| B. longum subsp. longum OF01-16 | − | − |
| B. longum subsp. longum AM11-2 | − | − |
| B. longum subsp. longum AF11-12 | − | − |
| B. longum subsp. longum AM21-20 | − | − |
| B. longum subsp. longum N2F05 | − | − |
| B. longum subsp. longum APC1466 | − | − |
| B. longum subsp. longum AM20-39 | − | − |
| B. longum subsp. longum OM05-2BH | − | − |
| B. longum subsp. longum DS1_3 | − | − |
| B. longum subsp. longum NCC2705 | − | − |
| B. longum subsp. longum APC1468 | − | − |
| B. longum subsp. longum SC596 | − | − |
| B. longum subsp. longum AM31-13LB | − | − |
| B. longum subsp. longum APC1476 | − | − |
| B. longum subsp. longum TF07-34 | − | − |
| B. longum subsp. longum APC1482 | − | − |
| B. longum subsp. longum AF14-22 | − | − |
| B. longum subsp. longum 157F | − | − |
| B. longum subsp. longum CCUG 52486 | − | − |
| B. longum subsp. longum 1-5B | − | − |
| B. longum subsp. longum 1886B | − | − |
| B. longum subsp. longum CECT 7347 | − | − |
| B. longum subsp. longum F8 | − | − |
| B. longum subsp. longum TM04-48B | − | − |
| B. longum subsp. longum NCTC11818 | − | − |
| B. longum subsp. longum AM20-19AC | − | − |
| B. longum subsp. longum strain NCIMB8809 | − | − |
| B. longum subsp. longum 1898B | − | − |
| B. longum subsp. longum E18 | − | − |
| B. longum subsp. longum EK5 | − | − |
| B. longum subsp. longum AM12-16 | − | − |
| B. longum subsp. longum AF36-1 | − | − |
| B. longum subsp. longum AM34-3 | − | − |
| B. longum subsp. longum TF08-4AC | − | − |
| B. longum subsp. longum DS18_3 | − | − |
| B. longum subsp. longum APC1478 | − | − |
| B. longum subsp. longum TM01-1 | − | − |
| B. longum subsp. longum BBMN68 | − | − |
| B. longum subsp. longum TM06-1 | − | − |
| B. longum subsp. longum DPC6316 | − | − |
| B. longum subsp. longum AM39-8AC | − | − |
| B. longum subsp. longum strain VKPM Ac-1636 | − | − |
| B. longum subsp. longum AF03-10 | − | − |
| B. longum subsp. longum DPC6321 | − | − |
| B. longum subsp. longum AM42-13AT | − | − |
| B. longum subsp. longum APC1465 | − | − |
| B. longum subsp. longum 1890B | − | − |
| B. longum subsp. longum DS15_3 | − | − |
| B. longum subsp. longum strain 296B | − | − |
| B. longum subsp. longum strain W11 | − | − |
| B. longum subsp. longum AM20-3 | − | − |
| B. longum subsp. longum AM30-9LB | − | − |
| B. longum subsp. longum strain LO-10 | − | − |
| B. longum subsp. longum JCM 1217 | − | − |
| B. longum subsp. longum TM05-14 | − | − |
| B. longum subsp. longum DS9_3 | − | − |
| B. longum subsp. longum KACC 91563 | − | − |
| B. longum subsp. longum AF04-13 | − | − |
| B. longum subsp. longum BG7 | − | − |
| B. longum subsp. longum N5E04 | − | − |
| B. longum subsp. longum AF13-41 | − | − |
| B. longum subsp. longum ATCC 55813 | − | − |
| B. longum subsp. longum CECT 7210 | − | − |
| B. longum subsp. longum strain LMG 13197 | − | − |
| B. longum subsp. longum 35624 | − | − |
| B. longum subsp. longum TM02-7 | − | − |
| B. longum subsp. longum AF30-11 | − | − |
| B. longum subsp. longum TF07-31 | − | − |
| B. longum subsp. longum AF34-9AC | − | − |
| B. longum subsp. longum AM39-10AC | − | − |
| B. longum subsp. longum APC1503 | − | − |
| B. longum subsp. longum EK13 | − | − |
| B. longum subsp. longum N3E01-2 | − | − |
| B. longum subsp. longum AF35-13AC | − | − |
| B. longum subsp. longum TM04-17 | − | − |
| B. longum subsp. longum APC1473 | − | − |
| B. longum subsp. longum strain CCUG30698 | − | − |
| B. longum subsp. longum APC1477 | − | − |
| B. longum subsp. longum AM10-15B | − | − |
| B. longum subsp. longum APC1464 | − | − |
| B. longum subsp. longum GT15 | − | − |
| B. longum subsp. longum strain LO-K29b | − | − |
| B. longum subsp. longum AF05-2 | − | − |
| B. longum subsp. longum AF11-41 | − | − |
| B. longum subsp. longum N3A01 | − | − |
| B. longum subsp. longum 2-2B | − | − |
| B. longum subsp. longum AM16-2 | − | − |
| B. longum subsp. longum AF14-34 | − | − |
| B. longum subsp. longum 1 | − | − |
| B. longum subsp. longum UMB0788 | − | − |
| B. longum subsp. longum 105-A | − | − |
| B. longum subsp. longum AF03-27 | − | − |
| B. longum subsp. longum N2G10 | − | − |
| B. longum subsp. longum DJO10A | − | − |
| B. longum subsp. longum 1-6B | − | − |
| B. longum subsp. longum strain 9 | − | − |
| B. longum subsp. longum 44B | − | − |
| B. longum subsp. longum CAG:69 | − | − |
| B. longum subsp. longum strain LO-K29a | − | − |
| B. longum subsp. longum strain MC-42 | − | − |
| B. longum subsp. longum 12_1_47BFAA | − | − |
| B. longum subsp. longum APC1504 | − | − |
| B. longum subsp. longum TF06-12AC | − | − |
| B. longum subsp. suis APC1461 | − | − |
| B. longum subsp. suis CMCC P0001 | − | − |
| B. longum subsp. suis BXY01 | − | − |
| B. longum subsp. suis JDM301 | − | − |
| B. longum subsp. suis AGR2137 | − | − |
| B. longum subsp. suis PS 131.S11.17_F6 Bang_JG | − | − |
| B. pseudocatenulatum strain 1896B | − | − |
| B. pseudocatenulatum strain 1001271st1_F3 | − | − |
| B. pseudocatenulatum strain CA-C29 | − | − |
| B. pseudocatenulatum strain Bif4 | − | − |
| B. pseudocatenulatum strain CA-K29b | − | − |
| B. pseudocatenulatum strain AM43-10 | − | − |
| B. pseudocatenulatum strain NBRC 113353 | − | − |
| strain NBRC 13719 | | |
| B. pseudocatenulatum DSM 20438 = JCM 1200 = | − | − |
| LMG 10505 | | |
| B. pseudocatenulatum strain ca_0067 | − | − |
| B. pseudocatenulatum IPLA36007 | − | − |
| B. pseudocatenulatum strain CECT 7765 | − | − |
| B. pseudocatenulatum strain AF11-18 | − | − |
| B. pseudocatenulatum strain LH_663 | − | − |
| B. pseudocatenulatum strain AF26-1 | − | − |
| B. pseudocatenulatum strain TF05-2AC | − | − |
| B. pseudocatenulatum LFYP29 | − | − |
| B. pseudocatenulatum strain LH_662 | − | − |
| B. pseudocatenulatum strain CA-05 | − | − |
| B. pseudocatenulatum strain MGYG-HGUT-03683 | − | − |
| B. pseudocatenulatum strain AM18-42 | − | − |
| B. pseudocatenulatum strain CA-D29 | − | − |
| B. pseudocatenulatum strain CA-B29 | − | − |
| B. pseudocatenulatum strain CA-K29a | − | − |
| B. pseudocatenulatum strain LH_659 | − | − |
| B. pseudocatenulatum strain 12 | − | − |
| B. pseudocatenulatum strain TM10-1 | − | − |
| B. pseudocatenulatum strain LII_658 | − | − |
| B. pseudolongum PV8-2 | − | − |
| B. scardovii JCM 12489 - DSM 13734 | − | + |
| B. scardovii 981_BLON | − | + |
| B. thermophilum RBL67 | − | − |

The Bgl cluster contains (i) three glycoside hydrolases (GHs) [a hypothetical glucan endo-β-1,6-glucosidase belonging to glycoside hydrolase family 30 (GH30), an exo-β-1,4/6-glucosidase (GH3), and a hypothetical β-galactosidase (GH2 family)]; (ii) an ABC transport system [encoded by bglY, bglZ, bglX] and (iii) a TetR family transcriptional regulator [bgl7]. Among 34 published B. infantis genomes (Davis J. J. et al. The PATRIC Bioinformatics Resource Center: expanding data and analysis capabilities. Nucleic Acids Res. 48, D606-D612 (2020)), including those in this report, only three (Bg_2D9, Bg_2C3, and Malawi_MC1; all described here) possess this locus (Table 13). Given that (i) β-1,3-linked glucosides are common constituents of plant cell wall polysaccharides and (ii) approximately 60% by weight of the Mirpur-6 diet is plant-based (Table 9), it was reasoned that the representation of a unique beta-glucoside utilization cluster in B. infantis Bg_2D9 could provide one explanation for its fitness advantage over the other members of the B. infantis consortium introduced into gnotobiotic mice.

Asparagine-linked glycans (N-glycans) have structural similarities to HMOs and are abundant in human and bovine milk where they decorate numerous proteins including lactoferrin and immunoglobulins. B. infantis ATCC 15697 is capable of utilizing a wide array of N-glycans both in vitro and in vivo (Garrido D. et al., Endo-β-N-acetylglucosaminidases from infant gut-associated bifidobacteria release complex N-glycans from human milk glycoproteins. Mol. Cell Proteomics 11, 775-785 (2012), Karav S et al., Oligosaccharides released from milk glycoproteins are selective growth substrates for infant-associated Bifidobacteria. Appl. Environ. Microbiol. 82, 3622-3630 (2016)). However, details of this process remain poorly understood with only the first step of N-glycan utilization described; namely the release of the sugar moiety by membrane-bound endo-β-N-acetylglucosaminidases (EndoBI-1 and EndoBI-2, GH18) acting on N,N-diacetylchitobiose core of N-linked glycans.

In this study, the Ngl cluster in the Bg_2D9 genome was identified that contains two endo-β-N-acetylglucosaminidases: EndoBI-2 and EndoBB-2 (GH85) (FIG. 5A, Table 11). The enzymatic activity of EndoBI-2 has been characterized biochemically (Garrido D. et al., Endo-β-N-acetylglucosaminidases from infant gut-associated bifidobacteria release complex N-glycans from human milk glycoproteins. Mol. Cell Proteomics 11, 775-785 (2012)) while the function of EndoBB-2 is predicted. The Ngl cluster also contains genes encoding (i) an ABC transport system (NglABC) predicted to transport N-glycans; (ii) GHs involved in degradation of (complex)N-glycans, namely a-mannosidase, Mna_38 (GH38), a homolog of the biochemically-characterized β-mannosidase, BIMan5B (GH5_18; 41), a β-N-acetylglucosaminidase, Hex3 (GH20), (iii) a transcriptional regulator (NglR) from the ROK family, NgIR (FIG. 5A). The glycan effector for NgIR is unknown but predicted to be a degradation product of complex N-glycan metabolism. Extending analysis to the database of 336 bifidobacterial genomes, including the aforementioned 34 B. infantis genomes, revealed that the Bg_2D9 strain was one of only six that contained the Ngl cluster (Table 13).

EVC001 is predicted to have N-glycan metabolizing capabilities via an alternative pathway that includes EndoBI-1, B-mannosidase BIMan5B coupled with another predicted N-glycan transporter (Blon_2378-2380) (Cordeiro R. L. et al. N-glycan utilization by B. gut symbionts involves a specialist β-Mannosidase. J. Mol. Biol. 431, 732-747 (2019)), and a-mannosidase Mna_125 (GH125) linked to mannose isomerase ManI under control of a LacI-family transcriptional regulator MnaR. The reconstructed MnaR regulon includes the mna 125-manI, mnaR, and Blon_2380-2378-blMan5B operons (present in all five B. infantis strains used in the gnotobiotic mouse experiment), mna_38 genes in four strains (except Bg_2D9), and endoBI-1 in two strains (FIG. 5A). While Bg_2D9 strain does not have an ortholog of EndoBI-1, it does contain an endo-β-N-acetylglucosaminidase (EndoBI-2), a predicted endo-β-N-acetylglucosaminidase (EndoBB-2) and the MnaR-regulated Blon_2380-2378-blMan5B operon (FIG. 5A).

These observations indicated that among the strains evaluated, Bg_2D9 strain has the greatest endowment of GHs and candidate transporters for N-glycan utilization. As summarized in FIG. 5C, comparative genomic analysis suggest that (i) its endo-β-N-acetylglucosaminidases EndoBI-2 and EndoBB-2 are available to release sugar moieties from N-glycans, which are further transported into the cell via NglABC or Blon_2378-2380, (ii) these ABC transport systems may exhibit different preferences for various N-glycan structures and (iii) internalized sugar moieties are degraded from the non-reducing end by an orchestrated action of multiple intracellular exo-acting GHs. Many GHs may also be involved in HMO utilization, namely, Bga2A, Hex1, Hex2, NanH2, BiAfcA, BiAfcB may contribute to utilization of complex N-glycans (containing GlcNAc, fucose, and NANa residues) given that these enzymes are known to act on glycosidic bonds found in both HMOs and N-glycans.

Other unique genomic clusters in Bg_2D9-Besides the Ngl and Bgl loci, other loci that distinguished the Bg_2D9 strain from other B. infantis strains in the consortium were (i) a locus encoding an ABC carbohydrate transporter with unknown specificity (BILO543B32D0_04140_BILO543B32D0_04165) and (ii) a locus encoding enzymes, mostly glycosyltransferases, involved in exopolysaccharide (EPS) biosynthesis (BILO543B32D0_09145_BILO543B32D0_09175). The presence of the latter locus is of particular interest, since, among bifidobacteria, synthesis of EPS is characteristic of B. breve, but typically not B. infantis (Fanning S. et al., Bifidobacterial surface-exopolysaccharide facilitates commensal-host interaction through immune modulation and pathogen protection. Proc. Natl Acad Sci. USA 109, 2108-2113 (2012)).

Analysis of gene expression of consortium members-Microbial RNA-Seq of cecal contents harvested at the time of euthanasia of mice colonized with the five B. infantis strains with or without B. bifidum was performed. This was to examine expression of the β-glucoside utilization (Bgl) and N-glycan utilization (Ngl) loci, as well as other genes involved in LNT and LNnT utilization, as a function of the presence or absence of these HMOs in the drinking water. Transcript counts were normalized and analyzed using DESeq2 and mapped to the genomes of consortium members.

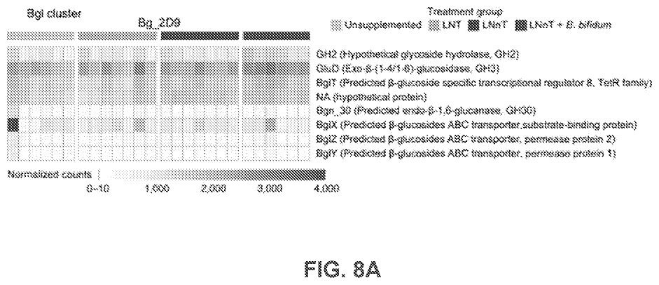

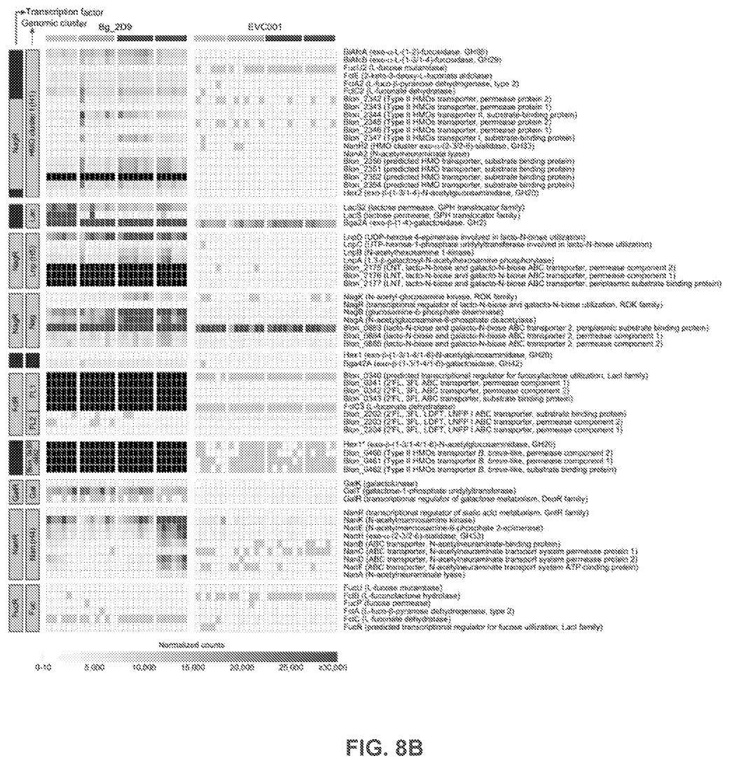

All eight genes comprising the Bgl cluster were expressed in the Bg_2D9 strain; there were no statistically significant differences in their expression in the presence versus absence of either LNT or LNnT (FIG. 8A, Tables 14a and 14b). As noted above, this cluster comprises a regulon controlled by a TetR family regulator, BgIT (FIG. 5A, Table 8). This regulator likely responds to β-gluco-oligosaccharides originating from the grain components (rice, lentil) of the Mirpur-6 diet.

There were no statistically significant effects of HMO supplementation on expression of genes within the Ngl cluster in Bg_2D9 regulated by the ROK family transcription factor NgIR (Mna_38, exo-a-mannosidase and NgIA, N-glycan ABC transport system 2 substrate-binding protein) or by the LacI-family transcriptional factor MnaR [ManI (D-mannose isomerase) and Blon_2380 (predicted N-glycan substrate binding protein)]; all of these genes were expressed to varying degrees in Bg_2D9 (FIG. 5B, Tables 8, 14a and 14b).

Compared to animals receiving the Mirpur-6 diet alone, supplementation with LNT produced a significant increase in expression (log 2-fold difference >1.5 and FDR-adjusted P<0.05) of genes in the H1 cluster encoding six type II HMO transporter proteins (orthologs of Blon_2342-2347) in the Bg_2D9 strain, and two in the EVC001 strain (Blon_2343 and Blon_2346) (see FIG. 1, FIG. 8B and Tables 14b). In LNnT-supplemented mice, with the exception of Blon_2345, expression of these genes was also elevated in Bg_2D9 compared to their expression in unsupplemented animals (FIG. 8B, Tables 14b). The absence of significant induction of other glycan transporter genes in the presence of LNT raises the possibility that this H1 cluster encodes a transport system capable of importing LNT as well as LNnT.

LNnT supplementation also significantly increased levels of expression of several genes required for HMO metabolism in the Bg_2D9 strain, including nagA (N-acetylglucosamine-6-phosphate deacetylase) and nagB (glucosamine-6-phosphate deaminase) which are involved in GlcNAc catabolismthe Inp cluster (H5) involved in lacto-N-biose/galacto-N-biose catabolismand predicted HMO transporters Blon_2350 and Blon_2351 in the H1 cluster (log 2-fold difference >1.5 and FDR-adjusted P<0.05; FIG. 8B; Tables 14b, 16). In contrast, expression of genes involved in sialic acid utilization including the Nan cluster (H4) sialic acid ABC transporter and a N-acetylneuraminate lyase (nanA) were reduced in Bg_2D9 in both LNT- and LNnT-supplemented animals (log 2-fold change >1.2, FDR-adjusted P<0.05; FIG. 8B; Tables 14b).

Finally, when comparing the LNnT experimental groups with or without B. bifidum, expression of most genes involved in HMO and N-glycan metabolism did not differ significantly (FIG. 8B, Tables 14a and 14b). However, there was a significant increase in the expression of H4 cluster genes involved in sialic acid catabolismin the Bg_2D9 genome when B. bifidum was present (log 2-fold change >1.8, FDR-adjusted P<0.5; Table 14a and 14b).

TABLE 14a

| | | | | B. longum | | |
|---|---|---|---|---|---|---|
| | | | | subsp. Infantis | | |
| | Genomic | | | ATCC 15697 | Gene annotation | mcSEED |
| Strain | cluster | Regulon | Locus tag | locus tag | (mcSEED) | pathway |
| Bifidobacterium | Bgl | BglT | BILO543B32D0_06875 | NA | GH2 (Hypothetical | Beta- |
| longum subsp. infantis | | | | | glycoside hydrolase, | glucosides |
| Bg40721_2D9_SN_2018 | | | | | GH2) | catabolism |
| | | BglT | BILO543B32D0_06880 | NA | GluD (Exo-beta-(1- | Beta- |
| | | | | | 4/1-6)-glucosidase, | glucosides |
| | | | | | GH3) | catabolism |
| | | BglT | BILO543B32D0_06885 | NA | BglT (Predicted beta- | Beta- |
| | | | | | glucoside specific | glucosides |
| | | | | | transcriptional | regulation |
| | | | | | regulator 8, TetR | |
| | | | | | family) | |
| | | BglT | BILO543B32D0_06890 | NA | NA (hypothetical | NA |
| | | | | | protein) | |
| | | BglT | BILO543B32D0_06895 | NA | Bgn_30 (Predicted | Beta- |
| | | | | | endo-β-1,6-glucanase, | glucosides |
| | | | | | GH30) | catabolism |
| | | BglT | BILO543B32D0_06900 | NA | BglX (Predicted beta- | Beta- |
| | | | | | glucosides ABC | glucosides |
| | | | | | transporter, substrate- | uptake |
| | | | | | binding protein) | |
| | | BglT | BILO543B32D0_06905 | NA | BglZ (Predicted beta- | Beta- |
| | | | | | glucosides ABC | glucosides |
| | | | | | transporter, permease | uptake |
| | | | | | protein 2) | |
| | | BglT | BILO543B32D0_06910 | NA | BglY (Predicted beta- | Beta- |
| | | | | | glucosides ABC | glucosides |
| | | | | | transporter, permease | uptake |
| | | | | | protein 1) | |
| | FL2 | FclR | BILO543B32D0_09610 | Blon_2202 | Blon_2202 (2′FL, 3FL, | HMO uptake |
| | | | | | LDFT, LNFP I ABC | |
| | | | | | transporter, substrate- | |
| | | | | | binding protein) | |
| | | FclR | BILO543B32D0_09615 | Blon 2203 | Blon_2203 (2′FL, 3FL, | HMO uptake |
| | | | | | LDFT, LNFP I ABC | |
| | | | | | transporter, permease | |
| | | | | | component 2) | |
| | | FclR | BILO543B32D0_09620 | Blon_2204 | Blon_2204 (2′FL, 3FL, | HMO uptake |
| | | | | | LDFT, LNFP I ABC | |
| | | | | | transporter, permease | |
| | | | | | component 1) | |
| | Fuc | FucR | BILO543B32D0_10225 | Blon_2305 | FucU (L-fucose | Fucose |
| | | | | | mutarotase) | catabolism |
| | | FucR | BILO543B32D0_10230 | Blon_2306 | FclB (L-fuconolactone | Fucose |
| | | | | | hydrolase) | catabolism |
| | | FucR | BILO543B32D0_10235 | Blon_2307 | FucP (Fucose | Fucose uptake |
| | | | | | permease) | |
| | | FucR | BILO543B32D0_10240 | Blon_2308 | FclA (L-fuco-beta- | Fucose |
| | | | | | pyranose | catabolism |
| | | | | | dehydrogenase, type 2) | |
| | | FucR | BILO543B32D0_10245 | Blon_2309 | FclC (L-fuconate | Fucose |
| | | | | | dehydratase) | catabolism |
| | | FucR | BILO543B32DQ_10250 | Blon_2310 | FucR (Predicted | Fucose |
| | | | | | transcriptional | regulation |
| | | | | | regulator for fucose | |
| | | | | | utilization, LacI | |
| | | | | | family) | |
| | Gal | GalR | BILO543B32D0_08805 | Blon_2062 | GalK (Galactokinase) | Galactose |
| | | | | | | catabolism |
| | | GalR | BILO543B32D0_08810 | Blon_2063 | GalT (Galactose-1- | Galactose |
| | | | | | phosphate | catabolism |
| | | | | | uridylyltransferase) | |
| | | GalR | BILO543B32D0_08815 | Blon_2064 | GalR (Transcriptional | Galactose |
| | | | | | regulator of galactose | regulation |
| | | | | | metabolism, DeoR | |
| | | | | | family) | |
| | HMO_{—} | NA | BILO543B32D0_10380 | Blon 2335 | BiAfcA (Exo-alpha-L- | HMO |
| | cluster I | | | | (1-2)-fucosidase, | catabolism; N- |
| | | | | | GH95) | glycan |
| | | | | | | catabolism |
| | | NA | BILO543B32D0_10385 | Blon_2336 | BiAfcB (Exo-alpha-L- | HMO |
| | | | | | (1-3/1-4)-fucosidase, | catabolism; N- |
| | | | | | GH29) | glycan |
| | | | | | | catabolism |
| | | NA | BILO543B32D0_10390 | Blon_2337 | FucU2 (L-fucose | Fucose |
| | | | | | mutarotase) | catabolism |
| | | NA | BILO543B32D0_10395 | Blon_2338 | FelE (Predicted 2- | Fucose |
| | | | | | keto-3-deoxy-L- | catabolism |
| | | | | | fuconate aldolase) | |
| | | NA | BILO543B32D0_10400 | Blon_2339 | FclA2 (L-fuco-beta- | Fucose |
| | | | | | pyranose | catabolism |
| | | | | | dehydrogenase, type 2) | |
| | | NA | BILO543B32D0_10405 | Blon_2340 | FclC2 (L-fuconate | Fucose |
| | | | | | dehydratase) | catabolism |
| | | NagR | BILO543B32D0_10415 | Blon_2342 | Blon_2342 (Type II | HMO uptake |
| | | | | | HMOs transporter, | |
| | | | | | permease protein 2) | |
| | | NagR | BILO543B32D0_10420 | Blon_2343 | Blon_2343 (Type II | HMO uptake |
| | | | | | HMOs transporter, | |
| | | | | | permease protein 1) | |
| | | NagR | BILO543B32D0_10425 | Blon 2344 | Blon_2344 (Type II | HMO uptake |
| | | | | | HMOs transporter, | |
| | | | | | substrate-binding | |
| | | | | | protein) | |
| | | NagR | BILO543B32D0_10430 | Blon_2345 | Blon_2345 (Type II | HMO uptake |
| | | | | | HMOs transporter, | |
| | | | | | permease protein 2) | |
| | | NagR | BILO543B32D0_10435 | Blon_2346 | Blon_2346 (Type II | HMO uptake |
| | | | | | HMOs transporter, | |
| | | | | | permease protein 1) | |
| | | NagR | BILO543B32D0_10440 | Blon_2347 | Blon_2347 (Type II | HMO uptake |
| | | | | | HMOs transporter | |
| | | | | | (Blon_2347) I, | |
| | | | | | substrate-binding | |
| | | | | | protein) | |
| | | NagR | BILO543B32D0_10445 | Blon_2348 | NanH2 (HMO cluster | HMO |
| | | | | | exo-alpha-(2-3/2-6)- | catabolism; N- |
| | | | | | sialidase, GH33) | glycan |
| | | | | | | catabolism |
| | | NagR | BILO543B32D0_10450 | Blon_2349 | NanA2 (N- | Sialic_acid_{—} |
| | | | | | acetylneuraminate | catabolism |
| | | | | | lyase) | |
| | | NagR | BILO543B32D0_10455 | Blon_2350 | Blon_2350 (Predicted | HMO uptake |
| | | | | | HMO transporter, | |
| | | | | | substrate-binding | |
| | | | | | protein) | |
| | | NagR | BILO543B32D0_10460 | Blon_2351 | Blon_2351 (Predicted | HMO uptake |
| | | | | | HMO transporter, | |
| | | | | | substrate-binding | |
| | | | | | protein) | |
| | | NagR | BILO543B32D0_10465 | Blon_2354 | Blon_2354 (Predicted | HMO uptake |
| | | | | | HMO transporter, | |
| | | | | | substrate-binding | |
| | | | | | protein) | |
| | | NA | BILO543B32D0_10470 | Blon 2355 | Hex2 (Exo-beta-(1- | HMO |
| | | | | | 3/1-4)-N- | catabolism; N- |
| | | | | | acetylglucosaminidase, | glycan |
| | | | | | GH20) | catabolism |
| | Lac | NA | BILO543B32D0_10365 | Blon_2331 | LacS2 (Lactose | Lactose |
| | | | | | permease, GPH | uptake |
| | | | | | translocator family) | |
| | | NA | BILO543B32D0_10370 | Blon_2332 | LacS (Lactose | Lactose |
| | | | | | permease, GPH | uptake |
| | | | | | translocator family) | |
| | | NA | BILO543B32D0_10375 | Blon_2334 | Bga2A (Exo-beta-(1- | HMO |
| | | | | | 4)-galactosidase, GH2) | catabolism; N- |
| | | | | | | glycan |
| | | | | | | catabolism; |
| | | | | | | Lactose |
| | | | | | | catabolism |
| | Lnp | NagR | BILO543B32D0_09485 | Blon_2171 | LnpD (UDP-hexose 4- | Lacto-N-biose |
| | | | | | epimerase involved in | and Galacto- |
| | | | | | lacto-N-biose | N-biose |
| | | | | | utilization) | catabolism |
| | | NagR | BILO543B32D0_09490 | Blon_2172 | LnpC (UTP-hexose-1- | Lacto-N-biose |
| | | | | | phosphate | and Galacto- |
| | | | | | uridylyltransferase | N-biose |
| | | | | | involved in lacto-N- | catabolism |
| | | | | | biose utilization, | |
| | | | | | predicted) | |
| | | NagR | BILO543B32D0_09495 | Blon_2173 | LnpB (N- | Lacto-N-biose |
| | | | | | acetylhexosamine 1- | and Galacto- |
| | | | | | kinase) | N-biose |
| | | | | | | catabolism |
| | | NagR | BILO543B32D0_09500 | Blon_2174 | LnpA (1,3-beta- | Lacto-N-biose |
| | | | | | galactosyl-N- | and Galacto- |
| | | | | | acetylhexosamine | N-biose |
| | | | | | phosphorylase) | catabolism |
| | NA | NA | BILO543B32D0_00385 | Blon_0732 | Hex1 (Exo-beta-(1- | HMO |
| | | | | | 3/1-4/1-6)-N- | catabolism; N- |
| | | | | | acetylglucosaminidase, | glycan |
| | | | | | GH20) | catabolism |
| | | NA | BILO543B32D0_04600 | NA | Mna_125* (exo-alpha- | N-glycan |
| | | | | | 1,6-mannosidase, | catabolism |
| | | | | | GH125 family)_1 | |
| | | NA | BILO543B32D0_04620 | NA | EndoBI-2 (Endo-beta- | N-glycan |
| | | | | | N- | catabolism |
| | | | | | acetylglucosaminidase | |
| | | | | | 2, GH18) | |
| | | NA | BILO543B32D0_04625 | NA | EndoBB-2 (Predicted | N-glycan |
| | | | | | endo-beta-N- | catabolism |
| | | | | | acetylglucosaminidase, | |
| | | | | | GH85)_1 | |
| | | NA | BILO543B32D0_04630 | NA | EndoBB-2 (Predicted | N-glycan |
| | | | | | endo-beta-N- | catabolism |
| | | | | | acetylglucosaminidase, | |
| | | | | | GH85)_2 | |
| | | NA | BILO543B32D0_04635 | NA | EndoBB-2 (Predicted | N-glycan |
| | | | | | endo-beta-N- | catabolism |
| | | | | | acetylglucosaminidase, | |
| | | | | | GH85)_3 | |
| | | NA | BILO543B32D0_08590 | Blon_2016 | Bga42A (Exo-beta-(1- | HMO |
| | | | | | 3/1-4/1-6)- | catabolism; |
| | | | | | galactosidase, GH42) | Galactooligosaccharides |
| | | | | | | catabolism |
| | Nag | NagR | BILO543B32D0_04665 | Blon_0879 | NagK (Predicted N- | N- |
| | | | | | acetyl-glucosamine | Acetylglucosamine |
| | | | | | kinase 2, ROK family) | catabolism |
| | | NagR | BILO543B32D0_04670 | Blon_0880 | NagR (Transciptional | N- |
| | | | | | regulator of lacto-N- | Acetylglucosamine |
| | | | | | biose and galacto-N- | regulation; |
| | | | | | biose utilization, ROK | Lacto-N-biose |
| | | | | | family) | and Galacto- |
| | | | | | | N-biose |
| | | | | | | regulation; |
| | | | | | | HMO |
| | | | | | | regulation |
| | | NagR | BILO543B32D0_04675 | Blon_0881 | NagB (Glucosamine- | N- |
| | | | | | 6-phosphate | Acetylglucosamine |
| | | | | | deaminase) | catabolism |
| | | NagR | BILO543B32D0_04680 | Blon_0882 | NagA (N- | N- |
| | | | | | acetylglucosamine-6- | Acetylglucosamine |
| | | | | | phosphate deacetylase) | catabolism |
| | | NagR | BILO543B32D0_04685 | Blon_0883 | Blon_0883 (Lacto-N- | Lacto-N-biose |
| | | | | | biose and Galacto-N- | and Galacto- |
| | | | | | biose ABC transporter | N-biose |
| | | | | | 2, periplasmic | uptake |
| | | | | | substrate-binding | |
| | | | | | protein) | |
| | | NagR | BILO543B32D0_04690 | Blon_0884 | Blon_0884 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 1) | |
| | | NagR | BILO543B32D0_04695 | Blon_0885 | Blon_0885 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 2) | |
| | Nan | NanR | BILO543B32D0_00785 | Blon_0642 | NanR (Transcriptional | Sialic acid |
| | | | | | regulator of sialic acid | regulation |
| | | | | | metabolism, GntR | |
| | | | | | family) | |
| | | NanR | BILO543B32D0_00775 | Blon_0644 | NanK (N- | Sialic acid |
| | | | | | acetylmannosamine | catabolism |
| | | | | | kinase) | |
| | | NanR | BILO543B32D0_00770 | Blon_0645 | NanE (N- | Sialic acid |
| | | | | | acetylmannosamine-6- | catabolism |
| | | | | | phosphate 2- | |
| | | | | | epimerase) | |
| | | NanR | BILO543B32D0_00765 | Blon_0646 | NanH (Exo-alpha-(2- | NA |
| | | | | | 3/2-6)-sialidase, | |
| | | | | | GH33) | |
| | | NanR | BILO543B32D0_00760 | Blon_0647 | NanB (ABC | Sialic acid |
| | | | | | transporter, predicted | uptake |
| | | | | | N-acetylneuraminate- | |
| | | | | | binding protein) | |
| | | NanR | BILO543B32D0_00755 | Blon_0648 | NanC (ABC | Sialic acid |
| | | | | | transporter, predicted | uptake |
| | | | | | N-acetylneuraminate | |
| | | | | | transport system | |
| | | | | | permease protein 1) | |
| | | NanR | BILO543B32D0_00750 | Blon_0649 | NanD (ABC | Sialic acid |
| | | | | | transporter, predicted | uptake |
| | | | | | N-acetylneuraminate | |
| | | | | | transport system | |
| | | | | | permease protein 2) | |
| | | NanR | BILO543B32D0_00745 | Blon_0650 | NanF (ABC | Sialic acid |
| | | | | | transporter, predicted | uptake |
| | | | | | N-acetylneuraminate | |
| | | | | | transport system ATP- | |
| | | | | | binding protein) | |
| | | NanR | BILO543B32D0_00740 | Blon_0651 | NanA (N- | Sialic acid |
| | | | | | acetylneuraminate | catabolism |
| | | | | | lyase) | |
| | Ngl | MnaR | BILO543B32D0_04560 | Blon_0869 | Mna_38 (Exo-alpha- | N-glycan |
| | | | | | mannosidase, GH38) | catabolism |
| | | NglR | BILO543B32D0_04565 | NA | NglA (Predicted N- | N-glycan |
| | | | | | glycan ABC transport | uptake |
| | | | | | system 2, substrate- | |
| | | | | | binding protein) | |
| | | NglR | BILO543B32D0_04570 | NA | NglB (Predicted N- | N-glycan |
| | | | | | glycan ABC transport | uptake |
| | | | | | system 2, permease | |
| | | | | | protein 1) | |
| | | NglR | BILO543B32D0_04575 | NA | NglC (Predicted N- | N-glycan |
| | | | | | glycan ABC transport | uptake |
| | | | | | system 2, permease | |
| | | | | | protein 2) | |
| | | NglR | BILO543B32D0_04580 | NA | Hex3 (Predicted N- | N-glycan |
| | | | | | glycan-acting exo- | catabolism |
| | | | | | beta-N- | |
| | | | | | acetylglucosaminidase, | |
| | | | | | GH20) | |
| | | NglR | BILO543B32D0_04585 | NA | BlMan5B* (N-glycan | N-glycan |
| | | | | | acting exo-beta- | catabolism |
| | | | | | mannosidase, | |
| | | | | | GH5_18)_1 | |
| | | NglR | BILO543B32D0_04590 | NA | NglR (Predicted | N-glycan |
| | | | | | transcriptional | regulation |
| | | | | | regulator of N-glycan | |
| | | | | | utilization, ROK | |
| | | | | | family) | |
| | Nglyc_{—} | MnaR | BILO543B32D0_04640 | Blon_0874 | MnaR (Predicted | N-glycan |
| | conserved | | | | transcriptional | regulation |
| | | | | | regulator of N-glycan | |
| | | | | | utilization, Lacl | |
| | | | | | family) | |
| | | MnaR | BILO543B32D0_04645 | Blon_0875 | ManI (D-mannose | Mannose |
| | | | | | isomerase) | catabolism |
| | | MnaR | BILO543B32D0_04650 | Blon_0876 | Mna_125 (exo-alpha- | N-glycan |
| | | | | | 1, 6-mannosidase, | catabolism |
| | | | | | GH125 family) | |
| | | MnaR | BILO543B32D0_10575 | Blon_2378 | Blon_2378 (Predicted | N-glycan |
| | | | | | N-glycan ABC | uptake |
| | | | | | transport system, | |
| | | | | | permease protein 2) | |
| | | MnaR | BILO543B32D0_10580 | Blon_2379 | Blon_2379 (Predicted | N-glycan |
| | | | | | N-glycan ABC | uptake |
| | | | | | transport system, | |
| | | | | | permease protein 1) | |
| | | MnaR | BILO543B32D0_10585 | Blon_2380 | Blon_2380 (Predicted | N-glycan |
| | | | | | N-glycan ABC | uptake |
| | | | | | transport system, | |
| | | | | | substrate-binding | |
| | | | | | protein) | |
| | | MnaR | BILO543B32D0_10570 | Blon_2377 | BlMan5B (N-glycan | N-glycan |
| | | | | | acting exo-beta- | catabolism |
| | | | | | mannosidase, | |
| | | | | | GH5_18) | |
| Bifidobacterium | FL1 | FclR | BILO9e02a2a1_01768 | Blon_0343 | Blon_0343 (2′FL, 3FL | HMO uptake |
| longum subsp. infantis | | | | | ABC transporter, | |
| Bg41721_1G8_SN_2018 | | | | | substrate-binding | |
| | | | | | protein) | |
| | | FclR | BILO9e02a2a1_01767 | Blon_0344 | FclC3 (L-fuconate | Fucose |
| | | | | | dehydratase) | catabolism |
| | FL2 | FclR | BILO9e02a2a1_01614 | Blon_2202 | Blon_2202 (2′FL, 3FL, | HMO uptake |
| | | | | | LDFT, LNFP I ABC | |
| | | | | | transporter, substrate- | |
| | | | | | binding protein) | |
| | | FclR | BILO9e02a2a1_01613 | Blon_2203 | Blon_2203 (2′FL, 3FL, | HMO uptake |
| | | | | | LDFT, LNFP I ABC | |
| | | | | | transporter, permease | |
| | | | | | component 2) | |
| | Fuc | FucR | BILO9e02a2a1_01813 | Blon_2306 | FclB (L-fuconolactone | Fucose |
| | | | | | hydrolase) | catabolism |
| | | FucR | BILO9e02a2a1_01812 | Blon_2307 | FucP (Fucose | Fucose uptake |
| | | | | | permease) | |
| | | FucR | BILO9e02a2a1_01810 | Blon_2309 | FclC (L-fuconate | Fucose |
| | | | | | dehydratase) | catabolism |
| | | FucR | BILO9e02a2a1_01809 | Blon_2310 | FucR (Predicted | Fucose |
| | | | | | transcriptional | regulation |
| | | | | | regulator for fucose | |
| | | | | | utilization, LacI | |
| | | | | | family) | |
| | Gal | GalR | BILO9e02a2a1_02346 | Blon_2062 | GalK (Galactokinase) | Galactose |
| | | | | | | catabolism |
| | | GalR | BILO9e02a2a1_02347 | Blon_2063 | GalT (Galactose-1- | Galactose |
| | | | | | phosphate | catabolism |
| | | | | | uridylyltransferase) | |
| | | GalR | BILO9e02a2a1_02348 | Blon_2064 | GalR (Transcriptional | Galactose |
| | | | | | regulator of galactose | regulation |
| | | | | | metabolism, DeoR | |
| | | | | | family) | |
| | HMO_{—} | NA | BILO9e02a2a1_01775 | Blon_2335 | BiAfcA (Exo-alpha-L- | HMO |
| | cluster I | | | | (1-2)-fucosidase, | catabolism; N- |
| | | | | | GH95) | glycan |
| | | | | | | catabolism |
| | | NA | BILO9e02a2a1_01774 | Blon_2336 | BiAfcB (Exo-alpha-L- | HMO |
| | | | | | (1-3/1-4)-fucosidase, | catabolism; N- |
| | | | | | GH29) | glycan |
| | | | | | | catabolism |
| | | NA | BILO9e02a2a1_01773 | Blon_2337 | FucU2 (L-fucose | Fucose |
| | | | | | mutarotase) | catabolism |
| | | NA | BILO9e02a2a1_01772 | Blon_2338 | FclE (Predicted 2- | Fucose |
| | | | | | keto-3-deoxy-L- | catabolism |
| | | | | | fuconate aldolase) | |
| | | NA | BILO9e02a2a1_01770 | Blon_2340 | FclC2 (L-fuconate | Fucose |
| | | | | | dehydratase) | catabolism |
| | | NagR | BILO9e02a2a1_02383 | Blon_2345 | Blon_2345 (Type II | HMO uptake |
| | | | | | HMOs transporter, | |
| | | | | | permease protein 2) | |
| | | NagR | BILO9c02a2a1_02381 | Blon_2346 | Blon_2346 (Type II | HMO uptake |
| | | | | | HMOs transporter, | |
| | | | | | permease protein 1) | |
| | | NagR | BILO9e02a2a1_01313 | Blon_2348 | NanH2 (HMO cluster | HMO |
| | | | | | exo-alpha-(2-3/2-6)- | catabolism; N- |
| | | | | | sialidase, GH33) | glycan |
| | | | | | | catabolism |
| | | NagR | BILO9e02a2a1_01312 | Blon_2349 | NanA2 (N- | Sialic_acid_{—} |
| | | | | | acetylneuraminate | catabolism |
| | | | | | lyase) | |
| | | NagR | BILO9e02a2a1_01311 | Blon_2350 | Blon_2350 (Predicted | HMO uptake |
| | | | | | HMO transporter, | |
| | | | | | substrate-binding | |
| | | | | | protein) | |
| | | NagR | BILO9e02a2a1_01310 | Blon 2352 | Blon_2352 (Predicted | HMO uptake |
| | | | | | HMO transporter, | |
| | | | | | substrate-binding | |
| | | | | | protein) | |
| | | NagR | BILO9e02a2a1_01309 | Blon_2354 | Blon_2354 (Predicted | HMO uptake |
| | | | | | HMO transporter, | |
| | | | | | substrate-binding | |
| | | | | | protein) | |
| | | NA | BILO9e02a2a1_01308 | Blon_2355 | Hex2 (Exo-beta-(1- | HMO |
| | | | | | 3/1-4)-N- | catabolism; N- |
| | | | | | acetylglucosaminidase, | glycan |
| | | | | | GH20) | catabolism |
| | Lac | NA | BILO9e02a2a1_01778 | Blon_2331 | LacS2 (Lactose | Lactose |
| | | | | | permease, GPH | uptake |
| | | | | | translocator family) | |
| | | NA | BILO9e02a2a1_01777 | Blon_2332 | LacS (Lactose | Lactose |
| | | | | | permease, GPH | uptake |
| | | | | | translocator family) | |
| | | NA | BILO9e02a2a1_01776 | Blon_2334 | Bga2A (Exo-beta-(1- | HMO |
| | | | | | 4)-galactosidase, GH2) | catabolism; N- |
| | | | | | | glycan |
| | | | | | | catabolism; |
| | | | | | | Lactose |
| | | | | | | catabolism |
| | Lnp | NagR | BILO9e02a2a1_01639 | Blon_2171 | LnpD (UDP-hexose 4- | Lacto-N-biose |
| | | | | | epimerase involved in | and Galacto- |
| | | | | | lacto-N-biose | N-biose |
| | | | | | utilization) | catabolism |
| | | NagR | BILO9e02a2a1_01638 | Blon_2172 | LnpC (UTP-hexose-1- | Lacto-N-biose |
| | | | | | phosphate | and Galacto- |
| | | | | | uridylyltransferase | N-biose |
| | | | | | involved in lacto-N- | catabolism |
| | | | | | biose utilization, | |
| | | | | | predicted) | |
| | | NagR | BILO9e02a2a1_01637 | Blon_2173 | LnpB (N- | Lacto-N-biose |
| | | | | | acetylhexosamine 1- | and Galacto- |
| | | | | | kinase) | N-biose |
| | | | | | | catabolism |
| | | NagR | BILO9e02a2a1_01636 | Blon_2174 | LnpA (1,3-beta- | Lacto-N-biose |
| | | | | | galactosyl-N- | and Galacto- |
| | | | | | acetylhexosamine | N-biose |
| | | | | | phosphorylase) | catabolism |
| | NA | NA | BILO9e02a2a1_01597 | Blon 0732 | Hex1 (Exo-beta-(1- | HMO |
| | | | | | 3/1-4/1-6)-N- | catabolism; N- |
| | | | | | acetylglucosaminidase, | glycan |
| | | | | | GH20) | catabolism |
| | | NA | BILO9e02a2a1_00270 | Blon_2016 | Bga42A (Exo-beta-(1- | HMO |
| | | | | | 3/1-4/1-6)- | catabolism |
| | | | | | galactosidase, GH42) | Galactooligosaccharides |
| | | | | | | catabolism |
| | Nag | NagR | BILO9e02a2a1_00979 | Blon_0879 | NagK (Predicted N- | N- |
| | | | | | acetyl-glucosamine | Acetylglucosamine |
| | | | | | kinase 2, ROK family) | catabolism |
| | | NagR | BILO9e02a2a1_00978 | Blon_0880 | NagR (Transciptional | N- |
| | | | | | regulator of lacto-N- | Acetylglucosamine |
| | | | | | biose and galacto-N- | regulation; |
| | | | | | biose utilization, ROK | Lacto-N-biose |
| | | | | | family) | and Galacto- |
| | | | | | | N-biose |
| | | | | | | regulation; |
| | | | | | | HMO |
| | | | | | | regulation |
| | | NagR | BILO9e02a2a1_00977 | Blon_0881 | NagB (Glucosamine- | N- |
| | | | | | 6-phosphate | Acetylglucosamine |
| | | | | | deaminase) | catabolism |
| | | NagR | BILO9e02a2a1_00976 | Blon_0882 | NagA (N- | N- |
| | | | | | acetylglucosamine-6- | Acetylglucosamine |
| | | | | | phosphate deacetylase) | catabolism |
| | | NagR | BILO9e02a2a1_00974 | Blon_0884 | Blon_0884 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 1) | |
| | | NagR | BILO9e02a2a1_00973 | Blon_0885 | Blon_0885 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 2) | |
| | Nan | NanR | BILO9e02a2a1_00325 | Blon_0642 | NanR (Transcriptional | Sialic acid |
| | | | | | regulator of sialic acid | regulation |
| | | | | | metabolism, GntR | |
| | | | | | family) | |
| | | NanR | BILO9e02a2a1_00323 | Blon_0644 | NanK (N- | Sialic acid |
| | | | | | acetylmanno samine | catabolism |
| | | | | | kinase) | |
| | | NanR | BILO9e02a2a1_00322 | Blon_0645 | NanE (N- | Sialic acid |
| | | | | | acetylmanno samine-6- | catabolism |
| | | | | | phosphate 2- | |
| | | | | | epimerase) | |
| | | NanR | BILO9e02a2a1_00321 | Blon_0646 | NanH (Exo-alpha-(2- | NA |
| | | | | | 3/2-6)-sialidase, | |
| | | | | | GH33) | |
| | | NanR | BILO9e02a2a1_00320 | Blon_0647 | NanB (ABC | Sialic acid |
| | | | | | transporter, predicted | uptake |
| | | | | | N-acetylneuraminale- | |
| | | | | | binding protein) | |
| | | NanR | BILO9e02a2a1_00319 | Blon_0648 | NanC (ABC | Sialic acid |
| | | | | | transporter, predicted | uptake |
| | | | | | N-acetylneuraminate | |
| | | | | | transport system | |
| | | | | | permease protein 1) | |
| | | NanR | BILO9e02a2a1_00317 | Blon_0650 | NanF (ABC | Sialic acid |
| | | | | | transporter, predicted | uptake |
| | | | | | N-acetylneuraminate | |
| | | | | | transport system ATP- | |
| | | | | | binding protein) | |
| | | NanR | BILO9e02a2a1_00316 | Blon_0651 | NanA (N- | Sialic acid |
| | | | | | acetylneuraminate | catabolism |
| | | | | | lyase) | |
| | Ngl | MnaR | BILO9e02a2a1_01967 | Blon_0869 | Mna_38 (Exo-alpha- | N-glycan |
| | | | | | mannosidase, GH38) | catabolism |
| | Nglyc_{—} | MnaR | BILO9e02a2a1_01968 | Blon_0868 | Mna_38* (Exo-alpha- | N-glycan |
| | conserved | | | | mannosidase, | catabolism |
| | | | | | GH38)_1 | |
| | | MnaR | BILO9e02a2a1_01962 | Blon_0874 | MnaR (Predicted | N-glycan |
| | | | | | transcriptional | regulation |
| | | | | | regulator of N-glycan | |
| | | | | | utilization, LacI | |
| | | | | | family) | |
| | | MnaR | BILO9e02a2a1_00983 | Blon_0875 | ManI (D-mannose | Mannose |
| | | | | | isomerase) | catabolism |
| | | MnaR | BILO9e02a2a1_00982 | Blon_0876 | Mna_125 (exo-alpha- | N-glycan |
| | | | | | 1,6-mannosidase, | catabolism |
| | | | | | GH125 family) | |
| | | MnaR | BILO9e02a2a1_01284 | Blon_2378 | Blon_2378 (Predicted | N-glycan |
| | | | | | N-glycan ABC | uptake |
| | | | | | transport system, | |
| | | | | | permease protein 2) | |
| | | MnaR | BILO9c02a2a1_01283 | Blon_2379 | Blon_2379 (Predicted | N-glycan |
| | | | | | N-glycan ABC | uptake |
| | | | | | transport system, | |
| | | | | | permease protein 1) | |
| | | MnaR | BILO9e02a2a1_01282 | Blon_2380 | Blon_2380 (Predicted | N-glycan |
| | | | | | N-glycan ABC | uptake |
| | | | | | transport system, | |
| | | | | | substrate-binding | |
| | | | | | protein) | |
| Bifidobacterium | FL2 | FclR | BILO16373828_00633 | Blon_2202 | Blon_2202 (2′FL, 3FL, | HMO uptake |
| longum subsp. infantis | | | | | LDFT, LNFP I ABC | |
| JG_Bg463.m5.93_JG | | | | | transporter, substrate- | |
| | | | | | binding protein) | |
| | | FclR | BILO16373828_00634 | Blon_2203 | Blon_2203 (2′FL, 3FL, | HMO uptake |
| | | | | | LDFT, LNFP I ABC | |
| | | | | | transporter, permease | |
| | | | | | component 2) | |
| | | FclR | BILO16373828_00635 | Blon_2204 | Blon_2204 (2′FL, 3FL, | HMO uptake |
| | | | | | LDFT, LNFP I ABC | |
| | | | | | transporter, permease | |
| | | | | | component 1) | |
| | Fuc | FucR | BILO16373828_00954 | Blon_2305 | FucU (L-fucose | Fucose |
| | | | | | mutarotase) | catabolism |
| | | FucR | BILO16373828_00955 | Blon_2306 | FclB (L-fuconolactone | Fucose |
| | | | | | hydrolase) | catabolism |
| | | FucR | BILO16373828_00956 | Blon_2307 | FucP (Fucose | Fucose uptake |
| | | | | | permease) | |
| | | FucR | BILO16373828_00957 | Blon_2308 | FclA (L-fuco-beta- | Fucose |
| | | | | | pyranose | catabolism |
| | | | | | dehydrogenase, type 2) | |
| | | FucR | BILO16373828_00958 | Blon_2309 | FclC (L-fuconate | Fucose |
| | | | | | dehydratase) | catabolism |
| | | FucR | BILO16373828_00959 | Blon_2310 | FucR (Predicted | Fucose |
| | | | | | transcriptional | regulation |
| | | | | | regulator for fucose | |
| | | | | | utilization, LacI | |
| | | | | | family) | |
| | Gal | GalR | BILO16373828_01643 | Blon_2062 | GalK (Galactokinase) | Galactose |
| | | | | | | catabolism |
| | | GalR | BILO16373828_01644 | Blon_2063 | GalT (Galactose-1- | Galactose |
| | | | | | phosphate | catabolism |
| | | | | | uridylyltransferase) | |
| | | GalR | BILO16373828_01645 | Blon 2064 | GalR (Transcriptional | Galactose |
| | | | | | regulator of galactose | regulation |
| | | | | | metabolism, DeoR | |
| | | | | | family) | |
| | HMO_{—} | NA | BILO16373828_00994 | Blon_2335 | BiAfcA (Exo-alpha-L- | HMO |
| | cluster I | | | | (1-2)-fucosidase, | catabolism; N- |
| | | | | | GH95) | glycan |
| | | | | | | catabolism |
| | | NA | BILO16373828_00995 | Blon_2336 | BiAfcB (Exo-alpha-L- | HMO |
| | | | | | (1-3/1-4)-fucosidase, | catabolism; N- |
| | | | | | GH29) | glycan |
| | | | | | | catabolism |
| | | NA | BILO16373828_00996 | Blon_2337 | FucU2 (L-fucose | Fucose |
| | | | | | mutarotase) | catabolism |
| | | NA | BILO16373828_00997 | Blon_2338 | FclE (Predicted 2- | Fucose |
| | | | | | keto-3-deoxy-L- | catabolism |
| | | | | | fuconate aldolase) | |
| | | NA | BILO16373828_00998 | Blon_2339 | FclA2 (L-fuco-beta- | Fucose |
| | | | | | pyranose | catabolism |
| | | | | | dehydrogenase, type 2) | |
| | | NA | BILO16373828_00999 | Blon_2340 | FclC2 (L-fuconate | Fucose |
| | | | | | dehydratase) | catabolism |
| | | NagR | BILO16373828_01001 | Blon_2342 | Blon_2342 (Type II | HMO uptake |
| | | | | | HMOs transporter, | |
| | | | | | permease protein 2) | |
| | | NagR | BILO16373828_01002 | Blon_2343 | Blon_2343 (Type II | HMO uptake |
| | | | | | HMOs transporter, | |
| | | | | | permease protein 1) | |
| | | NagR | BILO16373828_01003 | Blon_2344 | Blon_2344 (Type II | HMO uptake |
| | | | | | HMOs transporter, | |
| | | | | | substrate-binding | |
| | | | | | protein) | |
| | | NagR | BILO16373828_01004 | Blon_2345 | Blon_2345 (Type II | HMO uptake |
| | | | | | HMOs transporter, | |
| | | | | | permease protein 2) | |
| | | NagR | BILO16373828_01005 | Blon_2346 | Blon_2346 (Type II | HMO uptake |
| | | | | | HMOs transporter, | |
| | | | | | permease protein 1) | |
| | | NagR | BILO16373828_01006 | Blon_2347 | Blon_2347 (Type II | HMO uptake |
| | | | | | HMOs transporter | |
| | | | | | (Blon_2347) I, | |
| | | | | | substrate-binding | |
| | | | | | protein) | |
| | | NagR | BILO16373828_01007 | Blon_2348 | NanH2 (HMO cluster | HMO |
| | | | | | exo-alpha-(2-3/2-6)- | catabolism; N- |
| | | | | | sialidase, GH33) | glycan |
| | | | | | | catabolism |
| | | NagR | BILO16373828_01008 | Blon_2349 | NanA2 (N- | Sialic_acid_{—} |
| | | | | | acelylneuraminate | catabolism |
| | | | | | lyase) | |
| | | NagR | BILO16373828_01009 | Blon_2350 | Blon_2350 (Predicted | HMO uptake |
| | | | | | HMO transporter, | |
| | | | | | substrate-binding | |
| | | | | | protein) | |
| | | NagR | BILO16373828_01010 | Blon_2351 | Blon_2351 (Predicted | HMO uptake |
| | | | | | HMO transporter, | |
| | | | | | substrate-binding | |
| | | | | | protein) | |
| | | NagR | BILO16373828_01011 | Blon_2354 | Blon 2354 (Predicted | HMO uptake |
| | | | | | HMO transporter, | |
| | | | | | substrate-binding | |
| | | | | | protein) | |
| | | NA | BILO16373828_01012 | Blon_2355 | Hex2 (Exo-beta-(1- | HMO |
| | | | | | 3/1-4)-N- | catabolism; N- |
| | | | | | acetylglucosaminidase, | glycan |
| | | | | | GH20) | catabolism |
| | Lac | NA | BILO16373828_00991 | Blon_2331 | LacS2 (Lactose | Lactose |
| | | | | | permease, GPH | uptake |
| | | | | | translocator family) | |
| | | NA | BILO16373828_00992 | Blon_2332 | LacS (Lactose | Lactose |
| | | | | | permease, GPH | uptake |
| | | | | | translocator family) | |
| | | NA | BILO16373828_00993 | Blon_2334 | Bga2A (Exo-beta-(1- | HMO |
| | | | | | 4)-galactosidase, GH2) | catabolism; N- |
| | | | | | | glycan |
| | | | | | | catabolism |
| | | | | | | Lactose |
| | | | | | | catabolism |
| | Lmp | NagR | BILO16373828_00605 | Blon_2171 | LnpD (UDP-hexose 4- | Lacto-N-biose |
| | | | | | epimerase involved in | and Galacto- |
| | | | | | lacto-N-biose | N-biose |
| | | | | | utilization) | catabolism |
| | | NagR | BILO16373828_00606 | Blon_2172 | LnpC (UTP-hexose-1- | Lacto-N-biose |
| | | | | | phosphate | and Galacto- |
| | | | | | uridylyltransferase | N-biose |
| | | | | | involved in lacto-N- | catabolism |
| | | | | | biose utilization, | |
| | | | | | predicted) | |
| | | NagR | BILO16373828_00607 | Blon_2173 | LupB (N- | Lacto-N-biose |
| | | | | | acetylhexosamine 1- | and Galacto- |
| | | | | | kinase) | N-biose |
| | | | | | | catabolism |
| | | NagR | BILO16373828_00608 | Blon_2174 | LnpA (1,3-beta- | Lacto-N-biose |
| | | | | | galactosyl-N- | and Galacto- |
| | | | | | acetylhexosamine | N-biose |
| | | | | | phosphorylase) | catabolism |
| | | NagR | BILO16373828_00609 | Blon_2175 | Blon_2175 (Lacto-N- | HMO uptake; |
| | | | | | biose and Galacto-N- | Lacto-N-biose |
| | | | | | biose ABC transporter | and Galacto- |
| | | | | | 1, permease | N-biose |
| | | | | | component 2) | uptake |
| | | NagR | BILO16373828_00610 | Blon_2176 | Blon_2176 (Lacto-N- | HMO uptake; |
| | | | | | biose and Galacto-N- | Lacto-N-biose |
| | | | | | biose ABC transporter | and Galacto- |
| | | | | | 1, permease | N-biose |
| | | | | | component 1) | uptake |
| | | NagR | BILO16373828_00611 | Blon_2177 | Blon_2177 (Lacto-N- | HMO uptake; |
| | | | | | biose and Galacto-N- | Lacto-N-biose |
| | | | | | biose ABC transporter | and Galacto- |
| | | | | | 1, periplasmic | N-biose |
| | | | | | substrate-binding | uptake |
| | | | | | protein) | |
| | NA | NA | BILO16373828_00492 | Blon_0732 | Hex1 (Exo-beta-(1- | HMO |
| | | | | | 3/1-4/1-6)-N- | catabolism; N- |
| | | | | | acetylglucosaminidase, | glycan |
| | | | | | GH20) | catabolism |
| | | NA | BILO16373828_01180 | NA | EndoBI-2 (Endo-beta- | N-glycan |
| | | | | | N- | catabolism |
| | | | | | acetylglucosaminidase | |
| | | | | | 2, GH18) | |
| | | NA | BILO16373828_01181 | NA | EndoBB-2 (Predicted | N-glycan |
| | | | | | endo-beta-N- | catabolism |
| | | | | | acetylglucosaminidase, | |
| | | | | | GH85)_1 | |
| | | NA | BILO16373828_01182 | NA | EndoBB-2 (Predicted | N-glycan |
| | | | | | endo-beta-N- | catabolism |
| | | | | | acetylglucosaminidase, | |
| | | | | | GH85) 2 | |
| | | NA | BILO16373828_01183 | NA | EndoBB-2 (Predicted | N-glycan |
| | | | | | endo-beta-N- | catabolism |
| | | | | | acetylglucosaminidase, | |
| | | | | | GH85) 3 | |
| | | NA | BILO16373828_01600 | Blon_2016 | Bga42A (Exo-beta-(1- | HMO |
| | | | | | 3/1-4/1-6)- | catabolism |
| | | | | | galactosidase, GH42) | Galactooligosaccharides |
| | | | | | | catabolism |
| | Nag | NagR | BILO16373828_01189 | Blon_0879 | NagK (Predicted N- | N- |
| | | | | | acetyl-glucosamine | Acetylglucosamine |
| | | | | | kinase 2, ROK family) | catabolism |
| | | NagR | BILO16373828_01190 | Blon_0880 | NagR (Transciptional | N- |
| | | | | | regulator of lacto-N- | Acetylglucosamine |
| | | | | | biose and galacto-N- | regulation; |
| | | | | | biose utilization, ROK | Lacto-N-biose |
| | | | | | family) | and Galacto- |
| | | | | | | N-biose |
| | | | | | | regulation; |
| | | | | | | HMO |
| | | | | | | regulation |
| | | NagR | BILO16373828_01191 | Blon_0881 | NagB (Glucosamine- | N- |
| | | | | | 6-phosphate | Acetylglucosamine |
| | | | | | deaminase) | catabolism |
| | | NayR | BILO16373828_01192 | Blon_0882 | NagA (N- | N- |
| | | | | | acetylglucosamine-6- | Acetylglucosamine |
| | | | | | phosphate deacetylase) | catabolism |
| | Nan | NanR | BILO16373828_01882 | Blon_0642 | NanR (Transcriptional | Sialic acid |
| | | | | | regulator of sialic acid | regulation |
| | | | | | metabolism, GntR | |
| | | | | | family) | |
| | | NanR | BILO16373828_01880 | Blon_0644 | NanK (N- | Sialic acid |
| | | | | | acetylmannosamine | catabolism |
| | | | | | kinase) | |
| | | NanR | BILO16373828_01879 | Blon_0645 | NanE (N- | Sialic acid |
| | | | | | acetylmannosamine-6- | catabolism |
| | | | | | phosphate 2- | |
| | | | | | epimerase) | |
| | | NanR | BILO16373828_01878 | Blon_0646 | NanH (Exo-alpha-(2- | NA |
| | | | | | 3/2-6)-sialidase, | |
| | | | | | GH33) | |
| | | NanR | BILO16373828_01877 | Blon_0647 | NanB (ABC | Sialic acid |
| | | | | | transporter, predicted | uptake |
| | | | | | N-acetylneuraminate- | |
| | | | | | binding protein) | |
| | | NanR | BILO16373828_01876 | Blon_0648 | NanC (ABC | Sialic acid |
| | | | | | transporter, predicted | uptake |
| | | | | | N-acetylneuraminate | |
| | | | | | transport system | |
| | | | | | permease protein 1) | |
| | | NanR | BILO16373828_01875 | Blon_0649 | NanD (ABC | Sialic acid |
| | | | | | transporter, predicted | uptake |
| | | | | | N-acetylneuraminate | |
| | | | | | transport system | |
| | | | | | permease protein 2) | |
| | | NanR | BILO16373828_01874 | Blon_0650 | NanF (ABC | Sialic acid |
| | | | | | transporter, predicted | uplake |
| | | | | | N-acetylneuraminate | |
| | | | | | transport system ATP- | |
| | | | | | binding protein) | |
| | | NanR | BILO16373828_01873 | Blon_0651 | NanA (N- | Sialic acid |
| | | | | | acetylneuraminate | catabolism |
| | | | | | lyase) | |
| | Ngl | MnaR | BILO16373828_01174 | Blon_0869 | Mna_38 (Exo-alpha- | N-glycan |
| | | | | | mannosidase, GH38) | catabolism |
| | Nglyc_{—} | MnaR | BILO16373828_01173 | Blon_0868 | Mna_38* (Exo-alpha- | N-glycan |
| | conserved | | | | mannosidase, | catabolism |
| | | | | | GH38) 1 | |
| | | MnaR | BILO16373828_01184 | Blon_0874 | MnaR (Predicted | N-glycan |
| | | | | | transcriptional | regulation |
| | | | | | regulator of N-glycan | |
| | | | | | utilization, LacI | |
| | | | | | family) | |
| | | MnaR | BILO16373828_01185 | Blon_0875 | ManI (D-mannose | Mannose |
| | | | | | isomerase) | catabolism |
| | | MnaR | BILO16373828_01186 | Blon_0876 | Mna_125 (exo-alpha- | N-glycan |
| | | | | | 1,6-mannosidase, | catabolism |
| | | | | | GH125 family) | |
| | | MnaR | BILO16373828_01177 | Blon_2378 | Blon_2378 (Predicted | N-glycan |
| | | | | | N-glycan ABC | uptake |
| | | | | | transport system, | |
| | | | | | permease protein 2) | |
| | | MnaR | BILO16373828_01176 | Blon_2379 | Blon_2379 (Predicted | N-glycan |
| | | | | | N-glycan ABC | uptake |
| | | | | | transport system, | |
| | | | | | permease protein 1) | |
| | | MnaR | BILO16373828_01175 | Blon_2380 | Blon_2380 (Predicted | N-glycan |
| | | | | | N-glycan ABC | uplake |
| | | | | | transport system, | |
| | | | | | substrate-binding | |
| | | | | | protein) | |
| | | MnaR | BILO16373828_01178 | Blon_2377 | BlMan5B (N-glycan | N-glycan |
| | | | | | acting exo-beta- | catabolism |
| | | | | | mannosidase, | |
| | | | | | GH5_18) | |
| Bifidobacterium | FL1 | FclR | BILO145876ef_00441 | Blon_0340 | Blon_0340 (Predicted | HMO |
| longum subsp. infantis | | | | | transcriptional | regulation |
| PS064_13.C6_Bang_JG | | | | | regulator for | |
| | | | | | fucosyllactose | |
| | | | | | utilization, LacI | |
| | | | | | family) | |
| | | FclR | BILO145876ef_00442 | Blon_0341 | Blon 0341 (2′FL, 3FL | HMO uptake |
| | | | | | ABC transporter, | |
| | | | | | permease component | |
| | | | | | 1) | |
| | | FclR | BILO145876ef_00443 | Blon_0342 | Blon_0342 (2′FL, 3FL | HMO uptake |
| | | | | | ABC transporter, | |
| | | | | | permease component | |
| | | | | | 2) | |
| | | FclR | BILO145876ef_00444 | Blon_0343 | Blon_0343 (2TL, 3FL | HMO uptake |
| | | | | | ABC transporter, | |
| | | | | | substrate-binding | |
| | | | | | protein) | |
| | | FclR | BILO145876ef_00445 | Blon_0344 | FclC3 (L-fuconate | Fucose |
| | | | | | dehydratase) | catabolism |
| | FL2 | FclR | BILO145876ef_01994 | Blon 2202 | Blon_2202 (2′FL, 3FL, | HMO uptake |
| | | | | | LDFT, LNFP I ABC | |
| | | | | | transporter, substrate- | |
| | | | | | binding protein) | |
| | | FclR | BILO145876ef_01995 | Blon_2203 | Blon_2203 (2′FL, 3FL, | HMO uptake |
| | | | | | LDFT, LNFP I ABC | |
| | | | | | transporter, permease | |
| | | | | | component 2) | |
| | | FclR | BILO145876ef_01996 | Blon_2204 | Blon_2204 (2′FL, 3FL, | HMO uptake |
| | | | | | LDFT, LNFP I ABC | |
| | | | | | transporter, permease | |
| | | | | | component 1) | |
| | Fuc | FucR | BILO145876ef_02113 | Blon_2305 | FucU (L-fucose | Fucose |
| | | | | | mutarotase) | catabolism |
| | | FucR | BILO145876ef_02114 | Blon_2306 | FclB (T-fuconolactone | Fucose |
| | | | | | hydrolase) | catabolism |
| | | FucR | BILO145876ef_02115 | Blon_2307 | TucP (Fucose | Fucose uptake |
| | | | | | permease) | |
| | | FucR | BILO145876cf_02116 | Blon_2308 | FclA (L-fuco-beta- | Fucose |
| | | | | | pyranose | catabolism |
| | | | | | dehydrogenase, type 2) | |
| | | FucR | BILO145876ef_02117 | Blon_2309 | FclC (L-fuconate | Fucose |
| | | | | | dehydratase) | catabolism |
| | | FucR | BILO145876ef_02118 | Blon_2310 | FucR (Predicted | Fucose |
| | | | | | transcriptional | regulation |
| | | | | | regulator for fucose | |
| | | | | | utilization, LacI | |
| | | | | | family) | |
| | Gal | GalR | BILO145876ef_01870 | Blon_2062 | GalK (Galactokinase) | Galactose |
| | | | | | | catabolism |
| | | GalR | BILO145876ef_01871 | Blon_2063 | GalT (Galactose-1- | Galactose |
| | | | | | phosphate | catabolism |
| | | | | | uridylyltransferase) | |
| | | GalR | BILO145876ef_01872 | Blon 2064 | GalR (Transcriptional | Galactose |
| | | | | | regulator of galactose | regulation |
| | | | | | metabolism, DeoR | |
| | | | | | family) | |
| | HMO_{—} | NA | BILO145876ef_02152 | Blon_2335 | BiAfcA (Exo-alpha-L- | HMO |
| | cluster I | | | | (1-2)-fucosidase, | catabolism; N- |
| | | | | | GH95) | glycan |
| | | | | | | catabolism |
| | | NA | BILO145876ef_02153 | Blon_2336 | BiAfcB (Exo-alpha-L- | HMO |
| | | | | | (1-3/1-4)-fucosidase, | catabolism; N- |
| | | | | | GH29) | glycan |
| | | | | | | catabolism |
| | | NA | BILO145876ef_02154 | Blon_2337 | FucU2 (L-fucose | Fucose |
| | | | | | mutarotase) | catabolism |
| | | NA | BILO145876el_02155 | Blon_2338 | FclE (Predicted 2- | Fucose |
| | | | | | keto-3-deoxy-L- | catabolism |
| | | | | | fuconate aldolase) | |
| | | NA | BILO145876cf_02156 | Blon_2339 | FclA2 (L-fuco-beta- | Fucose |
| | | | | | pyranose | catabolism |
| | | | | | dehydrogenase, type 2) | |
| | | NA | BILO145876ef_02157 | Blon_2340 | FclC2 (L-fuconate | Fucose |
| | | | | | dehydratase) | catabolism |
| | | NagR | BILO145876ef_02159 | Blon_2342 | Blon_2342 (Type II | HMO uptake |
| | | | | | HMOs transporter, | |
| | | | | | permease protein 2) | |
| | | NagR | BILO145876ef_02160 | Blon_2343 | Blon_2343 (Type II | HMO uptake |
| | | | | | HMOs transporter, | |
| | | | | | permease protein 1) | |
| | | NagR | BILO145876ef_02161 | Blon_2344 | Blon_2344 (Type II | HMO uptake |
| | | | | | HMOs transporter, | |
| | | | | | substrate-binding | |
| | | | | | protein) | |
| | | NagR | BILO145876ef_02162 | Blon_2345 | Blon_2345 (Type II | HMO uptake |
| | | | | | HMOs transporter, | |
| | | | | | permease protein 2) | |
| | | NagR | BILO145876cf_02163 | Blon_2346 | Blon_2346 (Type II | HMO uptake |
| | | | | | HMOs transporter, | |
| | | | | | permease protein 1) | |
| | | NagR | BILO145876ef_02164 | Blon_2347 | Blon_2347 (Type II | HMO uptake |
| | | | | | HMOs transporter | |
| | | | | | (Blon_2347) I, | |
| | | | | | substrate-binding | |
| | | | | | protein) | |
| | | NagR | BILO145876ef_02165 | Blon_2348 | NanH2 (HMO cluster | HMO |
| | | | | | exo-alpha-(2-3/2-6)- | catabolism; N- |
| | | | | | sialidase, GH33) | glycan |
| | | | | | | catabolism |
| | | NagR | BILO145876ef_02166 | Blon_2349 | NanA2 (N- | Sialic_acid_{—} |
| | | | | | acetylneuraminate | catabolism |
| | | | | | lyase) | |
| | | NagR | BILO145876ef_02167 | Blon_2350 | Blon_2350 (Predicted | HMO uptake |
| | | | | | HMO transporter, | |
| | | | | | substrate-binding | |
| | | | | | protein) | |
| | | NagR | BILO145876ef_02168 | Blon_2351 | Blon_2351 (Predicted | HMO uptake |
| | | | | | HMO transporter, | |
| | | | | | substrate-binding | |
| | | | | | protein) | |
| | | NagR | BILO145876ef_02169 | Blon_2354 | Blon 2354 (Predicted | HMO uptake |
| | | | | | HMO transporter, | |
| | | | | | substrate-binding | |
| | | | | | protein) | |
| | | NA | BILO145876ef_02170 | Blon_2355 | Hex2 (Exo-beta-(1- | HMO |
| | | | | | 3/1-4)-N- | catabolism; N- |
| | | | | | acetylglucosaminidase, | glycan |
| | | | | | GH20) | catabolism |
| | Lac | NA | BILO145876ef_02149 | Blon_2331 | LacS2 (Lactose | Lactose |
| | | | | | permease, GPH | uptake |
| | | | | | translocator family) | |
| | | NA | BILO145876ef_02150 | Blon_2332 | LacS (Lactose | Lactose |
| | | | | | permease, GPH | uptake |
| | | | | | translocator family) | |
| | | NA | BILO145876ef_02151 | Blon_2334 | Bga2A (Exo-beta-(1- | HMO |
| | | | | | 4)-galactosidase, GH2) | catabolism; N- |
| | | | | | | glycan |
| | | | | | | catabolism |
| | | | | | | Lactose |
| | | | | | | catabolism |
| | Lnp | NagR | BILO145876ef_01969 | Blon_2171 | LnpD (UDP-hexose 4- | Lacto-N-biose |
| | | | | | epimerase involved in | and Galacto- |
| | | | | | lacto-N-biose | N-biose |
| | | | | | utilization) | catabolism |
| | | NagR | BILO145876ef_01970 | Blon_2172 | LnpC (UTP-hexose-1- | Lacto-N-biose |
| | | | | | phosphate | and Galacto- |
| | | | | | uridylyltransferase | N-biose |
| | | | | | involved in lacto-N- | catabolism |
| | | | | | biose utilization, | |
| | | | | | predicted) | |
| | | NagR | BILO145876ef_01971 | Blon_2173 | LupB (N- | Lacto-N-biose |
| | | | | | acetylhexosamine 1- | and Galacto- |
| | | | | | kinase) | N-biose |
| | | | | | | catabolism |
| | | NayR | BILO145876ef_01972 | Blon_2174 | LnpA (1,3-beta- | Lacto-N-biose |
| | | | | | galactosyl-N- | and Galacto- |
| | | | | | acetylhexosamine | N-biose |
| | | | | | phosphorylase) | catabolism |
| | NA | NA | BILO145876ef_00859 | Blon_0732 | Hex1 (Exo-beta-(1- | HMO |
| | | | | | 3/1-4/1-6)-N- | catabolism; N- |
| | | | | | acetylglucosaminidase, | glycan |
| | | | | | GH20) | catabolism |
| | | NA | BILO145876ef_01810 | Blon_2016 | Bga42A (Exo-beta-(1- | HMO |
| | | | | | 3/1-4/1-6)- | catabolism |
| | | | | | galactosidase, GH42) | Galactooligosaccharides |
| | | | | | | catabolism |
| | | MnaR | BILO145876ef_00034 | Blon_2468 | EndoBI-1 (Endo-beta- | N-glycan |
| | | | | | N- | catabolism |
| | | | | | acetylglucosaminidase, | |
| | | | | | GH18) | |
| | Nag | NagR | BILO145876ef_01016 | Blon_0879 | NagK (Predicted N- | N- |
| | | | | | acetyl-glucosamine | Acetylglucosamine |
| | | | | | kinase 2, ROK family) | catabolism |
| | | NagR | BILO145876ef_01017 | Blon_0880 | NagR (Transciptional | N- |
| | | | | | regulator of lacto-N- | Acetylglucosamine |
| | | | | | biose and galacto-N- | regulation; |
| | | | | | biose utilization, ROK | Lacto-N-biose |
| | | | | | family) | and Galacto- |
| | | | | | | N-biose |
| | | | | | | regulation; |
| | | | | | | HMO |
| | | | | | | regulation |
| | | NagR | BILO145876ef_01018 | Blon_0881 | NagB (Glucosamine- | N- |
| | | | | | 6-phosphate | Acetylglucosamine |
| | | | | | deaminase) | catabolism |
| | | NagR | BILO145876ef_01019 | Blon_0882 | NagA (N- | N- |
| | | | | | acetylglucosamine-6- | Acetylglucosamine |
| | | | | | phosphate deacetylase) | catabolism |
| | | NagR | BILO145876ef_01020 | Blon_0883 | Blon_0883 (Lacto-N- | Lacto-N-biose |
| | | | | | biose and Galacto-N- | and Galacto- |
| | | | | | biose ABC transporter | N-biose |
| | | | | | 2, periplasmic | uptake |
| | | | | | substrate-binding | |
| | | | | | protein) | |
| | | NagR | BILO145876ef_01021 | Blon_0884 | Blon_0884 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 1) | |
| | | NagR | BILO145876ef_01022 | Blon_0885 | Blon_0885 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 2) | |
| | Nan | NanR | BILO145876ef_00779 | Blon_0642 | NanR (Transcriptional | Sialic acid |
| | | | | | regulator of sialic acid | regulation |
| | | | | | metabolism, GntR | |
| | | | | | family) | |
| | | NanR | BILO145876ef_00781 | Blon_0644 | NanK (N- | Sialic acid |
| | | | | | acetylmannosamine | catabolism |
| | | | | | kinase) | |
| | | NanR | BILO145876ef_00783 | Blon_0646 | NanH (Exo-alpha-(2- | NA |
| | | | | | 3/2-6)-sialidase, | |
| | | | | | GH33) | |
| | | NanR | BILO145876ef_00784 | Blon_0647 | NanB (ABC | Sialic acid |
| | | | | | transporter, predicted | uptake |
| | | | | | N-acetylneuraminate- | |
| | | | | | binding protein) | |
| | | NanR | BILO145876ef_00785 | Blon_0648 | NanC (ABC | Sialic acid |
| | | | | | transporter, predicted | uptake |
| | | | | | N-acetylneuraminate | |
| | | | | | transport system | |
| | | | | | permease protein 1) | |
| | | NanR | BILO145876ef_00786 | Blon_0649 | NanD (ABC | Sialic acid |
| | | | | | transporter, predicted | uptake |
| | | | | | N-acetylneuraminate | |
| | | | | | transport system | |
| | | | | | permease protein 2) | |
| | | NanR | BILO145876ef_00787 | Blon_0650 | NanF (ABC | Sialic acid |
| | | | | | transporter, predicted | uptake |
| | | | | | N-acetylneuraminate | |
| | | | | | transport system ATP- | |
| | | | | | binding protein) | |
| | | NanR | BILO145876ef_00788 | Blon_0651 | NanA (N- | Sialic acid |
| | | | | | acetylneuraminate | catabolism |
| | | | | | lyase) | |
| | Ngl | MnaR | BILO145876ef_01006 | Blon_0869 | Mna_38 (Exo-alpha- | N-glycan |
| | | | | | mannosidase, GH38) | catabolism |
| | Nglyc_{—} | MnaR | BILO145876ef_01005 | Blon_0868 | Mna_38* (Exo-alpha- | N-glycan |
| | conserved | | | | mannosidase, | catabolism |
| | | | | | GH38) 1 | |
| | | MuaR | BILO145876ef_01011 | Blon_0874 | MnaR (Predicted | N-glycan |
| | | | | | transcriptional | regulation |
| | | | | | regulator of N-glycan | |
| | | | | | utilization, LacI | |
| | | | | | family) | |
| | | MnaR | BILO145876ef_01012 | Blon_0875 | ManI (D-mannose | Mannose |
| | | | | | isomerase) | catabolism |
| | | MnaR | BILO145876ef_01013 | Blon_0876 | Mna_125 (exo-alpha- | N-glycan |
| | | | | | 1,6-mannosidase, | catabolism |
| | | | | | GH125 family) | |
| | | MnaR | BILO145876ef_02186 | Blon_2378 | Blon 2378 (Predicted | N-glycan |
| | | | | | N-glycan ABC | uptake |
| | | | | | transport system, | |
| | | | | | permease protein 2) | |
| | | MnaR | BILO145876ef_02187 | Blon_2379 | Blon_2379 (Predicted | N-glycan |
| | | | | | N-glycan ABC | uptake |
| | | | | | transport system, | |
| | | | | | permease protein 1) | |
| | | MnaR | BILO145876ef_02188 | Blon_2380 | Blon_2380 (Predicted | N-glycan |
| | | | | | N-glycan ABC | uptake |
| | | | | | transport system, | |
| | | | | | substrate-binding | |
| | | | | | protein) | |
| | | MnaR | BILO145876ef_02185 | Blon_2377 | BIMan5B (N-glycan | N-glycan |
| | | | | | acting exo-beta- | catabolism |
| | | | | | mannosidase, | |
| | | | | | GH5_18) | |
| Bifidobacterium longum | Blon_{—} | NA | N_02388 | Blon_0459 | Hex1* (Exo-beta-(1- | HMO |
| subsp. infantis EVC001 | 0459-0462 | | | | 3/1-4/1-6)-N- | catabolism |
| | | | | | acetylglucosaminidase, | |
| | | | | | GH20)_1 | |
| | | NA | N_02387 | Blon_0460 | Blon_0460 (Type II | HMO uptake |
| | | | | | HMOs transporter B. | |
| | | | | | breve like, permease | |
| | | | | | component 2) | |
| | | NA | N_02386 | Blon_0461 | Blon_0461 (Type II | HMO uptake |
| | | | | | HMOs transporter B. | |
| | | | | | breve like, permease | |
| | | | | | component 1) | |
| | | NA | N_02385 | Blon_0462 | Blon_0462 (Type II | HMO uptake |
| | | | | | HMOs transporter B. | |
| | | | | | breve, substrate- | |
| | | | | | binding protein) | |
| | FL1 | FclR | N_02513 | Blon_0340 | Blon_0340 (Predicted | HMO |
| | | | | | transcriptional | regulation |
| | | | | | regulator for | |
| | | | | | fucosyllactose | |
| | | | | | utilization, LacI | |
| | | | | | family) | |
| | | FclR | N_02512 | Blon_0341 | Blon_0341 (2′FL, 3FL | HMO uptake |
| | | | | | ABC transporter, | |
| | | | | | permease component | |
| | | FclR | N_02511 | Blon_0342 | Blon_0342 (2TL, 3FL | HMO uptake |
| | | | | | ABC transporter, | |
| | | | | | permease component | |
| | | | | | 2) | |
| | | FclR | N_02510 | Blon_0343 | Blon_0343 (2′FL, 3FL | HMO uptake |
| | | | | | ABC transporter, | |
| | | | | | substrate-binding | |
| | | | | | protein) | |
| | | FclR | N_02509 | Blon_0344 | FclC3 (L-fuconate | Fucose |
| | | | | | dehydratase) | catabolism |
| | FL2 | FclR | N_00533 | Blon_2202 | Blon_2202 (2′FL, 3FL, | HMO uptake |
| | | | | | LDFT, LNFP I ABC | |
| | | | | | transporter, substrate- | |
| | | | | | binding protein) | |
| | | FclR | N_00532 | Blon_2203 | Blon_2203 (2′FL, 3FL, | HMO uptake |
| | | | | | LDFT, LNFP I ABC | |
| | | | | | transporter, permease | |
| | | | | | component 2) | |
| | | FclR | N_00531 | Blon_2204 | Blon_2204 (2′FL, 3FL, | HMO uptake |
| | | | | | LDFT, LNFP I ABC | |
| | | | | | transporter, permease | |
| | | | | | component 1) | |
| | Fuc | FucR | N_00418 | Blon_2305 | FucU (L-fucose | Fucose |
| | | | | | mutarotase) | catabolism |
| | | FucR | N_00417 | Blon_2306 | FclB (L-fuconolactone | Fucose |
| | | | | | hydrolase) | catabolism |
| | | FucR | N_00416 | Blon_2307 | FucP (Fucose | Fucose uptake |
| | | | | | permease) | |
| | | FucR | N_00415 | Blon_2308 | FclA (L-fuco-beta- | Fucose |
| | | | | | pyranose | catabolism |
| | | | | | dehydrogenase, type 2) | |
| | | FucR | N_00414 | Blon_2309 | FclC (L-fuconate | Fucose |
| | | | | | dehydratase) | catabolism |
| | | FucR | N_00413 | Blon_2310 | FucR (Predicted | Fucose |
| | | | | | transcriptional | regulation |
| | | | | | regulator for fucose | |
| | | | | | utilization, LacI | |
| | | | | | family) | |
| | Gal | GalR | N_00672 | Blon_2062 | GalK (Galactokinase) | Galactose |
| | | | | | | catabolism |
| | | GalR | N_00671 | Blon_2063 | GalT (Galactose-1- | Galactose |
| | | | | | phosphate | catabolism |
| | | | | | uridylyltransferase) | |
| | | GalR | N_00670 | Blon_2064 | GalR (Transcriptional | Galactose |
| | | | | | regulator of galactose | regulation |
| | | | | | metabolism, DeoR | |
| | | | | | family) | |
| | HMO_{—} | NA | N_00387 | Blon_2335 | BiAfcA (Exo-alpha-L- | HMO |
| | cluster I | | | | (1-2)-fucosidase, | catabolism; N- |
| | | | | | GH95) | glycan |
| | | | | | | catabolism |
| | | NA | N_00386 | Blon_2336 | BiAfcB (Exo-alpha-L- | HMO |
| | | | | | (1-3/1-4)-fucosidase, | catabolism; N- |
| | | | | | GH29) | glycan |
| | | | | | | catabolism |
| | | NA | N_00385 | Blon_2337 | FucU2 (L-fucose | Fucose |
| | | | | | mutarotase) | catabolism |
| | | NA | N_00384 | Blon_2338 | FclE (Predicted 2- | Fucose |
| | | | | | keto-3-deoxy-L- | catabolism |
| | | | | | fuconate aldolase) | |
| | | NA | N_00383 | Blon_2339 | FclA2 (L-fuco-beta- | Fucose |
| | | | | | pyranose | catabolism |
| | | | | | dehydrogenase, type 2) | |
| | | NA | N_00382 | Blon_2340 | FclC2 (L-fuconate | Fucose |
| | | | | | dehydratase) | catabolism |
| | | NagR | N_00380 | Blon_2342 | Blon_2342 (Type II | HMO uptake |
| | | | | | HMOs transporter, | |
| | | | | | permease protein 2) | |
| | | NagR | N_00379 | Blon_2343 | Blon_2343 (Type II | HMO uptake |
| | | | | | HMOs transporter, | |
| | | | | | permease protein 1) | |
| | | NagR | N_00378 | Blon_2344 | Blon_2344 (Type II | HMO uptake |
| | | | | | HMOs transporter, | |
| | | | | | substrate-binding | |
| | | | | | protein) | |
| | | NagR | N_00377 | Blon_2345 | Blon_2345 (Type II | HMO uptake |
| | | | | | HMOs transporter, | |
| | | | | | permease protein 2) | |
| | | NagR | N_00376 | Blon_2346 | Blon_2346 (Type II | HMO uptake |
| | | | | | HMOs transporter, | |
| | | | | | permease protein 1) | |
| | | NagR | N_00375 | Blon_2347 | Blon_2347 (Type II | HMO uptake |
| | | | | | HMOs transporter | |
| | | | | | (Blon_2347) I, | |
| | | | | | substrate-binding | |
| | | | | | protein) | |
| | | NagR | N_00374 | Blon_2348 | NanH2 (HMO cluster | HMO |
| | | | | | exo-alpha-(2-3/2-6)- | catabolism; N- |
| | | | | | sialidase, GH33) | glycan |
| | | | | | | catabolism |
| | | NagR | N_00373 | Blon_2349 | NanA2 (N- | Sialic acid_{—} |
| | | | | | acetylneuraminate | catabolism |
| | | | | | lyase) | |
| | | NagR | N_00372 | Blon_2350 | Blon_2350 (Predicted | HMO uptake |
| | | | | | HMO transporter, | |
| | | | | | substrate-binding | |
| | | | | | protein) | |
| | | NagR | N_00371 | Blon_2351 | Blon_2351 (Predicted | HMO uptake |
| | | | | | HMO transporter, | |
| | | | | | substrate-binding | |
| | | | | | protein) | |
| | | NagR | N_00370 | Blon_2352 | Blon_2352 (Predicted | HMO uptake |
| | | | | | HMO transporter, | |
| | | | | | substrate-binding | |
| | | | | | protein) | |
| | | NagR | N_00369 | Blon_2354 | Blon_2354 (Predicted | HMO uptake |
| | | | | | HMO transporter, | |
| | | | | | substrate-binding | |
| | | | | | protein) | |
| | | NA | N_00368 | Blon_2355 | Hex2 (Exo-beta-(1- | HMO |
| | | | | | 3/1-4)-N- | catabolism; N- |
| | | | | | acetylglucosaminidase, | glycan |
| | | | | | GH20) | catabolism |
| | Lac | NA | N_00391 | Blon 2331 | LacS2 (Lactose | Lactose |
| | | | | | permease, GPH | uptake |
| | | | | | translocator family) | |
| | | NA | N_00390 | Blon_2332 | LacS (Lactose | Lactose |
| | | | | | permease, GPH | uptake |
| | | | | | translocator family) | |
| | | NA | N_00388 | Blon_2334 | Bga2A (Exo-beta-(1- | HMO |
| | | | | | 4)-galactosidase, GH2) | catabolism; N- |
| | | | | | | glycan |
| | | | | | | catabolism; |
| | | | | | | Lactose |
| | | | | | | catabolism |
| | Lnp | NagR | N_00563 | Blon_2171 | LnpD (UDP-hexose 4- | Lacto-N-biose |
| | | | | | epimerase involved in | and Galacto- |
| | | | | | lacto-N-biose | N-biose |
| | | | | | utilization) | catabolism |
| | | NagR | N_00562 | Blon_2172 | LnpC (UTP-hexose-1- | Lacto-N-biose |
| | | | | | phosphate | and Galacto- |
| | | | | | uridylyltransferase | N-biose |
| | | | | | involved in lacto-N- | catabolism |
| | | | | | biose utilization, | |
| | | | | | predicted) | |
| | | NagR | N_00561 | Blon_2173 | LnpB (N- | Lacto-N-biose |
| | | | | | acetylhexosamine 1- | and Galacto- |
| | | | | | kinase) | N-biose |
| | | | | | | catabolism |
| | | NagR | N_00560 | Blon_2174 | LnpA (1,3-beta- | Lacto-N-biose |
| | | | | | galactosyl-N- | and Galacto- |
| | | | | | acetylhexosamine | N-biose |
| | | | | | phosphorylase) | catabolism |
| | | NagR | N_00559 | Blon_2175 | Blon_2175 (Lacto-N- | HMO uptake; |
| | | | | | biose and Galacto-N- | Lacto-N-biose |
| | | | | | biose ABC transporter | and Galacto- |
| | | | | | 1, permease | N-biose |
| | | | | | component 2) | uptake |
| | | NagR | N_00558 | Blon_2176 | Blon_2176 (Lacto-N- | HMO uptake; |
| | | | | | biose and Galacto-N- | Lacto-N-biose |
| | | | | | biose ABC transporter | and Galacto- |
| | | | | | 1, permease | N-biose |
| | | | | | component 1) | uptake |
| | | NagR | N_00557 | Blon_2177 | Blon_2177 (Lacto-N- | HMO uptake; |
| | | | | | biose and Galacto-N- | Lacto-N-biose |
| | | | | | biose ABC transporter | and Galacto- |
| | | | | | 1, periplasmic | N-biose |
| | | | | | substrate-binding | uptake |
| | | | | | protein) | |
| | NA | NA | N_02109 | Blon_0732 | Hex1 (Exo-beta-(1- | HMO |
| | | | | | 3/1-4/1-6)-N- | catabolism; N- |
| | | | | | acetylglucosaminidase, | glycan |
| | | | | | GH20) | catabolism |
| | | NA | N_00717 | Blon_2016 | Bga42A (Exo-beta-(1- | HMO |
| | | | | | 3/1-4/1-6)- | catabolism |
| | | | | | galactosidase, GH42) | Galactooligosaccharides |
| | | | | | | catabolism |
| | | MnaR | N_00253 | Blon_2468 | EndoBI-1 (Endo-beta- | N-glycan |
| | | | | | N- | catabolism |
| | | | | | acetyglucosaminidase, | |
| | | | | | GH18) | |
| | Nag | NagR | N_01960 | Blon_0879 | NagK (Predicted N- | N- |
| | | | | | acelyl-glucosamine | Acetylglucosamine |
| | | | | | kinase 2, ROK family) | catabolism |
| | | NagR | N_01959 | Blon_0880 | NagR (Transciptional | N- |
| | | | | | regulator of lacto-N- | Acetylglucosamine |
| | | | | | biose and galacto-N- | regulation; |
| | | | | | biose utilization, ROK | Lacto-N-biose |
| | | | | | family) | and Galacto- |
| | | | | | | N-biose |
| | | | | | | regulation; |
| | | | | | | HMO |
| | | | | | | regulation |
| | | NagR | N_01958 | Blon_0881 | NagB (Glucosamine- | N- |
| | | | | | 6-phosphate | Acetylglucosamine |
| | | | | | deaminase) | catabolism |
| | | NagR | N_01957 | Blon_0882 | NagA (N- | N- |
| | | | | | acetylglucosamine-6- | Acetylglucosamine |
| | | | | | phosphate deacetylase) | catabolism |
| | | NagR | N_01956 | Blon_0883 | Blon_0883 (Lacto-N- | Lacto-N-biose |
| | | | | | biose and Galacto-N- | and Galacto- |
| | | | | | biose ABC transporter | N-biose |
| | | | | | 2, periplasmic | uptake |
| | | | | | substrate-binding | |
| | | | | | protein) | |
| | | NagR | N_01955 | Blon_0884 | Blon_0884 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 1) | |
| | | NagR | N_01954 | Blon_0885 | Blon_0885 (Lacto-N- | Lacto-N-biose |
| | | | | | biose and Galacto-N- | and Galacto- |
| | | | | | biose ABC transporter | N-biose |
| | | | | | 2, permease | uptake |
| | | | | | component 2) | |
| | Nan | NanR | N_02202 | Blon_0642 | NanR (Transcriptional | Sialic acid |
| | | | | | regulator of sialic acid | regulation |
| | | | | | metabolism, GntR | |
| | | | | | family) | |
| | | NanR | N_02200 | Blon_0644 | NanK (N- | Sialic acid |
| | | | | | acetylmannosamine | catabolism |
| | | | | | kinase) | |
| | | NanR | N_02199 | Blon_0645 | NanE (N- | Sialic acid |
| | | | | | acetylmannosamine-6- | catabolism |
| | | | | | phosphate 2- | |
| | | | | | epimerase) | |
| | | NanR | N_02198 | Blon_0646 | NanH (Exo-alpha-(2- | NA |
| | | | | | 3/2-6)-sialidase, | |
| | | | | | GH33) | |
| | | NanR | N_02197 | Blon_0647 | NanD (ABC | Sialic acid |
| | | | | | transporter, predicted | uptake |
| | | | | | N-acetylneuraminate- | |
| | | | | | binding protein) | |
| | | NanR | N_02196 | Blon_0648 | NanC (ABC | Sialic acid |
| | | | | | transporter, predicted | uptake |
| | | | | | N-acetylneuraminate | |
| | | | | | transport system | |
| | | | | | permease protein 1) | |
| | | NanR | N_02195 | Blon_0649 | NanD (ABC | Sialic acid |
| | | | | | transporter, predicted | uptake |
| | | | | | N-acetylneuraminate | |
| | | | | | transport system | |
| | | | | | permease protein 2) | |
| | | NanR | N_02194 | Blon_0650 | NanF (ABC | Sialic acid |
| | | | | | transporter, predicted | uptake |
| | | | | | N-acetylneuraminate | |
| | | | | | transport system ATP- | |
| | | | | | binding protein) | |
| | | NanR | N_02193 | Blon_0651 | NanA (N- | Sialic acid |
| | | | | | acetylneuraminate | catabolism |
| | | | | | lyase) | |
| | Nglyc_{—} | MnaR | N_01971 | Blon_0868 | Mna 38* (Exo-alpha- | N-glycan |
| | conserved | | | | mannosidase, | catabolism |
| | | | | | GH38)_1 | |
| | | MnaR | N_01965 | Blon 0874 | MuaR (Predicted | N-glycan |
| | | | | | transcriptional | regulation |
| | | | | | regulator of N-glycan | |
| | | | | | utilization, LacI | |
| | | | | | family) | |
| | | MnaR | N_01964 | Blon_0875 | ManI (D-mannose | Mannose |
| | | | | | isomerase) | catabolism |
| | | MnaR | N_01963 | Blon_0876 | Mna_125 (exo-alpha- | N-glycan |
| | | | | | 1,6-mannosidase, | catabolism |
| | | | | | GH125 family) | |
| | | MnaR | N_00347 | Blon_2378 | Blon_2378 (Predicted | N-glycan |
| | | | | | N-glycan ABC | uptake |
| | | | | | transport system, | |
| | | | | | permease protein 2) | |
| | | MnaR | N_00346 | Blon_2379 | Blon_2379 (Predicted | N-glycan |
| | | | | | N-glycan ABC | uptake |
| | | | | | transport system, | |
| | | | | | permease protein 1) | |
| | | MnaR | N_00345 | Blon_2380 | Blon_2380 (Predicted | N-glycan |
| | | | | | N-glycan ABC | uptake |
| | | | | | transport system, | |
| | | | | | substrate-binding | |
| | | | | | protein) | |
| | | MnaR | N_00348 | Blon_2377 | BlMan5B (N-glycan | N-glycan |
| | | | | | acting exo-beta- | catabolism |
| | | | | | mannosidase, | |
| | | | | | GH5 18) | |

TABLE 14b

| | | | | | Log2-fold | | | Log2-fold | | | Log2-fold | |
|---|---|---|---|---|---|---|---|---|---|---|---|---|
| | | | | | change | | | change | | | change | |
| | | | | Log2-fold | Standard | FDR adjusted | Log2-fold | Standard | FDR adjusted | Log2-fold | Standard | FDR adjusted |
| Strain | Genomic cluster | Regulon | Locus tag | change | Error | p-value | change | Error | p-value | change | Error | p-value |
| Bifidobacterium | Bgl | BglT | BILO543B32D0_06875 | 0.17 | 0.24 | 0.81 | 0.17 | 0.24 | 0.72 | 0.50 | 0.23 | 0.21 |
| longum | | BglT | BILO543B32D0_06880 | 0.09 | 0.14 | 0.84 | 0.15 | 0.14 | 0.55 | 0.25 | 0.14 | 0.32 |
| subsp. infantis | | BglT | BILO543B32D0_06885 | −0.09 | 0.14 | 0.85 | 0.10 | 0.14 | 0.72 | 0.14 | 0.14 | 0.65 |
| Bg407212D9_SN_2018 | | BglT | BILO543B32D0_06890 | −0.03 | 0.13 | 0.96 | 0.27 | 0.12 | 0.14 | 0.03 | 0.12 | 0.94 |
| | | BglT | BILO543B32D0_06895 | −0.68 | 0.46 | 0.48 | −0.85 | 0.46 | 0.23 | −0.02 | 0.46 | 0.98 |
| | | BglT | BILO543B32D0_06900 | −0.32 | 0.45 | 0.81 | −0.40 | 0.45 | 0.63 | 0.07 | 0.45 | 0.96 |
| | | BglT | BILO543B32D0_06905 | −0.25 | 0.35 | 0.82 | −0.39 | 0.35 | 0.52 | 0.27 | 0.35 | 0.76 |
| | | BglT | BILO543B32D0_06910 | −0.61 | 0.37 | 0.42 | −0.53 | 0.37 | 0.39 | 0.07 | 0.37 | 0.94 |
| | FL2 | FclR | BILO543B32D0_09610 | 0.43 | 0.73 | 0.86 | −0.16 | 0.73 | 0.93 | 0.85 | 0.71 | NA |
| | | FclR | BILO543B32D0_09615 | −2.14 | 0.59 | 0.01 | 0.20 | 0.57 | 0.87 | 0.80 | 0.56 | NA |
| | | FclR | BILO543B32D0_09620 | 2.52 | 0.47 | 0.00 | 2.03 | 0.47 | 0.00 | 0.32 | 0.46 | 0.79 |
| | Fuc | FucR | BILO543B32D0_10225 | −0.15 | 0.39 | 0.91 | −0.14 | 0.39 | 0.87 | −0.48 | 0.39 | 0.54 |
| | | FucR | BILO543B32D0_10230 | 0.35 | 0.48 | 0.81 | 1.04 | 0.48 | 0.15 | 0.13 | 0.48 | 0.92 |
| | | FucR | BILO543B32D0_10235 | −0.79 | 0.48 | 0.42 | −0.28 | 0.48 | 0.77 | 0.29 | 0.48 | 0.81 |
| | | FucR | BILO543B32D0_10240 | −0.54 | 0.53 | 0.67 | 0.40 | 0.53 | 0.70 | 0.69 | 0.53 | 0.51 |
| | | FucR | BILO543B32D0_10245 | 0.82 | 3.38 | 0.95 | −5.26 | 3.46 | 0.36 | 0.00 | 3.55 | NA |
| | | FucR | BILO543B32D0_10250 | 0.42 | 0.23 | 0.35 | 0.47 | 0.23 | 0.16 | 0.52 | 0.23 | 0.19 |
| | Gal | GalR | BILO543B32D0_08805 | 0.39 | 0.20 | 0.29 | −0.04 | 0.20 | 0.94 | −0.33 | 0.20 | 0.38 |
| | | GalR | BILO543B32D0_08810 | 0.15 | 0.23 | 0.83 | −0.06 | 0.23 | 0.90 | −0.48 | 0.23 | 0.23 |
| | | GalR | BILO543B32D0_08815 | −2.32 | 3.06 | 0.80 | −2.45 | 3.06 | 0.68 | 2.88 | 3.06 | NA |
| | IIMO_cluster | NA | BILO543B32D0_10380 | 0.48 | 0.14 | 0.02 | 0.68 | 0.14 | 0.00 | −0.63 | 0.14 | 0.00 |
| | I | NA | BILO543B32D0_10385 | 0.53 | 0.21 | 0.12 | 0.88 | 0.21 | 0.00 | −0.39 | 0.21 | 0.28 |
| | | NA | BILO543B32D0_10390 | 0.40 | 0.20 | 0.28 | 0.45 | 0.20 | 0.12 | −0.24 | 0.20 | 0.56 |
| | | NA | BILO543B32D0_10395 | 0.26 | 0.19 | 0.54 | 0.46 | 0.19 | 0.09 | −0.22 | 0.19 | 0.58 |
| | | NA | BILO543B32D0_10400 | 0.61 | 0.21 | 0.06 | 0.58 | 0.21 | 0.04 | −0.03 | 0.21 | 0.95 |
| | | NA | BILO543B32D0_10405 | 0.27 | 0.22 | 0.59 | 0.51 | 0.22 | 0.11 | −0.08 | 0.22 | 0.89 |
| | | NagR | BILO543B32D0_10415 | 1.59 | 0.34 | 0.00 | 1.61 | 0.34 | 0.00 | −0.62 | 0.34 | 0.31 |
| | | NagR | BILO543B32D0_10420 | 1.87 | 0.35 | 0.00 | 1.54 | 0.35 | 0.00 | −0.47 | 0.35 | 0.50 |
| | | NagR | BILO543B32D0_10425 | 3.16 | 0.49 | 0.00 | 2.21 | 0.49 | 0.00 | −0.52 | 0.49 | 0.64 |
| | | NagR | BILO543B32D0_10430 | 2.16 | 0.38 | 0.00 | 0.76 | 0.38 | 0.19 | −0.06 | 0.38 | 0.96 |
| | | NagR | BILO543B32D0_10435 | 1.87 | 0.35 | 0.00 | 1.54 | 0.35 | 0.00 | −0.47 | 0.35 | 0.50 |
| | | NagR | BILO543B32D0_10440 | 1.24 | 0.33 | 0.01 | 2.53 | 0.33 | 0.00 | −1.09 | 0.33 | 0.03 |
| | | NagR | BILO543B32D0_10445 | 0.54 | 0.29 | 0.33 | 0.81 | 0.29 | 0.04 | −0.12 | 0.29 | 0.88 |
| | | NagR | BILO543B32D0_10450 | 0.87 | 0.29 | 0.05 | 1.37 | 0.29 | 0.00 | −0.33 | 0.29 | 0.59 |
| | | NagR | BILO543B32D0_10455 | 0.76 | 0.28 | 0.08 | 1.49 | 0.27 | 0.00 | −0.20 | 0.27 | 0.79 |
| | | NagR | BILO543B32D0_10460 | 0.61 | 0.28 | 0.21 | 1.42 | 0.28 | 0.00 | −0.14 | 0.28 | 0.85 |
| | | NagR | BILO543B32D0_10465 | 0.46 | 0.30 | 0.47 | 0.39 | 0.30 | 0.45 | 0.25 | 0.30 | 0.75 |
| | | NA | BILO543B32D0_10470 | 0.40 | 0.25 | 0.43 | 0.40 | 0.25 | 0.32 | 0.17 | 0.25 | 0.79 |
| | Lac | NA | BILO543B32D0_10365 | −1.15 | 0.25 | 0.00 | −1.46 | 0.25 | 0.00 | 0.49 | 0.25 | 0.28 |
| | | NA | BILO543B32D0_10370 | −2.97 | 0.63 | 0.00 | −4.57 | 0.63 | 0.00 | 0.59 | 0.63 | 0.70 |
| | | NA | BILO543B32D0_10375 | −1.42 | 0.48 | 0.06 | −1.99 | 0.48 | 0.00 | 0.97 | 0.48 | 0.25 |
| | Lnp | NagR | BILO543B32D0_09485 | 0.38 | 0.23 | 0.42 | 0.79 | 0.23 | 0.01 | −0.40 | 0.23 | 0.36 |
| | | NagR | BILO543B32D0_09490 | 0.47 | 0.36 | 0.56 | 1.17 | 0.36 | 0.01 | −0.16 | 0.36 | 0.87 |
| | | NagR | BILO543B32D0_09495 | 0.20 | 0.32 | 0.84 | 1.33 | 0.32 | 0.00 | 0.07 | 0.32 | 0.94 |
| | | NagR | BILO543B32D0_09500 | 0.24 | 0.32 | 0.80 | 1.31 | 0.32 | 0.00 | 0.07 | 0.32 | 0.94 |
| | NA | NA | BILO543B32D0_00385 | 0.11 | 0.22 | 0.89 | 0.03 | 0.22 | 0.95 | −0.05 | 0.22 | 0.94 |
| | | NA | BILO543B32D0_04600 | −2.27 | 0.85 | 0.10 | −1.77 | 0.84 | 0.16 | −0.65 | 0.84 | NA |
| | | NA | BILO543B32D0_04620 | −0.38 | 0.53 | 0.81 | −0.36 | 0.53 | 0.73 | −0.03 | 0.53 | NA |
| | | NA | BILO543B32D0_04625 | −0.07 | 0.52 | 0.97 | −0.72 | 0.52 | 0.41 | 0.01 | 0.51 | NA |
| | | NA | BILO543B32D0_04630 | −1.32 | 0.74 | 0.36 | −1.86 | 0.74 | 0.08 | −0.16 | 0.74 | NA |
| | | NA | BILO543B32D0_04635 | 0.58 | 0.45 | 0.56 | 0.68 | 0.45 | 0.37 | −0.74 | 0.44 | NA |
| | | NA | BILO543B32D0_08590 | 0.39 | 0.15 | 0.13 | 0.29 | 0.15 | 0.22 | −0.12 | 0.15 | 0.77 |
| | Nag | NagR | BILO543B32D0_04665 | 1.00 | 0.27 | 0.01 | 2.74 | 0.27 | 0.00 | −0.83 | 0.27 | 0.04 |
| | | NagR | BILO543B32D0_04670 | 0.16 | 0.49 | 0.93 | −0.64 | 0.49 | 0.45 | −0.74 | 0.49 | NA |
| | | NagR | BILO543B32D0_04675 | −0.03 | 0.26 | 0.98 | 1.99 | 0.26 | 0.00 | −0.73 | 0.26 | 0.07 |
| | | NagR | BILO543B32D0_04680 | 0.68 | 0.27 | 0.13 | 2.14 | 0.27 | 0.00 | −1.18 | 0.27 | 0.00 |
| | | NagR | BILO543B32D0_04685 | −0.15 | 0.36 | 0.90 | 0.85 | 0.36 | 0.10 | −0.39 | 0.36 | 0.62 |
| | | NagR | BILO543B32D0_04690 | −0.10 | 0.31 | 0.93 | 0.25 | 0.31 | 0.67 | 0.40 | 0.31 | 0.51 |
| | | NagR | BILO543B32D0_04695 | 0.18 | 0.63 | 0.94 | 0.64 | 0.63 | 0.57 | 0.32 | 0.63 | 0.85 |
| | Nan | NanR | BILO543B32D0_00785 | −0.98 | 0.25 | 0.00 | −0.75 | 0.25 | 0.03 | 0.37 | 0.25 | 0.45 |
| | | NanR | BILO543B32D0_00775 | −0.80 | 0.34 | 0.17 | −0.10 | 0.34 | 0.90 | 1.78 | 0.34 | 0.00 |
| | | NanR | BILO543B32D0_00770 | −0.76 | 0.33 | 0.17 | −0.25 | 0.33 | 0.69 | 1.89 | 0.33 | 0.00 |
| | | NanR | BILO543B32D0_00765 | −1.11 | 0.32 | 0.02 | −0.61 | 0.32 | 0.22 | 2.00 | 0.32 | 0.00 |
| | | NanR | BILO543B32D0_00760 | −1.72 | 0.36 | 0.00 | −1.82 | 0.36 | 0.00 | 3.41 | 0.36 | 0.00 |
| | | NanR | BILO543B32D0_00755 | −2.02 | 0.67 | 0.05 | −1.50 | 0.67 | 0.12 | 3.36 | 0.67 | 0.00 |
| | | NanR | BILO543B32D0_00750 | −1.92 | 0.47 | 0.00 | −1.55 | 0.47 | 0.01 | 3.24 | 0.47 | 0.00 |
| | | NanR | BILO543B32D0_00745 | −1.62 | 0.35 | 0.00 | −0.99 | 0.35 | 0.04 | 2.93 | 0.35 | 0.00 |
| | | NanR | BILO543B32D0_00740 | −1.36 | 0.42 | 0.03 | −1.18 | 0.42 | 0.04 | 3.00 | 0.42 | 0.00 |
| | Ngl | MnaR | BILO543B32D0_04560 | 0.35 | 0.25 | 0.52 | 0.47 | 0.25 | 0.24 | 0.28 | 0.25 | 0.60 |
| | | NglR | BILO543B32D0_04565 | 0.27 | 0.28 | 0.70 | 0.55 | 0.28 | 0.19 | 0.55 | 0.28 | 0.25 |
| | | NglR | BILO543B32D0_04570 | 0.08 | 0.34 | 0.95 | 0.46 | 0.34 | 0.42 | 0.70 | 0.34 | 0.23 |
| | | NglR | BILO543B32D0_04575 | −0.14 | 0.39 | 0.92 | 0.12 | 0.39 | 0.89 | 0.62 | 0.39 | 0.41 |
| | | NglR | BILO543B32D0_04580 | 0.63 | 0.40 | 0.45 | 0.46 | 0.40 | 0.51 | 0.82 | 0.40 | 0.23 |
| | | NglR | BILO543B32D0_04585 | 0.42 | 0.43 | 0.69 | 0.04 | 0.43 | 0.97 | 0.64 | 0.43 | 0.44 |
| | | NglR | BILO543B32D0_04590 | 0.40 | 0.61 | 0.84 | 0.41 | 0.61 | 0.73 | 0.68 | 0.61 | 0.60 |
| | Nglyc_conserved | MnaR | BILO543B32D0_04640 | −0.28 | 0.15 | 0.36 | −0.28 | 0.15 | 0.25 | 0.20 | 0.15 | 0.50 |
| | | MnaR | BILO543B32D0_04645 | −0.30 | 0.24 | 0.59 | −0.11 | 0.24 | 0.83 | 0.87 | 0.24 | 0.01 |
| | | MnaR | BILO543B32D0_04650 | −0.02 | 0.33 | 0.99 | −0.05 | 0.33 | 0.95 | 1.43 | 0.33 | 0.00 |
| | | MnaR | BILO543B32D0_10575 | −0.88 | 0.47 | 0.33 | −0.02 | 0.47 | 0.99 | 1.22 | 0.47 | 0.10 |
| | | MnaR | BILO543B32D0_10580 | −0.31 | 0.38 | 0.78 | −0.26 | 0.38 | 0.73 | 1.22 | 0.38 | 0.03 |
| | | MnaR | BILO543B32D0_10585 | −0.47 | 0.34 | 0.53 | −0.53 | 0.34 | 0.34 | 0.86 | 0.34 | 0.11 |
| | | MnaR | BILO543B32D0_10570 | −0.57 | 0.33 | 0.38 | −0.21 | 0.33 | 0.74 | 1.19 | 0.33 | 0.01 |
| Bifidobacterium | FL1 | FclR | BILO9e02a2a1_01768 | 6.41 | 2.63 | 0.20 | 1.23 | 2.71 | 0.87 | 3.97 | 2.59 | 0.57 |
| longum | | FclR | BILO9e02a2a1_01767 | 0.90 | 0.66 | 0.66 | 1.16 | 0.66 | 0.31 | −0.18 | 0.65 | 0.99 |
| subsp. infantis | FL2 | FclR | BILO9e02a2a1_01614 | 0.44 | 0.73 | 0.94 | 0.08 | 0.73 | 0.98 | 0.90 | 0.72 | 0.72 |
| Bg41721_1G8_SN_2018 | | FclR | BILO9c02a2a1_01613 | −1.42 | 2.49 | NA | −0.14 | 2.45 | NA | −4.84 | 2.57 | NA |
| | Fuc | FucR | BILO9e02a2a1_01813 | −0.67 | 2.50 | 0.98 | −2.33 | 2.51 | 0.69 | 1.70 | 2.51 | 0.93 |
| | | FucR | BILO9e02a2a1_01812 | −4.28 | 2.50 | 0.50 | 0.41 | 2.39 | 0.97 | 2.02 | 2.37 | 0.88 |
| | | FucR | BILO9e02a2a1_01810 | 7.60 | 2.89 | 0.15 | 7.79 | 2.89 | 0.06 | 1.83 | 2.77 | 0.94 |
| | | FucR | BILO9e02a2a1_01809 | −4.07 | 2.09 | NA | −2.44 | 2.01 | 0.55 | −2.12 | 2.11 | 0.85 |
| | Gal | GalR | BILO9c02a2a1_02346 | −0.50 | 2.00 | 0.98 | −1.68 | 2.01 | 0.73 | 0.51 | 2.01 | 1.00 |
| | | GalR | BILO9e02a2a1_02347 | 3.34 | 2.12 | 0.55 | 4.45 | 2.12 | 0.19 | 0.25 | 2.03 | 1.00 |
| | | GalR | BILO9e02a2a1_02348 | −1.77 | 1.54 | 0.77 | 0.27 | 1.53 | 0.97 | 2.76 | 1.52 | 0.48 |
| | HMO_cluster | NA | BILO9e02a2a1_01775 | 3.98 | 2.18 | 0.46 | 3.48 | 2.19 | 0.38 | 1.21 | 2.07 | 0.95 |
| | I | NA | BILO9e02a2a1_01774 | 0.00 | 4.25 | NA | 0.00 | 4.25 | NA | 3.83 | 4.18 | NA |
| | | NA | BILO9e02a2a1_01773 | 0.51 | 4.12 | NA | −4.37 | 4.18 | NA | 0.00 | 4.25 | NA |
| | | NA | BILO9e02a2a1_01772 | 1.11 | 3.11 | NA | −1.83 | 3.16 | 0.83 | 3.74 | 3.13 | 0.76 |
| | | NA | BILO9e02a2a1_01770 | −1.35 | 2.69 | 0.96 | 1.72 | 2.67 | 0.80 | −2.78 | 2.67 | 0.84 |
| | | NagR | BILO9e02a2a1_02383 | 1.48 | 0.79 | NA | 1.05 | 0.79 | NA | −1.03 | 0.72 | 0.64 |
| | | NagR | BILO9e02a2a1_02381 | 2.01 | 0.40 | 0.00 | 1.75 | 0.40 | 0.00 | −0.41 | 0.40 | 0.84 |
| | | NagR | BILO9e02a2a1_01313 | 2.64 | 4.08 | NA | 0.91 | 4.11 | NA | −0.44 | 4.11 | NA |
| | | NagR | BILO9e02a2a1_01312 | −0.78 | 2.83 | NA | −1.16 | 2.83 | NA | −0.02 | 2.83 | NA |
| | | NagR | BILO9e02a2a1_01311 | 6.17 | 2.82 | 0.31 | 4.23 | 2.83 | 0.43 | 1.80 | 2.71 | 0.94 |
| | | NagR | BILO9e02a2a1_01310 | 0.29 | 1.79 | NA | 5.32 | 1.64 | NA | −1.47 | 1.43 | NA |
| | | NagR | BILO9e02a2a1_01309 | 1.17 | 3.16 | NA | −2.85 | 3.22 | 0.71 | 2.38 | 3.21 | 0.91 |
| | | NA | BILO9e02a2a1_01308 | 2.17 | 2.91 | NA | 2.43 | 2.89 | NA | 3.17 | 2.78 | NA |
| | Lac | NA | BILO9e02a2a1_01778 | −2.41 | 4.21 | NA | −2.70 | 4.21 | NA | 0.00 | 4.25 | NA |
| | | NA | BILO9e02a2a1_01777 | 0.16 | 0.78 | 0.98 | 0.17 | 0.77 | 0.95 | −0.80 | 0.76 | 0.83 |
| | | NA | BILO9e02a2a1_01776 | −1.18 | 0.53 | 0.29 | 0.20 | 0.53 | 0.90 | 0.47 | 0.53 | 0.88 |
| | Lnp | NagR | BILO9e02a2a1_01639 | −4.39 | 1.83 | 0.22 | 2.01 | 1.67 | 0.56 | −0.46 | 1.64 | 0.99 |
| | | NagR | BILO9e02a2a1_01638 | 1.08 | 2.49 | NA | 2.29 | 2.44 | NA | 1.27 | 2.31 | NA |
| | | NagR | BILO9e02a2a1_01637 | −0.45 | 1.60 | NA | −0.45 | 1.60 | NA | −0.05 | 1.58 | NA |
| | | NagR | BILO9e02a2a1_01636 | 0.64 | 1.42 | 0.98 | 1.35 | 1.41 | 0.67 | 0.39 | 1.37 | 0.99 |
| | NA | NA | BILO9e02a2a1_01597 | 0.22 | 0.25 | 0.87 | 0.28 | 0.25 | 0.60 | 0.03 | 0.25 | 1.00 |
| | | NA | BILO9e02a2a1_00270 | −0.42 | 3.35 | NA | 0.37 | 3.34 | 0.98 | −0.63 | 3.33 | 1.00 |
| | Nag | NagR | BILO9e02a2a1_00979 | −1.06 | 2.25 | NA | 0.29 | 2.21 | NA | −0.90 | 2.20 | NA |
| | | NagR | BILO9e02a2a1_00978 | 0.41 | 0.70 | 0.94 | 0.38 | 0.70 | 0.84 | 0.29 | 0.69 | 0.97 |
| | | NagR | BILO9c02a2a1_00977 | 4.64 | 2.85 | 0.54 | 7.49 | 2.84 | 0.07 | −0.03 | 2.72 | 1.00 |
| | | NagR | BILO9e02a2a1_00976 | 0.58 | 1.73 | NA | 3.99 | 1.58 | NA | −0.51 | 1.38 | NA |
| | | NagR | BILO9e02a2a1_00974 | 0.24 | 1.42 | 0.98 | −1.87 | 1.45 | 0.52 | 2.32 | 1.44 | 0.56 |
| | | NagR | BILO9c02a2a1_00973 | 0.08 | 2.16 | 0.99 | 0.09 | 2.15 | 1.00 | 0.49 | 2.15 | 1.00 |
| | Nan | NanR | BILO9e02a2a1_00325 | 3.01 | 4.20 | NA | 0.00 | 4.25 | NA | 3.75 | 4.18 | NA |
| | | NanR | BILO9e02a2a1_00323 | −2.61 | 1.46 | 0.48 | −0.95 | 1.44 | 0.80 | 1.42 | 1.44 | 0.85 |
| | | NanR | BILO9e02a2a1_00322 | 4.45 | 2.73 | NA | 3.52 | 2.73 | NA | 0.88 | 2.61 | NA |
| | | NanR | BILO9e02a2a1_00321 | −1.88 | 1.25 | 0.58 | −0.89 | 1.24 | 0.77 | 1.05 | 1.23 | 0.88 |
| | | NanR | BILO9e02a2a1_00320 | 0.24 | 2.91 | NA | −3.51 | 2.99 | NA | 1.80 | 3.01 | NA |
| | | NanR | BILO9e02a2a1_00319 | 2.17 | 2.36 | NA | −1.94 | 2.47 | NA | −1.35 | 2.55 | NA |
| | | NanR | BILO9e02a2a1_00317 | −2.94 | 2.56 | NA | −0.44 | 2.47 | NA | −1.18 | 2.48 | NA |
| | | NanR | BILO9e02a2a1_00316 | 5.87 | 3.84 | NA | 0.43 | 3.91 | 0.98 | 3.20 | 3.83 | 0.89 |
| | Ngl | MnaR | BILO9e02a2a1_01967 | 0.00 | 3.95 | NA | 3.86 | 3.88 | NA | 0.45 | 3.80 | NA |
| | Nglyc_conserved | MnaR | BILO9c02a2a1_01968 | 3.11 | 4.08 | NA | −1.40 | 4.14 | 0.91 | 3.75 | 4.10 | 0.87 |
| | | MnaR | BILO9e02a2a1_01962 | 0.38 | 4.10 | NA | 3.42 | 4.05 | NA | −0.51 | 3.97 | NA |
| | | MnaR | BILO9e02a2a1_00983 | 0.85 | 3.71 | NA | 0.43 | 3.71 | NA | 4.59 | 3.62 | NA |
| | | MnaR | BILO9e02a2a1_00982 | 3.68 | 4.10 | NA | 0.65 | 4.13 | NA | 0.18 | 4.08 | NA |
| | | MnaR | BILO9e02a2a1_01284 | −0.93 | 2.92 | NA | −0.54 | 2.90 | NA | −4.18 | 3.00 | NA |
| | | MnaR | BILO9c02a2a1_01283 | 0.00 | 4.25 | NA | 0.77 | 4.25 | NA | −1.77 | 4.25 | NA |
| | | MnaR | BILO9e02a2a1_01282 | 2.17 | 1.98 | 0.78 | 3.55 | 1.98 | 0.29 | −0.62 | 1.92 | 0.99 |
| Bifidobacterium | FL2 | FclR | BILO16373828_00633 | 0.94 | 0.61 | 0.86 | 1.45 | 0.61 | 0.23 | −0.46 | 0.59 | 0.93 |
| longum | | FclR | BILO16373828_00634 | 0.54 | 0.50 | 0.98 | 0.44 | 0.51 | 0.81 | −1.11 | 0.50 | 0.28 |
| subsp. infantis | | FclR | BILO16373828_00635 | −2.61 | 2.11 | 0.93 | −3.04 | 2.11 | 0.62 | −5.96 | 2.27 | 0.13 |
| JG_BG463.m5.93_JG | Fuc | FucR | BILO16373828_00954 | −0.27 | 0.48 | 1.00 | 0.80 | 0.48 | 0.54 | −0.23 | 0.48 | 0.98 |
| | | FucR | BILO16373828_00955 | 0.93 | 1.25 | 1.00 | −0.27 | 1.25 | 0.96 | 0.09 | 1.25 | 1.00 |
| | | FucR | BILO16373828_00956 | −0.27 | 0.63 | 1.00 | −0.94 | 0.64 | 0.60 | 0.35 | 0.63 | 0.97 |
| | | FucR | BILO16373828_00957 | 0.56 | 4.24 | 1.00 | 0.00 | 4.25 | NA | 0.00 | 4.25 | NA |
| | | FucR | BITO16373828_00958 | −0.60 | 2.01 | 1.00 | 1.38 | 2.01 | 0.85 | −0.69 | 2.01 | 0.99 |
| | | FucR | BILO16373828_00959 | −0.17 | 1.21 | 1.00 | 0.26 | 1.21 | 0.96 | −0.29 | 1.20 | 0.99 |
| | Gal | GalR | BILO16373828_01643 | 2.30 | 1.24 | 0.66 | 0.37 | 1.25 | 0.94 | −3.93 | 1.30 | 0.06 |
| | | GalR | BILO16373828_01644 | −0.12 | 1.35 | 1.00 | −0.93 | 1.35 | 0.85 | −2.40 | 1.36 | 0.48 |
| | | GalR | BILO16373828_01645 | 0.05 | 1.12 | 1.00 | −0.87 | 1.12 | 0.83 | −1.28 | 1.12 | 0.79 |
| | HMO_cluster | NA | BILO16373828_00994 | 0.91 | 0.46 | 0.58 | 0.15 | 0.46 | 0.94 | −0.66 | 0.46 | 0.65 |
| | I | NA | BILO16373828_00995 | 0.55 | 0.38 | 0.89 | −0.36 | 0.38 | 0.78 | −0.35 | 0.38 | 0.90 |
| | | NA | BILO16373828_00996 | −0.31 | 4.06 | 1.00 | −4.73 | 4.13 | NA | 0.00 | 4.20 | NA |
| | | NA | BILO16373828_00997 | 0.81 | 1.06 | 1.00 | −0.37 | 1.06 | 0.94 | 0.53 | 1.06 | 0.98 |
| | | NA | BILO16373828_00998 | 0.23 | 1.12 | 1.00 | 0.39 | 1.12 | 0.94 | −0.50 | 1.11 | 0.98 |
| | | NA | BILO16373828_00999 | 0.85 | 0.43 | 0.58 | 0.67 | 0.43 | 0.58 | −0.23 | 0.43 | 0.97 |
| | | NagR | BILO16373828_01001 | 2.20 | 0.73 | 0.14 | 0.75 | 0.75 | 0.76 | −0.52 | 0.71 | 0.95 |
| | | NagR | BILO16373828_01002 | 1.74 | 0.44 | 0.01 | 1.81 | 0.44 | 0.00 | −0.31 | 0.44 | 0.95 |
| | | NagR | BILO16373828_01003 | 1.72 | 0.91 | 0.65 | −0.18 | 0.91 | 0.96 | −0.06 | 0.91 | 1.00 |
| | | NagR | BILO16373828_01004 | 2.20 | 0.73 | 0.14 | 0.75 | 0.75 | 0.76 | −0.52 | 0.71 | 0.95 |
| | | NagR | BILO16373828_01005 | 1.74 | 0.44 | 0.01 | 1.81 | 0.44 | 0.00 | −0.31 | 0.44 | 0.95 |
| | | NagR | BILO16373828_01006 | 2.14 | 1.83 | 0.94 | 0.73 | 1.83 | 0.93 | −0.97 | 1.83 | 0.97 |
| | | NagR | BILO16373828_01007 | 3.95 | 1.42 | 0.19 | 2.20 | 1.44 | 0.59 | −0.10 | 1.33 | 1.00 |
| | | NagR | BILO16373828_01008 | 0.36 | 1.03 | 1.00 | −0.74 | 1.04 | 0.85 | −1.25 | 1.04 | 0.77 |
| | | NagR | BILO16373828_01009 | 0.46 | 0.45 | 0.99 | 1.25 | 0.45 | 0.13 | −0.31 | 0.44 | 0.95 |
| | | NagR | BILO16373828_01010 | 0.50 | 0.85 | 1.00 | −0.28 | 0.85 | 0.94 | −0.38 | 0.85 | 0.98 |
| | | NagR | BILO16373828_01011 | 1.01 | 0.80 | 0.93 | 0.08 | 0.80 | 0.99 | −0.06 | 0.80 | 1.00 |
| | | NA | BILO16373828_01012 | 1.08 | 0.46 | 0.40 | 0.34 | 0.46 | 0.85 | −0.19 | 0.46 | 0.98 |
| | Lac | NA | BILO16373828_00991 | −0.80 | 0.51 | 0.85 | −1.14 | 0.51 | 0.27 | 0.33 | 0.51 | 0.96 |
| | | NA | BILO16373828_00992 | −3.03 | 1.20 | 0.30 | −8.01 | 1.21 | 0.00 | 0.17 | 1.22 | 1.00 |
| | | NA | BILO16373828_00993 | −2.04 | 0.69 | 0.14 | −3.49 | 0.69 | 0.00 | 0.32 | 0.69 | 0.98 |
| | Lnp | NagR | BILO16373828_00605 | −0.05 | 0.73 | 1.00 | −1.14 | 0.73 | 0.58 | −1.63 | 0.73 | 0.27 |
| | | NagR | BILO16373828_00606 | 0.13 | 0.98 | 1.00 | −0.78 | 0.98 | 0.82 | −2.47 | 0.99 | 0.18 |
| | | NagR | BILO16373828_00607 | −0.07 | 0.62 | 1.00 | −0.88 | 0.62 | 0.64 | −0.96 | 0.63 | 0.61 |
| | | NagR | BILO16373828_00608 | −0.55 | 0.69 | 1.00 | −1.91 | 0.69 | 0.13 | −0.61 | 0.69 | 0.91 |
| | | NagR | BILO16373828_00609 | −0.86 | 1.33 | 1.00 | −1.77 | 1.33 | 0.67 | −0.99 | 1.34 | 0.95 |
| | | NagR | BILO16373828_00610 | 0.34 | 0.95 | 1.00 | −2.51 | 0.96 | 0.17 | 0.13 | 0.97 | 1.00 |
| | | NagR | BILO16373828_00611 | −0.14 | 0.70 | 1.00 | −2.14 | 0.70 | 0.08 | −0.32 | 0.70 | 0.98 |
| | NA | NA | BILO16373828_00492 | 0.11 | 0.34 | 1.00 | −0.29 | 0.34 | 0.81 | 0.01 | 0.34 | 1.00 |
| | | NA | BILO16373828_01180 | −0.06 | 0.52 | 1.00 | 0.82 | 0.51 | 0.56 | 1.94 | 0.51 | 0.01 |
| | | NA | BILO16373828_01181 | 0.83 | 1.32 | 1.00 | 2.77 | 1.22 | 0.27 | 1.61 | 0.92 | NA |
| | | NA | BILO16373828_01182 | 0.00 | 3.86 | 1.00 | 0.49 | 3.86 | NA | 2.38 | 3.79 | NA |
| | | NA | BILO16373828_01183 | −1.30 | 1.36 | 1.00 | −2.04 | 1.38 | 0.60 | −0.60 | 1.40 | 0.98 |
| | | NA | BILO16373828_01600 | 0.05 | 0.67 | 1.00 | −2.37 | 0.68 | 0.02 | −1.86 | 0.69 | 0.12 |
| | Nag | NagR | BILO16373828_01189 | 1.89 | 1.88 | 1.00 | 1.85 | 1.88 | 0.77 | −0.97 | 1.87 | 0.97 |
| | | NagR | BILO16373828_01190 | −0.24 | 0.58 | 1.00 | −0.85 | 0.58 | 0.61 | −0.36 | 0.59 | 0.96 |
| | | NagR | BILO16373828_01191 | 1.16 | 0.78 | 0.87 | −0.11 | 0.79 | 0.98 | −0.01 | 0.79 | 1.00 |
| | | NagR | BILO16373828_01192 | 0.38 | 0.50 | 1.00 | −0.29 | 0.50 | 0.89 | −0.10 | 0.50 | 1.00 |
| | Nan | NanR | BILO16373828_01882 | 0.22 | 0.92 | 1.00 | 0.53 | 0.92 | 0.89 | −0.66 | 0.91 | 0.95 |
| | | NanR | BILO16373828_01880 | −0.48 | 0.60 | 1.00 | −0.12 | 0.60 | 0.96 | −0.32 | 0.60 | 0.97 |
| | | NanR | BITO16373828_01879 | −2.27 | 0.72 | 0.12 | −1.96 | 0.72 | 0.15 | 0.06 | 0.72 | 1.00 |
| | | NanR | BILO16373828_01878 | 0.69 | 1.11 | 1.00 | 1.13 | 1.11 | 0.76 | −0.55 | 1.11 | 0.98 |
| | | NanR | BILO16373828_01877 | −0.75 | 1.43 | 1.00 | −1.64 | 1.43 | 0.72 | −0.54 | 1.43 | 0.98 |
| | | NanR | BILO16373828_01876 | −1.50 | 1.99 | 1.00 | −2.82 | 1.99 | 0.64 | −1.37 | 2.01 | 0.96 |
| | | NanR | BILO16373828_01875 | −0.54 | 1.24 | 1.00 | −1.23 | 1.24 | 0.77 | −0.73 | 1.24 | 0.97 |
| | | NanR | BILO16373828_01874 | 0.40 | 1.63 | 1.00 | −0.08 | 1.63 | 1.00 | −1.07 | 1.63 | 0.96 |
| | | NanR | BILO16373828_01873 | −1.00 | 2.36 | 1.00 | 0.25 | 2.36 | 0.99 | −2.47 | 2.36 | 0.83 |
| | Ngl | MnaR | BILO16373828_01174 | −1.01 | 0.39 | 0.27 | −0.61 | 0.39 | 0.58 | 2.41 | 0.39 | 0.00 |
| | Nglyc_conserved | MnaR | BILO16373828_01173 | −0.81 | 0.45 | 0.72 | −0.26 | 0.45 | 0.89 | 2.54 | 0.45 | 0.00 |
| | | MnaR | BILO16373828_01184 | −0.65 | 0.68 | 1.00 | −1.36 | 0.68 | 0.37 | −1.62 | 0.71 | 0.25 |
| | | MnaR | BILO16373828_01185 | −0.88 | 0.54 | 0.85 | 0.41 | 0.54 | 0.84 | 0.23 | 0.54 | 0.98 |
| | | MnaR | BILO16373828_01186 | −0.42 | 0.75 | 1.00 | 1.23 | 0.75 | 0.54 | 0.43 | 0.75 | 0.97 |
| | | MnaR | BILO16373828_01177 | −1.22 | 0.44 | 0.19 | −0.35 | 0.44 | 0.82 | 2.19 | 0.43 | 0.00 |
| | | MnaR | BILO16373828_01176 | −0.92 | 0.38 | 0.37 | −0.61 | 0.38 | 0.56 | 2.51 | 0.38 | 0.00 |
| | | MnaR | BILO16373828_01175 | −0.65 | 0.46 | 0.90 | −0.67 | 0.46 | 0.62 | 2.31 | 0.46 | 0.00 |
| | | MnaR | BILO16373828_01178 | −0.86 | 0.32 | 0.24 | −0.36 | 0.32 | 0.73 | 2.32 | 0.32 | 0.00 |
| Bifidobacterium | FL1 | FclR | BILO145876ef_00441 | −1.40 | 3.61 | 0.99 | −4.20 | 3.63 | 0.73 | 0.72 | 3.70 | NA |
| longum | | FclR | BILO145876ef_00442 | −0.59 | 0.85 | 0.97 | −1.37 | 0.85 | 0.55 | −1.79 | 0.87 | 0.38 |
| subsp. infantis | | FclR | BILO145876ef_00443 | −0.68 | 1.03 | 0.97 | −1.36 | 1.03 | 0.66 | −0.37 | 1.03 | NA |
| PS064_13.C6_Bang_JG | | FclR | BILO145876ef_00444 | 0.78 | 1.87 | 0.98 | 0.04 | 1.87 | 0.99 | −0.61 | 1.86 | NA |
| | | FclR | BILO145876ef_00445 | 0.00 | 4.24 | 1.00 | 0.00 | 4.25 | NA | 1.06 | 4.24 | NA |
| | FL2 | FclR | BILO145876ef_01994 | 0.52 | 0.76 | 0.97 | −0.15 | 0.76 | 0.97 | 1.35 | 0.74 | NA |
| | | FclR | BILO145876ef_01995 | 0.00 | 0.68 | 1.00 | −0.37 | 0.68 | 0.92 | −0.52 | 0.68 | 0.93 |
| | | FclR | BITO145876ef_01996 | −0.59 | 0.85 | 0.97 | −1.37 | 0.85 | 0.55 | −1.79 | 0.87 | 0.38 |
| | Fuc | FucR | BILO145876ef_02113 | −0.24 | 0.46 | 0.98 | −0.35 | 0.46 | 0.86 | 0.07 | 0.46 | 1.00 |
| | | FucR | BILO145876ef_02114 | 0.60 | 0.57 | 0.89 | 1.61 | 0.56 | 0.09 | −0.54 | 0.56 | 0.89 |
| | | FucR | BILO145876ef_02115 | −0.10 | 0.57 | 1.00 | −1.19 | 0.57 | 0.34 | 0.67 | 0.57 | NA |
| | | FucR | BILO145876ef_02116 | −0.07 | 0.28 | 0.99 | −0.23 | 0.28 | 0.84 | −0.14 | 0.28 | 0.97 |
| | | FucR | BITO145876ef_02117 | −1.06 | 0.82 | 0.82 | −0.33 | 0.82 | 0.95 | 0.03 | 0.82 | 1.00 |
| | | FucR | BILO145876ef_02118 | 1.47 | 1.03 | 0.76 | 0.36 | 1.03 | 0.95 | 0.80 | 1.02 | NA |
| | Gal | GalR | BILO145876ef_01870 | −0.01 | 0.30 | 1.00 | 0.00 | 0.30 | 1.00 | −0.52 | 0.30 | 0.50 |
| | | GalR | BILO145876ef_01871 | 0.80 | 0.32 | 0.28 | −0.47 | 0.32 | 0.61 | −0.21 | 0.32 | 0.95 |
| | | GalR | BILO145876ef_01872 | −2.70 | 1.86 | 0.74 | −2.21 | 1.85 | 0.71 | 3.37 | 1.85 | 0.47 |
| | HMO_cluster | NA | BITO145876ef_02152 | 0.31 | 0.37 | 0.96 | 0.55 | 0.37 | 0.59 | −1.10 | 0.37 | 0.09 |
| | I | NA | BILO145876ef_02153 | −0.20 | 0.30 | 0.97 | 0.03 | 0.30 | 0.98 | −0.88 | 0.30 | 0.10 |
| | | NA | BILO145876ef_02154 | −0.35 | 0.59 | 0.97 | −0.07 | 0.59 | 0.98 | −0.82 | 0.59 | 0.69 |
| | | NA | BILO145876ef_02155 | −0.17 | 0.66 | 0.99 | −0.27 | 0.66 | 0.95 | −0.50 | 0.66 | 0.93 |
| | | NA | BILO145876ef_02156 | 0.02 | 0.46 | 1.00 | −0.37 | 0.46 | 0.84 | −0.56 | 0.46 | 0.80 |
| | | NA | BITO145876ef_02157 | 0.38 | 1.03 | 0.99 | 1.13 | 1.03 | 0.74 | −0.45 | 1.02 | 0.97 |
| | | NagR | BILO145876ef_02159 | −0.50 | 0.58 | 0.95 | −0.71 | 0.57 | 0.69 | −0.27 | 0.57 | NA |
| | | NagR | BILO145876ef_02160 | 1.72 | 0.39 | 0.00 | 1.25 | 0.39 | 0.04 | 0.07 | 0.39 | 1.00 |
| | | NagR | BILO145876ef_02161 | 0.35 | 1.00 | 0.99 | 0.45 | 1.00 | 0.94 | −0.30 | 1.00 | 0.98 |
| | | NagR | BILO145876ef_02162 | −0.50 | 0.58 | 0.95 | −0.71 | 0.57 | 0.69 | −0.27 | 0.57 | NA |
| | | NagR | BITO145876ef_02163 | 1.72 | 0.39 | 0.00 | 1.25 | 0.39 | 0.04 | 0.07 | 0.39 | 1.00 |
| | | NagR | BILO145876ef_02164 | 0.05 | 0.38 | 1.00 | −0.04 | 0.38 | 0.98 | −0.31 | 0.37 | 0.90 |
| | | NagR | BILO145876ef_02165 | 1.36 | 1.07 | 0.83 | 0.93 | 1.07 | 0.82 | 0.03 | 1.06 | 1.00 |
| | | NagR | BILO145876ef_02166 | 0.19 | 0.57 | 0.99 | 0.43 | 0.57 | 0.86 | −0.02 | 0.56 | 1.00 |
| | | NagR | BILO145876ef_02167 | 0.12 | 0.46 | 0.99 | 0.02 | 0.46 | 0.99 | 0.02 | 0.46 | 1.00 |
| | | NagR | BILO145876ef_02168 | 0.16 | 0.36 | 0.98 | 0.13 | 0.36 | 0.95 | −0.12 | 0.36 | 0.98 |
| | | NagR | BILO145876ef_02169 | −0.23 | 0.48 | 0.98 | −0.74 | 0.48 | 0.57 | −0.14 | 0.48 | 0.98 |
| | | NA | BILO145876ef_02170 | −0.23 | 0.35 | 0.97 | −0.29 | 0.35 | 0.83 | −0.15 | 0.35 | 0.97 |
| | Lac | NA | BILO145876ef_02149 | 1.10 | 0.86 | 0.83 | 0.57 | 0.86 | 0.89 | 0.19 | 0.85 | 1.00 |
| | | NA | BILO145876ef_02150 | 0.20 | 0.78 | 0.99 | −0.12 | 0.77 | 0.98 | −0.46 | 0.76 | NA |
| | | NA | BILO145876ef_02151 | 0.30 | 0.25 | 0.85 | 0.86 | 0.25 | 0.02 | −0.35 | 0.24 | 0.64 |
| | Lnp | NagR | BILO145876ef_01969 | 0.30 | 0.29 | 0.91 | 0.26 | 0.29 | 0.80 | −0.86 | 0.29 | 0.10 |
| | | NagR | BILO145876ef_01970 | 0.42 | 0.80 | 0.98 | 0.31 | 0.80 | 0.95 | −0.75 | 0.80 | 0.89 |
| | | NagR | BILO145876ef_01971 | 0.10 | 0.57 | 1.00 | 0.04 | 0.57 | 0.99 | 0.02 | 0.57 | 1.00 |
| | | NagR | BILO145876ef_01972 | 1.12 | 0.71 | 0.66 | 0.82 | 0.71 | 0.72 | 0.12 | 0.70 | 1.00 |
| | NA | NA | BILO145876ef_00859 | −0.06 | 0.23 | 0.99 | −0.02 | 0.23 | 0.99 | 0.20 | 0.23 | 0.90 |
| | | NA | BILO145876ef_01810 | 0.18 | 0.24 | 0.97 | 0.11 | 0.24 | 0.94 | −0.48 | 0.24 | 0.41 |
| | | MnaR | BILO145876ef_00034 | 0.24 | 0.96 | 0.99 | 0.34 | 0.95 | 0.95 | 0.72 | 0.95 | 0.93 |
| | Nag | NagR | BILO145876ef_01016 | −1.87 | 1.41 | 0.80 | −0.50 | 1.38 | 0.95 | 0.94 | 1.37 | NA |
| | | NagR | BILO145876ef_01017 | 0.17 | 0.82 | 1.00 | 0.20 | 0.82 | 0.96 | −1.55 | 0.82 | 0.44 |
| | | NagR | BILO145876ef_01018 | −0.22 | 0.26 | 0.95 | 0.26 | 0.26 | 0.77 | −0.61 | 0.26 | 0.24 |
| | | NagR | BILO145876ef_01019 | −0.06 | 0.36 | 1.00 | −0.03 | 0.36 | 0.98 | −0.66 | 0.36 | 0.45 |
| | | NagR | BILO145876ef_01020 | −0.50 | 0.28 | 0.58 | −0.27 | 0.28 | 0.79 | −0.48 | 0.28 | 0.52 |
| | | NagR | BILO145876ef_01021 | 1.00 | 1.22 | 0.96 | 0.40 | 1.22 | 0.96 | −0.03 | 1.22 | 1.00 |
| | | NagR | BILO145876ef_01022 | −0.53 | 1.15 | 0.98 | −0.83 | 1.15 | 0.87 | −0.48 | 1.15 | 0.97 |
| | Nan | NanR | BILO145876ef_00779 | −0.07 | 0.96 | 1.00 | 0.26 | 0.95 | 0.96 | −0.58 | 0.95 | NA |
| | | NanR | BILO145876ef_00781 | −0.18 | 0.39 | 0.98 | 0.50 | 0.39 | 0.69 | −1.17 | 0.39 | 0.10 |
| | | NanR | BILO145876ef_00783 | −0.76 | 0.65 | 0.86 | 0.10 | 0.64 | 0.98 | −1.72 | 0.65 | 0.16 |
| | | NanR | BILO145876ef_00784 | −1.68 | 1.24 | 0.78 | 0.23 | 1.23 | 0.98 | −0.89 | 1.23 | 0.94 |
| | | NanR | BILO145876ef_00785 | 0.71 | 1.77 | 0.99 | 1.90 | 1.76 | 0.76 | −1.57 | 1.75 | NA |
| | | NanR | BILO145876ef_00786 | −1.12 | 1.37 | 0.96 | −1.10 | 1.36 | 0.84 | 0.08 | 1.36 | 1.00 |
| | | NanR | BILO145876ef_00787 | −2.40 | 1.33 | 0.58 | 1.69 | 1.30 | 0.67 | −0.07 | 1.29 | 1.00 |
| | | NanR | BILO145876ef_00788 | −0.07 | 1.39 | 1.00 | 0.42 | 1.39 | 0.96 | 0.25 | 1.38 | 1.00 |
| | Ngl | MnaR | BILO145876ef_01006 | −0.26 | 0.54 | 0.98 | 0.24 | 0.53 | 0.94 | −0.02 | 0.53 | 1.00 |
| | Nglyc_conserved | MnaR | BILO145876ef_01005 | −0.04 | 0.92 | 1.00 | 0.77 | 0.92 | 0.83 | 0.49 | 0.92 | 0.97 |
| | | MnaR | BILO145876ef_01011 | −1.68 | 1.15 | 0.74 | 0.39 | 1.14 | 0.95 | −0.77 | 1.14 | 0.95 |
| | | MnaR | BILO145876ef_01012 | −0.72 | 0.88 | 0.96 | 0.58 | 0.88 | 0.89 | −0.35 | 0.88 | 0.97 |
| | | MnaR | BILO145876ef_01013 | −0.89 | 1.44 | 0.97 | −0.08 | 1.42 | 0.99 | 1.12 | 1.41 | NA |
| | | MnaR | BILO145876ef_02186 | 4.60 | 1.89 | 0.31 | 3.10 | 1.90 | 0.55 | 1.61 | 1.72 | NA |
| | | MnaR | BILO145876ef_02187 | 0.74 | 0.98 | 0.97 | 1.17 | 0.97 | 0.71 | 0.47 | 0.96 | NA |
| | | MnaR | BILO145876ef_02188 | 0.28 | 0.43 | 0.97 | 0.62 | 0.43 | 0.61 | 0.48 | 0.43 | 0.84 |
| | | MnaR | BILO145876ef_02185 | 0.39 | 0.96 | 0.99 | 1.35 | 0.95 | 0.62 | 0.04 | 0.93 | NA |
| Bifidobacterium | Blon_0459- | NA | N_02388 | −0.96 | 1.32 | 0.86 | −1.24 | 1.32 | 0.74 | −0.57 | 1.33 | 0.90 |
| longum | 0462 | NA | N_02387 | −1.43 | 0.90 | 0.56 | −0.34 | 0.88 | 0.92 | −0.87 | 0.88 | NA |
| subsp. infantis | | NA | N_02386 | −1.89 | 1.50 | 0.69 | −1.11 | 1.47 | 0.81 | −2.17 | 1.55 | NA |
| EVC001 | | NA | N_02385 | −2.92 | 1.43 | 0.36 | −0.48 | 1.35 | 0.93 | −1.73 | 1.38 | NA |
| | FL1 | FclR | N_02513 | −2.21 | 3.09 | NA | −4.50 | 3.11 | NA | 3.72 | 3.11 | NA |
| | | FclR | N_02512 | −0.12 | 0.21 | 0.91 | −0.07 | 0.21 | 0.93 | 0.10 | 0.21 | 0.90 |
| | | FclR | N_02511 | 0.23 | 0.25 | 0.80 | −0.07 | 0.25 | 0.95 | 0.38 | 0.24 | 0.53 |
| | | FclR | N_02510 | 0.26 | 0.28 | 0.80 | 0.84 | 0.28 | 0.07 | −0.18 | 0.28 | 0.85 |
| | | FclR | N_02509 | 1.79 | 3.45 | NA | 0.12 | 3.49 | NA | 0.64 | 3.47 | NA |
| | FL2 | FclR | N_00533 | 0.05 | 0.22 | 0.97 | −0.06 | 0.22 | 0.95 | 0.45 | 0.22 | 0.35 |
| | | FclR | N_00532 | 0.23 | 0.25 | 0.80 | −0.07 | 0.25 | 0.95 | 0.38 | 0.24 | 0.53 |
| | | FclR | N_00531 | −0.12 | 0.21 | 0.91 | −0.07 | 0.21 | 0.93 | 0.10 | 0.21 | 0.90 |
| | Fuc | FucR | N_00418 | 0.11 | 0.70 | 0.98 | −0.79 | 0.70 | 0.69 | −0.30 | 0.70 | 0.90 |
| | | FucR | N_00417 | 0.47 | 2.55 | 0.97 | −6.02 | 2.65 | 0.26 | 0.00 | 2.77 | NA |
| | | FucR | N_00416 | −0.31 | 0.49 | 0.90 | −0.27 | 0.49 | 0.88 | 0.36 | 0.49 | 0.83 |
| | | FucR | N_00415 | −0.36 | 0.35 | 0.78 | −0.06 | 0.35 | 0.96 | 0.81 | 0.35 | 0.27 |
| | | FucR | N_00414 | −0.03 | 0.77 | 0.99 | −0.51 | 0.77 | 0.84 | 0.69 | 0.77 | 0.78 |
| | | FucR | N_00413 | 1.02 | 0.75 | 0.65 | 1.05 | 0.75 | 0.57 | 0.01 | 0.74 | 0.99 |
| | Gal | GalR | N_00672 | −0.03 | 0.37 | 0.99 | −0.32 | 0.37 | 0.77 | −0.94 | 0.37 | 0.20 |
| | | GalR | N_00671 | 0.18 | 0.25 | 0.87 | −0.08 | 0.25 | 0.94 | −0.75 | 0.25 | 0.11 |
| | | GalR | N_00670 | −0.03 | 0.54 | 0.99 | 1.14 | 0.54 | 0.31 | 0.25 | 0.54 | 0.90 |
| | HMO_cluster | NA | N_00387 | 0.12 | 0.23 | 0.91 | −0.25 | 0.23 | 0.69 | −0.86 | 0.23 | 0.01 |
| | I | NA | N_00386 | 0.48 | 0.24 | 0.37 | −0.38 | 0.24 | 0.50 | −0.45 | 0.24 | 0.39 |
| | | NA | N_00385 | 0.18 | 3.71 | 0.99 | −4.93 | 3.78 | NA | 0.00 | 3.86 | NA |
| | | NA | N_00384 | −0.01 | 0.37 | 1.00 | −0.71 | 0.37 | 0.38 | −0.71 | 0.37 | 0.39 |
| | | NA | N_00383 | −0.02 | 0.64 | 0.99 | 0.94 | 0.64 | 0.55 | −0.48 | 0.64 | 0.83 |
| | | NA | N_00382 | 0.19 | 0.58 | 0.96 | 0.36 | 0.58 | 0.86 | −0.26 | 0.58 | 0.90 |
| | | NagR | N_00380 | 1.10 | 0.59 | 0.43 | 0.50 | 0.59 | 0.78 | −0.41 | 0.58 | 0.84 |
| | | NagR | N_00379 | 1.96 | 0.52 | 0.02 | 1.46 | 0.52 | 0.12 | 0.19 | 0.52 | 0.91 |
| | | NagR | N_00378 | 1.11 | 0.49 | 0.29 | 0.96 | 0.49 | 0.36 | −0.46 | 0.48 | 0.77 |
| | | NagR | N_00377 | 1.10 | 0.59 | 0.43 | 0.50 | 0.59 | 0.78 | −0.41 | 0.58 | 0.84 |
| | | NagR | N_00376 | 1.96 | 0.52 | 0.02 | 1.46 | 0.52 | 0.12 | 0.19 | 0.52 | 0.91 |
| | | NagR | N_00375 | 0.68 | 0.68 | 0.78 | 0.56 | 0.68 | 0.79 | −0.61 | 0.68 | 0.78 |
| | | NagR | N_00374 | −0.48 | 0.78 | 0.90 | −0.18 | 0.77 | 0.95 | −0.40 | 0.77 | 0.89 |
| | | NagR | N_00373 | 1.26 | 0.45 | 0.15 | 0.80 | 0.45 | 0.43 | −0.35 | 0.44 | 0.81 |
| | | NagR | N_00372 | 0.68 | 0.39 | 0.48 | 0.47 | 0.39 | 0.66 | −0.52 | 0.39 | 0.63 |
| | | NagR | N_00371 | 0.42 | 0.33 | 0.69 | 0.57 | 0.33 | 0.44 | −0.39 | 0.33 | 0.68 |
| | | NagR | N_00370 | 0.24 | 0.38 | 0.90 | 0.07 | 0.38 | 0.96 | −0.21 | 0.38 | 0.89 |
| | | NagR | N_00369 | 0.66 | 0.35 | 0.42 | 0.16 | 0.35 | 0.90 | −0.25 | 0.35 | 0.84 |
| | | NA | N_00368 | −0.17 | 0.36 | 0.92 | −0.06 | 0.36 | 0.96 | −0.43 | 0.36 | 0.68 |
| | Lac | NA | N_00391 | −0.65 | 0.44 | 0.60 | −0.35 | 0.44 | 0.80 | −0.15 | 0.44 | 0.92 |
| | | NA | N_00390 | −0.58 | 0.32 | 0.45 | −0.36 | 0.32 | 0.69 | −0.47 | 0.32 | 0.56 |
| | | NA | N_00388 | 0.20 | 0.22 | 0.81 | 0.47 | 0.22 | 0.29 | −0.24 | 0.22 | 0.71 |
| | Lnp | NagR | N_00563 | 0.14 | 0.22 | 0.90 | −0.14 | 0.22 | 0.85 | −0.13 | 0.22 | 0.87 |
| | | NagR | N_00562 | 0.62 | 0.59 | 0.78 | 0.17 | 0.59 | 0.95 | −0.31 | 0.59 | 0.89 |
| | | NagR | N_00561 | 0.34 | 0.36 | 0.80 | 0.35 | 0.36 | 0.74 | −0.48 | 0.36 | 0.65 |
| | | NagR | N_00560 | 0.11 | 0.35 | 0.95 | −0.19 | 0.35 | 0.88 | −0.54 | 0.35 | 0.54 |
| | | NagR | N_00559 | 0.44 | 0.69 | 0.90 | 0.29 | 0.69 | 0.92 | −0.03 | 0.69 | 0.99 |
| | | NagR | N_00558 | 0.27 | 0.31 | 0.83 | 0.49 | 0.31 | 0.51 | −0.24 | 0.31 | 0.82 |
| | | NagR | N_00557 | −0.06 | 0.26 | 0.97 | 0.01 | 0.26 | 0.99 | −0.17 | 0.26 | 0.85 |
| | NA | NA | N_02109 | 0.36 | 0.36 | 0.78 | −0.25 | 0.36 | 0.83 | 0.14 | 0.36 | 0.91 |
| | | NA | N_00717 | 0.80 | 0.54 | 0.60 | 0.10 | 0.54 | 0.96 | −0.10 | 0.54 | 0.95 |
| | | MnaR | N_00253 | −0.49 | 0.35 | 0.63 | −0.41 | 0.35 | 0.68 | 0.41 | 0.35 | 0.68 |
| | Nag | NagR | N_01960 | 1.10 | 0.97 | 0.75 | 0.72 | 0.97 | 0.81 | −0.43 | 0.97 | 0.90 |
| | | NagR | N_01959 | −0.48 | 0.57 | 0.84 | 0.47 | 0.57 | 0.79 | 0.69 | 0.57 | 0.67 |
| | | NagR | N_01958 | −0.24 | 0.24 | 0.78 | 0.14 | 0.24 | 0.88 | −0.28 | 0.24 | 0.68 |
| | | NagR | N_01957 | 0.16 | 0.29 | 0.91 | −0.20 | 0.29 | 0.82 | −0.48 | 0.29 | 0.48 |
| | | NagR | N_01956 | −0.04 | 0.28 | 0.99 | −0.18 | 0.28 | 0.85 | −0.12 | 0.28 | 0.90 |
| | | NagR | N_01955 | 0.25 | 0.31 | 0.85 | −0.22 | 0.31 | 0.83 | 0.10 | 0.31 | 0.93 |
| | | NagR | N_01954 | −0.19 | 0.21 | 0.81 | −0.11 | 0.21 | 0.88 | 0.20 | 0.21 | 0.76 |
| | Nan | NanR | N_02202 | −0.39 | 0.42 | 0.80 | −0.74 | 0.42 | 0.44 | 0.88 | 0.42 | 0.34 |
| | | NanR | N_02200 | −1.00 | 0.48 | 0.34 | −0.95 | 0.48 | 0.36 | −0.52 | 0.48 | 0.71 |
| | | NanR | N_02199 | −1.13 | 0.46 | 0.26 | −1.06 | 0.46 | 0.26 | −0.67 | 0.46 | 0.57 |
| | | NanR | N_02198 | −1.03 | 0.50 | 0.35 | −0.92 | 0.50 | 0.40 | −1.00 | 0.50 | 0.37 |
| | | NanR | N_02197 | −1.77 | 0.84 | 0.34 | −1.16 | 0.84 | 0.59 | −1.72 | 0.85 | 0.36 |
| | | NanR | N_02196 | 0.51 | 1.67 | 0.96 | −1.66 | 1.68 | 0.73 | −0.67 | 1.69 | 0.91 |
| | | NanR | N_02195 | −1.58 | 0.67 | 0.27 | −1.92 | 0.67 | 0.10 | −0.13 | 0.67 | 0.95 |
| | | NanR | N_02194 | −3.52 | 1.05 | 0.06 | −1.15 | 1.03 | 0.70 | −0.42 | 1.03 | 0.91 |
| | | NanR | N_02193 | −0.87 | 0.51 | 0.50 | −0.77 | 0.51 | 0.53 | −0.60 | 0.51 | 0.68 |
| | Nglyc_conserved | MnaR | N_01971 | −0.24 | 0.56 | 0.93 | 0.01 | 0.56 | 1.00 | 0.27 | 0.56 | 0.89 |
| | | MnaR | N_01965 | 0.10 | 0.26 | 0.94 | −0.25 | 0.26 | 0.73 | 0.25 | 0.25 | 0.76 |
| | | MnaR | N_01964 | −0.25 | 0.22 | 0.76 | −0.13 | 0.22 | 0.87 | 0.11 | 0.22 | 0.89 |
| | | MnaR | N_01963 | −0.65 | 0.57 | 0.75 | −0.22 | 0.57 | 0.92 | 0.63 | 0.57 | 0.71 |
| | | MnaR | N_00347 | −0.54 | 0.49 | 0.77 | −0.43 | 0.49 | 0.76 | 0.07 | 0.49 | 0.96 |
| | | MnaR | N_00346 | −0.62 | 0.45 | 0.65 | −0.67 | 0.45 | 0.55 | −0.28 | 0.45 | 0.87 |
| | | MnaR | N_00345 | −0.13 | 0.46 | 0.96 | −0.33 | 0.46 | 0.81 | −0.37 | 0.46 | 0.81 |
| | | MnaR | N_00348 | −0.50 | 0.44 | 0.75 | −0.71 | 0.44 | 0.49 | −0.05 | 0.44 | 0.96 |

Collectively, this analysis indicated that among the strains evaluated, Bg_2D9 has the greatest endowment of glycoside hydrolases and candidate transporters for N-glycan utilization. As summarized in FIG. 5C, it was postulated that (i) its endo-β-N-acetylglucosaminidases EndoBI-2 and EndoBB-2 are able to release sugar moieties from N-glycans, which are further transported into the cell via nglABC or Blon_2378-2380, (ii) these ABC transport systems may exhibit different preferences for various N-glycan structures, and (iii) internalized sugar moieties are degraded from the non-reducing end by an orchestrated action of multiple intracellular exo-acting glycoside hydrolases. It was surmised that many glycoside hydrolases involved in HMO utilization, namely, Bga2A, Hex1, Hex2, NanH2, BiAfcA, BiAfcB may also contribute to utilization of complex N-glycans (containing GlcNAc, fucose, and NANa residues) given that these enzymes are known to act on glycosidic bonds found in both HMOs and N-glycans.

### Example 6: Prevalence of B. Infantis Strains Possessing the Ngl Transporter in Bangladeshi Children

A qPCR assay that used primers against the nglA component of the ABC transport system identified in the genome of the B. infantis Bg_2D9 strain was used on the fecal DNA samples from the cross-sectional survey of Bangladeshi infants/children. This assay disclosed that fecal levels of nglA were on average 2-3 orders of magnitude lower in 3- to 13-month-old children with SAM compared to their healthy age-matched counterparts (P<0.05, generalized additive model; red-bounded region in FIG. 2D), while no significant differences were evident between 14- to 24-month-old children (P>0.05; Generalized additive model). Notably, of the samples for which sufficient DNA was available to assay, 55 of 117 (47%) from healthy children and only 18 out of 83 (22%) of those from SAM children were positive for nglA (P<0.05, two proportion Z-test). Importantly, there were no fecal samples that were positive for nglA and negative for the characteristic B. infantis Blon_2348/nanH2 sialidase gene, suggesting that when detected, nglA is present within genomes belonging to strains of B. infantis.

### Example 7: Competition Between B. Infantis Bg_2D9 and EVC001 in Gnotobiotic Mice Harboring a SAM Microbiota

Gnotobiotic mice were used to test the relative capacities of B. infantis Bg_2D9 and EVC001 to establish themselves in a fecal microbiota sample obtained from a 5-month-old infant with SAM in the SYNERGIE trial prior to the probiotic intervention. The experimental design is summarized in FIG. 4F). Germ-free pregnant C57BL/6J dams were initially housed in the same isolator which contained 2 cages with 2 dams/cage. Animals were fed a standard breeder chow. On day 2 after parturition, both groups of dams were switched to the Mirpur-6 diet. On postpartum day 4, both dams in each group were gavaged with the fecal community from the SAM infant. The gavage was repeated three days later and on day 11, one of the two cages was moved to a separate isolator. On postpartum days 12 and 14, another type of gavage was performed, this one consisting of a mixture containing equivalent concentrations of Bg_2D9 and EVC001 that was administered to both dams in one of the two groups; both dams in the other group received a sham gavage. Pups (n=11-12/treatment group) were maintained with their dams until postnatal day 23 (time of weaning), after which they were provided the Mirpur-6 diet, exclusively.

Pups were weighed on postnatal days 18 (P18), P21, P32 and P35 (day of euthanasia). Animals in the B. infantis-treated group had significantly greater weights at all time points compared to pups whose mothers had only received the SAM microbiota (P<0.01, 2-way repeated measures ANOVA with Šidák's correction for multiple comparisons; Table 15, FIG. 4G).

TABLE 15
Body weights of pups whose mothers had been colonized with intact
SAM microbiota with or without B. infantis Bg_2D9 and EVC001

| | | | | | | | | | | | | | Group 2: SAM |
|---|---|---|---|---|---|---|---|---|---|---|---|---|---|
| | | | | | | | | | | | | | microbiota + |
| | | | | | | | | | | | | | B. infantis |
| Age | Group A: SAM microbiota | | | | | | | | | | | | Bg_2D9 and EVC001 |
| (Postnatal | m | m | m | m | m | m | m | m | m | m | m | mean ± | m |
| day) | 1 | 2 | 3 | 4 | 5 | 6 | 7 | 8 | 9 | 10 | 11 | SD | 1 |
| 18 | 8.1 | 8.3 | 7.7 | 8.5 | 7.6 | 8 | 7.9 | 7.5 | 8.2 | 7.5 | 8 | 7.9 ± | 8.8 |
| | | | | | | | | | | | | 0.3 | |
| 21 | 8.9 | 9.1 | 8.7 | 9.2 | 8.1 | 8.6 | 8.5 | 8 | 8.8 | 8.3 | 8.6 | 8.6 ± | 9.9 |
| | | | | | | | | | | | | 0.4 | |
| 32 | 16.6 | 15.3 | 14.8 | 15.4 | 13.4 | 14.7 | 13.7 | 13.8 | 15.5 | 13.9 | 14.1 | 14.7 ± | 18.1 |
| | | | | | | | | | | | | 1 | |
| 35 | 17.7 | 17 | 15.9 | 16.4 | 14.4 | 15.4 | 14.2 | 14.5 | 16.9 | 15.3 | 14.9 | 15.7 ± | 19.6 |
| | | | | | | | | | | | | 1.2 | |

| Age | Group 2: SAM microbiota + B. infantis Bg_2D9 and EVC001 | | | | | | | | | | | |
|---|---|---|---|---|---|---|---|---|---|---|---|---|
| (Postnatal | m | m | m | m | m | m | m | m | m | m | m | mean ± |
| day) | 2 | 3 | 4 | 5 | 6 | 7 | 8 | 9 | 10 | 11 | 12 | SD |
| 18 | 9.9 | 8.8 | 9.2 | 8.5 | 9 | 8.6 | 8.8 | 8.7 | 8.4 | 8.7 | 7.8 | 8.8 ± |
| | | | | | | | | | | | | 0.5 |
| 21 | 11 | 10 | 10.3 | 9.3 | 10.4 | 9.7 | 9.7 | 9.4 | 9.8 | 9.9 | 9.1 | 9.9 ± |
| | | | | | | | | | | | | 0.5 |
| 32 | 17.1 | 16.1 | 17.8 | 16.7 | 16.1 | 16.4 | 16.3 | 16.1 | 16.1 | 16 | 15.7 | 16.5 ± |
| | | | | | | | | | | | | 0.8 |
| 35 | 17 | 17.7 | 17.8 | 18.2 | 16.9 | 17.2 | 17.9 | 16.8 | 16.5 | 16.5 | 16.6 | 17.4 ± |
| | | | | | | | | | | | | 0.9 |

Fecal samples were collected from the four dams on P11, P21 and P28 and from their pups on P21, P28 and P35. Sequencing amplicons generated from the 16S rRNA genes present in fecal samples from the four dams collected prior to the B. infantis gavage on P11 revealed that they were colonized almost exclusively (>99% relative abundance) by Enterobacteriaceae; specifically, ASVs belonging to Enterococcus (61±11%; mean±SD) Escherichia/Shigella (24±12%) and Klebsiella (14±7%). In the dams that received B. infantis gavages, Enterobacteriaceae were reduced to 64±9% relative abundance in postpartum day 28 fecal samples, with Bifidobacteria accounting for 35±9% of their communities (Table 16, FIG. 4H).

TABLE 16
Fractional abundance of amplicon sequence variants (ASVs) in fecal samples of dams/pups colonized with intact SAM microbiota with or without B. infantis Bg_2D9 and EVC001

| | | | | ASV2_{—} | | | | | | | | | |
|---|---|---|---|---|---|---|---|---|---|---|---|---|---|
| Postnatal | | | ASV1_{—} | Escherichia/ | ASV3_{—} | ASV4_{—} | ASV5_{—} | ASV6_{—} | ASV7_{—} | ASV8_{—} | ASV9_{—} | ASV10_{—} | ASV11_{—} |
| day | Isolator | Dam/Pup | Klebsiella | Shigella | Enterococcus | Bifidobacterium | Bifidobacterium | Pediococcus | Staphylococcus | Weissella | Fructobacillus | Bacillus | Fructobacillus |
| P11 | A - SAM | Dam | 0.063 | 0.399 | 0.526 | 0.000 | 0.000 | 0.006 | 0.004 | 0.002 | 0.000 | 0.000 | 0.000 |
| | microbiota | | | | | | | | | | | | |
| P11 | A - SAM | Dam | 0.088 | 0.185 | 0.719 | 0.000 | 0.000 | 0.004 | 0.002 | 0.002 | 0.000 | 0.000 | 0.000 |
| | microbiota | | | | | | | | | | | | |
| P21 | A - SAM | Dam | 0.218 | 0.436 | 0.331 | 0.000 | 0.000 | 0.006 | 0.005 | 0.003 | 0.001 | 0.000 | 0.000 |
| | microbiota | | | | | | | | | | | | |
| P21 | A - SAM | Dam | 0.416 | 0.138 | 0.426 | 0.000 | 0.000 | 0.010 | 0.005 | 0.005 | 0.001 | 0.000 | 0.000 |
| | microbiota | | | | | | | | | | | | |
| P28 | A - SAM | Dam | 0.214 | 0.537 | 0.242 | 0.000 | 0.000 | 0.003 | 0.002 | 0.002 | 0.000 | 0.000 | 0.000 |
| | microbiota | | | | | | | | | | | | |
| P28 | A - SAM | Dam | 0.128 | 0.627 | 0.239 | 0.000 | 0.000 | 0.002 | 0.001 | 0.002 | 0.000 | 0.000 | 0.000 |
| | microbiota | | | | | | | | | | | | |
| P11 | B - SAM | Dam | 0.222 | 0.267 | 0.504 | 0.000 | 0.000 | 0.003 | 0.003 | 0.002 | 0.000 | 0.000 | 0.000 |
| | microbiota + | | | | | | | | | | | | |
| | B. infantis | | | | | | | | | | | | |
| P11 | B - SAM | Dam | 0.173 | 0.109 | 0.709 | 0.000 | 0.000 | 0.004 | 0.003 | 0.002 | 0.000 | 0.000 | 0.000 |
| | microbiota + | | | | | | | | | | | | |
| | B. infantis | | | | | | | | | | | | |
| P21 | B - SAM | Dam | 0.022 | 0.178 | 0.094 | 0.469 | 0.222 | 0.006 | 0.005 | 0.004 | 0.001 | 0.000 | 0.000 |
| | microbiota + | | | | | | | | | | | | |
| | B. infantis | | | | | | | | | | | | |
| P21 | B - SAM | Dam | 0.029 | 0.164 | 0.066 | 0.489 | 0.241 | 0.005 | 0.004 | 0.003 | 0.000 | 0.000 | 0.000 |
| | microbiota + | | | | | | | | | | | | |
| | B. infantis | | | | | | | | | | | | |
| P28 | B - SAM | Dam | 0.044 | 0.571 | 0.091 | 0.152 | 0.133 | 0.005 | 0.003 | 0.003 | 0.000 | 0.000 | 0.000 |
| | microbiota + | | | | | | | | | | | | |
| | B. infantis | | | | | | | | | | | | |
| P28 | B - SAM | Dam | 0.069 | 0.380 | 0.121 | 0.226 | 0.190 | 0.006 | 0.003 | 0.003 | 0.001 | 0.001 | 0.000 |
| | microbiota + | | | | | | | | | | | | |
| | B. infantis | | | | | | | | | | | | |
| P21 | A - SAM | Pup | 0.700 | 0.129 | 0.147 | 0.000 | 0.000 | 0.009 | 0.009 | 0.005 | 0.002 | 0.000 | 0.000 |
| | microbiota | | | | | | | | | | | | |
| P21 | A - SAM | Pup | 0.527 | 0.145 | 0.306 | 0.000 | 0.000 | 0.007 | 0.008 | 0.005 | 0.002 | 0.000 | 0.000 |
| | microbiota | | | | | | | | | | | | |
| P21 | A - SAM | Pup | 0.784 | 0.088 | 0.103 | 0.000 | 0.000 | 0.009 | 0.008 | 0.007 | 0.001 | 0.000 | 0.000 |
| | microbiota | | | | | | | | | | | | |
| P21 | A - SAM | Pup | 0.564 | 0.166 | 0.252 | 0.000 | 0.000 | 0.008 | 0.005 | 0.005 | 0.000 | 0.000 | 0.000 |
| | microbiota | | | | | | | | | | | | |
| P21 | A - SAM | Pup | 0.589 | 0.118 | 0.270 | 0.000 | 0.000 | 0.009 | 0.008 | 0.005 | 0.002 | 0.000 | 0.000 |
| | microbiota | | | | | | | | | | | | |
| P21 | A - SAM | Pup | 0.675 | 0.245 | 0.073 | 0.000 | 0.000 | 0.003 | 0.002 | 0.002 | 0.000 | 0.000 | 0.000 |
| | microbiota | | | | | | | | | | | | |
| P21 | A - SAM | Pup | 0.631 | 0.144 | 0.204 | 0.000 | 0.000 | 0.008 | 0.007 | 0.005 | 0.002 | 0.000 | 0.000 |
| | microbiota | | | | | | | | | | | | |
| P21 | A - SAM | Pup | 0.614 | 0.132 | 0.228 | 0.000 | 0.000 | 0.009 | 0.009 | 0.006 | 0.002 | 0.000 | 0.000 |
| | microbiota | | | | | | | | | | | | |
| P21 | A - SAM | Pup | 0.792 | 0.130 | 0.069 | 0.000 | 0.000 | 0.004 | 0.004 | 0.002 | 0.000 | 0.000 | 0.000 |
| | microbiota | | | | | | | | | | | | |
| P21 | A - SAM | Pup | 0.601 | 0.157 | 0.218 | 0.000 | 0.000 | 0.008 | 0.008 | 0.005 | 0.001 | 0.000 | 0.000 |
| | microbiota | | | | | | | | | | | | |
| P21 | A - SAM | Pup | 0.575 | 0.137 | 0.245 | 0.000 | 0.000 | 0.014 | 0.017 | 0.010 | 0.002 | 0.001 | 0.000 |
| | microbiota | | | | | | | | | | | | |
| P28 | A - SAM | Pup | 0.291 | 0.495 | 0.197 | 0.000 | 0.000 | 0.007 | 0.005 | 0.004 | 0.001 | 0.000 | 0.000 |
| | microbiota | | | | | | | | | | | | |
| P28 | A - SAM | Pup | 0.276 | 0.631 | 0.087 | 0.000 | 0.000 | 0.002 | 0.002 | 0.002 | 0.000 | 0.000 | 0.001 |
| | microbiota | | | | | | | | | | | | |
| P28 | A - SAM | Pup | 0.300 | 0.465 | 0.224 | 0.000 | 0.000 | 0.004 | 0.004 | 0.002 | 0.000 | 0.000 | 0.000 |
| | microbiota | | | | | | | | | | | | |
| P28 | A - SAM | Pup | 0.508 | 0.352 | 0.132 | 0.000 | 0.000 | 0.004 | 0.003 | 0.002 | 0.000 | 0.000 | 0.000 |
| | microbiota | | | | | | | | | | | | |
| P28 | A - SAM | Pup | 0.388 | 0.266 | 0.322 | 0.000 | 0.000 | 0.009 | 0.011 | 0.004 | 0.000 | 0.000 | 0.000 |
| | microbiota | | | | | | | | | | | | |
| P28 | A - SAM | Pup | 0.163 | 0.407 | 0.413 | 0.000 | 0.000 | 0.007 | 0.005 | 0.004 | 0.001 | 0.000 | 0.000 |
| | microbiota | | | | | | | | | | | | |
| P28 | A - SAM | Pup | 0.369 | 0.242 | 0.367 | 0.000 | 0.000 | 0.008 | 0.008 | 0.005 | 0.001 | 0.000 | 0.000 |
| | microbiota | | | | | | | | | | | | |
| P28 | A - SAM | Pup | 0.259 | 0.587 | 0.147 | 0.000 | 0.000 | 0.003 | 0.003 | 0.001 | 0.000 | 0.000 | 0.000 |
| | microbiota | | | | | | | | | | | | |
| P28 | A - SAM | Pup | 0.269 | 0.427 | 0.287 | 0.000 | 0.000 | 0.007 | 0.005 | 0.004 | 0.001 | 0.000 | 0.000 |
| | microbiota | | | | | | | | | | | | |
| P28 | A - SAM | Pup | 0.551 | 0.382 | 0.061 | 0.000 | 0.000 | 0.003 | 0.001 | 0.002 | 0.000 | 0.000 | 0.000 |
| | microbiota | | | | | | | | | | | | |
| P28 | A - SAM | Pup | 0.200 | 0.326 | 0.456 | 0.000 | 0.000 | 0.007 | 0.006 | 0.004 | 0.001 | 0.000 | 0.000 |
| | microbiota | | | | | | | | | | | | |
| P21 | B - SAM | Pup | 0.013 | 0.094 | 0.126 | 0.327 | 0.419 | 0.008 | 0.006 | 0.005 | 0.001 | 0.000 | 0.000 |
| | microbiota + | | | | | | | | | | | | |
| | B. infantis | | | | | | | | | | | | |
| P21 | B - SAM | Pup | 0.012 | 0.077 | 0.084 | 0.459 | 0.346 | 0.008 | 0.009 | 0.005 | 0.001 | 0.000 | 0.000 |
| | microbiota + | | | | | | | | | | | | |
| | B. infantis | | | | | | | | | | | | |
| P21 | B - SAM | Pup | 0.033 | 0.156 | 0.086 | 0.318 | 0.387 | 0.008 | 0.006 | 0.006 | 0.001 | 0.000 | 0.000 |
| | microbiota + | | | | | | | | | | | | |
| | B. infantis | | | | | | | | | | | | |
| P21 | B - SAM | Pup | 0.006 | 0.051 | 0.056 | 0.375 | 0.499 | 0.006 | 0.004 | 0.002 | 0.000 | 0.000 | 0.000 |
| | microbiota + | | | | | | | | | | | | |
| | B. infantis | | | | | | | | | | | | |
| P21 | B - SAM | Pup | 0.012 | 0.068 | 0.077 | 0.311 | 0.518 | 0.006 | 0.005 | 0.003 | 0.001 | 0.000 | 0.000 |
| | microbiota + | | | | | | | | | | | | |
| | B. infantis | | | | | | | | | | | | |
| P21 | B - SAM | Pup | 0.018 | 0.149 | 0.067 | 0.296 | 0.459 | 0.005 | 0.003 | 0.003 | 0.001 | 0.000 | 0.000 |
| | microbiota + | | | | | | | | | | | | |
| | B. infantis | | | | | | | | | | | | |
| P21 | B - SAM | Pup | 0.020 | 0.085 | 0.079 | 0.379 | 0.417 | 0.007 | 0.006 | 0.005 | 0.001 | 0.000 | 0.000 |
| | microbiota + | | | | | | | | | | | | |
| | B. infantis | | | | | | | | | | | | |
| P21 | B - SAM | Pup | 0.015 | 0.104 | 0.124 | 0.619 | 0.124 | 0.006 | 0.004 | 0.003 | 0.000 | 0.000 | 0.000 |
| | microbiota + | | | | | | | | | | | | |
| | B. infantis | | | | | | | | | | | | |
| P21 | B - SAM | Pup | 0.012 | 0.112 | 0.091 | 0.446 | 0.325 | 0.005 | 0.005 | 0.003 | 0.001 | 0.000 | 0.000 |
| | microbiota + | | | | | | | | | | | | |
| | B. infantis | | | | | | | | | | | | |
| P21 | B - SAM | Pup | 0.011 | 0.067 | 0.062 | 0.349 | 0.495 | 0.006 | 0.006 | 0.003 | 0.001 | 0.000 | 0.000 |
| | microbiota + | | | | | | | | | | | | |
| | B. infantis | | | | | | | | | | | | |
| P21 | B - SAM | Pup | 0.011 | 0.032 | 0.060 | 0.375 | 0.500 | 0.007 | 0.008 | 0.005 | 0.002 | 0.000 | 0.000 |
| | microbiota + | | | | | | | | | | | | |
| | B. infantis | | | | | | | | | | | | |
| P28 | B - SAM | Pup | 0.014 | 0.107 | 0.077 | 0.374 | 0.419 | 0.004 | 0.003 | 0.002 | 0.000 | 0.000 | 0.000 |
| | microbiota + | | | | | | | | | | | | |
| | B. infantis | | | | | | | | | | | | |
| P28 | B - SAM | Pup | 0.009 | 0.049 | 0.089 | 0.393 | 0.449 | 0.005 | 0.003 | 0.003 | 0.000 | 0.000 | 0.000 |
| | microbiota + | | | | | | | | | | | | |
| | B. infantis | | | | | | | | | | | | |
| P28 | B - SAM | Pup | 0.015 | 0.149 | 0.079 | 0.388 | 0.356 | 0.006 | 0.004 | 0.003 | 0.000 | 0.000 | 0.000 |
| | microbiota + | | | | | | | | | | | | |
| | B. infantis | | | | | | | | | | | | |
| P28 | B - SAM | Pup | 0.007 | 0.033 | 0.065 | 0.479 | 0.400 | 0.005 | 0.006 | 0.005 | 0.000 | 0.000 | 0.000 |
| | microbiota + | | | | | | | | | | | | |
| | B. infantis | | | | | | | | | | | | |
| P28 | B - SAM | Pup | 0.024 | 0.140 | 0.094 | 0.360 | 0.371 | 0.004 | 0.005 | 0.002 | 0.000 | 0.000 | 0.000 |
| | microbiota + | | | | | | | | | | | | |
| | B. infantis | | | | | | | | | | | | |
| P28 | B - SAM | Pup | 0.009 | 0.047 | 0.103 | 0.382 | 0.447 | 0.005 | 0.005 | 0.004 | 0.000 | 0.000 | 0.000 |
| | microbiota + | | | | | | | | | | | | |
| | B. infantis | | | | | | | | | | | | |
| P28 | B - SAM | Pup | 0.015 | 0.142 | 0.103 | 0.383 | 0.345 | 0.005 | 0.004 | 0.003 | 0.001 | 0.000 | 0.000 |
| | microbiota + | | | | | | | | | | | | |
| | B. infantis | | | | | | | | | | | | |
| P28 | B - SAM | Pup | 0.041 | 0.042 | 0.069 | 0.496 | 0.344 | 0.004 | 0.003 | 0.002 | 0.000 | 0.000 | 0.000 |
| | microbiota + | | | | | | | | | | | | |
| | B. infantis | | | | | | | | | | | | |
| P28 | B - SAM | Pup | 0.012 | 0.043 | 0.095 | 0.443 | 0.393 | 0.004 | 0.006 | 0.003 | 0.001 | 0.000 | 0.000 |
| | microbiota + | | | | | | | | | | | | |
| | B. infantis | | | | | | | | | | | | |
| P28 | B - SAM | Pup | 0.009 | 0.052 | 0.095 | 0.413 | 0.420 | 0.004 | 0.004 | 0.004 | 0.000 | 0.000 | 0.000 |
| | microbiota + | | | | | | | | | | | | |
| | B. infantis | | | | | | | | | | | | |
| P28 | B - SAM | Pup | 0.009 | 0.091 | 0.094 | 0.448 | 0.342 | 0.006 | 0.006 | 0.003 | 0.001 | 0.000 | 0.000 |
| | microbiota + | | | | | | | | | | | | |
| | B. infantis | | | | | | | | | | | | |
| P28 | B - SAM | Pup | 0.007 | 0.035 | 0.091 | 0.470 | 0.388 | 0.004 | 0.004 | 0.002 | 0.000 | 0.000 | 0.000 |
| | microbiota + | | | | | | | | | | | | |
| | B. infantis | | | | | | | | | | | | |

No Bifidobacteria were detected in dams (or their pups) that did not receive the B. infantis gavage. Suppression of Enterobacteriaceae was even more pronounced in weaned (P28) pups of the B. infantis recipients [18±5% relative abundance; Enterococcus, Escherichia/Shigella and Klebsiella ASVs combined), compared to 99±1% aggregate relative abundance of these taxa in pups of mothers that received only the SAM microbiota, FIG. 4H]. These data are consistent with the observations in the SYNERGIE trial, though the magnitude of suppression of Enterobacteriaceae achieved was larger in the mouse study where, as noted below, B. infantis achieved approximately 2-orders of magnitude higher levels of absolute abundance than in the probiotic-treated SYNERGIE infants.

Strain-specific qPCR revealed that dams that had received the B. infantis gavage were colonized with both strains at P35, with Bg_2D9 being significantly more abundant than EVC001 (8.17 vs 7.67 log₁₀ genome equivalents/μg DNA; P=0.012, paired t-test). Pups of mothers that had received the Bg_2D9 plus EVC001 strain mixture were colonized with high levels (>8 log₁₀ genome equivalents/μg DNA) of B. infantis at the earliest time point sampled (P21) (FIG. 4I), with the absolute abundance of Bg_2D9 attaining significantly higher levels of EVC001; moreover, the difference persisted until euthanasia (P35) (P<0.01, two-tailed Wilcoxon matched-pairs signed rank test). Thus, this maternal-pup transmission model provided preclinical evidence of the superior competitiveness of the Bg_2D9 strain over the EVC001 in the context of a SAM donor microbiota and the Mirpur-6 diet.

1-12. (canceled)

13. A formulation comprising strain of Bifidobacterium longum subsp. infantis comprising at least one DNA sequence from Bifidobacterium longum subsp. infantis such that the bacteria has enhanced uptake, utilization, or both, of N-glycans, or plant derived polysaccharides, or both.

14. The formulation of claim 13, wherein the at least one DNA sequence is selected from one or more polynucleotide sequences with more than 60% sequence identity to at least one of SEQ ID NOs. 2-23.

15. The formulation of claim 14, wherein the strain comprises at least two, at least three, at least 4, at least 5, at least 6, at least 7, at least 8, at least 9, at least 10, at least 11, at least 12, at least 13, at least 14, at least 15, at least 16, at least 17, at least 18 at least 19 or at least 20 polynucleotide sequences with more than 60% sequence identity to SEQ ID NOs. 2-23.

16. The formulation of claim 14, wherein the strain comprises one or more polynucleotide sequences with more than 60% sequence identity to each of SEQ ID NOS. 2-23.

17. The formulation of claim 13, wherein the strain of Bifidobacterium longum subsp. infantis is present in an amount of more than 102 cfu per gram of the formulation.

18. The formulation claim 13, wherein the Bifidobacterium longum subsp. infantis strain is in the form of viable cells.

19. The formulation claim 13, wherein the Bifidobacterium longum subsp. infantis strain is in the form of a mixture of viable and non-viable cells.

20-32. (canceled)

33. A combination, the combination comprising an engineered strain of Bifidobacterium longum subspecies infantis comprising one or more polynucleotide sequences comprising any of SEQ ID NOs. 2-23 and a food formulation comprising at least one carbohydrate that can be metabolized by members of the gut microbiota.

34. The combination of claim 33, wherein the food formulation comprises chickpea flour, peanut flour, soy flour, green banana, and a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months; wherein the composition contains no milk, powdered milk or milk product; wherein the composition has about 300 to about 560 kcal per 100 g of the composition, a protein energy ratio (PER) of about 8% to about 20%, and a fat energy ratio (FER) of about 30% to about 60%, and wherein the amount of protein is at least 11 g per 100 g of the composition and the amount of fat is not more than 36 g per 100 g of the composition; and wherein the chickpea flour, the peanut flour, the soy flour, and the green banana, in total, provide at least 9 g of protein per 100 g of the composition.

35-37. (canceled)

38. A method of treatment, the method comprising administering to a subject in need thereof, a therapeutically effective quantity of a formulation of claim 13.

39-65. (canceled)

66. A method for enhancing uptake, or utilization or both of milk N-glycans, or plant-based polysaccharides, or both, the method comprising administering to a subject in need thereof a therapeutically effective quantity of a formulation of claim 6.

67. The method for enhancing uptake, or utilization or both of milk N-glycans, or plant-based polysaccharides, or both, of claim 66, wherein the subject is exhibiting symptoms of or diagnosed with Severe Acute Malnutrition (SAM).

68-93. (canceled)

94. A method for modifying the gut microbiota of a subject in need thereof, the method comprising administering to a subject a therapeutically effective quantity of a formulation of claim 6.

95. The method for modifying the gut microbiota of claim 94, wherein the subject is exhibiting symptoms of or diagnosed with Severe Acute Malnutrition (SAM).

96-124. (canceled)
